(19) United States
(12) Patent Application Publication
(10) Pub. No.: US 2025/0325508 A1
(43) Pub. Date: Oct. 23, 2025

(54) MODULATORS OF PROTEASOME DYNAMICS AND/OR FUNCTION, COMPOSITIONS, METHODS, AND THERAPEUTIC USES THEREOF

(71) Applicant: TECHNION RESEARCH & DEVELOPMENT FOUNDATION LIMITED, Haifa (IL)

(72) Inventors: Aaron CIECHANOVER, Haifa (IL); Ido LIVNEH, Haifa (IL); Victoria COHEN-KAPLAN, Haifa (IL); Bertrand Francois FABRE, Haifa (IL)

(21) Appl. No.: 19/211,080

(22) Filed: May 16, 2025

Related U.S. Application Data
(63) Continuation of application No. PCT/IL2023/051191, filed on Nov. 17, 2023.
(60) Provisional application No. 63/384,297, filed on Nov. 18, 2022.
(60) Provisional application No. 63/580,427, filed on Sep. 4, 2023.

(51) Int. Cl. A61K 31/198; A61K 31/405; A61P 35/00; A61P 35/04

(52) U.S. Cl. CPC A61K 31/198; A61K 31/405; A61P 35/00; A61P 35/04

(57) ABSTRACT

The present disclosure provides modulators of proteasome dynamics and/or function in a mammalian cell, compositions and uses thereof. The disclosed modulating compounds are characterized by affecting at least one of: mammalian target of rapamycin (mTOR) activation and/or lysosomal association, proteasome cellular localization, the activity and/or level/s and/or the post translational modification/s (PTM/s), and/or subcellular localization of at least one signaling molecule participating directly or indirectly in at least one pathway mediating said proteasome dynamics/function.

### CROSS REFERENCE TO RELATED APPLICATIONS

This application is a Bypass Continuation of PCT Patent Application No. PCT/IL2023/051191 having International filing date of Nov. 17, 2023, which claims the benefit of priority of U.S. Provisional Patent Application Nos. 63/384,297, filed Nov. 18, 2022, and 63/580,427, filed Sep. 4, 2023, the contents of which are all incorporated herein by reference in their entirety.

### REFERENCE TO AN ELECTRONIC SEQUENCE LISTING

The contents of the electronic sequence listing (2979665-JDB.xml; Size: 105,892 bytes; and Date of Creation: Nov. 16, 2023) is herein incorporated by reference in its entirety.

### FIELD OF THE INVENTION

The invention relates to the field of personalized medicine. More specifically, the invention provides compositions and methods modulating mTOR, Sestrin3, p38 and/or p62 and NBR1, and/or NUP93-mediated proteasome dynamics, and uses thereof for treating, prognosing and monitoring conditions affected by proteasome activity and/or cellular localization, specifically, neoplastic disorders.

### BACKGROUND ART

References considered to be relevant as background to the presently disclosed subject matter are listed below:
[1] D. Finley et al., Trends Biochem. Sci. 41, 77-93 (2016).
[2] M. Bochtler et al., Annu. rev. biophys biomol. 28, 295-317 (1999).
[3] D. Voges et al., Annu. Rev. Biochem. 68, 1015-1068 (1999).
[4] S. Yasuda et al., Nature 578, 296-300 (2020).
[5] R. S. Marshall et al., Cell Rep. 16, 1-16 (2016).
[6] J. Li et al., Curr. Genet. 66, 683-687 (2020).
[7] D. Laporte et al., J. Cell Biol. 181, 737-745 (2008).
[8] R. S. Marshall et al., Elife 7, e34532 (2018).
[9] R. A. Saxton et al., Cell 168, 960-976 (2017).
[10] S. Wullschleger et al., Cell 124, 471-484 (2006).
[11] T. Takahara et al., J. Biomed. Sci. 27, 1-16 (2020).
[12] A. Ho et al., Trends Biochem. Sci. 41, 621-632 (2016).
[13] A. Parmigiani et al., Cell Rep. 9, 1281-1291 (2014).
[14] L. Chantranupong et al., Cell Rep. 9, 1-8 (2014).
[15] M. Wang et al., Int. J. Clin. Exp. Pathol. 9, 8075-8082 (2016).
[16] J. F. Linares et al., Cell Rep. 12, 1339-1352 (2015).
[17] WO2022/009212.

Acknowledgement of the above references herein is not to be inferred as meaning that these are in any way relevant to the patentability of the presently disclosed subject matter.

### BACKGROUND OF THE INVENTION

The proteasome is largely responsible for selective removal of ubiquitinated proteins [1-3]. While several aspects of proteasome regulation (e.g., assembly, composition and post-translational modifications) have been largely unraveled, the question of its compartmentalization and adaptive concentration in response to stress in mammalian cells is just starting to emerge [4]. In yeast, glucose starvation was shown to induce proteasome removal by autophagy [5, 6] or sequestration in protective granules [7, 8]. Yet, in none of these cases, proteasome dynamics was shown to involve its proteolytic function as a coping mechanism to mitigate stress or to act as a player in determining cell fate.

A key regulator of various stress conditions, including amino acid shortage, is the target of rapamycin (TOR), and its mammalian homolog—the mechanistic TOR (mTOR). While mTOR is activated and localized to the lysosomal membrane in the presence of nutrients, their absence results in its dissociation from the lysosome, inhibition of its kinase activity, and among other downstream effects-upregulation of autophagy which in turn supplies the cell with recycled building blocks [9, 10]. While a large body of evidence regarding mTOR role in proteolysis regulation is concerned with autophagy, it was shown that during short amino acid deprivation, the proteasome is the key proteolytic machinery responsible for amino acid recycling [Vabulas, et al. 2005. Science 310, 1960-1963]. Characterization of the direct sensors through which the level of different amino acids is relayed to mTOR is still in its early stage, and only a handful of such proteins have been identified. Unlike the regulation of autophagy and translation, no specific amino acids were linked to the activity of the ubiquitin proteasome system via the mTOR pathway [11]. In fact, only a handful of amino acids were specifically shown to activate it. With regard to the known sensors and agonistic amino acids, there is some degree of redundancy: different amino acids can activate mTOR through the same mediator, and a single amino acid can activate mTOR via more than one mediator. Leu, for example, is sensed by both Sestrin2 (SESN2) and Leu-tRNA [11].

SESN2 is a member of a family including also SESN1 and SESN3. While the three share some characteristics, it was shown that they do not overlap in all of their functions. In some cases, one Sestrin plays a unique role, while in others, two of them seem to have some degree of redundancy [12]. For example, knockout of SESN2 was shown to partially rescue mTOR activity under starvation in the context of its role as a regulator of translation [13]. Silencing of both SESN2 and SESN1 enhanced this effect, while additional silencing of SESN3 had little additive effect [14]. Importantly, simultaneous silencing of SESN1 and SESN3 had little effect on translation [14], underscoring the critical role of SESN2 in this context, the small contribution made by SESN1, and the negligible role of SESN3. SESN3 was also shown to interact with the GTPase-activating protein (GAP) towards Rags 2 (GATOR2) complex to a significantly lesser extent, compared with SESN2 and SESN1 [14]. The Sestrins were shown to differ in their involvement in pathophysiological states also in human diseases, for example, in heart failure [15], further demonstrating their non-overlapping roles in health and disease.

While the Sestrins were shown to inhibit mTOR activity, including in the context of amino acid sensing, the p38 MAPK was shown to act as an activator of mTORC1 in response to amino acid supplementation [16]. p38 is phosphorylated and activated by MEK3 in the presence of amino acids, which results in activation of mTORC1 and its localization to the lysosomal membrane [16]. The present inventors recently identified triad of mTOR-agonistic amino acids-Tyr, Trp, and Phe (YWF) [WO2022/009212] [17]. These aromatic amino acid residues YWF effectively inhibited proteasome recruitment, and also induce active import, both in cultured cells and tumors. More importantly, systemic as well as local administration of the YWF triad significantly and synergistically inhibited tumor growth. There is therefore need for powerful selective modulators of proteasome dynamics for use in therapy. These unmet needs are addressed by the present disclosure.

### SUMMARY OF THE INVENTION

A first aspect of the present disclosure relates to a method for treating, preventing, inhibiting, reducing, eliminating, protecting or delaying the onset of at least one condition or at least one pathologic disorder in a subject in need thereof. More specifically, the method comprising the step of administering to the subject a therapeutic effective amount of at least one compound that modulates proteasome dynamics and/or function in a mammalian cell. In some embodiments, the compound is characterized by affecting at least one of: mammalian target of rapamycin (mTOR) activation and/or lysosomal association, the activity and/or level/s and/or the post translational modification/s (PTM/s), and/or subcellular localization of at least one signaling molecule participating directly or indirectly in at least one pathway mediating said protcasome dynamics/function. Optionally, the modulating compound may further modulate protcasome cellular localization.

A further aspect of the present disclosure relates to a therapeutic effective amount of at least one compound that modulates the proteasome dynamics and/or function in a mammalian cell, for use in a method for treating, preventing, inhibiting, reducing, eliminating, protecting or delaying the onset of at least one condition or at least one pathologic disorder in a subject in need thereof. In some embodiments, a compound applicable in the disclosed uses, is a compound characterized by affecting at least one of: mTOR activation and/or lysosomal association, the activity and/or level/s and/or PTM/s, and/or subcellular localization of at least one signaling molecule participating directly or indirectly in at least one pathway mediating said proteasome dynamics and/or function. Optionally, the modulating compound may further modulate proteasome cellular localization. A further aspect of the present disclosure relates to a method for determining a personalized treatment regimen for a subject suffering from a pathologic disorder, by assessing responsiveness of the subject to a treatment regimen comprising at least one therapeutic compound, determining dosage of the compound, and/or monitoring disease progression of the subject. More specifically, the personalized methods disclosed herein comprise the following steps. In one step (a), the method involves determining in at least one sample of the subject, at least one of: (i) mTOR activation and/or lysosomal association; (ii) activation of p38, specifically, p38 delta; (iii) phosphorylation of Tyr705 of STAT3; and (iv) Sestrin3 levels, and/or activation and/or the interaction of Sestrin3 with at least one regulatory complex; and optionally, (v) the proteasome subcellular localization in at least one cell of the at least one sample, or in any fraction thereof. In step (b), the disclosed method provides classifying the subject. In some embodiments, the subject is classified as (I), a responder subject to the treatment regimen, if at least one of: (i) mTOR is activated and/or localized to the lysosomal membrane; (ii) p38 is activated, specifically, p38 delta in the sample is phosphorylated in at least of T180/Y182; (iii) phosphorylation of Tyr705 of STAT3 is inhibited or reduced; and (iv) Sestrin levels, and/or activation and/or the interaction of Sestrin3 with at least one regulatory complex are reduced; and optionally, (v) the ratio of nuclear to cytosolic proteasome subcellular localization is greater than 1. Alternatively, the subject may be classified as (II), a non-responder subject or a poor responder to said treatment regimen if at least one of: (i) mTOR is inactivated and/or dissociated from the lysosomal membrane; (ii) p38, specifically, p38 delta, is inactivated (dephosphorylation of at least of T180/Y182); (iii) Tyr705 of STAT3 is phosphorylated; and (iv) Sestrin3 levels, and/or activation and/or the interaction of Sestrin3 with at least one regulatory complex, are increased or maintained; and optionally, (v) the ratio of nuclear to cytosolic proteasome subcellular localization is smaller than, or equal to 1. In step (c) of the disclosed methods, the treatment regimen is maintained for a subject classified as a responder. Alternatively, for subject exhibiting a mild or poor response, the dose of the therapeutic compound in the treatment regimen is increased. In some embodiments, for a subject classified as a non-responder or poor responder, the treatment regimen may be ceased, thereby determining a treatment regimen to the subject.

A further aspect of the present disclosure relates to a screening method for identifying at least one modulator of protcasome dynamics and/or function. More specifically, the method comprising the following steps. One step (a), involves determining in at least one cell contacted with a candidate compound, or in any fraction of the cell, or in any sample thereof, at least one of the following parameters. In some embodiments (i), mTOR activation, and/or lysosomal association in the presence and/or absence of the candidate compound is examined. In yet some additional or alternative embodiments, (ii) activation of p38 in the presence and/or absence of the candidate compound is examined. Still further in some alternative or additional embodiments (iii), phosphorylation of Tyr705 of STAT3 in the presence and/or absence of the candidate compound is examined. In some further additional or alternative embodiments (iv), the cell viability, or in other words, the cytotoxicity, in the presence and/or absence of the candidate compound is examined. In some embodiments, cytotoxicity of the candidate compound may be evaluated by determining apoptosis in the cells. Still further, in some alternative or additional embodiments (v), the level of at least one cytosolic and/or nuclear substrate of the proteasome in the presence and/or absence of the candidate compound is examined. In some alternative or additional embodiments (vi), Sestrin3 levels, and/or activation and/or the interaction of Sestrin3 with at least one regulatory complex, in the presence and/or absence of the candidate compound are determined. Still further, in some optional or additional embodiments (vii), proteasome subcellular localization in the presence and/or absence of the candidate compound is examined. In another step (b), the method involves determining that the candidate compound is:

Either (I), an inhibitor of proteasome translocation/recruitment and/or of proteasome assembly, if at least one of: (i) mTOR is activated and/or is localized to the lysosomal membrane; (ii) p38 is activated (e.g., phosphorylated in at least of T180/Y182); (iii) phosphorylation of Tyr705 of STAT3 is inhibited or reduced; (iv) the cell display reduced viability; (v) the level of at least one cytosolic substrate of the proteasome is maintained; (vi) Sestrin3 levels, and/or activation and/or the interaction of Sestrin3 with at least one regulatory complex, are reduced; and optionally, (vii) the ratio of nuclear to cytosolic proteasome subcellular localization is greater than 1. Alternatively (II), the candidate compound is determine as an enhancer of proteasome translocation/recruitment and/or of proteasome assembly, if at least one of: (i) mTOR is inactivated and/or dissociated from the lysosomal membrane; (ii) p38 is inactivated (e.g., de-phosphorylation of at least of T180/Y182); (iii) Tyr705 of STAT3 is phosphorylated; (iv) the cell is viable; (v) the level of at least one cytosolic substrate of the proteasome is reduced; (vi) Sestrin3 levels, and/or activation and/or the interaction of Sestrin3 with at least one regulatory complex, are maintained or increased and (vi) the ratio of nuclear to cytosolic proteasome subcellular localization is smaller than or equal to 1.

A further aspect of the present disclosure relates to a method for modulating proteolysis in at least one cell. More specifically, the method comprising the step of contacting the cell with an effective amount of at least one compound that modulates proteasome dynamics and/or function or subjecting the cell to conditions that modulate the proteasome dynamics/function. In some embodiments, the compound and/or conditions are characterized by affecting at least one of: mTOR activation and/or lysosomal association, the activity and/or level/s, and/or PTMs and/or localization of at least one signaling molecule participating directly or indirectly in at least one signaling pathway mediating the proteasome dynamics and/or function. Optionally, the modulating compound may further modulate proteasome cellular localization.

A further aspect of the present disclosure relates to a method for treating, preventing, inhibiting, reducing, eliminating, protecting or delaying the onset of at least one condition or at least one pathologic disorder in a subject in need thereof. In some embodiments, the method comprises the steps of: In step (a), determining in at least one sample of the subject, at least one of: (i) mTOR activation and/or lysosomal association; (ii) activation of p38, specifically, p38 delta; (iii) phosphorylation of Tyr705 of STAT3; and/or (iv) Sestrin3 levels, and/or activity and/or the interaction of Sestrin3 with at least one regulatory complex; and optionally, (v) the proteasome subcellular localization in at least one cell of the at least one sample, or in any fraction thereof.

The next step (b), involves classifying the subject as: (I) a responder subject to the treatment regimen, if at least one of: (i) mTOR is activated and/or localized to the lysosomal membrane; (ii) p38 is activated; (iii) phosphorylation of Tyr705 of STAT3 is inhibited or reduced; and/or (iv) the Sestrin3 levels, and/or activity and/or the interaction of Sestrin3 with at least one regulatory complex are reduced; and optionally, (v) the ratio of nuclear to cytosolic proteasome subcellular localization is greater than 1; or (II) a non-responder subject or a poor responder to said treatment regimen if at least one of: (i) mTOR is inactivated and/or dissociated from the lysosomal membrane; (ii) p38 is inactivated; (iii) Tyr705 of STAT3 is phosphorylated; and/or (iv) Sestrin3 levels, and/or activation and/or the interaction of Sestrin3 with at least one regulatory complex are maintained or increased; and optionally, (v) the ratio of nuclear to cytosolic proteasome subcellular localization is smaller than, or equal to 1. The next step (c), involves administering to a subject classified as a responder a treatment regimen comprising a therapeutic effective amount of at least one compound that modulates proteasome dynamics and/or function in a mammalian cell, increasing the dose of the compound in subject exhibiting a mild or poor response, or ceasing the treatment regimen for a subject classified as a non-responder or poor responder; thereby treating the subject.

A further aspect relates to a therapeutic compound that modulates proteasome dynamics and/or function in a mammalian cell, or any composition thereof. More specifically, the compound is characterized by affecting at least one of: mammalian target of rapamycin (mTOR) activation and/or lysosomal association, the activity and/or level/s and/or the post translational modification/s (PTM/s), and/or subcellular localization of at least one signaling molecule participating directly or indirectly in at least one pathway mediating said proteasome dynamics/function. Optionally, the modulating compound may further modulate proteasome cellular localization.

A further aspect of the present disclosure relates to a combination or a combined composition comprising any combination of at least two of the proteasome dynamics and/or function modulators disclosed by the present disclosure. These and other aspects of the invention will become apparent by the hand of the following drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

In order to better understand the subject matter that is disclosed herein and to exemplify how it may be carried out in practice, embodiments will now be described, by way of non-limiting example only, with reference to the accompanying drawings, in which:

FIG. 1A-1N: Stress-induced translocation of the 26S proteasome from the nucleus to the cytosol is active and specific

FIG. 1A-B. HeLa cells were incubated for 8 hours in either complete medium (Cont.), starvation medium in the absence (St.), or presence of Leptomycin B (St.+LMB). The α6 (FIG. 1A) and β4 (FIG. 1B) proteasome subunits and the nucleus were visualized using fluorescent confocal microscopy. The α6 proteasome subunit was stained via indirect immunofluorescence. The β4 proteasome subunit was expressed with a GFP fused to its C-termini.

FIG. 1C (i)-(iv). Proteasome subunits from nuclei (Nuc, (i) and (iii)) and cytosol (Cyto, (ii) and (iv)) derived from HeLa (1C(i) and 1C(ii)) and RT4 (1C(iii) and 1C(iv)) cells treated as indicated, were blotted with the appropriate antibodies.

FIG. 1D. Immunofluorescence of fruit fly gut following feeding the flies for 6 hours with either complete medium (Cont.) or a solution of 5% sucrose (St.). The α6 proteasome subunit and the nucleus were visualized using fluorescent confocal microscopy. Scale bar −20 μm.

FIG. 1E-1F. HeLa cells were treated with either LMB (Cont.+LMB), or Ivermectin (Cont.+Iver.). The α6 (FIG. 1E) and β4 (FIG. 1F) proteasome subunits and the nucleus were visualized using fluorescent confocal microscopy. α6 was stained via indirect immunofluorescence; β4 was expressed with a GFP fused to its C-terminus. Scale bar −20 μm.

FIG. 1G. HeLa cells were starved for the indicated times, and the α6 and Rpn2 proteasome subunits were visualized following blotting with the appropriate antibodies.

FIG. 1H (i)-(ii). HeLa cells were starved for the indicated times, and the α6 (1H(i)) and Rpn2 (1H(ii)) proteasome subunits were stained via indirect immunofluorescence. Scale bars −20 μm.

FIG. 1I (i)-(ii). HeLa cells were starved for 8 h, then replenished with complete medium for the indicated times, and the nuclear (Nuc) (1I(i)) and cytosol (Cyto) (1I(ii)) fractions were blotted for the indicated proteasome subunits and loading controls.

FIG. 1J. HeLa cells were starved and then replenished with a complete medium in the absence or presence of CHX. The α6 was stained via indirect immunofluorescence and visualized using fluorescent confocal microscopy. Scale bar −20 μm.

FIG. 1K. HeLa cells were transfected with the B4 proteasome subunit fused to the photoconvertible fluorescent protein Dendra2 at its C-terminus. The green Dendra2 was converted to red using UV light, and the cells were then starved for 8 h, after which they were replenished with a complete medium. The same cells were monitored along the entire experiment. Scale bar −20 μm.

FIG. 1L. HeLa Cells expressing B4 with GFP fused to its C-terminus were incubated for 24 h at either 21% (Cont.) or 1% O₂ (Hypoxia).

FIG. 1M. Cells as in IL were incubated for 8 h at either 37° C. (Cont.), or 43° C. (Heat-Shock).

FIG. 1N. HeLa Cells were treated with either 2-deoxyglucose (2-DG), ionomycin (Iono.), or phenformin (Phen.). α6 was stained via indirect immunofluorescence.

FIG. 2A-2E: Stress-induced translocation of the 26S proteasome from the nucleus to the cytosol is active and specific

FIG. 2A. Cells were treated as in FIG. 1A, and multiple replicates were analyzed based on Pearson's co-localization coefficient to quantify proteasome localization in the visualized cells.

FIG. 2B (i)-(iii). U2OS cells were incubated for 8 h in either complete (Cont.) or starvation medium (St.). The α6 and Rpn2 proteasome subunits were stained via indirect immunofluorescence (2B(i)). Western blot of nuclear (Nuc) (2B(ii)) and cytosol (Cyto) (2B(iii)) fractions from the corresponding cells displaying the indicated proteasome subunits and loading controls.

FIG. 2C. HEK293 cells were treated as indicated, and α6 was stained via indirect immunofluorescence. Scale bars −20 μm.

FIG. 2D (i)-(iv). MDA-MB-231 (2D(i)), HAP1 (2D(ii)), MCF10A (2D(iii)), and MEF cells (2D(iv)) were incubated for 8 h in either complete (Cont.) or starvation medium (St.), and the indicated proteasome subunits were stained via indirect immunofluorescence. Scale bars −20 μm.

FIG. 2E. HeLa cells were starved for 8 h (St.) and then replenished with a complete medium for additional 4 h. The Rpn2 and B4 proteasome subunits were expressed with GFP fused to their C-termini. Scale bars −20 μm.

FIG. 3A-3E: Stress-induced proteasome translocation is mediated by mTOR and regulated by Tyr, Trp, and Phe

FIG. 3A (i)-(ii). Hella cells were incubated with the mTOR inhibitor Torin1. The α6 (3A(i)) and B4 (3A(ii)) proteasome subunits and the nucleus were visualized using fluorescent confocal microscopy. α6 was stained via indirect immunofluorescence; β4 was expressed with GFP fused to its C-terminus.

FIG. 3B (i)-(iv). Protcasome subunits from nuclei (Nuc, (i) and (iii)) and cytosol (Cyto, (ii) and (iv)) derived from Hela cells (3B(i) and 3B(ii)) and RT4 cells (3B(iii) and 3B(iv)) treated as indicated, were visualized following blotting with the appropriate antibodies.

FIG. 3C (i)-(ii). Immunofluorescence of HeLa cells following silencing of mTOR using shRNA. The α6 (3C(i)) and β4 (3C(ii)) proteasome subunits and the nucleus were visualized using fluorescent confocal microscopy. α6 was stained via indirect immunofluorescence; β4 was expressed with GFP fused to its C-terminus. Scale bars −20 μm.

FIG. 3D. HeLa cells were incubated with either a complete medium (Cont.), starvation medium (St.), starvation medium supplemented with only Gln, Leu, and Arg (St.+QLR) or Tyr, Trp, and Phe (St.+YWF), or with a medium lacking only Tyr, Trp, and Phe (−YWF). The α6, β4, and α2 proteasome subunits were visualized using fluorescent confocal microscopy. α6 and α2 were stained via indirect immunofluorescence; β4 was expressed with GFP fused to its C-terminus. Scale bars −20 μm.

FIG. 3E(i)-(ii). Low magnification of HeLa cells (3E(i)) and RT4 cells (3E(ii)) treated as indicated. α6 was stained via indirect immunofluorescence. Scale bars −40 μm.

FIG. 4A-4E: mTOR signaling branch regulates stress-induced proteasome recruitment to the cytosol

FIG. 4A. Cells were treated as in FIG. 3A, and multiple replicates were analyzed based on Pearson's co-localization coefficient to quantify proteasome localization in the visualized cells.

FIG. 4B. Hela cells were infected with either a control shRNA (shCont.) or shRNA targeting mTOR (shmTOR). Western blot was used to monitor the silencing at the protein level.

FIG. 4C. HeLa cells were infected with either a control shRNA (shCont.) or shRNAs targeting the uncharged-tRNA sensor GCN2 (shGCN2_1-3). Western blot was used to monitor the silencing at the protein level.

FIG. 4D. HeLa cells were infected with either a control shRNA (shCont.) or shRNAs targeting the protein kinase PIK3CA (shPIK3CA_1-3) or control shRNA (shCont.). Western blot was used to monitor the silencing at the protein level.

FIG. 4E. HeLa cells were infected with either a control shRNA (shCont.) or shRNAs targeting the protein kinase AKT1 (shAKT1_1-2). Western blot was used to monitor the silencing at the protein level.

FIG. 5A-5J: Stress-induced proteasome translocation is mediated by mTOR and regulated by Tyr, Trp, and Phe

FIG. 5A. Hela cells were infected with shRNA targeting GCN2 (shGCN2) and treated as indicated. The β4 proteasome subunit was visualized using fluorescent confocal microscopy. Scale bar −20 μm.

FIG. 5B. HeLa cells were infected with control shRNA (shCont.) or shRNAs targeting the uncharged-tRNA sensor GCN2 (shGCN2_1, shGCN2_2, shGCN2_3). Nuclear fractions (Nuclear fr.) were isolated from the cells following 8 h incubation in a complete (Cont.) or a starvation medium (St.) and were blotted for the α6 proteasome subunit.

FIG. 5C. HeLa cells were infected with shRNAs targeting the protein kinase PIK3CA (shPIK3CA_1, shPIK3CA_2, shPIK3CA_3) or control shRNA (shCont.) and were treated as indicated. The α6 proteasome subunit was stained via indirect immunofluorescence.

FIG. 5D. HeLa cells were infected with shRNAs targeting the protein kinase AKT1 (shAKT1_1, shAKT1_2) and were treated as indicated. The α6 proteasome subunit was stained via indirect immunofluorescence.

FIG. 5E. HeLa cells were incubated for 8 h in a medium lacking amino acids, and the effect of added individual amino acids on the translocation of the proteasome was monitored via indirect immunofluorescence of α6. Single letters denote the one letter code of amino acids. Scale bar-20 μm.

FIG. 5F. Quantitative analysis of nuclear export following the indicated treatments. Cells were treated as indicated, and multiple replicates were analyzed based on Pearson's co-localization coefficient to quantify proteasome localization in the visualized cells.

FIG. 5G. HeLa cells expressing β4 with GFP fused to its C-terminus were treated as indicated. The exportin1 substrates p65 subunit of NF-κB and APC were stained via indirect immunofluorescence. Scale bar −20 μm.

FIG. 5H. HeLa cells infected with GFP fused to a nuclear export signal (NES) were incubated for 8 h under the indicated conditions. The GFP was visualized. Scale bar −20 μm.

FIG. 5I (i)-(ii). Western blot of HeLa cells (5I(i)) and RT4 cells (5I(ii) for the indicated proteasome subunits following the indicated treatments.

FIG. 5J. Immunoprecipitation of the proteasome using an antibody for the α6 proteasome subunit, followed by mass spectrometric analysis for proteasome sub-complexes' subunits under the indicated conditions.

FIG. 6A-6P: mTOR-mediated proteasome dynamics is regulated by SESN3, p38, and STAT3, and is dependent on mTOR localization to the lysosome

FIG. 6A (i)-(ii). (6A(i), 6A(ii) and 6A(iii)) Cells were infected with sgRNA against the indicated proteins, and the proteins were visualized via Western blot using the appropriate antibodies.

FIG. 6B (i)-(ii). HeLa cells were transfected with siRNA against TSC1 and TSC2, and silencing was assessed via Western blot (6B(i)). The cells were treated as indicated, and α6 was stained via indirect immunofluorescence (6B(ii)). Scale bars −20 μm.

FIG. 6C. RT4 cells infected with sgRNA against SESN3 (sgSESN3_1, sgSESN3_2, sgSESN3_3) were treated as indicated, and the phosphorylation of p70-S6K was monitored via Western blot.

FIG. 6D (i)-(ii). RT4 cells infected with sgRNA against SESN3 were transfected with the indicated constructs (6D(i)). The cells were treated as indicated and α6 was stained via indirect immunofluorescence (6D(ii)). Scale bars −20 μm.

FIG. 6E. Western blot analysis of HeLa cell lysates for phosphorylated p38 MAPK following the indicated treatments.

FIG. 6F. RT4 cells were transfected with either an empty vector (V0) or a constitutively active MEK3 (CA-MEK3), and expression was monitored via Western blot.

FIG. 6G. RT4 cells were transfected with siRNA against the p388 isoform, and silencing was assessed via Western blot.

FIG. 6H. RT4 cells infected with sgRNA against SESN3 were analyzed for the level of p38 phosphorylation (P-p38) via Western blot.

FIG. 6I. HeLa cells were treated as indicated, stained for lysosomes using Lysotracker and for mTOR via indirect immunofluorescence. Presented is co-localization of mTOR to the lysosomal membrane.

FIG. 6J. Complementary to FIG. 7D. RT4 cells infected with sgRNA against SESN3 (sgSESN3_2 and sgSESN3_3) were treated as indicated, and mTOR was stained via indirect immunofluorescence, followed by staining for the lysosomal protein LAMP1 using an Alexa-Fluor 647-conjugated antibody. Presented is co-localization of mTOR to the lysosomal membrane. Scale bars −4 μm.

FIG. 6K. HeLa cells were treated as indicated and blotted for total and STAT 3 phosphorylated at Tyr705 (P-STAT3 Y705). Presented is a quantification of the fold-change in phosphorylation relative to control and normalized to total STAT3.

FIG. 6L. RT4 cells were transfected with siRNA against p62 and NBR1, and silencing was assessed via Western blot.

FIG. 6M. HeLa cells were transfected with siRNA against AKIRIN2, and silencing was assessed via Western blot.

FIG. 6N. MDA-MB-231 cells were infected with shRNA targeting the NPC protein NUP93. Silencing was assessed via Western blot.

FIG. 6O. Cells as in FIG. 6N were further infected with GFP-NLS, and GFP localization was monitored using confocal live microscopy.

FIG. 6P. HeLa cells were infected with either control siRNA (siCont) or siRNA against SESN3. The cells were treated as indicated and α6 was stained via indirect immunofluorescence. Scale bars −20 μm.

FIG. 7A-7Q:-mTOR-mediated proteasome dynamics is regulated by SESN3, which binds GATOR2 in a YWF-dependent manner

FIG. 7A. HeLa cells were infected with either control sgRNA (sgV0) or sgRNA against SESN3 or SESN2. The cells were treated as indicated and α6 was stained via indirect immunofluorescence. Scale bars −20 μm.

FIG. 7B. RT4 cells infected with either control sgRNA (sgV0) or sgRNA against SESN3 (sgSESN3_1, sgSESN3_2, sgSESN3_3) were treated as indicated, and α6 was stained via indirect immunofluorescence. Scale bars −20 μm.

FIG. 7C. RT4 cells infected with sgRNA against SESN3 (sgSESN3_1, sgSESN3_2) were then transfected with a plasmid expressing SESN3 and treated as indicated. α6 was stained via indirect immunofluorescence. Scale bars −20 μm.

FIG. 7D. RT4 cells infected with either control sgRNA (sgV0) or sgRNA against SESN3 (sgSESN3_1) were treated as indicated, and mTOR was stained via indirect immunofluorescence, followed by staining for the lysosomal protein LAMP1 using an Alexa-Fluor 647-conjugated antibody. Presented is co-localization of mTOR to the lysosomal membrane. Scale bars −4 μm.

FIG. 7E(i)-(ii). Western blot analysis of immunoprecipitated lysates of Flag-SESN3 expressing Hela cells (7E(i)) or RT4 cells (7e(ii)), that were starved in the presence or absence of YWF. Interaction of SESN3 with members of the GATOR2 complex, Mios and WDR59, is significantly inhibited in the presence of YWF.

FIG. 7F. Western blot analysis of HeLa cell lysates for phosphorylated p38 MAPK following the indicated treatments.

FIG. 7G. RT4 and Hela cells were treated as indicated and α6 was stained via indirect immunofluorescence.

FIG. 7H. RT4 Cells expressing either an empty vector (V0) or the constitutively active form of the protein kinase MEK3 (CA-MEK3) were treated as indicated, and α6 was stained via indirect immunofluorescence.

FIG. 7I. RT4 Cells were transfected with siRNA against the p388 isoform and were treated as indicated. α6 was stained via indirect immunofluorescence.

FIG. 7J. RT4 Cells were treated as indicated and mTOR was stained via indirect immunofluorescence, followed by a staining for the lysosomal protein LAMP1 using an Alexa-Fluor 647-conjugated antibody. Presented is co-localization of mTOR to the lysosomal membrane. Scale bars −4 μm.

FIG. 7K. RT4 Cells were treated as indicated and α6 was stained via indirect immunofluorescence. Scale bars −20 μm.

FIG. 7L. RT4 cells infected with either control sgRNA (sgV0) or sgRNA against SESN3 (sgSESN3_1) were treated as indicated, and α6 was stained via indirect immunofluorescence.

FIG. 7M. RT4 Cells were treated as indicated and 6 was stained via indirect immunofluorescence. Scale bars −20 μm.

FIG. 7N (i)-(ii). RT4 cells (7N(i)) and HeLa cells (7N(ii)) were treated as indicated and their extracts blotted for total and phosphorylated Tyr705 of STAT3 (P-STAT3 Y705). Presented is a quantification of the fold-change in phosphorylation relative to control and normalized to total STAT3.

FIG. 7O. RT4 Cells were transfected with siRNA against p62 and NBR1 and were treated as indicated. α6 was stained via indirect immunofluorescence. Scale bars −20 μm.

FIG. 7P. HeLa Cells were transfected with siRNA against AKIRIN2, and were treated as indicated. α6 was stained via indirect immunofluorescence.

FIG. 7Q. MDA-MB-231 cells were infected with shRNA against the NPC protein NUP93 and treated as indicated. α6 was stained via indirect immunofluorescence. Scale bars −5 μm.

FIG. 8A-8I: YWF stimulate mTOR activity towards its bona fide substrates which are involved in proteolysis regulation

FIG. 8A. Western blot analysis of HeLa cell lysates for phosphorylated p62 following the indicated treatments.

FIG. 8B. Western blot analysis of HeLa cell lysates for phosphorylated TFEB following the indicated treatments.

FIG. 8C. Cells were transfected with siRNA against TFEB and were treated as indicated. The α6 proteasome subunit and the nucleus were visualized using fluorescent confocal microscopy. Scale bar −20 μm.

FIG. 8D. Western blot analysis of LC3 following the indicated treatment.

FIG. 8E-8F. HeLa cells stably expressing the protein fusion RFP-GFP-LC3 were treated as indicated, and autophagic flux was monitored qualitatively (FIG. 8E) and quantitatively (FIG. 8F).

FIG. 8G. Western blot analyses of the phosphorylation of p70-S6K and 4EBP1 following the indicated treatments.

FIG. 8H (i)-(ii). HeLa cells were treated with either complete medium (Cont.), a medium lacking all amino acids (St.), or a medium lacking all amino acids that was supplemented with YWF (St.+YWF). Proteins in cell lysates were digested with trypsin and enriched for ubiquitinated peptides, and ubiquitinated proteasomal subunits were identified via LC-MSMS. Presented are subunits of the 19S (8H(i)) and 20S (8H(ii)) proteasome.

FIG. 8I. Cells were transfected with siRNA against TFEB, and silencing was assessed via Western blot.

FIG. 9A-9B: Stimulation of mTOR by YWF involves blocking/reduction of the inhibitory interaction of SESN3 with GATOR2

FIG. 9A. Illustrates the mTOR mediated satiety signaling. mTOR is inactive under amino acids deprivation. More specifically, mTOR inhibition is mediated by SESN2, that interacts with the GATOR2 complex and suppresses its inhibitory action on the GATOR1 complex, thereby leading to mTOR inhibition. In the presence of amino acids, specifically, Leucine, the inhibitory interaction of SESN2 with GATOR2 is blocked. Released from SESN2 inhibitory effect, the GATOR2 complex suppresses the inhibitory function of GATOR1 on mTOR, thereby acting as mTOR agonist. Similarly, the interaction of YWF with SESN3 blocks its inhibitory interaction with GATOR2, thereby leading to suppression of the inhibitory function of GATOR1 on mTOR. The figure has been partially adapted from Youheng Wei, et al., (Oct. 25, 2019 https://doi.org/10.7554/cLife.42149).

FIG. 9B (i)-(vi). shows mass spectrometric analysis of immunoprecipitated lysates of Flag-SESN3 expressing Hela or RT4 cells, that were starved in the presence or absence of YWF (Control=complete medium; Starvation=no amino acids; Starvation+YWF-only the aromatic amino acids). Interaction of SESN3 with members of the GATOR2 complex (Mios (9B(iv), WDR59 (9B(ii), SEHIL (9B(i) and SEC13 (9B(v)) is elevated in starved cells. This interaction is inhibited by the presence of YWF.

FIG. 10A-10L: Proteasome translocation is required for amino acid supplementation mediated via stimulated proteolysis, and is essential for cell survival

FIG. 10A. Degradation of radiolabeled proteins was measured in Hela cells following the indicated treatments.

FIG. 10B. Measurement of degradation of the fluorogenic proteasome substrate Suc-LLVY-AMC in nuclear and cytosolic fractions in Hela cells, treated as indicated.

FIG. 10C. Western blot of HeLa cells' extracts (treated as indicated) for the cytosolic proteasomal substrate HMGCS1. Presented is the quantification of HMGCS1, normalized to GAPDH.

FIG. 10D. Western blot of extracts of Hela cells treated as indicated for the overexpressed cytosolic protein NES-GFP-CLI and RFP. Presented are quantifications of the blots of each antibody, normalized to Tubulin.

FIG. 10E. Live imaging of the proteasome activity probe Me4BodipyFL-Ahx3Leu3VS in HeLa cells treated as indicated.

FIG. 10F. Ubiquitin conjugates levels at different time points as monitored in Hela cells treated as indicated.

FIG. 10G (i)-(ii). Changes in the level of individual cellular proteins in Hela cells treated as indicated, determined by proteomic mass-spectrometric analysis. 10G(i) St.+LMB/Cont. 10G(ii) St.+YWF/Cont.

FIG. 10H (i)-(ii). Changes in the levels of individual amino acids as determined by metabolomic mass-spectrometric analysis. FIG. 10H(i) Hela cells incubated in the presence of the mTOR inhibitor Torin1, either in the absence (TI) or presence of the Exportin-1 inhibitor LMB (T1+LMB), relative to control. FIG. 10H(ii) HeLa cells incubated in a medium lacking the aromatic amino acids YWF, relative to control.

FIG. 10I (i)-(ii). Time course of HeLa (10I(i)) and RT4 (10I(ii)) cell survival under the indicated conditions.

FIG. 10J. MDA-MB-231 cells infected with shRNA against the NPC protein NUP93 were treated as indicated for 8 h. Presented are cell survival rates relative to control.

FIG. 10K. Hela cells were treated as indicated and α6 was stained via indirect immunofluorescence.

FIG. 10L (i)-(ii). HeLa (i), or RT4 (ii) cells were treated as indicated and the α6 proteasome subunit and the nucleus were visualized using fluorescent confocal microscopy. Scale bar −40 μm.

FIG. 11A-11F: Proteasome translocation is required for enhanced proteolysis of cytosolic proteins and subsequent amino acid provision

FIG. 11A. Hela cells infected with cDNA coding for NES-GFP-CLI were incubated for the indicated times in the presence of either CHX, MG132 or Chloroquine (Cq.). Cells were lysed, resolved via SDS-PAGE, and blotted with an antibody against GFP.

FIG. 11B. The proteins that are most affected by the inhibition of proteasome export using LMB or YWF (uppermost 10%; FIG. 10G), were classified according to their cellular distribution-cytoplasmic, nuclear, and proteins shared between the two compartments.

FIG. 11C. The proteins that are most affected by the inhibition of proteasome export (uppermost 10%), were classified using Gene Ontology and KEGG pathways.

FIG. 11D. The proteins that are least affected by the inhibition of proteasome export (lowermost 10%), were classified using Gene Ontology and KEGG pathways.

FIG. 11E. Monitoring the stability of ribosomal proteins under the indicated treatments.

FIG. 11F (i)-(ii). Survival rates of Hela cells (11F(i)) and RT4 cells (11F(ii)) under the indicated treatments, relative to control.

FIG. 12A-12J: Proteasome recruitment is characteristic to stressed cells in xenografts, and is required for tumor growth

FIGS. 12A and 12B. Immunohistochemistry of the proteasome in MDA-MB-231 (FIG. 12A) and RT4 (FIG. 12B) xenograft tumor sections following the indicated treatments. Periphery and core denote the corresponding regions in the tumor. Areas with no staining in ‘core’ fields of view are the result of apoptosis and necrosis with and subsequent discontinuity of tumor tissue and invasion of the host tissue.

FIG. 12C. Detection of apoptosis in RT4 xenograft tumor section using TUNEL staining.

FIG. 12D. Detection of apoptosis in RT4 xenograft tumor section using staining for cleaved Caspase3.

FIG. 12E (i)-(ii) and 12F (i)-(ii). Tumors originating from MDA-MB-231 (FIG. 12E(i)) or RT4 (FIG. 12F(ii)) cells following the indicated injected treatments (photographed for scale on a graph paper). Plotted are tumor weights at the time of mouse sacrificing (MDA-MB-231 (12E(ii)) and RT4 (12F(ii))).

FIG. 12G (i)-(ii). Tumors originating from RT4 cells following administration of the indicated amino acids in drinking water. Analyses were carried out as in FIGS. 12E and 12F.

FIG. 12H. Tumors originating from RT4 cells infected with either control sgRNA (sgV0) or sgRNA against SESN3 (photographed for scale on a graph paper).

FIG. 12I. Plotting of tumor weights originating from RT4 cells infected with the indicated sgRNAs.

FIG. 12J. Immunohistochemistry of the proteasome in the tumors described under FIGS. 12H and 12I.

FIG. 13A-13I: Proteasome recruitment is characteristic to stressed cells in xenografts, and is required for tumor growth

FIG. 13A (i)-(ii) and 13B. Immunohistochemistry of the proteasome from RT4 (FIG. 13A(i) and 13A(ii)) and MDA-MB-231 (FIG. 12B) cells in xenograft tumor sections following the indicated treatments. Periphery and core relate to the corresponding regions in the tumor.

FIG. 13C. Tumors originating from RT4 cells following treatment initiated at the indicated times (photographed for scale on a graph paper). Left and right most columns are presented also under FIG. 12G.

FIG. 13D. Plotting of weights of tumors described under 13C at the time of mouse sacrificing. The ‘Cont. 18 d’ and ‘YWF 18 d’ groups are presented also under FIG. 12G.

FIG. 13E. Average reduction in weight of tumors described under 13C (relative to control).

FIG. 13F. Immunohistochemistry of the proteasome in tumors described under 13C-13E in the indicated experimental groups.

FIG. 13G. Plotting of tumor weights following treatment with the indicated amino acid combinations at the time of mouse sacrificing.

FIG. 13H. Average reduction in weight of tumors described under 13F (relative to control).

FIG. 13I. Average reduction in tumor weight following treatment with YWF (relative to each indicated treatment).

FIG. 14A-14M: Preventing proteasome recruitment inhibits endogenous tumor growth and metastasis

FIG. 14A. Colons and ceca from either non-induced mice, mice in which the loss of the tumor suppressor APC was induced and were either untreated or treated with YWF dissolved in their drinking water.

FIG. 14B. Low magnification of ceca from mice from the indicated groups stained for the high-grade dysplasia marker PROX1.

FIG. 14C. Immunohistochemistry of the proteasome in the tumors described under FIGS. 14A and 14B.

FIG. 14D (i)-(iii). Plotting of average cecum weight (14D(i)), number of tumors along the colon (14D(ii)), and their total volume (14D(iii)) at the time of mouse sacrificing.

FIG. 14E. Macroscopic monitoring of bladders from mice treated with the carcinogen BBN. YWF were added to the drinking water where indicated.

FIG. 14F. Low magnification of H&E staining of bladders from the different experimental groups.

FIG. 14G. Immunohistochemistry of the proteasome in endogenous bladder tumors.

FIG. 14H. Plotting of the bladder weights at the time of mice sacrificing.

FIG. 14I. Sarcomas from mince treated as indicated and photographed for scale on a graph paper.

FIG. 14J. Immunohistochemistry of the proteasome in the sarcomas derived from mice that were treated as indicated.

FIG. 14K. Plotting of the sarcoma weights at the time of mice sacrificing.

FIG. 14L. IVIS analysis of mCherry intensity of liver metastases at the time of mice sacrificing.

FIG. 14M. Livers originating from the different groups as visualized by IVIS.

FIG. 15A-15C: Preventing proteasome recruitment inhibits endogenous tumor growth and metastasis

FIG. 15A. Immunohistochemistry of the proteasome in tumors described under FIG. 14A.

FIG. 15B. Immunohistochemistry of the proteasome in tumors described under FIG. 14E.

FIG. 15C. Immunohistochemistry of the proteasome in tumors described under FIG. 14I.

### DETAILED DESCRIPTION OF THE INVENTION

The proteasome, the catalytic arm of the ubiquitin system, is largely responsible for protein degradation under basal conditions, while autophagy is recruited mostly under stress. The present inventors found that following starvation to amino acids, the proteasome is translocated from its large nuclear pool into the cytoplasm. This response is regulated by the triad of mTOR-agonistic amino acids—Tyr, Trp, and Phe (YWF), recently disclosed by the present inventors [17]. The inventors now show that this response is dependent on (i) Sestrin3—a less characterized mTORC1 interactor which is now shown by the present disclosure to be required for the complex dissociation from the lysosome, and (ii) the proteolysis-promoting transcription factor STAT3. Proteasome recruitment stimulates proteolysis to enable survival under stress. In contrast, its nuclear sequestration in response to mTORC1 activation by YWF, which is mediated by p38 MAPK, inhibits this proteolytic stress-coping mechanism, leading to cell death. Importantly, the nuclear sequestration inhibits growth of xenograft, spontaneous, and metastatic mouse tumor models. This newly identified approach for hijacking the cellular “satiety center” carries therefore potential therapeutic implications for cancer.

Thus, a first aspect of the present disclosure relates to a method for treating, preventing, inhibiting, reducing, eliminating, protecting or delaying the onset of at least one condition or at least one pathologic disorder in a subject in need thereof. More specifically, the method comprising the step of administering to the subject a therapeutic effective amount of at least one compound that modulates proteasome dynamics and/or function in a mammalian cell, specifically, a cell of the treated subject. In some embodiments, the compound is characterized by affecting at least one of: mammalian target of rapamycin (mTOR) activation and/or lysosomal association, the activity and/or level/s and/or the post translational modification/s (PTM/s), and/or subcellular localization of at least one signaling molecule participating directly or indirectly in at least one pathway mediating said proteasome dynamics/function. Optionally, the modulating compound may further modulate proteasome cellular localization.

The compounds and methods disclosed herein modulate proteasome dynamics, for example as reflected by the cellular proteasome localization, the proteasome activity and/or assembly. More specifically, Proteasomes, as used herein, are protein complexes which degrade unneeded or damaged proteins by proteolysis, a chemical reaction that breaks peptide bonds, mediated by proteases. Proteasomes are part of a major mechanism by which cells regulate the concentration of particular proteins and degrade misfolded proteins. Proteins are tagged for degradation with a small protein called ubiquitin. The tagging reaction is catalyzed by ubiquitin ligases. The degradation process yields peptides of about seven to eight amino acids long, which can then be further degraded into shorter amino acid sequences and used in synthesizing new proteins. Proteasomes are found inside all eukaryotes and archaea, and in some bacteria. In structure, the proteasome is a cylindrical complex containing a “core” of four stacked rings forming a central pore. Each ring is composed of seven individual proteins. The inner two rings are made of seven 8 subunits that contain three to seven protease active sites. These sites are located on the interior surface of the rings, so that the target protein must enter the central pore before it is degraded. The outer two rings each contain seven a subunits whose function is to maintain a “gate” through which proteins enter the barrel. These a subunits are controlled by binding to “cap” structures or regulatory particles that recognize polyubiquitin tags attached to protein substrates and initiate the degradation process. The overall system of ubiquitination and proteasomal degradation is known as the ubiquitin-proteasome system (UPS).

The proteasome subcomponents are often referred to by their Svedberg sedimentation coefficient (denoted S). The proteasome most exclusively used in mammals is the cytosolic 26S proteasome, which is about 2000 kilodaltons (kDa) containing one 20S protein subunit (also referred to herein as the core proteasome, or CP) and two 19S regulatory cap subunits (also referred to herein as the regulatory proteasome or RP). The core is hollow and provides an enclosed cavity in which proteins are degraded. Openings at the two ends of the core allow the target protein to enter. Each end of the core particle associates with a 19S regulatory subunit that contains multiple ATPase active sites and ubiquitin binding sites. This structure recognizes polyubiquitinated proteins and transfers them to the catalytic core. An alternative form of regulatory subunit called the 11S particle may play a role in degradation of foreign peptides and can associate with the core in essentially the same manner as the 19S particle. The proteasomal degradation pathway is essential for many cellular processes, including the cell cycle, the regulation of gene expression, and responses to oxidative stress.

In some embodiments, the compounds and methods disclosed herein modulate proteasome dynamics and/or function, and as such, modulate translocation and shuttling of the proteasome between the nucleus and cytosol. In some embodiments, Proteasome dynamics and/or proteasome compartmentalization as used herein is meant the transport and shuttling of the proteasome between cellular compartments, specifically, the cytoplasm and nucleus. In some embodiments, such translocation involves dissociation into proteolytic core and regulatory complexes, and upon translocation re-assembly of the subunits to form the assembled proteasome, in the relevant cellular compartments. Translocation of the proteasome affect its function on its cellular substrates (e.g., degradation thereof), thereby affecting the proteasome function. In some embodiment, the compounds of the present disclosure act in selective modulation of translocation and shuttling of the proteasome thereby resulting in nuclear or predominant nuclear localization. In some embodiments, the modulating compounds of the present disclosure may act as selective inhibitors of translocation of the proteasome from the nucleus to the cytoplasm. In yet some alternative or additional embodiments, the modulating compounds of the present disclosure act to enhance recruitment of the proteasome into the nucleus.

Still further, in some embodiments, the modulating compounds of the present disclosure act to retain, maintain or even enhance a nuclear or predominantly nuclear localization of the proteasome. In some embodiments, the modulator acts as a selective modulator. More specifically, a Selective modulator, as used herein is meant that the modulating compounds of the present disclosure act exclusively, mainly, specifically, and/or predominantly, on the proteasome, for example, on the translocation and/or shuttling of the proteasome between the nucleus and cytoplasm, while not affecting (or almost no affecting) the translocation, export or import of other cellular elements (e.g., other substrates of exportin or importin). In some embodiments, selective and specific modulators as indicated herein is meant that the modulating compounds of the present disclosure selectively and exclusively act on the proteasome more than about 10% to about 100%, specifically, 10, 15, 20, 25, 30, 35, 40, 45, 50, 55, 60, 65, 70, 75, 80, 85, 90, 95, 100%, or alternatively, at least about a 2-fold to at least about a 100-fold or grater, that any modulation or effect on the translocation between nucleus-cytoplasm, of other cellular elements (e.g., proteins, nucleic acids, etc.). The present disclosure provides compounds that modulate the proteasome dynamics and/or function in a cell, compositions and uses thereof in therapeutic and diagnostic applications. These compounds are referred to throughout the entire specification as “modulator/s”, “proteasome modulator/s”, “modulating compound/s”, “modulatory compound/s”, “proteasome modulating compound/s”, “proteasome modulatory compound/s”, and the like. It should be understood that these terms are interchangeably used herein, and they all refer to the compound that modulates proteasome dynamics and/or function. The term modulates or modulating refers to changing a certain phenotype to a certain direction, that is either increasing or decreasing said phenotype. For example, in some embodiments at least one compound modulates protcasome dynamics and/or function in a mammalian cell, wherein said compound is characterized by affecting at least one of: mammalian target of rapamycin (mTOR) activation, and/or lysosomal association, the activity and/or level/s and/or the post translational modification/s (PTM/s), and/or subcellular localization of at least one signaling molecule participating directly or indirectly in at least one pathway mediating said proteasome dynamics/function. Optionally, in addition to each of the characterizing features discussed herein, the modulating compound may further modulate proteasome cellular localization. The compound herein modulates that is either increases or decreases the activation and/or association of mTOR to the lysosome and/or increases or decreases the proteasome localization in the nucleus, and/or either increases or decreases the proteasome localization in the cytosol, and/or increases or decreases the activity/levels/PTMs/subcellular localization of a signaling molecule participating directly or indirectly in at least one pathway mediating said proteasome dynamics/function. Increase or enhancement may be an increase or elevation of between about 5% to 100%, specifically, 10% to 100%. The terms “increase”, “augmentation” and “enhancement” as used herein relate to the act of becoming progressively greater in size, amount, number, or intensity. Particularly, an increase of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the phenotype as compared to a suitable control, e.g., the activation and/or association of mTOR to the lysosome and/or proteasome localization in the nucleus, and/or proteasome localization in the cytosol, and/or the activity/levels/PTMs/subcellular localization of a signaling molecule participating directly or indirectly in at least one pathway mediating said proteasome dynamics/function before treatment with at least one compound of the present disclosure. Decrease or inhibit or attenuate may be a decrease or reduction of between about 5% to 100%, specifically, 10% to 100%. The terms “decrease”, “reduction” as used herein relate to the act of becoming progressively lower in size, amount, number, or intensity. Particularly, a decrease of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the phenotype as compared to a suitable control, e.g., the activation of mTOR and/or the association of mTOR to the lysosome and/or proteasome localization in the nucleus, and/or proteasome localization in the cytosol, and/or the activity/levels/PTMs/subcellular localization of a signaling molecule participating directly or indirectly in at least one pathway mediating said proteasome dynamics/function before treatment with at least one compound of the present disclosure. The present inventors revealed the role of various signaling molecules in proteasome dynamics, and moreover, the clinical role of proteasome localization in various pathologic disorders, and the present disclosure further provides compounds modulating the activation and/or lysosomal association of mTOR, demonstrating the role of mTOR in proteasome dynamics. The mammalian target of rapamycin (mTOR), sometimes also referred to as the mechanistic target of rapamycin and FK506-binding protein 12-rapamycin-associated protein 1 (FRAP1), is a kinase that in humans is encoded by the MTOR gene. mTOR is a member of the phosphatidylinositol 3-kinase-related kinase family of protein kinases. mTOR links with other proteins and serves as a core component of two distinct protein complexes, mTOR complex 1 and mTOR complex 2, which regulate different cellular processes. In particular, as a core component of both complexes, mTOR functions as a serine/threonine protein kinase that regulates cell growth, cell proliferation, cell motility, cell survival, protein synthesis, autophagy, and transcription. As a core component of mTORC2, mTOR also functions as a tyrosine protein kinase that promotes the activation of insulin receptors and insulin-like growth factor 1 receptors. mTORC2 is also implicated in the control and maintenance of the actin cytoskeleton. mTOR is the catalytic subunit of two structurally distinct complexes: mTORC1 and mTORC2. Both complexes localize to different subcellular compartments, thus affecting their activation and function. Upon activation by Rheb, mTORC1 localizes to the Regulator-Rag complex on the lysosome surface where it then becomes active in the presence of sufficient amino acids. mTOR Complex 1 (mTORC1) is composed of mTOR, regulatory-associated protein of mTOR (Raptor), mammalian lethal with SEC13 protein 8 (mLST8) and the non-core components PRAS40 and DEPTOR. This complex functions as a nutrient/energy/redox sensor and controls protein synthesis. The activity of mTORC1 is regulated by rapamycin, insulin, growth factors, phosphatidic acid, certain amino acids and their derivatives (e.g., l-leucine and β-hydroxy β-methylbutyric acid), mechanical stimuli, and oxidative stress.

mTOR Complex 2 (mTORC2) is composed of MTOR, rapamycin-insensitive companion of MTOR (RICTOR), MLST8, and mammalian stress-activated protein kinase interacting protein 1 (mSIN1). mTORC2 has been shown to function as an important regulator of the actin cytoskeleton through its stimulation of F-actin stress fibers, paxillin, RhoA, Rac1, Cdc42, and protein kinase Cα (PKCα). mTORC2 also phosphorylates the serine/threonine protein kinase Akt/PKB, thus affecting metabolism and survival. In addition, mTORC2 exhibits tyrosine protein kinase activity and phosphorylates the insulin-like growth factor 1 receptor (IGF-IR) and insulin receptor (InsR). In some embodiments mTOR as used herein, refers to the human mTOR. In some other embodiments the mTOR is encoded by a nucleic acid sequence comprising the sequence as denoted by CCDS 127.1. In yet some further embodiments, the nucleic acid sequence encoding mTOR is denoted by SEQ ID NO: 26, or any homologs or derivatives thereof. In yet some further embodiments, mTOR encoded by the disclosed nucleic acid sequence is the human mTOR protein that comprises the amino acid sequence as denoted by Uniprot number: P42345. In yet some further specific embodiments, the mTOR comprises the amino acid sequence as denoted by SEQ ID NO: 27.

As indicated above, the present disclosure provides compounds that modulate the lysosomal association of mTOR. In some embodiments, these compounds may be any agent or drug that increases the activation and/or lysosomal association of mTOR, thereby activating, stimulating, increasing, facilitating, enhancing activation, or up regulating the activity of the mTOR protein, to produce a biological response. According to some embodiments, wherein indicated “increasing” or “enhancing” the mTOR activity and/or lysosomal association, binding, localization, incorporation, engagement, and the resulting activity, as used herein in connection with the mTOR modulators disclosed herein, it is meant that such increase or enhancement may be an increase or elevation of between about 5% to 100%, specifically, 10% to 100% of the mTOR activity. The terms “increase”, “augmentation” and “enhancement” as used herein relate to the act of becoming progressively greater in size, amount, number, or intensity. Particularly, an increase of 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 70%, 800%, 900%, 1000% or more of the activity as compared to a suitable control, e.g., mTOR activation in the absence of the modulators of the present disclosure. As indicated herein, association of mTOR to the lysosomal membrane, reflects the activation thereof. Lysosomal membrane, which has a typical single phospholipid bilayer, controls the passage of material into and out of lysosomes, by its permeability and ability to fuse with digestive vacuoles or engulf cytosolic material.

As indicated above, the disclosed modulators affect the cellular localization of the proteasome, specifically, between the nucleus (nuclear localization), and the cytoplasm (cytoplasmic localization). In yet some further embodiments, the disclosed modulators may affect the cellular localization of any of the disclosed signaling molecules. Thus, the term encompasses the predominant presence and/or localization and/or the association of any of the disclosed molecules in one or more of the cellular compartments or organelles. More specifically, the term cellular or subcellular refers to membrane-bound cellular compartments. The cells of eukaryotic organisms are subdivided into functionally-distinct membrane-bound compartments, including plasma membrane, cytoplasm, nucleus, mitochondria, Golgi apparatus, endoplasmic reticulum (ER),peroxisome, vacuoles, cytoskeleton, nucleoplasm, nuclear matrix, and ribosomes. More specifically, the nucleus includes the nuclear matrix, a network within the nucleus that adds mechanical support and is surrounded by the nuclear envelope, a double membrane that encloses the entire organelle and isolates its contents from the cellular cytoplasm. In some embodiments, the term cytosol as used herein refers to all subcellular compartments of the cell excepts the nucleus. In some specific embodiments, a nuclear localization indicates the predominant presence of the proteasome, and/or any of the indicated signaling molecules in the nucleus, or in any compartment defined by or surrounded by the nuclear membrane. In yet some further embodiments, a cytosolic localization indicates the predominant presence of the proteasome, and/or any of the indicated signaling molecules in the cytoplasm, or in any compartment or organelle that is not included within, defined by, or surrounded by the nuclear membrane. More specifically, in some embodiments, cytosolic localization may include localization to any of the disclosed organelles or compartments present between the cytoplasm membrane and the nuclear membrane.

In some embodiments, to achieve the modulation of the proteasome dynamics and/or function, the disclosed modulators or modulating compounds used in the present disclosure, may affect the post translation modification of any of the signaling molecules that participate in any signaling pathway that modulates proteasome dynamics, as will be elaborated herein after. Thus, in addition to modulators of proteasome dynamics and/or function, the disclosed modulators may be further characterized as affecting PTMs of signaling molecules that mediate and/or participate in pathways that lead to or involved in proteasome dynamics. More specifically, post-translational modification/s (PTM/s) is the covalent process of changing proteins following protein biosynthesis. PTMs may involve enzymes or occur spontaneously. Post-translational modifications can occur on the amino acid side chains or at the protein's C- or N-termini. It should be understood that this term refers to reactions wherein a chemical moiety is covalently added to or alternatively removed from a protein, specifically, by enzymatic or non-enzymatic reaction. Many proteins can be post-translationally modified through the covalent addition of a chemical moiety (also referred to herein as a “modifying moiety”) after the initial synthesis (i.e., translation) of the polypeptide chain. Such chemical moieties usually are added by an enzyme to an amino acid side chain or to the carboxyl or amino terminal end of the polypeptide chain, and may be cleaved off by another enzyme. Single or multiple chemical moieties, either the same or different chemical moieties, can be added to a single protein molecule. It should be noted however that other forms of protein post-translational modification that include proteolytic cleavage of peptide bonds, removing the initiator methionine residue, as well as the formation of disulfide bonds using linking cysteine residues, and protein splicing are also encompassed by the invention.

PTM of a protein can alter its biological function, such as its enzyme activity, its binding to or activation of other proteins, its cellular localization or its turnover, and is important in cell signaling events, development of an organism, and disease. As will be described in more detail herein after, examples of PTM covered by the method of the invention include, but are not limited to phosphorylation, ubiquitination and ubiquitin-chain preference, as demonstrated herein, as well as to any PTM reaction performed by ubiquitin-like protein, for example, sumoylation, neddylation, pupylation, ISGylation, and the like. It should be appreciated that in some embodiments, the PTM reaction as defined by the invention further encompass the addition of Hydrophobic groups for membrane localization include myristolation, that involves the attachment of myristate (that is a C₁₄ saturated acid), palmitoylation, attachment of palmitate, a C₁₆ saturated acid, isoprenylation or prenylation, that involve the addition of an isoprenoid group (e.g. farnesol and geranylgeraniol), farnesylation, geranylgeranylation, glypiation, glycosylphosphatidylinositol (GPI) anchor formation via an amide bond to C-terminal tail, and the like. Still further, several modifications may enhance the enzymatic activity of a given enzyme. Such PTMs may include for example, lipoylation, that involves the attachment of a lipoate (Cs) functional group, covalent attachment of flavin moiety (FMN or FAD), attachment of heme C via thioether bonds with cysteins, phosphopantetheinylation, that involves the addition of a 4′-phosphopantetheinyl moiety from coenzyme A as well as retinylidene Schiff base formation. Still further embodiments of PTMs include diphthamide formation, ethanolamine phosphoglycerol attachment and hypusine formation. PTMs involving the attachment or removal of small chemical groups include acylation, e.g. O-acylation (esters), N-acylation (amides), S-acylation (thioesters), and crotonylation that involves for example, addition of crotonyl to histons and acetylation, that involves the addition of an acetyl group, either at the N-terminus of the protein or at lysine residues, or alternatively deacetylation involving the removal of said acetyl group and formylation. Still further PTMs relate to alkylation, that involve the addition of an alkyl group, e.g. methyl, ethyl, methylation or demethylation (addition or removal of at least one methyl group at lysine or arginine residues). Still further modifications include amide bond formation that may encompass amidation at C-terminus and amino acid addition that may include arginylation, a tRNA-mediation addition, polyglutamylation, that involves the covalent linkage of glutamic acid residues and polyglycylation, covalent linkage of at least one glycine residue. Still further, butyrylation, gamma-carboxylation and glycosylation, that involves the addition of a glycosyl group to either arginine, asparagine, cysteine, hydroxylysine, serine, threonine, tyrosine, or tryptophan. In further embodiments, PTMs may also include polysialylation, malonylation, hydroxylation, iodination, nucleotide addition such as ADP-ribosylation, oxidation, phosphate ester (O-linked) or phosphoramidate (N-linked) formation, phosphorylation, the addition of a phosphate group, usually to serine, threonine, and tyrosine (O-linked), or histidine (N-linked), adenylylation, the addition of an adenylyl moiety, usually to tyrosine (O-linked), or histidine and lysine (N-linked), propionylation, pyroglutamate formation, S-glutathionylation, S-nitrosylation, S-sulfenylation, succinylation that involves the addition of a succinyl group to lysine, sulfation, the addition of a sulfate group to a tyrosine and the like.

It should be further appreciated that the term PTM as used herein further encompasses non enzymatic modifications, for example, glycation, carbamylation the addition of Isocyanic acid to an N-terminus of either lysine, histidine, taurine, arginine, or cysteine, carbonylation the addition of carbon monoxide to other organic/inorganic compounds.

In some embodiments, the disclosed modulators may affect the phosphorylation of specific signaling molecules that participate in signaling pathways involved in proteasome dynamics, as reveled by the present disclosure. In some embodiments, modulator (the modulating compound) useful in the disclosed methods may affect (reduce or alternatively increase) the phosphorylation of any one of p38, STAT3, and/or p62, thereby modulating the effect of each of these signaling molecules on the proteasome dynamics.

In some embodiments, the compounds used by the disclosed methods, affects at least one signaling molecule participating directly or indirectly in at least one pathway mediating the proteasome dynamics and/or function. Such signaling molecule may be in some embodiments, at least one of: at least one stress-induced protein/s, at least one mediator of cellular response to environmental cues, at least one shuttle protein/s, and at least one Nuclear Pore Complex (NPC) protein. More specifically, in some embodiments, such signaling molecule affected by the compound used as a modulator in the methods of the present disclosure, may be at least one stress-induced protein/s.

Stress-induced protein/s (SPs) are a diverse group of proteins that are synthesized at increased levels when cells are exposed to either intracellular or extracellular stressful stimuli. They exhibit protective effects against stresses. Stress proteins include heat shock proteins (HSPs), RNA chaperone protein (RNPs), and proteins mainly function in the endoplasmic reticulum (ER): peptidyl-propyl isomerases, protein disulfide isomerases (PDIs) and the lectin-binding chaperone system. SPs are ubiquitously expressed in all kinds of cells, triggering signal cascades for neutralizing and eradicating the stresses occurring both extracellularly (e.g., starvation, stimulation by cytokines/chemokines or hormones) and intracellularly (e.g., pathogen invasion). Responses triggered by SPs can either activate pathways to promote cell survival or initiate cell death (i.e., apoptosis, necrosis, pyroptosis or autophagic cell death) for eliminating the damaged cells to protect a particular organ/tissue under given conditions.

In some embodiments, at least one signaling molecule participating directly or indirectly in the at least one signal transduction pathway mediating the proteasome dynamics and/or function may be at least one mediator of metabolite sensing, and/or at least one stress kinase, and/or at least one nucleo-cytosolic shuttle protein (specifically, ubiquitin and/or proteasome interacting shuttle proteins), and/or at least one Nuclear Pore Complex (NPC) protein. More specifically, sensing and responding to changes in nutrient levels, including those of metabolites such as glucose, lipids, and amino acids, by the body is necessary for survival. Accordingly, any molecule that participates either directly or indirectly in sensing the levels of such metabolites may be encompassed by the present disclosure. These nutrient-dependent cellular processes, broadly termed “nutrient sensing” contains a broad array of processes and pathways including nutrient transport, processing, and metabolic control. In some specific embodiments, a mediator of metabolite sensing is a mediator of amino acid sensing. More specifically, amino acids that are fundamental elements for protein and peptide synthesis, have been recently shown as important bioactive molecules that play key roles in signaling pathways and metabolic regulation. Different pathways that sense intracellular and extracellular levels of amino acids are integrated and coordinated at the organismal level, and, together, these pathways maintain whole metabolic homeostasis. In some specific embodiments of the disclosed methods, the mediator of metabolite sensing may be a mediator of amino acid sensing. To name but a few, amino acid sensing molecules include, but are not limited to the Sestrin family members, specifically, Sestrin 2, and to a lesser extent Sestrin 1 (sensing Leu), Uncharged tRNALeu senses Leu (via GCN2 and eIF2), SAR1B (sensing Leu), CASTOR1 (sensing Arg), and SAMTOR (sensing Met). Thus, in some embodiments, the methods of the present disclosure may use as a modulator any compound that affects any of the mediators of amino acid sensing, specifically, any of the mediators disclosed herein.

In some embodiments, at least one signaling molecule participating directly or indirectly in the at least one signal transduction pathway mediating the proteasome dynamics and/or function may be at least one stress kinase. Still further, in yet some additional or alternative embodiments, the stress kinase may be at least one member of the Mitogen-activated protein kinases (MAPKs). A mitogen-activated protein kinase (MAPK or MAP kinase) is a type of protein kinase that is specific to the amino acids serine and threonine(i.e., a serine/threonine-specific protein kinase). MAPKs are involved in directing cellular responses to a diverse array of stimuli, such as mitogens, osmotic stress, heat shock and proinflammatory cytokines. They regulate cell functions including proliferation, gene expression, differentiation, mitosis, cell survival, and apoptosis. MAPKs belong to the CMGC (CDK/MAPK/GSK3/CLK) kinase group. The closest relatives of MAPKs are the cyclin-dependent kinases (CDKs). Most MAPKs have a number of shared characteristics, such as the activation dependent on two phosphorylation events, a three-tiered pathway architecture and similar substrate recognition sites. These are the “classical” MAP kinases, however, the group further encompasses the use of “atypical” MAPKs. The mammalian MAPK family of kinases includes three subfamilies: Extracellular signal-regulated kinases (ERKs), c-Jun N-terminal kinases (JNKs), p38 mitogen-activated protein kinases (p38s). Generally, ERKs are activated by growth factors and mitogens, whereas cellular stresses and inflammatory cytokines activate JNKs and p38s.

As indicated herein, the modulator of the present disclosure targets at least one signaling molecule participating directly or indirectly in at least one pathway mediating the proteasome dynamics and/or function. In some embodiments, such signaling molecule may be at least one nucleo-cytosolic shuttle protein, or any protein participating in nucleocytoplasmic transport of proteins and protein complexes. More specifically, nucleocytoplasmic transport of protein including import to the nucleus and export to the cytoplasm is a complicated process that requires involvement and interaction of many proteins. In yet some more specific embodiments, the nucleocytoplasmic shuttling proteins as used herein may be any shuttle protein that participates in protein quality control (PQC). In some embodiments, shuttle protein that participates in protein quality control (PQC) include, but are not limited to SQSTM1 (p62) (Uniport number: Q13501), NBR1 (Uniport number: Q14596), VCP (p97) (Uniport number: P55072), OPTN (Optincurin); (Uniport number: Q96CV9), TAX1BP1 (Uniport number: Q86VP1), NDP52 (CACO2/CALCOCO2) (Uniport number: Q13137), RAD23A (Uniport number: P54725), RAD23B (Uniport number: P54727), UBQLN2 (DSK2 homolog) (Uniport number: Q9UHD9), UBQLN1 (Uniport number: Q5R684), UBQLN3 (Uniport number: Q9H347), UBQLN4 (Uniport number: Q9NRR5), DDI1 (Uniport number: Q8WTU0), and DDI2 (Uniport number: Q5TDH0).

In some embodiments of the disclosed methods, at least one of: (i) the at least one mediator of amino acid sensing is at least one member of the Sestrin family. In yet some further or additional embodiments, (ii), the at least one member of the MAPKs is at least one member of the p38 mitogen-activated protein kinases (p38 MAPK, p38). In yet some further additional or alternative embodiments, (iii), the at least one nucleo-cytosolic shuttle protein/s is at least one of Sequestosome 1 (SQSTM1, p62) and Neighbor of BRCA1 gene 1 protein (NBR1). Still further, in some additional or alternative embodiments (iv), the at least one NPC is Nucleoporin 93 (NUP93). In some embodiments, the disclosed signaling molecule/s affected by the disclosed modulator/s may be any member of the nuclear pore complex (NPC).

Still further, in some embodiments, the modulator of the present disclosure targets at least one signaling molecule participating directly or indirectly in at least one pathway mediating the proteasome dynamics and/or function that may be at least one Nuclear Pore Complex (NPC) protein. NPCs span the nuclear envelope, serving both as the main conduit for molecules between the nucleus and cytoplasm and as a permeability barrier to limit the passage of macromolecules and to ensure the maintenance of nuclear composition. The conserved Karyopherin-β (Kap) family of nuclear transport receptors mediates the majority of transport of macromolecules, especially of proteins, across the NPC into the nucleus (importins), out of the nucleus (exportins) or in both directions (biportins). Still further, the NPC comprises around 30 different proteins collectively called nucleoporins (NUPs). Generally, the nucleoporins are divided into the following three categories. (a) Membrane NUPs: Three membrane-spanning NUP proteins contain transmembrane helices that can fasten NPC to the nuclear envelope and they can strengthen interaction between outer and inner membranes of the envelope. (b) Scaffold NUPs: They serve as a linker between the membrane NUPs and NUPs with repeating amino acid sequences. (c) FG-NUPs: Characterized by repeated consensus FXFG and/or GLFG, which are the minimal domain for performing an important function in cells, most FG-NUPs reside within the central transport channel and construct the permeability barrier that can interact with transport receptors family, forming the route for nucleocytoplasmic transport.

In yet some further specific embodiments, NUP proteins applicable in the present disclosure may be any NUP NPC participating in cargo translocation. In some embodiments, NUP proteins in accordance with the present disclosure may be the Linker NUPs (e.g., NUP93, NUP88), the Nuclear NUPs and Basket (NUP153, TPR), the Cytoplasmic NUPs and filaments (NUP358, NUP214, NLP1), Central NUPs (NUP98, NUP62, NUP54, NUP58, NUP45).

Still further, in some embodiments, the signaling molecule affected by the compounds used in the disclosed methods may be a mediator of cellular response to environmental cues. More specifically, such mediator may be according to some embodiments, the Signal transducer and activator of transcription 3 (STAT3).

In some embodiments of the disclosed methods, the at least one member of the Sestrin family is Sestrin3 (SESN3). Thus, in such embodiments, the signaling molecule affected by the modulator used in the present disclosure, may be Sestrin3. In yet some further additional or alternative embodiments, the at least one member of the p38 MAPK family, is the p388 (p38 delta, MAPK13). Thus, in such embodiments, the signaling molecule affected by the modulator used in the present disclosure, may be the p388.

In some specific embodiments, a compound useful in the methods of the present disclosure may be any compound that leads to mTOR activation and/or localization to the lysosomal membrane, or a compound that prevents or reduces the dissociation of mTOR from the lysosomal membrane. Still further, in some additional or alternative embodiments, a compound useful in the disclosed methods may be a compound that leads to, or increases proteasome nuclear localization, also referred to herein as leading to a predominant nuclear localization. It should be noted that in some additional or alternative embodiments, such compound may increase the ratio of nuclear to cytosolic proteasome localization or lead to a ratio of nuclear to cytosolic proteasome localization that is greater than 1. Still further, in some additional or alternative embodiments, the compounds of the disclosed methods may be compounds that lead to reduction in Sestrin3 levels and/or activity. Still further, in some additional or alternative embodiments, the compound of the disclosed methods may be a compound that leads to activation of p38. In yet some further additional or alternative embodiments, a compound applicable in the disclosed methods may be a compound that leads to inhibition and/or reduction of Tyr705 of STAT3 phosphorylation. Thus, in such embodiments, the signaling molecule affected by the modulator used in the present disclosure, may be STAT3. In some additional or alternative embodiments, a compound applicable in the disclosed methods may be a compound that leads to a reduction in the levels and/or activity of p62 and/or NBR1. Thus, in such embodiments, the signaling molecule affected by the modulator used in the present disclosure, may be P62 and/or NBR1. In some additional or alternative embodiments, a compound applicable in the disclosed methods may be a compound that modulates NUP93. Thus, in such embodiments, the signaling molecule affected by the modulator used in the present disclosure, may be NUP93.

In some embodiments of the disclosed methods, the modulatory compound leads to, and is characterized by: (I) at least one of: (i) mTOR activation and/or localization to the lysosomal membrane; (ii) reduction in Sestrin3 levels and/or activity (e.g. association with signaling complex/es); (iii) activation of p38; (iv) reduction in the levels and/or activity of p62 and NBR1; and/or (v) modulation (specifically, activation) of NUP93. In yet some optional embodiments, the disclosed modulator of proteasome dynamics leads, in addition to at least one of the effects disclosed in (i), (ii), (iii), (i) and/or (v), also, (II), proteasome nuclear localization. In yet some further embodiments, the proteasome dynamics modulating compounds useful in the disclosed methods may lead to proteasome nuclear localization in a cell, and in addition, to at least one of the disclosed effects, specifically, (i) mTOR activation and localization to the lysosomal membrane; (ii) reduction in Sestrin3 levels and/or activity; (iii) activation of p38; (iv), reduction in the levels and/or activity of p62 and NBR1; and/or (v) modulation (specifically, activation) of NUP93, or any combinations thereof. In some embodiments, the disclosed modulating compound may lead to proteasome nuclear localization and in addition, to activation of mTOR, and/or to increased association of mTOR to the lysosomal membrane. In yet some further embodiments, the disclosed modulating compound may lead to proteasome nuclear localization and in addition, to reduction in Sestrin3 levels and/or activity. Still further, in some embodiments, the disclosed modulating compound may lead to proteasome nuclear localization and in addition, to activation of p38, specifically, p38 delta. Still further, in some embodiments, the disclosed modulating compound may lead to proteasome nuclear localization and in addition, to reduction in the levels and/or activity of p62 and NBR1. Still further, in some embodiments, the disclosed modulating compound may lead to proteasome nuclear localization and in addition, to activation of NUP93. In some embodiments, the disclosed methods may use any combination of the compounds indicated herein above. It should be further understood that in some further embodiments, the specified compounds may be functionally characterized by one or more of the disclosed features, and lead to one or more of the indicated outcomes. However, in other embodiments, any combination of the disclosed compounds is encompassed and useful in the methods of the present disclosure.

In some particular embodiments of the disclosed methods, any compound that leads to any of the discussed features and outcomes may be used, provided that the compound is not or does not comprise at least one aromatic amino acid residue, specifically, at least one of, Tyrosine (y, Tyr), Tryptophan (W, Trp) and/or Phenylalanine (F, Phe), or any combinations or mimetics thereof. Thus, in some particular embodiments, any compound can be used in the disclosed methods with the proviso that such compound that modulates the proteasome dynamics and/or function, is not the YWF triad.

In some embodiments, the disclosed compound that modulates proteasome dynamics and/or function (also referred to herein as the modulatory compound) useful in the disclosed methods may be, or may comprise at least one of: a nucleic acid-based molecule, an amino acid-based molecule, a small molecule or any combinations thereof. In yet some further additional or alternative embodiments, the modulatory compound may target at least one of the signaling molecule/s, as disclosed above (e.g., SESN3, p38, p62, NBR1, NUP93) at the nucleic acid sequence level or at the protein level. In some specific embodiments, the disclosed modulatory compound used in the methods of the present disclosure may target any one of the mediator/s of amino acid sensing (e.g., at least one member of the Sestrin family), the at least one member of the MAPKs, specifically, members of the p38 mitogen-activated protein kinases (p38 MAPK, p38), at least one nucleo-cytosolic shuttle protein/s, and/or at least one NPC, at the nucleic acid sequence level or at the protein level. In yet some more specific embodiments, the disclosed modulator useful in all methods and compositions of the present disclosure, may target any one of SESN3, p38 (particularly p38 delta), p62 and/or NBR1, NUP93, and/or STAT3 at the nucleic acid sequence level and/or at the protein level.

In more specific embodiments, the modulatory compounds of the present disclosure specifically target the at least one signaling molecule (e.g., SESN3, p38, p62, NBR1, NUP93) at the nucleic acid level, thereby affecting the expression, distribution and/or splicing of such target signaling molecule. In yet some additional or alternative embodiments, the disclosed modulatory compound may specifically target the at least one signaling molecule (e.g., SESN3, p38, p62, NBR1, NUP93) at the protein level, thereby affecting the stability, activity, PTMs, and/or the interactions of such target signaling molecule with other signaling molecules.

In more specific embodiments, the modulatory compound disclosed herein, targets at least one of the disclosed signaling molecule/s (e.g., SESN3, p38, p62, NBR1, NUP93), at the nucleic acid sequence level (a). In more specific embodiments, such compound may be, or may comprise at least one nucleic acid-based molecule. In some particular and non-limiting embodiments, such nucleic acid molecule may be at least one of: a nucleic acid guide, a double-stranded RNA (dsRNA), a single-stranded RNA (ssRNA), an antisense oligonucleotide, a Ribozyme, a deoxyribozymes (DNAzymes), and an aptamer.

As disclosed herein, the modulator of the present disclosure may comprise a molecule that targets the target signaling molecule at the nucleic acid sequence level. In yet some further embodiments, the disclosed modulators, may comprise nucleic acid-based molecule. Nucleic acid therapeutics are based on the provision of a sequence of nucleic acids to up-regulate, down-regulate or correct the target gene, and can be divided into two categories according to their compositions: DNA drugs and RNA drugs, among which RNA drugs can be divided into antisense oligonucleotides (ASOs), Small activating RNAs (saRNA), Small interfering RNA (siRNA), microRNAs (miRNAs), mRNA and aptamers. Still further, RNA interference (RNAi), is a general conserved eukaryotic pathway which down regulates gene expression in a sequence specific manner. It is the process of sequence-specific, post-transcriptional gene silencing in animals and plants, initiated by siRNA that is homologous in its duplex region to the sequence of the silenced gene. Gene silencing is induced and maintained by the formation of partly or perfectly double-stranded RNA (dsRNA) between the target RNA and the siRNA/shRNA derived ‘guide” RNA strand. The expression of the gene is cither completely or partially inhibited. As known in the art RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs/RNAs containing a sequence complementary to one of the siRNA strands. One of the strands of the double-stranded siRNA molecule (the “guide” strand) comprises a nucleotide sequence that is complementary to a nucleotide sequence of the target gene, or a portion thereof, and the second strand of the double-stranded siRNA molecule (the passenger” strand) comprises a nucleotide sequence substantially similar to the nucleotide sequence of the target gene, or a portion thereof. After binding to RISC, the guide strand is directed to the target mRNA cleaved between bases 10 and 11 relative to the 5′ end of the siRNA guide strand by the cleavage enzyme Argonaute-2 (AGO2). Thus, the process of mRNA translation can be interrupted by siRNA.

In more particular embodiments, siRNAs directed against any of the above target signaling molecules (e.g., SNS3, p38, p62, NBR1, NUP93), may comprise a duplex, or double-stranded region, of about 5-50 or more, 10-50 or more, 15-50 or more, 5-45, 10-45, 15-45, 5-40, 10-40, 15-40, 5-35, 10-35, 15-35, 5-30, 10-30 and 15-30 or more nucleotides long. In yet some more particular embodiments, the siRNAs of the present disclosure comprise a nucleic acid sequence comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. Often, siRNAs contain from about two to four unpaired nucleotides at the 3′ end of each strand. At least a portion of one strand of the duplex or double-stranded region of a siRNA is substantially homologous to or substantially complementary to a target sequence within the gene product (i.e., RNA) molecule as herein defined. The strand complementary to a target RNA molecule is the “antisense guide strand”, the strand homologous to the target RNA molecule is the “sense passenger strand” (which is also complementary to the siRNA antisense guide strand). siRNAs may also be contained within structured such as miRNA and shRNA which has additional sequences such as loops, linking sequences as well as stems and other folded structures. Non-limiting embodiments for siRNA molecules that may act as modulators of proteasome dynamics and/or function in accordance with some embodiments of the present disclosure may be the siRNA molecules that comprise the nucleic acid sequence as denoted by any one of SEQ ID NO: 49, 50, 51, 52, 53, 54, 55, 56, 57, 58, 59 and 60, and any combinations or compositions thereof. Each of the disclosed siRNA molecules cither alone or in any combinations thereof (also combination with any additional modulators), lead to modulation of the proteasome dynamics and/or function in the cell. Still further, the strands of a double-stranded interfering RNA (e.g., siRNA) may be connected to form a hairpin or stem-loop structure (e.g., shRNA). Thus, as mentioned above the at least one modulator of the present disclosure may also be short hairpin RNA (shRNA). Specific embodiments for shRNA molecules applicable as modulating compounds in the present disclosure may include any one of SEQ ID NO: 14 to 24.

According to other embodiments, the modulators of the present disclosure may be a micro-RNA (miRNA). miRNAs are small RNAs made from genes encoding primary transcripts of various sizes. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA. The pre-miRNA is a substrate for a form of dicer that removes the miRNA duplex from the precursor, after which, similarly to siRNAs, the duplex can be taken into the RISC complex. Unlike, siRNAs, miRNAs bind to transcript sequences with only partial complementarity and usually repress translation without affecting steady-state RNA levels. Both miRNAs and siRNAs are processed by Dicer and associate with components of the RNA-induced silencing complex (RISC). More specifically, microRNAs (miRNAs) form a class of endogenous, 20-22nt long regulatory RNA molecules. They exert their function of post-transcriptional gene regulation through mRNA cleavage, RNA degradation, and translation inhibition. Most canonical miRNAs are transcribed by RNA polymerase II (Pol II) to produce pri-miRNA transcripts, which are then cleaved by RNase III-type enzymes called Dicer-like proteins into stem-loop structured precursors in the nucleus. Stem-loop pre-miRNAs are subsequently cleaved into miRNA/miRNA* duplexes by Dicer or Dicer-like enzymes in the cytoplasm. The mature miRNAs are then incorporated into ARGONAUTE (AGO)-containing RNA-induced silencing complexes (RISC) in the cytoplasm to exert their regulatory effects by guiding the RISC to target transcripts through perfect or partially complementary base pairing. The modulator of the present disclosure may comprise miRNA-like RNAs. Still further, in some embodiments, the modulators of the present disclosure may comprise artificial miRNA (amiRNA). amiRNAs have been explored as alternative RNAi-triggering molecules and are designed to mimic primary miRNA stem-loops. The mature miRNA duplex in the central stem is replaced by sequences specifically designed for a specific target transcript, but the native flanking recognition sequences for cleavage by Drosha and Dicer are preserved. The artificial miRNAs are transcribed in larger transcripts and can be linked to RNA polymerase II-based expression systems.

More specific embodiments relate to the at least one modulator of the present disclosure that may be at least one antisense RNA. An “antisense RNA” is a single strand RNA (ssRNA) molecule that is complementary to an mRNA strand of a specific target gene product. Antisense RNA may inhibit the translation of a complementary mRNA by base-pairing to it and physically obstructing the translation machinery. By “complementary” it is meant the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. Still further, in some embodiments, at least one modulator of the present disclosure may comprise an antisense oligonucleotide, or any derivatives thereof. In more specific embodiments such oligonucleotide is an antisense oligonucleotide (ASO). As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides. As used herein, “modified oligonucleotide” means an oligonucleotide comprising at least one modified nucleoside and/or at least one modified internucleoside linkage.

Still further, in some embodiments, the disclosed modulators, specifically, modulators that target the disclosed signaling molecules (e.g., SNS3, p38, p62, NBR1, NUP93) at the nucleic acid level, may comprise at least one element of any gene editing system. More specifically, such modulator may comprise at least one nucleic acid-based element that recognize the target. The recognition of the target by the target recognition element is facilitated in some embodiments by base-pairing interactions. In some specific embodiments, the disclosed modulator may comprise at least one target recognition element, that may be a nucleic acid guide that targets a nucleic acid-modifier to the target site within any nucleic acid sequence that specifically targeting a nucleic acid sequence encoding the target signaling molecule (e.g., SNS3, p38, p62, NBR1, NUP93), or any parts thereof; or targeting any coding or non-coding nucleic acid sequence involved directly or indirectly in regulation or control of the expression and/or splicing of the target signaling molecule (e.g., SNS3, p38, p62, NBR1, NUP93). Still further, the target recognition element that may be also referred to herein as a nucleic acid guide, or in more specific embodiments as a guide RNA (gRNA), guides at least one nucleic acid modifier to the target site. The modifier modifies the target signaling molecule and thus affects the expression, activity, cellular localization and splicing thereof. In yet some further optional embodiments, the disclosed modulator may comprise in addition to the RNA guide, also at least one nucleic acid modifier, specifically, protein-based modifier, that can be provided by the disclosed modulator either as a protein, or as a nucleic acid sequence encoding the modifier.

It should be appreciated that the at least one nucleic acid guided genome modifier protein of the modulator of the present disclosure or any chimeric or fusion protein thereof, must comprise at least one effector or modifier component, or act as an effector or modifier component. In some embodiments, such effector or modifier component may be a protein-based modifier, a nucleic acid-based modifier or any combinations thereof. In some embodiments, “the nucleic acid modifier or effector” component may be any component, element or specifically protein, polypeptide or nucleic acid sequence or oligonucleotide that upon direct or indirect interaction with a target nucleic acid sequence (e.g., of any one of e.g., SNS3, p38, p62, NBR1, NUP93), modify or modulate the structure, function (e.g., expression), or stability thereof. Such modification may include the modification of at least one functional group, addition or deletion of at least one chemical group by modifying an existing functional group or introducing a new one such as methyl group. The modifications may include cleavage, methylation, demethylation, deamination and the like. Specific modifier component applicable in the present invention may include but are not limited to a protein-based modifier, for example, a nuclease, a methyltransferase, a methylated DNA binding factor, a transcription factor, transcription repressor, a chromatin remodeling factor, a polymerase, a demethylase, an acetylase, a deacetylase, a kinase, a phosphatase, an integrase, a recombinase, a ligase, a topoisomerase, a gyrase, a helicase, any combinations thereof or any fusion proteins comprising at least one of the modifier proteins disclosed by the invention. In some specific embodiments, the nucleic acid modifier component may be at least one nuclease. More specifically, as used herein, the term “nuclease” refers to an enzyme that in some embodiments display a nucleolytic activity, specifically, capable of cleaving the phosphodiester bonds between monomers of nucleic acids (e.g., DNA and/or RNA). Nucleases variously effect single and double stranded breaks in their target molecules.

In some specific embodiments, the at least one nucleic acid guided genome modifier protein used for the modulator disclosed herein may comprise at least one component of the CRISPR-Cas system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI. It should be understood that the present disclosure contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. In bacterial immunity, the CRISPR-Cas system, targets DNA molecules based on short homologous DNA sequences, called spacers that have previously been extracted by the bacterium from the foreign pathogen sequence and inserted between repeats as a memory system. These spacers are transcribed and processed and this RNA, named crRNA or guide-RNA (gRNA), guides CRISPR-associated (Cas) proteins to matching (and/or complementary) sequences within the target DNA, called proto-spacers, which are subsequently cleaved. The spacers, or other suitable constructs or RNAs can be rationally designed and produced to target any DNA sequence. Thus, in some embodiments, the gRNA used as the modulating compound in the methods of the present disclosure are designed and directed to a target sequence (a protospacer) located within the coding or non-coding sequences of any of the signaling molecules that participate in signaling that leads to or involved in proteasome dynamics (e.g., SESN3, p38, p62, NBR1, NUP93). Targeting the nucleic acid modifier by the gRNAs to the target sequence modify the sequence thereby affecting the expression, splicing and/or activation of the target signaling molecules. This manipulation by the modulatory compound (e.g. gRNA), allows the modulation of the proteasome dynamics.

In some specific embodiment, the CRISPR-Cas proteins used as the at least one nucleic acid guided genome modifier protein in the modulator of the present disclosure may be of a CRISPR Class 2 system. In yet some further particular embodiments, such class 2 system may be any one of CRISPR type II, and type V systems. In certain embodiments, the Cas applicable in the present disclosure may be any Cas protein of the CRISPR type II system. In more specific embodiments, the nucleic acid guided DNA binding protein nuclease may be CRISPR-associated endonuclease 9 (Cas9) system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein. It should be appreciated that any type II CRISPR-Cas systems may be applicable in the present invention, specifically, any one of type II-A or B. Thus, in yet some further and alternative embodiments, at least one cas gene used in the modulator of the invention may be at least one cas gene of type II CRISPR system (cither typeII-A or typeII-B). In more particular embodiments, at least one cas gene of type II CRISPR system used by the methods and systems of the invention may be the cas9 gene.

According to such embodiments, the CRISPR-Cas proteins used in the modulator of the invention is a CRISPR-associated endonuclease 9 (Cas9). Double-stranded DNA (dsDNA) cleavage by Cas9 is a hallmark of “type II CRISPR-Cas” immune systems. The CRISPR-associated protein Cas9 is an RNA-guided DNA endonuclease that uses RNA: DNA complementarity to a target site (proto-spacer). After recognition between Cas9 and the target sequence double stranded DNA (dsDNA) cleavage occur, creating the double strand breaks (DSBs).

CRISPR type II system as used herein requires the inclusion of two essential components: a “guide” RNA (gRNA) and a CRISPR-associated endonuclease (Cas9). The gRNA is an RNA molecule composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion or alternatively, annealing of the endogenous tracrRNA with a targeting sequence (also named crRNA), providing both scaffolding/binding ability for Cas9 nuclease and targeting specificity. Also referred to as “single guide RNA” or “sgRNA”.

In yet some further particular embodiments, the class 2 system in accordance with the invention, may be a CRISPR type V system. In a more specific embodiment, the RNA guided DNA binding protein nuclease may be CRISPR-associated endonuclease X (CasX) system or CRISPR-associated endonuclease 14 (Cas14) system or CRISPR-associated endonuclease F (CasF, also known as Cas12j) system. The type V CRISPR-Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF). The gRNA is a short synthetic RNA composed of a “scaffold” sequence necessary for CasX/Cas14/CasF-binding and about 20 nucleotide long “spacer” or “targeting” sequence, which defines the genomic target to be modified.

In yet some alternative embodiments, where the modifier used performs a modulation other than nucleolytic activity, directing the modifier to the target site may result in targeted modulation (e.g., activation or repression, methylation or demethylation and the like) of the target nucleic acid sequence targeted by the gRNA, thereby affecting the expression, distribution, stability, and/or activity of the target signaling molecule (e.g., SNS3, p38, p62, NBR1, NUP3). It should be noted that a target recognition element (e.g., the gRNA) may comprise between about 3 nucleotides to about 100 nucleotides, specifically, 3, 4, 5, 6, 7, 8, 9, 10, 15, 20, 25, 30, 35, 40, 45, 50, 55, 60, 65, 70, 75, 80, 85, 90, 95 100 or more. More specifically between about 10 nucleotides to 70 nucleotides or more.

It should be appreciated that any CRISPR/Cas proteins may be used by the present disclosure, in some embodiments of the present disclosure, the endonuclease may be a Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus β4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Rosciflexus castenholzii (DSM 13941), Roseiflexus RS1, Syncchocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiac equisimilis GGS 124, Streptococcus equi zooepidemicus MGCS10565, Streptococcus gallolyticus UCN34 (uid46061), Streptococcus gordonii Challis subst CHI, Streptococcus mutans NN2025 (uid46353), Streptococcus mutans, Streptococcus pyogenes MI GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Marce, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penctrans, Mycoplasma synoviac 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroscobacter shibac. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter ciseniac EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

In some embodiments, the at least one nucleic acid guided genome modifier protein of the modulator of the present disclosure, and specifically chimeras thereof, may comprise at least one defective enzyme. A defective enzyme (e.g., a defective mutant, variant or fragment) may relate to an enzyme that displays an activity reduced in about 1%, 10%, 50% to about 100%, as compared to the wild type active nuclease. The present disclosure therefore further encompasses the use of a defective Cas protein dCas, fused to a modifier that may be either a transcription factor or repressor, methyl transferase, thereby affecting the expression of the disclosed target signaling molecule.

Taken together, in some specific embodiment, useful modulatory compounds that may target the target signaling molecule at the nucleic acid level (a), thereby affecting the expression, distribution and/or splicing of the at least one target signaling molecules (thereby modulating proteasome dynamics in the cell), may be or may comprise gRNA, small interfering RNA (siRNA), short hairpin RNA (shRNA), microRNA (miRNA), MicroRNA-like RNAs (milRNA), and/or artificial miRNAs (amiRNA). Non-limiting embodiments for specific gRNAs, siRNAs, and/or shRNAs targeting each of the disclosed signaling molecules, are disclosed by the present disclosure. Non-limiting embodiments, include any of the compounds that comprise the nucleic acid sequences of any one of SEQ ID NO: 1 to 12, SEQ ID NO: 14 to 24, and SEQ ID NO: 49 to 60, or any derivatives and variants thereof.

In yet some further additional or alternative embodiments, the modulatory compounds that may target the target signaling molecule at the protein level (b). According to some further embodiments, the compound may reduce the stability of said target protein by targeted protein degradation (TPD).

As indicated herein, the disclosed modulator (a compound that modulates proteasome dynamics) of the present disclosure may be a compound that targets a signaling molecule that participates in proteasome dynamics at the protein level. In some embodiments, such modulators may be any targeted protein degrader (TPDs). In some embodiments, the TPDs applicable as the modulator of the present disclosure may be a targeted protein degrader based on proteasome. Bifunctional hybrid-molecules that target UPS mediated degradation of a target protein by bridging between the target and the E3 ligase, are also known as Proteolysis Targeting Chimeric ligands (PROTAC compounds) that induce ubiquitination by the use of a ligase, such as E3 ligase and degrade a protein of interest. Thus, in some embodiments thereof, the modulator of the present disclosure may be or may comprise a PROTAC molecule. More specifically, PROTACs, as used herein, are typically designed with three parts: (1) a ligand/molecule that binds to and/or modulates ubiquitin ligases; (2) a binding moiety that targets and recruits the protein of interest for proteolysis, e.g., any peptide that recruits any of the target signaling molecules (SNS3, p38, p63, NBR1, NUP93); and (3) a linker that links the two molecules together. PROTACs thus function by allowing the ligand/molecule to bind to the ubiquitin ligases, thereby recruiting the target of protein of interest to the ligase for ubiquitination and ultimately proteolysis and degradation.

Other names can however be found in the literature: e.g., specific and non-genetic IAP-dependent protein erasers (SNIPER); degrader; degronimids; PROtcolysis TArgeting Peptide (PROTAP); Protein Degradation Probe (PDP). PROTACs hijack the catalytic activity of ubiquitin E3 ligases to mediate proteasome dependent degradation of selected protein of interest (POI), by bringing the ligase and POI into close spatial proximity and initiating the poly-ubiquitination process. It should be appreciated that the present disclosure further encompasses also similar or corresponding CLIPTAC molecules. In some embodiments, the term “CLIPTAC” defines a proteolysis targeting chimeric molecule (PROTAC) formed from the intracellular self-assembly of precursors via bioorthogonal click chemistry (CLIckable Proteolysis TArgeting Chimera chimeric molecule), that refers to any chemical reaction that can occur inside of living systems. It should be understood that the present disclosure encompasses any PROTAC, CLIPTAC, or any bifunctional hybrid-molecule, conjugate or complexes that comprise any of the E3 ligands, any target (e.g., SNS3, p38, p62, NBR1, NUP93) recruiting peptides and any appropriate linker and any combinations thereof.

Still further, the present disclosure also encompasses the use of molecular glues degrader. More specifically, molecular glue degraders are a class of small molecules that induce novel interactions between E3 ubiquitin ligase substrate receptors and target proteins, leading to the degradation of the target protein. A notable example of molecular glue is the thalidomide-based compounds, which redirect the E3 ubiquitin ligase CRL4CRBN, thereby polyubiquitinating the target proteins, leading to the degradation thereof by the proteasome. Additional embodiments for degraders that may be applicable by the present disclosure may include CHAMP (Chaperone-mediated Protein Degradation/Degrader), LYTAC, ATAC, AbTAC, GlucTAC, AUTAC, ATTEC, AUTOTAC, etc.

As indicated above, in some further embodiments, mediators of amino acid sensing include the Sestrin family. The stress-indued proteins Sestrins are conserved proteins that accumulate in cells exposed to stress, potentiate adenosine monophosphate (AMP)-activated protein kinase (AMPK), and inhibit activation of Target of rapamycin (TOR). Members of the Sestrins family of stress-induced proteins include Sestrin 1 (SESN1), Sestrin 2 (SSEN2) and Sestrin 3 (SESN3). In some embodiments, the signaling molecule affected by the disclosed modulator used in the therapeutic methods may be Sestrin 3. Thus, in some embodiments, the modulating compound used in the disclosed methods is a compound that targets SESN3.

More specifically, Sestrin3 (also referred to herein as SESN3), is a protein that in humans is encoded by the SESN3 gene. The encoded protein reduces the levels of intracellular reactive oxygen species induced by activated Ras downstream of RAC-alpha serine/threonine-protein kinase (Akt) and FoxO transcription factor. The protein is required for normal regulation of blood glucose, insulin resistance and plays a role in lipid storage in obesity. Alternative splicing results in multiple transcript variants.

In some embodiments SESN3 is the human SESN3, and any isoforms thereof. In some other embodiments the human SESN3 is encoded by a nucleic acid sequence as denoted by CCDS 8303.1. In some other embodiments the human SESN3 is encoded by a nucleic acid sequence comprising the sequence as denoted by SEQ ID NO: 34, or any homologs or derivatives thereof. In yet some further embodiments, SESN3 encoded by the disclosed nucleic acid sequence is the human SESN3 protein that comprises the amino acid sequence as denoted by Uniport number: P58005-1. In yet some further embodiments, the SESN3 amino acid sequence comprise SEQ ID NO: 35. Still further, the present disclosure refers to SESN3 isoform encoded by the nucleic acid sequence as disclosed by CCDS 60938.1. In yet some further embodiments, the SESN3 second isoform is encoded by a nucleic acid sequence comprising the sequence as denoted by SEQ ID NO: 36. In yet some further embodiments, such SESN3 isoform comprises the amino acid sequence as denoted by SEQ ID NO: 37.

Accordingly, the disclosed methods comprise the step of administering to the subject at least one compound that reduces the level and/or activity of Sestrin3. In some embodiments, the compound used in the disclosed methods leads to reduction of Sestrin3 levels and/or activity by targeting SESN3 at the nucleic acid sequence level, and/or by targeting SESN3 at the protein level. In more specific embodiments the disclosed compound may act by at least one of: (i) specifically targeting a nucleic acid sequence encoding said Sestrin3, or any parts thereof; (ii) specifically targeting a nucleic acid sequence involves directly or indirectly in regulation of the Sestrin3 gene expression; (iii) reducing the stability (increasing degradation) of the Sesn3 protein; and/or (iv) interfering with the interaction of Sestrin3 with at least one regulatory complex.

As will be elaborated herein after, any compound that display one or more of the features discussed above, may be used in the disclosed methods. In some embodiments, for example when Sestrin3 is concerned, any inhibitory/modulatory non-coding nucleic acid molecule may be used to target and specifically reduce the expression and/or activity of such Sestrin3 target. In some embodiments, such nucleic acid molecules and/or sequences and/or compounds may include in some embodiments a ribonucleic acid (RNA) molecule, such RNA molecule may be at least one of a double-stranded RNA (dsRNA), an antisense RNA, a single-stranded RNA (ssRNA), and a Ribozyme specifically targeted at Sestrin3. In yet some further specific embodiments, at least one inhibitory/modulatory non-coding nucleic acid molecule may be at least one of a microRNA (miRNA), MicroRNA-like RNAs (milRNA), artificial miRNAs (amiRNA) and short hairpin RNA (shRNA).

Still further, in some embodiments the compound used in the methods of the present disclosure may comprise a gene editing system that targets the nucleic acid sequence encoding Sestrin3. Specifically, in some embodiments, the compound of the present disclosure is any gene editing system or any component/s thereof. Thus, the disclosed methods comprise the use of a gene editing system that targets the Sestrin3 thereby leading to reduction in the expression and/or activity thereof. In some specific embodiments, the compound used by the methods of the present disclosure comprise: (a) at least one RNA guide (gRNA) that guides least one nucleic acid guided genome modifier protein to at least one target sequence within the Sestrin3 encoding nucleic acid sequence, or within a nucleic acid sequence involves directly or indirectly in regulation of the Sestrin3 gene expression, or at least one nucleic acid sequence encoding said nucleic acid guide. The compound of the present disclosure may further comprise in some optional embodiments thereof (b), at least one nucleic acid guided genome modifier protein, or any chimeric protein, complex or conjugate thereof, or at least one nucleic acid sequence encoding the guided genome modifier protein or chimeric protein thereof. In some embodiments, mediators of metabolite sensing that may be applicable in the present disclosure may include mediators of amino acid sensing.

In some embodiments, the modulator of the present disclosure comprises a nucleic acid molecule that specifically targets the nucleic acid sequence encoding Sestrin3. Alternatively, the modulator of the present disclosure comprises a nucleic acid molecule THAT may target a regulatory sequence that controls the expression and/or distribution of Sestrin3. For example, any regulatory sequence such as promoter, enhancer, splicing site (donor and/or acceptor), miRNA, long non-coding RNA and the like, or any other sequence comprised within a non-coding sequence. In some embodiments, such modulator that is a nucleic acid-based molecule may comprise at least one of a single strand ribonucleic acid (RNA) molecule, a double strand RNA molecule, a single-strand DNA molecule (ssDNA), a double strand DNA (dsDNA), a modified deoxy ribonucleotide (DNA) molecule, a modified RNA molecule, a locked-nucleic acid molecule (LNA), a peptide-nucleic acid molecule (PNA) and any hybrids or combinations thereof.

In some specific embodiments, the compound of the method of the present disclosure may comprise at least one nucleic acid guide that is specifically targeted at nucleic acid sequences that encode the Sestrin3, or at any regulatory elements or sequences thereof. The guide nucleic acid sequence targets the modifier protein towards the target nucleic acid sequences, for example, Sestrin3. Consequently, the modifier modifies the target nucleic acid sequence so as to lead to reduction in the expression and/or activity of the target Sestrin3. In yet some further embodiments, the at least one nucleic acid guided genome modifier protein, chimeric protein, complex or conjugate, comprises at least one nucleic acid modifier component and at least one component capable of binding the at least one nucleic acid guide.

In more specific embodiments, the at least one nucleic acid modifier component is a protein-based modifier, a nucleic acid-based modifier or any combinations thereof. In yet some further embodiments, the protein-based modifier is at least one of a nuclease, a methyltransferase, a methylated DNA binding factor, a transcription factor, a transcription repressor, a chromatin remodeling factor, a polymerase, a demethylase, an acetylase, a deacetylase, a kinase, a phosphatase, an integrase, a recombinase, a ligase, a topoisomerase, a girase, a helicase, and any combinations thereof.

In more specific embodiments, the at least one nucleic acid modifier component comprises at least one clustered regularly interspaced short palindromic repeats (CRISPR)-Cas protein, cas protein derived domain and/or any variant and mutant thereof.

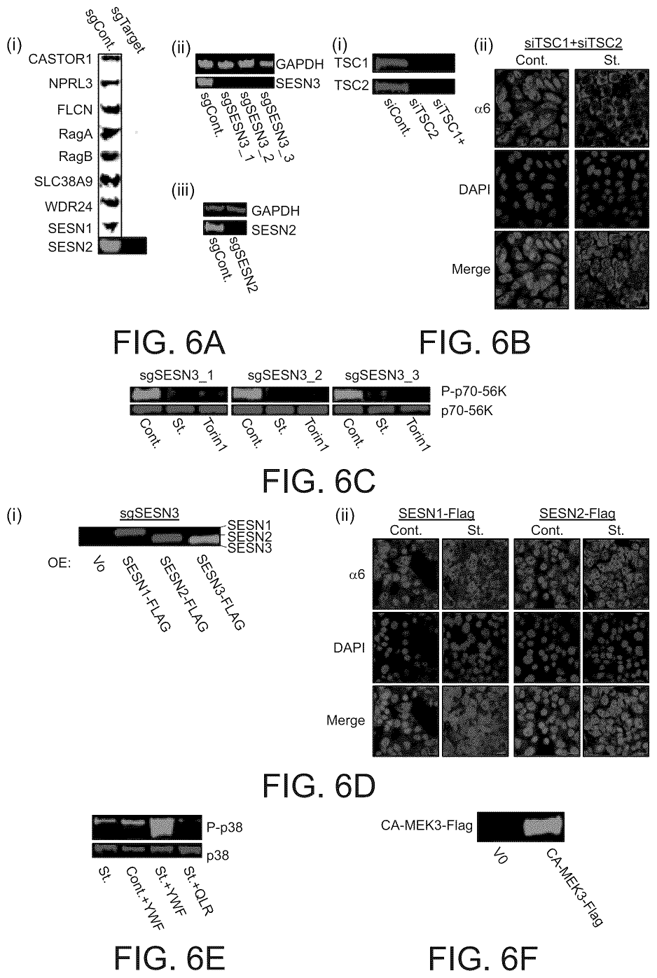

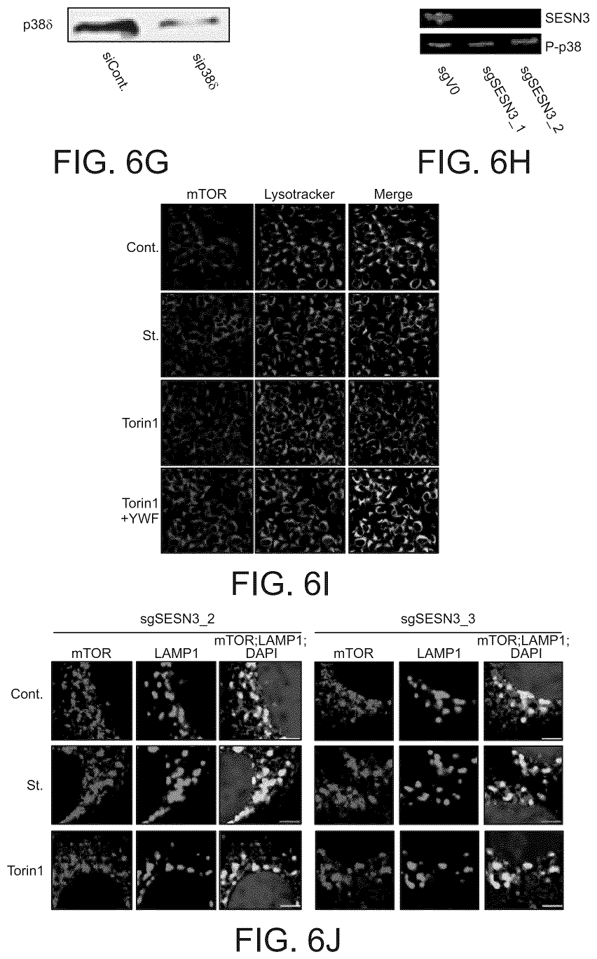

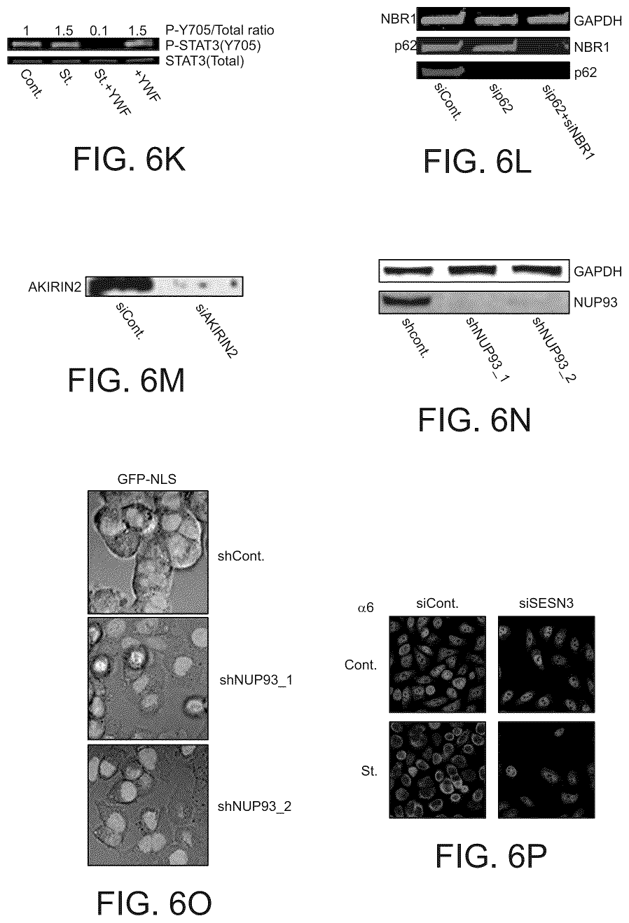

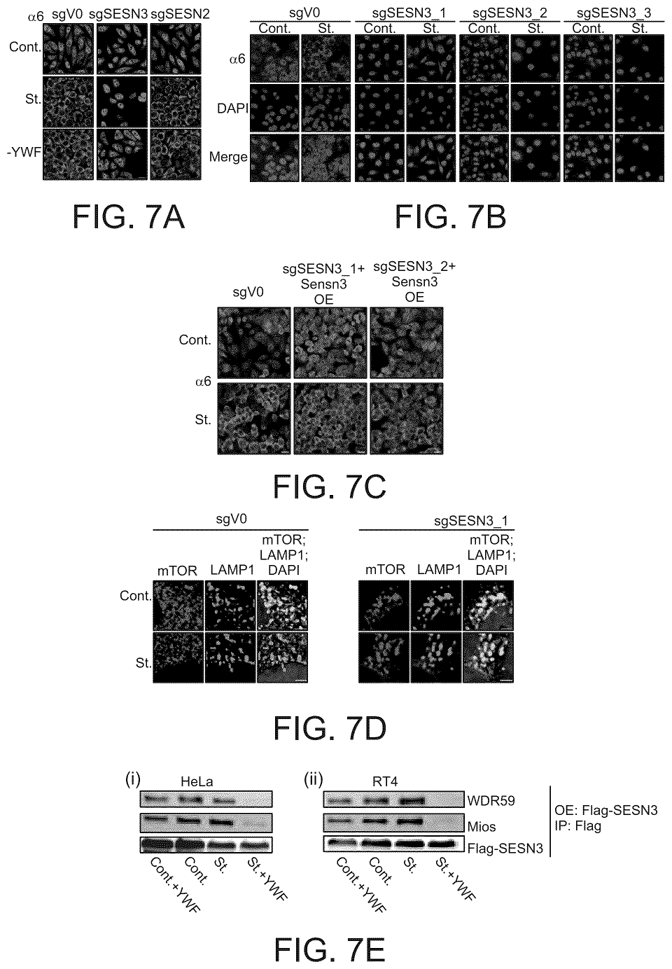

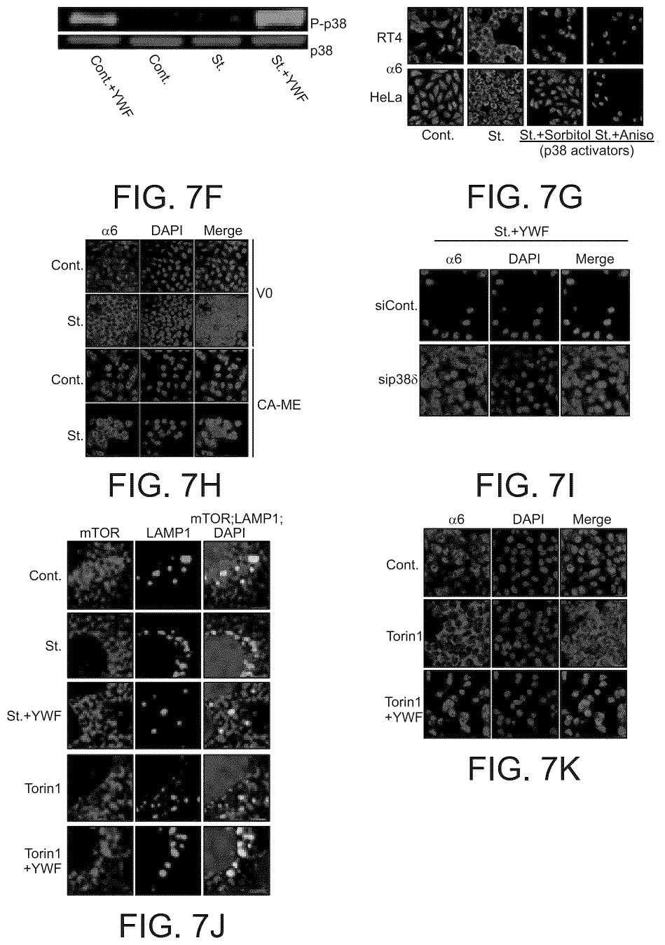

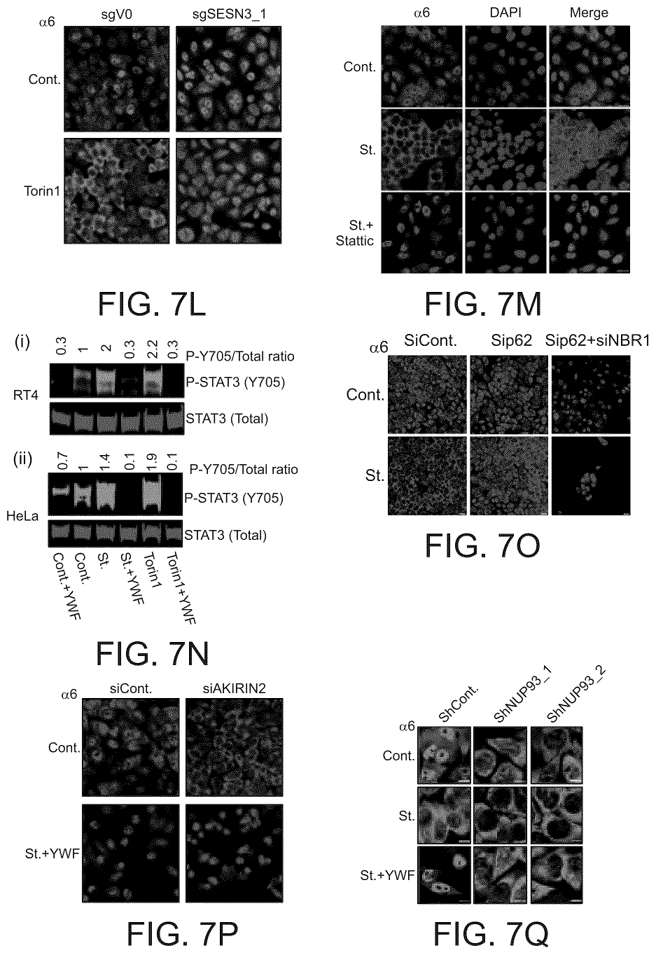

In some embodiments, the modulating compound used by the methods disclosed herein comprises at least one single guide RNA (sgRNA) that specifically recognizes and binds at least one target sequence within the Sestrin3 gene, or any nucleic acid sequence encoding these at least one sgRNA. In yet some specific and non-limiting embodiments, the sgRNA comprises the nucleic acid sequence as denoted by any one of SEQ ID NO: 1, 2, and 3, or any combinations thereof. The disclosed sgRNAs are designated herein as sgSESN3_1, 2 and 3, respectively. In some embodiments, the compound used by the disclosed methods may further comprise a Cas nuclease, or any nucleic acid sequence encoding such Cas nuclease, or any nucleic acid vector or vehicle that comprise nucleic acid sequences encoding the gRNA and/or the Cas protein. As shown by the Examples (e.g. FIGS. 6 and 7), the use of these gRNAs as modulating compounds, to guide the Cas9 nuclease to the target sequence within the Sesn3 gene, and abolish the expression thereof. Such manipulation resulted in a complete modulation of the proteasome dynamics, as reflected by nuclear localization of the proteasome. Examples for suitable expression vectors or vehicles suitable for the sgRNAs, will be disclosed herein after.

In yet some further embodiments, the compound used by the methods disclosed herein comprises at least one siRNA that targets the Sestrin3 nucleic acid sequence. In some embodiments, the siRNA may comprise the nucleic acid sequence as denoted by SEQ ID NO: 49, 50, 51, 52, or any combinations thereof.

In yet some additional or alternative embodiments, the Sestrin3 may be targeted functionally, by the compounds used by the methods of the present disclosure. More specifically, the compound used by the disclosed methods may interfere with Sestrin3 function, in some embodiments, by blocking any downstream pathways and/or interactions thereof. Thus, in some embodiments, the compound of the disclosed methods may be any compound that interferes and/or blocks, and/or prevents, and/or reduces the interaction of Sestrin3 with at least one regulatory complex. As firstly shown by Example 9, by physically interacting with GAP activity towards Rags 2 (GATOR2), Sestrin3 releases GATOR1 from GATOR2-mediated inhibition. GATOR1 then inhibits RagB GTPase and subsequently prevents mTORC1 activation by amino acids. In the presence of YWF, the inhibitory interaction of Sestrin3 with the GATOR2 complex is suppressed, and as such, YWF, or any other compound that interferes with the interaction of Sestrin3 with at least one member of the GATOR2 complexes, may lead to activation of mTOR. As shown in the present disclosure, blockage of the interaction of Sestrin3 with at least one of MIOS and/or WDR59, that are members of the GATOR2 complex, releases the complex, that can subsequently suppress GATOR1, thereby activating mTOR. In yet some alternative or additional embodiments, the modulatory compound used in the disclosed methods may target SESN3 at the protein level. Thus, according to some embodiments, the modulatory compound may (a), reduce the stability of SESN3 by targeted protein degradation (TPD). In yet some further additional and/or alternative embodiments, the compounds used in the disclosed method may affect the activity of SESN3 by affecting or modulating the interaction of Sestrin3 with a regulatory complex (b). Thus, in some embodiments, the compound/s used by the methods of the present disclosure may be any compound that interferes with, and/or blocks, and/or inhabits, and/or reduces, and/or decreases and/or prevents the inhibitory interaction of Sestrin3 with at least one member of the GATOR2 complex, specifically, with MIOS and/or WDR59, and optionally, with SEHIL and SEC13, but not with WDR24. In some specific and non-limiting embodiments, any compound that blocks the interaction of Sestrin3 and MIOS and/or WDR59, may be used by the disclosed methods, with the proviso that the compound is not the YWF triad.

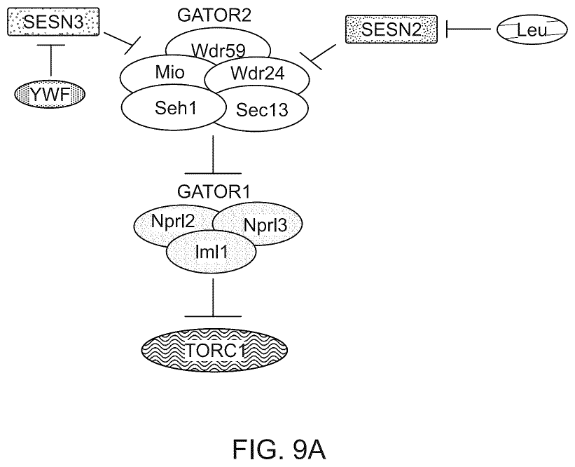

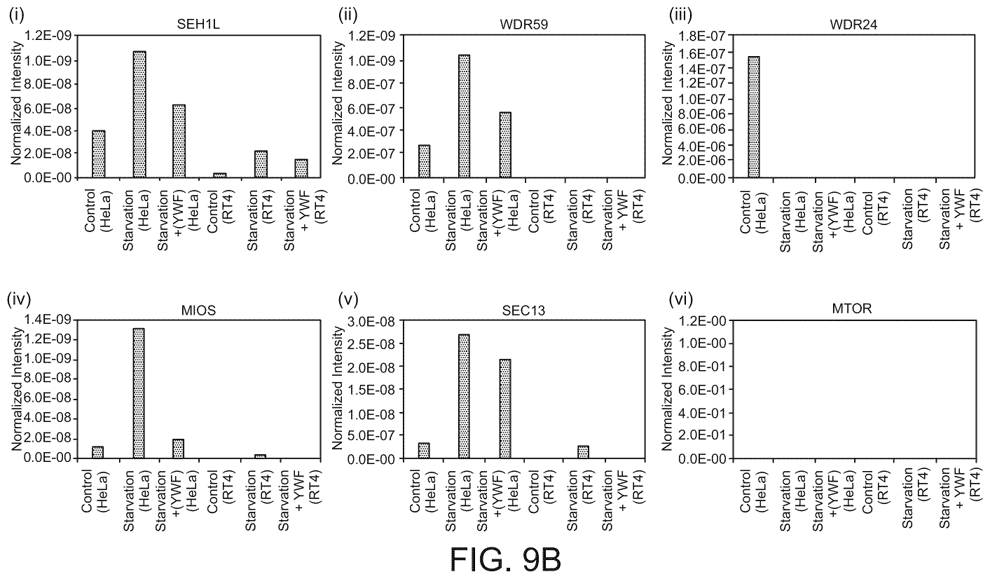

Still further, in some alternative embodiments it should be appreciated that where inhibition of mTOR is desired, any compound that enhances and/or increases the inhibitory interaction of Sestrin3 with members of the GATOR2 complex, may be used. The GTPase-activating protein (GAP) toward Rags (GATOR) signaling pathway acts upstream of TORC1 and is comprised of two subcomplexes. The trimeric GATOR1 complex (composed of DEPDC5, NPRL2, and NPRL3) inhibits mTORC1 (mammalian target for rapamycin complex 1 (mTORC1)) activity in response to amino acid limitation by serving as a GTPase-activating protein (GAP) for the TORC1 activator RagA/B, a component of the lysosomally located Rag GTPase. The multi-protein GATOR2 complex suppresses the inhibitory function of GATOR1 (GTPase activating protein) toward Rag GTPase. Therefore, GATOR2 functions upstream of GATOR1 as an activator of mTORC1 in amino acid signaling. GATOR2 is a protein complex composed of five different protein subunits, including Mios (meiosis regulator for oocyte development), WDR24 (WD repeat domain 24), WDR59 (WD repeat domain 59), Sch1L and Scc13. Mios, WDR24, WDR59 Sch1L and SEC13 function within the amino acid-sensing branch of the TORC1 signaling pathway, as components of the GATOR subcomplex GATOR2. They indirectly activate mTORC1 and the TORC1 signaling pathway through the inhibition of the GATOR1 subcomplex. The inventors showed that the inhibitory interaction of SESN3 specifically with the GATOR2 complex members Mios, WDR59, SEHIL and SEC13 (more significantly with Mios and WDR59) is elevated following amino acid starvation of YWF (FIGS. 7E and 9A-B), leading to mTOR inhibition. On the other hand, the inhibitory interaction of SESN2 with the GATOR2 complex following amino acid starvation of Leu is through WDR24 subunit of GATOR2.

In yet some further embodiments, the modulatory compound targets as a signaling molecule that participates in proteasome dynamics, p38, specifically, p38 delta. Accordingly, the disclosed methods may comprise administering to the subject at least one compound that increases the level and/or activity of p38. p38 mitogen-activated protein kinases (MP kinase, MAPK), also called RK or CSBP (Cytokinin Specific Binding Protein), are a class of mitogen-activated protein kinases (MAPKs), that are activated by a variety of cellular stresses including osmotic shock, heat shock, inflammatory cytokines, lipopolysaccharides (LPS), ultraviolet light, and growth factors. MSPKs are involved in cell differentiation, apoptosis and autophagy. In some embodiments, the disclosed MAPKs affected by the modulator used in the disclosed methods, may be a member of the p38 MAPKs. More specifically, four p38 MAP kinases, p38-α (MAPK14), -β (MAPK11), -γ (MAPK12/ERK6), and -δ (MAPK13/SAPK4), have been identified. MKK3, MKK6 and SEK activate p38 MAP kinase by phosphorylation at Thr-180 and Tyr-182. Activated p38 MAP kinase may phosphorylate MAPKAP kinase 2 and the transcription factors ATF2, Mac, MEF2, and p53. p38 also has been shown to phosphorylate post-transcriptional regulating factors like TTP, and in fruit flies it plays a role in regulating the circadian clock. In some embodiments, the modulators of the present disclosure affect, the p38-δ (MAPK13/SAPK4). More specifically, p38δ (also known as SAPK4) has a TGY dual phosphorylation motif and is activated in response to cellular stresses and proinflammatory cytokines. Transcription factor ATF2, and microtubule dynamics regulator stathmin have been shown to be the substrates of this kinase. The inventors found that activated p388 MAP kinase activates mTOR, and induces the association of mTOR to the lysosomal membrane.

In some embodiments p38 is the p38 delta, and as used herein, is the human p38 delta. In some other embodiments p38 is encoded by a nucleic acid sequence comprising the sequence as denoted by CCDS 4818.1. In some specific embodiments, the p38 delta nucleic acid sequence comprises SEQ ID NO: 32, or any homologs or derivatives thereof. In yet some further embodiments, p388 encoded by the disclosed nucleic acid sequence is the human p388 protein that comprises the amino acid sequence as denoted by Uniprot number: O15264. In some specific embodiments, the p388 protein comprises the amino acid sequence as denoted by SEQ ID NO: 33. In some specific embodiments, the compound is a p38 activator. In yest some further specific embodiments, the modulator used herein leads to phosphorylation of at least one of Thr180 (T180) and/or Tyr 182 (Y182) of p38. Like all MAP kinases, p38 kinases are activated by dual kinases termed the MAP kinase kinases (MKKs). However, despite conserved dual phosphorylation sites among p38 isoforms, selective p38 activation by distinct MKKs has been observed. There are two main MAPKKs that are known to activate p38, MKK3 and MKK6. Despite 80% homology between these two MKKs, MKK3 is unable to effectively activate p38ß, while MKK6 is a potent activator. Additionally, MKK4, an upstream kinase of JNK, can aid in the activation of p38a and p388 in specific cell types.

Still further, in some specific and non-limiting embodiments, a p38 activator useful in the disclosed methods may be a compound elevating the levels and/or activity of MAP kinase kinase 3 (MKK3) and/or of MKK6. More specifically, Mitogen-activated protein kinase 3, also known as p44MAPK and ERK1, is an enzyme that in humans is encoded by the MAPK3 gene. The protein encoded by this gene is a member of the mitogen-activated protein kinase (MAP kinase) family. MAP kinases, also known as extracellular signal-regulated kinases (ERKs), act in a signaling cascade that regulates various cellular processes such as proliferation, differentiation, and cell cycle progression in response to a variety of extracellular signals. This kinase is activated by upstream kinases, resulting in its translocation to the nucleus where it phosphorylates nuclear targets. Alternatively spliced transcript variants encoding different protein isoforms have been described. In some embodiments MKK3 is the human MKK3. In some other embodiments the MKK3 comprises the amino acid sequence as denoted by Uniprot number: Q16644. Still further, in some embodiments, the MKK3 may comprise the amino acid sequence as denoted by SEQ ID NO: 47. Still further, in some embodiments a p38 delta activator as used herein may be the MKK6. MAPKK 6 is a member of the dual specificity protein kinase family, which functions as a mitogen-activated protein (MAP) kinase kinase. MAP kinases, also known as extracellular signal-regulated kinases (ERKs), act as an integration point for multiple biochemical signals. This protein phosphorylates and activates p38 MAP kinase in response to inflammatory cytokines or environmental stress. As an essential component of p38 MAP kinase mediated signal transduction pathway, this gene is involved in many cellular processes such as stress-induced cell cycle arrest, transcription activation and apoptosis.

In some embodiments MKK6 is the human MKK6. In some other embodiments, MKK6 comprises the amino acid sequence as denoted by Uniprot number: Q16644. Still further, in some embodiments, the MKK3 may comprise the amino acid sequence as denoted by SEQ ID NO: 48. In yet some further additional or alternative embodiments, a p38 activator useful in the disclosed methods may be at least one hyperosmotic agent. Non-limiting embodiments for such hyperosmotic agent, may be sorbitol. Thus, in some embodiments, any carbohydrates having a hyperosmotic effect may be used. To name but few, glycerin (glycerol), isosorbide, mannitol, hypertonic saline (HTS) and urea may be used as the disclosed compounds. In some embodiments, the compound used by the methods of the present disclosure may be sorbitol. Still further, in some additional or alternative embodiments, a p38 activator useful in the disclosed methods may be at least one DNA Synthesis Inhibitor. In some specific embodiments, such compound may be anisomycin. Additional p38 activators that may be useful in the present disclosure include AEBSF hydrochloride, Sappanone, Metformin HCl (1,1-Dimethylbiguanide HCl), ML141 (CID-2950007), Berberine (Natural Yellow 18) chloride hydrate and Asiatic acid (Dammarolic acid, Asiantic acid).

In yet some further embodiments, the modulatory compound targets p62, or NBR1. Since as shown by the present Examples, for modulating the proteasome dynamics silencing of both, p62 and NBR1 is required, the disclosed methods may comprise administering to the subject at least one compound that reduces the level and/or activity of p62 and at least one compound that reduces the level and/or activity of NBR1. As shown by the Examples, addition of siRNAs for p62 and for NBR1 that leads to silencing of both targets, led to nuclear localization of the proteasome. More specifically, Sequestosome-1 (Also known as the ubiquitin-binding protein p62) is a protein that in humans is encoded by the SQSTM1 gene. p62 is an autophagosome cargo protein that targets other proteins that bind to it for selective autophagy. The inventors found that p62 together with NBR1 is involved in proteasome shuttling from the cytosol to the nucleus.

In some embodiments p62 is the human p62. In some other embodiments p62 is encoded by a nucleic acid sequence comprising the sequence as denoted by CCDS 34317.1. In some specific embodiments, the p62 nucleic acid sequence comprises SEQ ID NO: 28, or any homologs or derivatives thereof. In yet some further embodiments, p62 encoded by the disclosed nucleic acid sequence is the human p62 protein that comprises the amino acid sequence as denoted by Uniprot Number: Q13501. In some specific embodiments, the p62 protein comprises the amino acid sequence as denoted by SEQ ID NO: 29, and any isoforms thereof. Still further, in some alternative embodiments, the human p62 as referred to herein relates to the p62 isoform that is encoded by the nucleic acid sequence as denoted by CCDS 47355.1. In some embodiments, the nucleic acid sequence is as denoted by SEQ ID NO: 30. Still further, in some embodiments, p62 isoform encoded by the disclosed nucleic acid sequence is the human p62 protein that comprises the amino acid sequence as denoted by SEQ ID NO: 31, and any isoforms thereof.

Still further, Neighbor of BRCA1 gene 1 protein is a protein that in humans is encoded by the NBR1 gene. The encoded protein contains a B-box/coiled coil motif, which is present in many genes with transformation potential. This gene is located on a region of chromosome 17q21.1 that is in close proximity to tumor suppressor gene BRCA1. Three alternatively spliced variants encoding the same protein have been identified for this gene. One implied function lies in autophagy, where it acts a cargo receptor in selective autophagy.

In some embodiments NBR1 is the human NBR1. In some other embodiments NBR1 is encoded by a nucleic acid sequence comprising the sequence as denoted by CCDS 45694.1. In some specific embodiments, the NBR1 nucleic acid sequence comprises SEQ ID NO: 38, or any homologs or derivatives thereof. In yet some further embodiments, NBR1 encoded by the disclosed nucleic acid sequence is the human NBR1 protein that comprises the amino acid sequence as denoted by Uniprot Number: Q14596. In some specific embodiments, the NBR1 protein comprises the amino acid sequence as denoted by SEQ ID NO: 39, and any isoforms thereof. Still further, in some alternative embodiments, the human NBR1 as referred to herein relates to the NBR1 isoform that is encoded by the nucleic acid sequence as denoted by CCDS77037.1. In some embodiments, the nucleic acid sequence is as denoted by SEQ ID NO: 40. Still further, in some embodiments, NBR1 isoform encoded by the disclosed nucleic acid sequence is the human NBR1 protein that comprises the amino acid sequence as denoted by SEQ ID NO: 41, and any isoforms thereof. In some embodiments the modulators of the present disclosure may comprise at least one nucleic acid molecule, specifically, at least one siRNA molecule specific for p62, and at least one siRNA molecule specific for NBR1. Non-limiting embodiments for such modulators that are applicable in the present disclosure include the siRNA molecules that comprise the nucleic acid sequence as denoted by SEQ ID NO: 53, 54, 55, 56, 57, 58, 59 and 60, or any combinations thereof.

In yet some further embodiments, the disclosed method may use any NUP93 activator for tilting towards nuclear localization of the proteasome. Nucleoporin 93 (Nup93) is a protein that in humans is encoded by the NUP93 gene. The encoded protein is a target of caspase cysteine proteases that play a central role in programmed cell death by apoptosis. The inventors found that silencing of NUP93 gene resulted in a predominant cytosolic distribution of the proteasome, indicating that NUP93 is involved in proteasome sequestration into the nucleus.

In some embodiments NUP93 is the human NUP93. In some other embodiments NUP93 is encoded by a nucleic acid sequence comprising the sequence as denoted by CCDS 10769.1. In some specific embodiments, the NUP93 nucleic acid sequence comprises SEQ ID NO: 42, or any homologs or derivatives thereof. In yet some further embodiments, NUP93 encoded by the disclosed nucleic acid sequence is the human NUP93 protein that comprises the amino acid sequence as denoted by Uniprot Number: Q8NIF7-1. In some specific embodiments, the NUP93 protein comprises the amino acid sequence as denoted by SEQ ID NO: 43, and any isoforms thereof. Still further, in some alternative embodiments, the human NUP93 as referred to herein relates to the NUP93 isoform that is encoded by the nucleic acid sequence as denoted by CCDS55996.1. In some embodiments, the nucleic acid sequence is as denoted by SEQ ID NO: 44. Still further, in some embodiments, NUP93 isoform encoded by the disclosed nucleic acid sequence is the human NUP93 protein that comprises the amino acid sequence as denoted by SEQ ID NO: 45, and any isoforms thereof.

In yet some further alternative or additional embodiments, the compound useful in the methods of the present disclosure may target STAT3. More specifically, in some embodiments, the compound used in the disclosed therapeutic methods may be any STAT3 inhibitor, for example, any compound that inhibits and/or reduces phosphorylation of STAT3. Signal transducer and activator of transcription 3 (STAT3) is a transcription factor which in humans is encoded by the STAT3 gene and is a member of the STAT protein family. STAT3 is phosphorylated by receptor-associated Janus kinases (JAK) in response to cytokines and growth factors, forms homo- or heterodimers, and translocate to the cell nucleus where it acts as a transcription activator. Specifically, STAT3 becomes activated after phosphorylation of tyrosine 705 in response to such ligands as interferons, epidermal growth factor (EGF), Interleukin (IL-) 5 and IL-6. Additionally, activation of STAT3 may occur via phosphorylation of serine 727 by Mitogen-activated protein kinases (MAPK) and through c-src non-receptor tyrosine kinase. STAT3 mediates the expression of a variety of genes in response to cell stimuli, and thus plays a key role in many cellular processes such as cell growth and apoptosis.

In some embodiments STAT3 is the human STAT3. In yet some further specific embodiments STAT3 as used herein comprises the amino acid sequence as denoted by Uniprot number: P40763-1. In more specific embodiments, STAT3 comprises the amino acid sequence as denoted by SEQ ID NO: 46.

In some particular embodiments, any compound that inhibits and/or reduces phosphorylation of Tyr705 of STAT3.

In some specific and non-limiting embodiments, STAT3 inhibitors that may be useful in the methods disclosed herein may include small molecule compounds, specifically, Stattic (Stat three inhibitory compound), S31-201/NSC74859, BP-1-102, Niclosamide, peptide inhibitors (e.g., the peptide aptamer APT STAT3-9R, and the like), Artesunate, Galicllalactone, HJC 0416 hydrochloride, 5, 15-DPP, Cucurbitacin I, Napabucasin, Colivelin (TFA). In some specific embodiments, Stattic may be used as the compound of the methods of the present disclosure.

By targeting various signaling molecules as discussed above, the present disclosure provides therapeutic methods useful in the treatment of any pathologic disorder. In some embodiments, the methods of the present disclosure may be applicable for any disorder affected by proteasomal activity and/or proteasomal cellular localization. In some specific embodiments, such disorder is at least one of: at least one neoplastic disorder and/or at least one protein misfolding disorder or deposition disorder.

Still further, in some embodiments, the disclosed methods may be applicable for any malignant and non-malignant neoplastic disorders. In some specific embodiments, the disclosed methods may be used for treating malignant neoplastic disorder.

The present disclosure provides therapeutic and prophylactic methods applicable for any condition or pathologic disorder that requires, is associated with, or is characterized by, cytosolic localization, accumulation and/or activity of the proteasome. More specifically, the methods discussed herein are applicable for any disorder or condition characterized with, or defined by, predominant proteasome cytosolic localization, or by accumulation of the proteasome in the cytosol and/or increased activity of the proteasome in the cytosol, specifically, as compared with cells of a healthy subject or of a subject not suffering from the indicated disorder. In some embodiments, the disorders discussed herein may be any disorders characterized with proteasome malfunction, that may refer in some embodiments to increased activity. As indicated herein, the increased amount and/or activity of the proteasome in the cytosol of cells of the subject, is essential for providing the unmet need, or demand of the cells for energy sources, amino acids and/or recycled building blocks required for cell survival, and activity. Still further, the proteasome activity, as referred to herein, refers to proteolytic degradation of various cytoplasmic and nuclear proteins. The proteasome activity can be measured by any known methods, that may include for example, the use of fluorescently tagged proteasome subunits and the use of activity-based proteasome probes. Methods for determining proteasome localization are discussed herein after in connection with other aspects of the invention.

Still further, the modulating compounds, compositions of the present disclosure may be applicable for any proliferative disorder that may be in some embodiments, any neoplastic disease, more specifically, any abnormal mass of tissue, also referred to herein as a tumor, that is formed due to uncontrolled or abnormal cell growth that results increased cell number. The methods of the present disclosure may be applicable in some embodiments for any neoplasms, either benign neoplasms, in situ neoplasms, or malignant neoplasms.

In some embodiments, the methods of the invention may be applicable for treating adenomas. More specifically, adenoma is a benign tumor of epithelial tissue with glandular origin, glandular characteristics, or both. Adenomas can grow from many glandular organs, including the adrenal glands, pituitary gland, thyroid, prostate, and others. Although adenomas are benign, they should be treated as pre-cancerous. Over time adenomas may transform to become malignant, at which point they are called adenocarcinomas. It should be understood that the present invention is further applicable to any metastatic tissue, organ or cavity of any of the disclosed proliferative disorders. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods, compositions and kits of the present invention may be applicable for a patient suffering from any one of non-solid and solid tumors.

Malignancy, as contemplated in the present invention may be any one of carcinomas, melanomas, lymphomas, leukemia, myeloma and sarcomas. Therefore, in some embodiments any of the methods of the invention (specifically, therapeutic, prognostic and non-therapeutic methods), and proteasomal dynamics modulators and any kits and compositions thereof, may be applicable for any of the malignancies disclosed by the present disclosure.

More specifically, carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas. Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

In some embodiments, the methods of the present disclosure may be applicable for any solid tumor. In more specific embodiments, the methods disclosed herein may be applicable for any malignancy that may affect any organ or tissue in any body cavity, for example, the peritoneal cavity (e.g., liposarcoma), the pleural cavity (e.g., mesothelioma, invading lung), any tumor in distinct organs, for example, the urinary bladder, ovary carcinomas, and tumors of the brain meninges. Particular and non-limiting embodiments of tumors applicable in the methods, compositions and kit of the present disclosure may include but are not limited to at least one of ovarian cancer, liver carcinoma, colorectal carcinoma, breast cancer, pancreatic cancer, brain tumors and any related conditions, as well as any metastatic condition, tissue or organ thereof.

In some other embodiments, the methods, the proteasomal dynamics modulators of the present disclosure or any compositions and kits thereof are applicable to colorectal carcinoma, or any malignancy that may affect all organs in the peritoneal cavity, such as liposarcoma for example. In some further embodiments, the methods, the proteasomal dynamics modulators of the present disclosure or any compositions thereof may be relevant to tumors present in the pleural cavity (mesothelioma, invading lung) the urinary bladder, and tumors of the brain meninges. In some particular embodiments, the methods, the proteasomal dynamics modulators of the present disclosure or any compositions and kits thereof, may be applicable for ovarian cancer. It should be further understood that the invention further encompasses any tissue, organ or cavity barring ovarian metastasis, as well as any cancerous condition involving metastasis in ovarian tissue. As used herein, the term “ovarian cancer” is used herein interchangeably with the term “fallopian tube cancer” or “primary peritoneal cancer” referring to a cancer that develops from ovary tissue, fallopian tube tissue or from the peritoneal lining tissue. Early symptoms can include bloating, abdominopelvic pain, and pain in the side. The most typical symptoms of ovarian cancer include bloating, abdominal or pelvic pain or discomfort, back pain, irregular menstruation or postmenopausal vaginal bleeding, pain or bleeding after or during sexual intercourse, difficulty cating, loss of appetite, fatigue, diarrhea, indigestion, heartburn, constipation, nausea, early satiety, and possibly urinary symptoms (including frequent urination and urgent urination). Typically, these symptoms are caused by a mass pressing on the other abdominopelvic organs or from metastases.

The most common type of ovarian cancer, comprising more than 95% of cases, is epithelial ovarian carcinoma. These tumors are believed to start in the cells covering the ovaries, and a large proportion may form at end of the fallopian tubes. Less common types of ovarian cancer include germ cell tumors and sex cord stromal tumors. Ovarian cancers are classified according to the microscopic appearance of their structures (histology or histopathology).

It should be appreciated that ovarian carcinoma as used herein may further include at least one of, Ovarian carcinosarcoma, Choriocarcinoma, Mature teratomas, Embryonal carcinomas and Primary ovarian squamous cell carcinomas.

In yet some other embodiments, the methods, and the proteasomal dynamics modulators of the present disclosure, or any compositions and kits thereof, may be suitable for liver cancer. It should be further understood that the invention further encompasses any tissue, organ or cavity barring liver originated metastasis, as well as any cancerous condition having metastasis of any origin in liver tissue. Liver cancer, also known as hepatic cancer and primary hepatic cancer, is cancer that starts in the liver. Cancer which has spread from elsewhere to the liver, known as liver metastasis, is more common than that which starts in the liver. Symptoms of liver cancer may include a lump or pain in the right side below the rib cage, swelling of the abdomen, yellowish skin, easy bruising, weight loss and weakness.

The leading cause of liver cancer is cirrhosis due to hepatitis B, hepatitis C or alcohol. Other causes include aflatoxin, non-alcoholic fatty liver disease and liver flukes. The most common types are hepatocellular carcinoma (HCC), which makes up 80% of cases, and cholangiocarcinoma. Less common types include mucinous cystic neoplasm and intraductal papillary biliary neoplasm. The diagnosis may be supported by blood tests and medical imaging, with confirmation by tissue biopsy. As used herein, HCC, is the most common type of primary liver cancer in adults and is the most common cause of death in people with cirrhosis. It occurs in the setting of chronic liver inflammation and is most closely linked to chronic viral hepatitis infection (hepatitis B or C) or exposure to toxins such as alcohol or aflatoxin. Certain diseases, such as hemochromatosis, Diabetes mellitus and alpha 1-antitrypsin deficiency, markedly increase the risk of developing HCC. Metabolic syndrome and NASH are also increasingly recognized as risk factors for HCC. Cholangiocarcinoma, also known as bile duct cancer, is a type of cancer that forms in the bile ducts. Symptoms of cholangiocarcinoma may include abdominal pain, yellowish skin, weight loss, generalized itching, and fever. Light colored stool or dark urine may also occur. Other biliary tract cancers include gallbladder cancer and cancer of the ampulla of Vater. Risk factors for cholangiocarcinoma include primary sclerosing cholangitis (an inflammatory disease of the bile ducts), ulcerative colitis, cirrhosis, hepatitis C, hepatitis B, infection with certain liver flukes, and some congenital liver malformations. The diagnosis is suspected based on a combination of blood tests, medical imaging, endoscopy, and sometimes surgical exploration. The disease is confirmed by examination of cells from the tumor under a microscope. It is typically an adenocarcinoma (a cancer that forms glands or secretes mucin).

In other embodiments, the methods, kits and compositions of the present disclosure may be applicable for pancreatic cancer. It should be further understood that the present disclosure further encompasses any tissue, organ or cavity barring pancreatic metastasis, as well as any cancerous condition having metastasis of any origin in the pancreas. Pancreatic cancer arises when cells in the pancreas, a glandular organ behind the stomach, begin to multiply out of control and form a mass. There are a number of types of pancreatic cancer. The most common, pancreatic adenocarcinoma, accounts for about 90% of cases. These adenocarcinomas start within the part of the pancreas which makes digestive enzymes. Several other types of cancer, which collectively represent the majority of the non-adenocarcinomas, can also arise from these cells. One to two percent of cases of pancreatic cancer are neuroendocrine tumors, which arise from the hormone-producing cells of the pancreas. These are generally less aggressive than pancreatic adenocarcinoma.

Signs and symptoms of the most-common form of pancreatic cancer may include yellow skin, abdominal or back pain, unexplained weight loss, light-colored stools, dark urine, and loss of appetite. There are usually no symptoms in the disease's early stages, and symptoms that are specific enough to suggest pancreatic cancer typically do not develop until the disease has reached an advanced stage. By the time of diagnosis, pancreatic cancer has often spread to other parts of the body.

Pancreatic cancer rarely occurs before the age of 40, and more than half of cases of pancreatic adenocarcinoma occur in those over 70. Risk factors for pancreatic cancer include tobacco smoking, obesity, diabetes, and certain rare genetic conditions. Pancreatic cancer is usually diagnosed by a combination of medical imaging techniques such as ultrasound or computed tomography, blood tests, and examination of tissue samples (biopsy).

It should be understood that the methods, compositions and kits of the present disclosure are applicable for any type and/or stage and/or grade of any of the malignant disorders discussed herein or any metastasis thereof. Still further, it must be appreciated that the methods, compositions and kits of the invention may be applicable for invasive as well as non-invasive cancers. When referring to “non-invasive” cancer it should be noted as a cancer that do not grow into or invade normal tissues within or beyond the primary location. When referring to “invasive cancers” it should be noted as cancer that invades and grows in normal, healthy adjacent tissues.

Still further, in some embodiments, the methods, and the proteasomal dynamics modulators of the present disclosure, or any compositions and kits thereof, are applicable for any type and/or stage and/or grade of any metastasis, metastatic cancer or status of any of the cancerous conditions disclosed herein.

As used herein the term “metastatic cancer” or “metastatic status” refers to a cancer that has spread from the place where it first started (primary cancer) to another place in the body. A tumor formed by metastatic cancer cells originated from primary tumors or other metastatic tumors, that spread using the blood and/or lymph systems, is referred to herein as a metastatic tumor or a metastasis. Further malignancies that may find utility in the present disclosure can comprise but are not limited to hematological malignancies (including lymphoma, leukemia, myeloproliferative disorders, Acute lymphoblastic leukemia; Acute myeloid leukemia), hypoplastic and aplastic anemia (both virally induced and idiopathic), myclodysplastic syndromes, all types of parancoplastic syndromes (both immune mediated and idiopathic) and solid tumors (including GI tract, colon, lung, liver, breast, prostate, pancreas and Kaposi's sarcoma. The present disclosure may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma, Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, cpendymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myclogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myclodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pincal astrocytoma; Pineal germinoma; Pincoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma-see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

Accordingly, in some embodiments, the neoplastic disorder is cancer.

A further aspect of the present disclosure relates to a therapeutic effective amount of at least one compound that modulates the proteasome dynamics and/or function in a mammalian cell, for use in a method for treating, preventing, inhibiting, reducing, eliminating, protecting or delaying the onset of at least one condition or at least one pathologic disorder in a subject in need thereof. In some embodiments, a compound applicable in the disclosed uses, is a compound characterized by affecting at least one of: mTOR lysosomal association, proteasome cellular localization, the activity and/or level/s and/or PTM/s, and/or subcellular localization of at least one signaling molecule participating directly or indirectly in at least one pathway mediating said proteasome dynamics and/or function.

In some embodiments of the disclosed use, at least one signaling molecule participating directly or indirectly in the at least one signal transduction pathway mediating the proteasome dynamics and/or function may be at least one mediator of metabolite sensing, and/or at least one stress kinase, and/or at least one nucleo-cytosolic shuttle protein, and/or at least one Nuclear Pore Complex (NPC) protein.

In some specific embodiments of the disclosed uses, the mediator of metabolite sensing may be a mediator of amino acid sensing. In yet some additional or alternative embodiments, the stress kinase may be at least one member of the Mitogen-activated protein kinases (MAPKs).

In some embodiments of the disclosed uses, at least one of: (i) the at least one mediator of amino acid sensing is at least one member of the Sestrin family. In yet some further or additional embodiments, (ii), the at least one member of the MAPKs is at least one member of the p38 mitogen-activated protein kinases (p38 MAPK, p38). In yet some further additional or alternative embodiments, (iii), the at least one nucleo-cytosolic shuttle protein/s is at least one of Sequestosome 1 (SQSTM1, p62) and Neighbor of BRCA1 gene 1 protein (NBR1). Still further, in some additional or alternative embodiments (iv), the at least one NPC is Nucleoporin 93 (NUP93).

In some embodiments of the disclosed uses, the at least one member of the Sestrin family is Sestrin3 (SESN3). In yet some further additional or alternative embodiments, the at least one member of the p38 MAPK family, is the p388 (p38 delta, MAPK13).

In some embodiments of the disclosed uses, the modulatory compound leads to: (I) at least one of: (i) mTOR activation and localization to the lysosomal membrane; (ii) reduction in Sestrin3 levels and/or activity; (iii) activation of p38; (iv) reduction in the levels and/or activity of p62 and NBR1; and/or (v) modulation (specifically, activation) of NUP93. In yet some optional embodiments, the disclosed modulator of proteasome dynamics leads, in addition to at least one of the effects disclosed in (i), (ii), (iii), (i) and/or (v), also (II), proteasome nuclear localization. In yet some further embodiments, the proteasome dynamics modulating compounds useful in the disclosed methods may lead to proteasome nuclear localization in a cell, and in addition, to at least one of the disclosed effects, specifically, (i) mTOR activation and localization to the lysosomal membrane; (ii) reduction in Sestrin3 levels and/or activity; (iii) activation of p38; (iv) reduction in the levels and/or activity of p62 and NBR1; and/or (v) modulation (specifically, activation) of NUP93, or any combinations thereof. In some embodiments, the disclosed modulating compound may lead to proteasome nuclear localization and in addition, to activation of mTOR, and/or to increased association of mTOR to the lysosomal membrane. In yet some further embodiments, the disclosed modulating compound may lead to proteasome nuclear localization and in addition, to reduction in Sestrin3 levels and/or activity. Still further, in some embodiments, the disclosed modulating compound may lead to proteasome nuclear localization and in addition, to activation of p38, specifically, p38 delta. Still further, in some embodiments, the disclosed modulating compound may lead to proteasome nuclear localization and in addition, to reduction in the levels and/or activity of p62 and NBR1. Still further, in some embodiments, the disclosed modulating compound may lead to proteasome nuclear localization and in addition, to activation of NUP93.

In some embodiments, the disclosed the compound that modulates proteasome dynamics and/or function (also referred to herein as the modulatory compound) applicable in the disclosed uses may be, or may comprise at least one of: a nucleic acid-based molecule, an amino acid-based molecule, a small molecule or any combinations thereof. In yet some further additional or alternative embodiments, the modulatory compound may target at least one of the signaling molecule/s, at the nucleic acid sequence level or at the protein level. In some specific embodiments, the disclosed modulatory compound used in the methods of the present disclosure my target any one of the mediator/s of amino acid sensing (e.g., at least one member of the Sestrin family), the at least one member of the MAPKs, specifically, members of the p38 mitogen-activated protein kinases (p38 MAPK, p38), at least one nucleo-cytosolic shuttle protein/s, and/or at least one NPC, at the nucleic acid sequence level or at the protein level. In yet some more specific embodiments, the disclosed modulator useful in all methods and compositions of the present disclosure, may target any one of SESN3, p38 (particularly p38 delta), p62 and/or NBR1, NUP93, and/or STAT3 at the nucleic acid sequence level or at the protein level.

In more specific embodiments, the modulatory compound disclosed herein, targets at least one of the disclosed signaling molecule/s (e.g., SESN3, p38, p62, NBR1, NUP93) at the nucleic acid sequence level (a). In more specific embodiments, such compound may be, or may comprise at least one nucleic acid-based molecule. In some particular and non-limiting embodiments, such nucleic acid molecule may be at least one of: a nucleic acid guide, a double-stranded RNA (dsRNA), a single-stranded RNA (ssRNA), an antisense oligonucleotide, a Ribozyme, a deoxyribozymes (DNAzymes), and an aptamer.

In some embodiments, the modulating compound used in the disclosed uses is a compound that targets SESN3. Accordingly, the disclosed uses comprise the step of administering to the subject at least one compound that reduces the level and/or activity of Sestrin3. In some embodiments, the compound used in the disclosed methods leads to reduction of Sestrin3 levels and/or activity by targeting SESN3 at the nucleic acid sequence level, and/or by targeting SESN3 at the protein level. In more specific embodiments the disclosed compound may act by at least one of: (i) specifically targeting a nucleic acid sequence encoding said Sestrin3, or any parts thereof; (ii) specifically targeting a nucleic acid sequence involves directly or indirectly in regulation of the Sestrin3 gene expression; (iii) reducing the stability (increasing degradation) of the Sesn3 protein; and/or (iv) interfering with the interaction of Sestrin3 with at least one regulatory complex.

In some embodiments, the compound comprises: (a) at least one RNA guide (gRNA) that guides least one nucleic acid guided genome modifier protein to at least one target sequence within the Sestrin3 encoding nucleic acid sequence, or within a nucleic acid sequence involves directly or indirectly in regulation of the Sestrin3 gene expression; or at least one nucleic acid sequence encoding the nucleic acid guide. In some optional embodiments, the disclosed compound or any composition thereof may further comprise (b), at least one nucleic acid guided genome modifier protein, or any chimeric protein, complex or conjugate thereof, or at least one nucleic acid sequence encoding the guided genome modifier protein or chimeric protein thereof.

In yet some alternative or additional embodiments, the modulatory compound used in the disclosed uses may target SESN3 at the protein level. Thus, according to some embodiments, the modulatory compound may (a), reduce the stability of SESN3 by targeted protein degradation (TPD). In yet some further additional and/or alternative embodiments, the compounds used in the present disclosure may affect the activity of SESN3 by affecting or modulating the interaction of Sestrin3 with a regulatory complex (b). In some specific embodiments, the regulatory complex is the GATOR2 complex. Accordingly, in some embodiments, the disclosed compound interferes and/or blocks and/or reduces the interaction of Sestrin3 with at least one member of the GATOR2 complex. In yet some further specific embodiments, it should be understood that since the interaction of Sestrin3 with at least one member of the GATOR2 complex is an inhibitory interaction, specifically, by interacting with at least one member of the GATOR2 complex, sestrin3 inhibits and/or prevents the inhibitory action of the GATOR2 complex on the GATOR1, complex, thereby reducing the inhibition of mTOR. In other words, by blocking the interaction of Sestrin3 with at least one member of the GATOR2 complex, the disclosed compound allows the activation of GATO R2, the inactivation of GATOR1, and activation of mTORC1.

In yet some further embodiments, the modulatory compound targets p38, specifically, p38 delta. Accordingly, the disclosed uses may relate to use of at least one compound that increases the level and/or activity of p38. In some specific embodiments, the compound is a p38 activator. In yest some further specific embodiments, the modulator used herein leads to phosphorylation of at least one of Thr180 (T180) and/or Tyr 182 (Y182) of p38.

In some embodiments, thep38 activator may be at least one of: a compound elevating the levels and/or activity of MAP kinase kinase 3 (MKK3) and/or MKK6; a hyperosmotic agent; and a DNA Synthesis Inhibitor.

In yet some further embodiments, the modulatory compound targets p62, or NBR1. Since as shown by the present Examples, for modulating the proteasome dynamics silencing of both, p62 and NBR1 is required, the disclosed uses may comprise the dual use of at least one compound that reduces the level and/or activity of p62 and at least one compound that reduces the level and/or activity of NBR1.

In some embodiments of the disclosed uses, the pathologic disorder is a disorder affected by proteasomal activity and/or cellular localization, said disorder is at least one of: at least one neoplastic disorder and/or at least one protein misfolding disorder or deposition disorder. In yet some further embodiments, the neoplastic disorder is cancer.

A further aspect of the present disclosure relates to a method for determining a personalized treatment regimen for a subject suffering from a pathologic disorder, by assessing responsiveness of the subject to a treatment regimen comprising at least one therapeutic compound, determining dosage of the compound, and/or monitoring disease progression of the subject. More specifically, the personalized methods disclosed herein comprise the following steps. In one step (a), the methods involve determining in at least one sample of the subject, at least one of: (i) mTOR activation and/or lysosomal association; (ii) activation of p38; (iii) phosphorylation of Tyr705 of STAT3; and/or (iv) Sestrin3 levels, and/or activity and/or the interaction of Sestrin3 with at least one regulatory complex; and optionally, (v) the proteasome subcellular localization; in at least one cell of the at least one sample, or in any fraction thereof. In step (b), the disclosed methods provide classifying the subject. In some embodiments, the subject is classified as (I), a responder subject to the treatment regimen, if at least one of: (i) mTOR is activated and/or localized to the lysosomal membrane; (ii) p38 is activated for example, p38 delta in the sample is phosphorylated in at least one of T180 and Y182; (iii) phosphorylation of Tyr705 of STAT3 is inhibited or reduced; and/or (iv) Sestrin3 levels, and/or activity and/or the interaction of Sestrin3 with at least one regulatory complex are reduced; and optionally, (v) the ratio of nuclear to cytosolic proteasome subcellular localization is greater than 1.

Alternatively, the subject may be classified as (II), a non-responder subject or a poor responder to said treatment regimen if at least one of: (i) mTOR is inactivated and/or dissociated from the lysosomal membrane; (ii) p38 is inactivated (dephosphorylation of at least one of T180 and Y182); (iii) Tyr705 of STAT3 is phosphorylated; and/or (iv) Sestrin3 levels, and/or activity and/or the interaction of Sestrin3 with at least one regulatory complex are increased or maintained; and optionally, (v) the ratio of nuclear to cytosolic proteasome subcellular localization is smaller than, or equal to 1.

In step (c) of the disclosed methods, the treatment regimen is maintained for a subject classified as a responder. Alternatively, for subject exhibiting a mild or poor response, the dose of the therapeutic compound in the treatment regimen is increased. In some embodiments, for a subject classified as a non-responder or poor responder, the treatment regimen may be ceased, thereby determining a treatment regimen to the subject.

It should be understood the disclosed aspect provides a personalized therapeutic approach incorporating various diagnostic parameters that were firstly discovered in the present invention as reflecting modulation of the proteasome dynamics. In some embodiments, a responder, in connection with the above-aspect, is a subject displaying a predominant nuclear localization of the proteasome in response to a given therapeutic compound or treatment regimen. Nuclear localization of the proteasome has been recently demonstrated by the inventors as being correlated to responsiveness to a treatment regimen or compound [17].

As shown herein, by reveling the cellular signaling pathway involved in the proteasome dynamics, the inventors provide additional effective diagnostic and therapeutic tools, enabling the provision of new therapeutic compounds targeting the newly discovered targets, and in addition, novel diagnostic parameters that facilitate and improve personalization of any therapeutic regimen. The newly discovered diagnostic parameters, specifically (i) mTOR lysosomal association; (ii) activation of p38; (iii) phosphorylation of Tyr705 of STAT3; and/or (iv) the interaction of Sestrin3 with at least one regulatory complex; that all show a clear correlation with the proteasome dynamics, can be determined either alone, in any combinations thereof, or in combination with determination of the proteasome subcellular localization in at least one cell of at least one sample, or in any fraction thereof. More specifically, mTOR activation, and/or lysosomal association may be determined using any histological approach, as disclosed by the present Examples. Non-limiting embodiment include indirect immunofluorescence, followed by staining for the lysosomal protein LAMP1 using an Alexa-Fluor 647-conjugated antibody. It should be noted that some of the cellular-localization methods disclosed herein below in connection with the proteasome localization, may be also applicable for determination of lysosomal localization. More specifically, in some embodiments, this can be done by staining of lysosomal proteins (such as LAMP1); over-expression of such proteins while fused to a fluorescent protein; or using common reagent which selectively stain the lysosome (e.g. LysoTracker). Still further, activation of mTOR can be analysed on each of its substrates as indicated above (phosphorylation and the like). Still further, the activation of p38 can be determined by detecting phosphorylation of p38, specifically, in at least one of Thr180 (T180) and/or Tyr 182 (Y182) of p38, as well as specific phosphorylation of STAT3, may be determined using specific antibodies. The interaction of Sestrin3 with at least one regulatory complex, specifically, with members of the GATOR2 complex, specifically, Mios and

WDR59, can be determined either by histological approach or, immunoprecipitation followed by Western blot as demonstrated by the present disclosure.

In some embodiments, the diagnostic step of the disclosed personalized therapeutic methods may comprise determining the proteasome nuclear localization and in addition, to activation of mTOR, and/or to increased association of mTOR to the lysosomal membrane. In yet some further embodiments, the diagnostic step of the disclosed personalized therapeutic methods may comprise determining proteasome nuclear localization and in addition, to reduction in Sestrin3 levels and/or activity, and/or association with at least one member of the GATO2 complex. Still further, in some embodiments, the diagnostic step of the disclosed personalized therapeutic methods may comprise determining proteasome nuclear localization and in addition, activation of p38, specifically, p38 delta, as reflected by phosphorylation at the disclosed residues. Still further, in some embodiments, the diagnostic step of the disclosed personalized therapeutic methods may comprise determining proteasome nuclear localization and in addition, the reduction in the levels and/or activity of p62 and/or NBR1. Still further, in some embodiments, the diagnostic step of the disclosed personalized therapeutic methods may comprise determining proteasome nuclear localization and in addition, the activation of NUP93. Still further, in some embodiments, the diagnostic step of the disclosed personalized therapeutic methods may comprise determining proteasome nuclear localization and in addition, the phosphorylation of STAT3, specifically in Y705.

According to some embodiments, in responsive subjects, which are also indicated herein as responders, the term predominantly nuclear (with respect to the proteasome localization), or predominantly localized to the lysosomal membrane (with respect to mTOR localization) as used herein means that most of the cells, e.g., more than 50% (e.g., 51%, 52%, 53%, 54%, 55%, 60%, 65%, 70%, 75%, 80%, 85%, 90%, 95%, 100%), in the at least one sample are classified as reflecting a ratio of nuclear to cytosolic proteasomal localization that is greater than 1. More specifically, a subject displaying a ratio that is between 1.0000001 to about 10¹⁰ or more, may be considered in accordance with some embodiments as a responder subject. The term predominantly cytosolic as used herein means that most of the cells, e.g., at least 50%, in the at least one sample are classified as reflecting a ratio of nuclear to cytosolic proteasomal localization that is either equal to 1 or smaller than 1. In yet some further embodiments, a non-responder subject may display a ratio that is between 1 to about 10-10 or less.

In some embodiments, a predominantly nuclear proteasomal localization is indicative of a positive prognosis, where a predominantly cytosolic proteasomal localization is indicative of a negative prognosis.

As indicated above, each of the newly identified prognostic/diagnostic parameters may be combined together with the proteasome localization. Determining the proteasome subcelular localization in at least one cell of at least one biological sample of said subject. Various methods are known in the art for determining the proteasome cellular localization, using any suitable means, and are all applicable in the present disclosure. In some embodiments, methods for determining the protcasome localization may include immunohistochemical methods and cell fractionation. More specifically, methods applicable in the present invention may include but are not limited to Immunohistochemistry, Live cell imaging of the proteasome activity probe (ABPs), Western blot of nuclear fractions (e.g., Western blot of cells for 20 and 19S subunits), Cell fractionation, Immunofluorescence microscopy and Cryo-electron tomographic imaging.

More specifically, Cell fractionation is the process used to separate cellular components while preserving individual functions of each component. Tissue is typically homogenized in a buffer solution that is isotonic to stop osmotic damage. Mechanisms for homogenization include grinding, mincing, chopping, pressure changes, osmotic shock, freeze-thawing, and ultra-sound. The samples are then kept cold to prevent enzymatic damage. Homogenous mass of cells (cell homogenate or cell suspension) is formed. It involves grinding of cells in a suitable medium in the presence of certain enzymes with correct pH, ionic composition, and temperature. A filtration step may then be applied. This step may not be necessary depending on the source of the cells. Animal tissue however is likely to yield connective tissue which must be removed. Commonly, filtration is achieved either by pouring through gauze or with a suction filter and the relevant grade ceramic filter. Purification is achieved by differential centrifugation—the sequential increase in gravitational force results in the sequential separation of organelles according to their density. In this connection, wherein the methods of the present disclosure involve the step of determining protcasome subcellular localization in a cell or in any fractions thereof, in some embodiments, such fractions of a cell may be a result of the cell fractionation process discussed herein. A cell fraction may be in some embodiments a nuclear reaction. In yet some further embodiments, a cell fraction may be a cytosolic fraction.

Western Blot as used herein, particularly when applied to cell fractions, involves separation of a substrate from other protein by means of an acryl amide gel followed by transfer of the substrate to a membrane (e.g., nitrocellulose, nylon, or PVDF). Presence of the substrate is then detected by antibodies specific to the substrate, which are in turn detected by antibody-binding reagents. Antibody-binding reagents may be, for example, protein A or secondary antibodies. Antibody-binding reagents may be radio labeled or enzyme-linked, as described hereinafter. Detection may be by autoradiography, colorimetric reaction, or chemiluminescence. This method allows both quantization of an amount of substrate and determination of its identity by a relative position on the membrane indicative of the protein's migration distance in the acryl amide gel during electrophoresis, resulting from the size and other characteristics of the protein.

Immuno-histochemical Analysis involves detection of a substrate in situ in fixed cells by substrate-specific antibodies. The substrate specific antibodies may be enzyme-linked or linked to fluorophore. Detection is by microscopy and is either subjective or by automatic evaluation. With enzyme-linked antibodies, a calorimetric reaction may be required. It will be appreciated that immunohistochemistry is often followed by counterstaining of the cell nuclei, using, for example, Hematoxyline or Giemsa stain.

Immunofluorescence microscopy enables visualization of proteasome subunits in the cells. In some embodiments, cells are seeded on glass cover slips and fixed with 4% PFA. Following appropriate treatment, the fixed cells are incubated with relevant first and secondary antibodies, washed and mounted. The fixed cells are then visualized using a confocal microscope (such as for example Zeiss LSM 700).

Live cell imaging of the proteasome consists in tagging the proteasomal subunits of living cells with a fluorescent probe, thereby allowing in vivo detection via confocal fluorescence microscopy. For example, the proteasomal subunits may be tagged with any tag such as GFP, e.g., the β4, Rpn2, Rpn6, and Rpn13 proteasome subunits may be C-terminally fused with GFP. Most proteasome subunits fully incorporate GFP tag into their appropriate sub-complexes, thus enabling live cell imaging of the 20S core protease (CP), the 19S regulatory particle (RP), and/or holo-26S particles. Cryo-electron tomographic imaging is a method that facilitates in situ structural biology on a protcomic scale. In a cryo-ET study, a biological sample, a cell, tissue, or organism, is flash frozen, thinned to an appropriate thickness, and then imaged using an electron microscope. The freezing process preserves the sample in a hydrated, close-to-native state. Multiple images are captured as the sample is tilted along an axis. The images are then aligned and merged using computational techniques to reconstruct a three-dimensional picture, or tomogram. This method has been successful for mapping the locations of relatively large structures such as proteasome as well as ribosomes.

As indicated above, Proteasome activity-based probes (ABPs) may also be employed for detecting proteasome localization and activity. ABPs are small molecules consisting of a proteasome inhibitor linked to a small fluorophore. Fluorescence labeling of proteasomes occurs via a nucleophilic attack of the catalytic N-terminal threonine toward the ABP, leading to a covalent, irreversible bond between the warhead of the ABP and the proteasome active site. Importantly, unlike fluorescently tagged proteasome subunits, the ABPs only label fully assembled, active proteasome complexes. ABPs react with proteasomes in a way that corresponds to their catalytic activity and because of their fluorescent properties, they can be imaged specifically and sensitively in cell lysates after gel-electrophoresis followed by fluorescent scanning or in living cells by fluorescence microscopy. With a few exceptions, most proteasome ABPs share a similar design, may comprise the following components:

(a) a reactive group (‘warhead’), typically an epoxyketone (EK) or vinyl sulfone (VS), at the C terminus; (b) a tri- or tetrapeptide recognition element; (c) a reporter tag for detection (often a fluorophore), typically appended at the N terminus via a linker. Consequently, the probes are frequently notated in the form label-linker-recognition element-warhead (e.g., BODIPY-Ahx3-L3-VS), or label-inhibitor (e.g., BODIPY-epoxomicin).

Proteasome ABPs may be divided into two categories: ‘broad-spectrum’, which are reactive toward most proteasome subunits, and ‘subunit-selective’, which show a strong preference for a single subunit type.

It should be understood that when referring to detection of the proteasome, the invention encompasses the detection of the 26S, or of any subunit thereof, specifically, at least one of the 20S and 19S subunits, as specified above.

The second step of the methods disclosed herein involves classifying the subject as a responsive (or responder) or a non-responsive (or non-responder) subject. In some embodiments, the classicication is based on (i) mTOR activation and/or lysosomal association; (ii) activation of p38; (iii) phosphorylation of Tyr705 of STAT3; and/or (iv) Sestrin3 levels, and/or activity and/or the interaction of Sestrin3 with at least one regulatory complex; and optionally, (v) the subcellular localization in at least one cell of the subject. As used herein, proteasome subcellular localization that is predominantly nuclear, or a predominant mTOR activation and/or lysosomal association, is meant that the proteasome in the examined cell is mostly, mainly and/or primaraly, localized to the nucleus, or that in most of the cells in the sample mTOR is activated and/or associated to the lysosomal membrane. Specifically, a predominant, preponderant, major and/or principle share of the cellular proteasome display nuclear localization in the cell, or principle share of the cellular mTOR display activation and/or lysosomal association. Similar indication is also applicabe to the other parameters. It should be understood that for brevity purpose, the following description indicates cellular localization of the proteasome, but the description can be applied for any of the other examined parameters, specifically, (i) mTOR activation and/or lysosomal association; (ii) activation of p38; (iii) phosphorylation of Tyr705 of STAT3; and/or (iv) Sestrin3 levels, and/or activity and/or the interaction of Sestrin3 with at least one regulatory complex. In some embodiments, if at least one of: (i) mTOR is activated and/or localized to the lysosomal membrane; (ii) p38 is activated; (iii) phosphorylation of Tyr705 of STAT3 is inhibited or reduced; and/or (iv) Sestrin3 levels, and/or activity and/or the interaction of Sestrin3 with at least one regulatory complex are reduced; and optionally, (v) the ratio of nuclear to cytosolic proteasome subcellular localization is greater than 1, the subject is classified as a responder to the treatment regimen. More specifically, more than 50% of the proteasome in the cell is localized to the nucleus, specifically, about 51% or more, about 52% or more, about 53% or more, about 54% or more, about 55% or more, 60%, 65%, 70%, 75%, 80%, 85%, 90%, 95%, or more, 97%, 98%, 99% or even 100%, of the proteasome in the cell display nuclear localization.

In some embodiments, if at least one of: (i) mTOR is inactivated and/or dissociated from the lysosomal membrane; (ii) p38 is inactivated; (iii) Tyr705 of STAT3 is phosphorylated; and/or (iv) Sestrin3 levels, and/or activity and/or the interaction of Sestrin3 with at least one regulatory complex are increased or maintained, the subject is classified as a non-responder or as a drug resistant. In some embodiments, such indication is further confirmed if the sample display (v) cytosolic loclization of 55% or more of the celular proteasome in at least one cell of the subject, indicates drug resistance to the treatment regimen.

In some embodiments, the subject/s diagnosed by the methods of the present disclosure may display both, nuclear and cytosolic proteasome localization in most cells of the sample. According to some embodiments, for such subjects, a nuclear localization of about 50% or less, of the proteasome in at least one cell of the sample examined, is indicative of drug resistance. Thus, as shown by the present disclosure and discussed herein, an equal distribution of the proteasome between both compartments (cytosolic and nuclear) reflects non-responsiveness or drug resistance. More specifically, in some specific embodiments of the present disclosure, cytosolic localization of about 50% or more of the proteasome, specifically, 50%, 55%, 60%, 65%, 70%, 75%, 80%, 85%, 90%, 95%, 97%, 98%, 99% or more, 100%, is referred to herein as cytosolic, and is indicative of non-responsiveness to a treatment regimen.

However, a nuclear distribution of about 51% or more, and more specifically, 55% or more, of the proteasome in the cell of a subject, is referred to herein as a predominantly nuclear or as a nuclear localization and reflects responsiveness. More specifically, nuclear localization of about 55%, 60%, 65%, 70%, 75%, 80%, 85%, 90%, 95%, 97%, 98%, 99% or even 100% of the proteasome in the cell, indicates that the subject is responsive to a treatment regimen.

It should be further understood that in some embodiments, at least one of: (i) mTOR is inactivated and/or dissociated from the lysosomal membrane; (ii) p38 is inactivated; (iii) Tyr705 of STAT3 is phosphorylated; and/or (iv) Sestrin3 levels, and/or activity and/or the interaction of Sestrin3 with at least one regulatory complex are maintained or increased; and optionally, (v) a cytosolic localization determined for between about 1%-100%, specifically about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, 55%-60%, 60%-65%, 65%-70%, 70%-75%, 75%-80%, 80%-85%, 85%-90%, 90%-95%, 95%-100%, of the cells in the sample, indicates that said subject belongs to a pre-established drug-resistant or non-responsive population of subjects. In other words, the subject is a non-responsive subject. In some particular embodiments, such drug-resistant subjects or population of subjects may be associated with relapse of the disease. In yet some further embodiments, at least one of: (i) mTOR is activated and/or localized to the lysosomal membrane; (ii) p38 is activated; (iii) phosphorylation of Tyr705 of STAT3 is inhibited or reduced; and/or (iv) Sestrin3 levels, and/or activity and/or the interaction of Sestrin3 with at least one regulatory complex are reduced; and optionally, (v) a nuclear localization determined for between about 1%-100%, specifically about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, 55%-60%, 60%-65%, 65%-70%, 70%-75%, 75%-80%, 80%-85%, 85%-90%, 90%-95%, 95%-100%, of the cells in the sample, indicates that said subject belongs to a pre-established drug-responsive or responder population of subjects. In other words, the subject is a responsive subject. In some particular embodiments, such drug-responsive subjects or population of subjects may be associated with good prognosis. Thus, in some embodiments, if 50% or more of the cells in the sample display at least one of: (i) mTOR inactivation and/or dissociation from the lysosomal membrane; (ii) p38 inactivation; (iii) Tyr705 of STAT3 phosphorylation; and/or (iv) increased or maintained Sestrin3 levels, and/or activity and/or interaction of Sestrin3 with at least one regulatory complex; and optionally, (v) cytosolic distribution of the proteasome (e.g., that about 45% or more of the cellular proteasome in the cell is cytosolic), the subject is classified as a non-responder, or drug resistant. In yet some further embodiments, if 50% or more of the cells in the sample display nuclear localization (e.g., that 51% or more, and specifically, 55% or more of the cellular proteasome is nuclear), the subject is classified as a responder.

As described hereinabove, in some embodiments, the methods of the present disclosure refer to determining at least one of (i) mTOR activation and/or lysosomal association; (ii) activation of p38; (iii) phosphorylation of Tyr705 of STAT3; and/or (iv) Sestrin3 levels, and/or activity and/or the interaction of Sestrin3 with at least one regulatory complex; and optionally, (v) the proteasome subcellular localization value based on the relative amounts of the mTOR or proteasome in the cell compartments, specifically, the lysosomal membrane, cytosol and the nucleus. An equivalent distribution between both compartments, reflects non-responsiveness, or drug resistance. In other words, an equal distribution (namely, 50% or more, and in some embodiments, even 45% or more) of the proteasome in the cytosol and the nucleus, indicates non-responsiveness. As such, a value of about 40% to 60%, specifically, 40%, 45%, 50%, 55%, 60% may be used as a cutoff value. In yet some further embodiments, a value of about 50% of the proteasome in the cell, may be considered as a cutoff value. It should be noted that a “cutoff value”, sometimes referred to simply as “cutoff” herein, is a value that in some embodiments of the present disclosure, meets the requirements for both high prognostic sensitivity (true positive rate) and high prognostic specificity (true negative rate). Simply put, “sensitivity” relates to the rate of identification of the responder patients (samples) as such, out of a group of samples, whereas “specificity” relates to the rate of correct identification of responder samples as such, out of a group of samples. It should be noted that cutoff values may be also provided as control sample/s or alternatively and/or additionally, as standard curve/s that display predetermined standard values for responders, non-responders, and for subjects that display responsiveness to a certain extent (level of responsiveness, e.g., low, moderate and high). More specifically, the cutoff values reflect the result of a statistical analysis of proteasome localization value/s differences in pre-established populations of responder or non-responder. Pre-established populations as used herein refer to population of patients known to be responsive to a treatment of interest (e.g., treatment comprising at least one proteasome inhibitor), or alternatively, population of patients known to be non-responsive or drug-resistant to a treatment of interest.

It should be emphasized that the nature of the invention is such that the accumulation of further patient data may improve the accuracy of the presently provided cutoff values, which are usually based on ROC (Receiver Operating Characteristic) curves generated according to the patient data using analytical software program.

It should be appreciated that “Standard”, or a “predetermined standard” as used herein, denotes either a single standard value or a plurality of standards with which the proteasome subcellular nuclear or cytosolic localization value determined for the tested sample is compared. The standards may be provided, for example, in the form of discrete numeric values or in the form of a chart for different values of proteasome localization, or alternatively, in the form of a comparative curve prepared on the basis of such standards (standard curve).

Thus, in certain embodiments, the prognostic methods of the present disclosure may optionally further involve the use of a calibration curve created by detecting and quantitating at least one of the parameters discussed herein, mTOR activity and/or lysosomal association, the activity and/or level/s and/or PTM/s, and/or subcellular localization of at least one signaling molecule participating directly or indirectly in at least one pathway mediating said proteasome dynamics and/or function, for example, p38, SESN3, STAT3, p62, NBR1, etc., and optionally, in addition proteasome cellular localization, in cells of known populations of responders and non-responders to the indicated treatment. Obtaining such a calibration curve may be indicative to provide standard values.

As noted above, in some embodiments of the present disclosure, at least one control sample may be provided and/or used by the methods discussed herein. A “control sample” as used herein, may reflect a sample of at least one subject (a subject that is known to be a non-responder, or alternatively, known to be a responder, or sample displaying known at least one of the parameters discussed herein, mTOR lysosomal association, the activity and/or level/s and/or PTM/s, and/or subcellular localization of at least one signaling molecule participating directly or indirectly in at least one pathway mediating said proteasome dynamics and/or function, for example, p38, SESN3, STAT3, p62, NBR1, etc., and optionally, in addition proteasome cellular localization, specifically, nuclear and/or cytosolic at a certain predetermined degree), and in some embodiments, a mixture at least two, at least three, at least four, at least five, at least six, at least seven, at least eight, at least nine, at least ten or more patients, specifically, 15, 20, 25, 30, 35, 40, 45, 50, 55, 60, 65, 70, 75, 80, 85, 90, 95, 100 or more patients. A control sample may alternatively, or additionally comprise known cytosolic or nuclear protein or other cellular component that display known at least one of the parameters discussed herein, mTOR activity and/or lysosomal association, the activity and/or level/s and/or PTM/s, and/or subcellular localization of at least one signaling molecule participating directly or indirectly in at least one pathway mediating said proteasome dynamics and/or function, for example, p38, SESN3, STAT3, p62, NBR1, etc., and optionally, in addition proteasome cellular localization that can be used as a reference for cytosolic or nuclear localization.

In some embodiments, the methods disclosed herein further provide monitoring of the subject. More specifically, in some embodiments, monitoring disease progression comprises predicting and determining disease relapse and/or assessing a remission interval. Thus, in some embodiments, the method further comprises the steps of: (d), repeating step (a) to determine at least one of the parameters defined in sections (i) to (iv) above, for at least one more temporally separated sample of the subject. Step (c) involves predicting and/or determining disease relapse in the subject, if the at least one temporally separated sample displays at least one of: (i) inactivation and/or dissociation of mTOR from the lysosomal membrane; (ii) loss of p38 activation (p38 delta), for example, T180/Y182 phosphorylation; (iii) increased and/or maintained phosphorylation of Tyr705 of STAT3; and/or (iv) increase in the Sestrin3 levels, and/or activity and/or the interaction of Sestrin3 with at least one regulatory complex is reduced; and optionally, (v) loss of proteasome nuclear localization or maintained cytosolic localization, and/or reduction in the ratio of nuclear to cytosolic proteasome localization in at least one cell of the sample.

In some embodiments, the methods of the invention may be particularly useful for monitoring disease progression. In some embodiments, monitoring disease progression by the methods of the invention may comprise at least one of, predicting and determining disease relapse, and assessing a remission interval. In such case, the method of the invention may comprise the steps of: repeating step (a) of the method of the invention to determine at least one of (i) mTOR activation and/or lysosomal association; (ii) activation of p38 (p38 delta); (iii) phosphorylation of Tyr705 of STAT3; and/or (iv) Sestrin3 levels, and/or activity and/or the interaction of Sestrin3 with at least one regulatory complex; and optionally, (v) proteasome subcellular localization in at least one cell of said at least one sample, or in any fraction thereof; for at least one cell of at least one more temporally-separated sample of the subject. More specifically, according to some embodiments, a method allowing monitoring disease progression as defined above may comprise first in step (a), determining at least one of (i) mTOR activation and/or lysosomal association; (ii) activation of p38; and (iii) phosphorylation of Tyr705 of STAT3; and/or (iv) change in Sestrin3 levels, and/or activity and/or the interaction of Sestrin3 with at least one regulatory complex; and optionally, (v)) proteasome subcellular localization in at least one cell of said at least one sample, or in any fraction thereof, in at least one cell of at least one biological sample of the subject or in any fraction of the cell. In some embodiments, the subject is being classified in the next step (b), as (I) a responder subject to the treatment regimen, if at least one of: (i) mTOR is activated and/or localized to the lysosomal membrane; (ii) p38 is activated; (iii) phosphorylation of Tyr705 of STAT3 is inhibited or reduced; and/or (iv) Sestrin3 levels, and/or activity and/or the interaction of Sestrin3 with at least one regulatory complex are reduced; and optionally, (v) the ratio of nuclear to cytosolic proteasome subcellular localization is greater than 1.

Alternatively, the subject is classified as (II), a non-responder subject or a poor responder to the treatment regimen if at least one of: (i) mTOR is inactivated and/or dissociated from the lysosomal membrane; (ii) p38 is inactivated; (iii) Tyr705 of STAT3 is phosphorylated; and/or (iv) Sestrin3 levels, and/or activity and/or the interaction of Sestrin3 with at least one regulatory complex are increased, maintained, and/or stable; and optionally, (v) the ratio of nuclear to cytosolic proteasome subcellular localization is smaller than, or equal to 1.

In some embodiments, at least one more temporally separated sample is obtained after the initiation of the least one treatment regimen comprising the at least one therapeutic compound.

The present disclosure thus provides a method for determining a personalized treatment regimen for a subject suffering from a pathologic disorder. A “Personal treatment”, as used herein, refers to treatment which is tailored to the individual patient based on their predicted response or risk of disease. This term further encompasses any future monitoring, prediction and management of relapse and chances for response during relapse.

Moreover, the present disclosure further provides prognostic methods for assessing responsiveness of a subject for a specific treatment regimen, for monitoring a disease progression and for predicting relapse of the disease in a subject. It should be noted that “Prognosis”, is defined as a forecast of the future course of a disease or disorder, based on medical knowledge. This highlights the major advantage of the present disclosure, namely, the ability to assess responsiveness or drug-resistance and thereby predict progression of the disease, based on at least one of: (i) mTOR activation and/or lysosomal association; (ii) activation of p38; (iii) phosphorylation of Tyr705 of STAT3; and/or (iv) Sestrin3 levels, and/or activity and/or the interaction of Sestrin3 with at least one regulatory complex; and optionally, (v) the proteasome dynamics evaluated in a cell of the prognosed subject. The term “relapse”, as used herein, relates to the re-occurrence of a condition, disease or disorder that affected a person in the past. Specifically, the term relates to the re-occurrence of a disease being treated with modulators of proteasome dynamics.

The term “response” or “responsiveness” to a certain treatment, specifically, treatment regimen that comprise any of the modulators disclosed by the present disclosure, refers to an improvement in at least one relevant clinical parameter as compared to an untreated subject diagnosed with the same pathology (e.g., the same type, stage, degree and/or classification of the pathology), or as compared to the clinical parameters of the same subject prior to treatment with the indicated medicament.

The term “non responder” or “drug resistance” to treatment with a specific medicament, specifically, treatment regimen that comprise the disclosed modulators, refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to treatment with the specific medicament. In yet some further embodiments the subject may be further sub classified with respect to the expected degree, depth or extent and/or duration of responsiveness, for example as a poor responder, a responder displaying mild response, a responder displaying a good response or even a responder displaying excellent response, and the like.

It should be appreciated that subject specific parameters (e.g., at least one of: mTOR activity and/or lysosomal association; activation of p38; phosphorylation of Tyr705 of STAT3; and/or Sestrin3 levels, and/or activity and/or the interaction of Sestrin3 with at least one regulatory complex; and optionally, the proteasome cellular localization), determined by the diagnostic and prognostic methods of the present disclosure, allows considering the heterogeneity of the cells within a single sample of the diagnosed subject, thereby providing an accurate and sensitive predictive tool that is not only limited to the determination of responders and non-responders, but also reflects the depth of the response. As such, the disclosed methods provide a powerful means for an accurate prediction allowing a more informative determination of the patient's prognosis, specifically with respect to the course of the disease. For example, responsiveness, relapse, length of disease-free period, survival, extent and/or severity and/or intensity of disease symptoms, side effects, disease related conditions and the like.

In some embodiments, the at least one more temporally separated sample may be obtained after the initiation of at least one treatment regimen comprising at least one therapeutic compound. In some embodiments such therapeutic compound may be any compound known in the art (chemotherapeutic compound, immunotherapeutic compound and the like). In yet some further embodiments, the compound may be any of the disclosed modulators. modulator of proteasome dynamics.

It should be understood that in some particular embodiments, at least one sample may be obtained prior to initiation of the treatment. Thus, in some embodiments, at least one sample is taken before treatment and at least one sample is obtained after treatment. However, in some embodiments, the methods disclosed herein may be applied to subjects already treated by a treatment regimen comprising at least one modulator as disclosed herein, or any other dug. Accordingly, the first and the second samples are obtained after the initiation of the treatment. Such monitoring may therefore provide a powerful therapeutic tool used for improving and personalizing the treatment regimen offered to the treated subject. In some embodiments, the at least two samples may be obtained on different time points after the initiation of a treatment regimen using any therapeutic compound. In yet some further embodiments the at least two samples may be obtained from a subject during various time points before the initiation of any treatment regimen.

As indicated above, in accordance with some embodiments of the present disclosure, in order to assess the patient condition, or monitor the disease progression, as well as responsiveness to a certain treatment (e.g., comprising at least one proteasome inhibitor), at least two “temporally-separated” test samples must be collected from the examined patient and compared thereafter, in order to determine if there is any change or difference in the any of the parameters discussed above, and optionally, proteasome localization values between the samples. Such change may reflect a change in the responsiveness of the subject. In practice, to detect a change having more accurate predictive value, at least two “temporally-separated” test samples and preferably more, must be collected from the patient.

The proteasome cellular localization value is determined using the method disclosed herein, applied for each sample. As detailed above, the change in localization is calculated by determining the change in cellular localization between at least two samples obtained from the same patient in different time-points or time intervals. This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least one week, at least two weeks, at least three weeks, at least one month, at least two months, at least three months, at least four months, at least five months, at least six months, at least one year, or even more.

The number of samples collected and used for evaluation and classification of the subject either as a responder or alternatively, as a drug resistant or as a subject that may experience relapse of the disease, may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the disease progression according to the present disclosure, it is understood that the change in at least one of: (i) mTOR activation and/or lysosomal association; (ii) activation of p38; (iii) phosphorylation of Tyr705 of STAT3; and/or (iv) Sestrin3 levels, and/or activity and/or the interaction of Sestrin3 with at least one regulatory complex; and optionally, (v) the nuclear or cytosolic proteasome localization value, may be calculated as an average change over at least three samples taken in different time points, or the change may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more.

In some specific and non-limiting embodiment, the compound used in the treatment regimen is a compound that modulates at least one pathway mediating proteasome dynamics and/or function. In some further embodiments, the compound is characterized by affecting at least one of: mTOR activity and/or lysosomal association, the activity and/or level/s and/or PTMs, and/or subcellular localization of at least one signaling molecule participating directly or indirectly in the at least one pathway mediating proteasome dynamics and/or function, and optionally, the proteasome cellular localization. However, as indicated above, any compound may be used.

More specifically, in some embodiments, such signaling molecule affected by the compound used as the therapeutic compound in the treatment regimen monitored by the personalized methods of the present disclosure, may be at least one stress-induced protein/s.

In some embodiments, the at least one signaling molecule participating directly or indirectly in the at least one pathway mediating the proteasome dynamics and/or function is at least one of: at least one mediator of metabolite sensing, at least one stress kinase, at least one nucleo-cytosolic shuttle protein, and/or at least one NPC protein.

Still further, in some embodiments of the disclosed personalized method, at least one of: (i) the mediator of metabolite sensing is a mediator of amino acid sensing. In yet some further alternative or additional embodiments, (ii), the stress kinase is at least one member of the MAPKs.

In some specific embodiments, the at least one mediator of amino acid sensing is at least one member of the Sestrin family. The at least one member of the MAPKs is at least one member of the p38 MAPKs, the at least one nucleo-cytosolic shuttle protein/s is p62 and NBR1, and/or wherein said at least one NPC is NUP93.

In some embodiments of the disclosed personalized methods, at least one of: (i) the at least one mediator of amino acid sensing is at least one member of the Sestrin family. In yet some further or additional embodiments, (ii), the at least one member of the MAPKs is at least one member of the p38 mitogen-activated protein kinases (p38 MAPK, p38). In yet some further additional or alternative embodiments, (iii), the at least one nucleo-cytosolic shuttle protein/s, specifically, ubiquitin and/or proteasome interacting shuttle proteins, is at least one of Sequestosome 1 (SQSTM1, p62) and Neighbor of BRCA1 gene 1 protein (NBR1). Still further, in some additional or alternative embodiments (iv), the at least one NPC is Nucleoporin 93 (NUP93).

In some embodiments, the at least one member of the Sestrin family is Sestrin3 (SESN3). In yet some additional and/or alternative embodiments, the at least one member of the p38 MAPK family (alpha, beta, gamma and delta), is p388.

As indicated above, the disclosed modulator may be any therapeutic agent at any therapeutic regimen. However, in some embodiments the compound may be characterized in proteasome dynamic modulation. In some embodiments of the disclosed personalized methods, the modulatory compound leads to: (I) at least one of: (i) mTOR activation and/or localization to the lysosomal membrane; (ii) reduction in Sestrin3 levels and/or activity; (iii) activation of p38; (iv) reduction in the levels and/or activity of p62 and NBR1; and/or (v) modulation of NUP93. In yet some optional embodiments, the disclosed modulator of proteasome dynamics leads, in addition to at least one of the effects disclosed in (i), (ii), (iii), (i) and/or (v), also to (II), proteasome nuclear localization. In yet some further embodiments, the proteasome dynamics modulating compounds useful in the disclosed methods may lead to proteasome nuclear localization in a cell, and in addition, to at least one of the disclosed effects, specifically, (i) mTOR activation and/or localization to the lysosomal membrane; (ii) reduction in Sestrin3 levels and/or activity (e.g., interaction of Sestrin3 with at least one regulatory complex); (iii) activation of p38; (iv) reduction in the levels and/or activity of p62 and NBR1; and/or (v) modulation (specifically, activation) of NUP93, or any combinations thereof. In some embodiments, the disclosed modulating compound may lead to proteasome nuclear localization and in addition, to activation of mTOR, and/or to increased association of mTOR to the lysosomal membrane. In yet some further embodiments, the disclosed modulating compound may lead to proteasome nuclear localization and in addition, to reduction in Sestrin3 levels and/or activity. Still further, in some embodiments, the disclosed modulating compound may lead to proteasome nuclear localization and in addition, to activation of p38, specifically, p38 delta. Still further, in some embodiments, the disclosed modulating compound may lead to proteasome nuclear localization and in addition, to reduction in the levels and/or activity of p62 and NBR1. Still further, in some embodiments, the disclosed modulating compound may lead to proteasome nuclear localization and in addition, to activation of NUP93. Still further, in some specific embodiments, a compound used as the therapeutic compound in the treatment regimen monitored by the personalized methods of the present disclosure may be any compound that leads to mTOR activation and/or localization to the lysosomal membrane, or a compound that prevents or reduces the dissociation of mTOR from the lysosomal membrane. Still further, in some additional or alternative embodiments, a compound useful in the disclosed methods may be a compound that leads to, or increases proteasome nuclear localization, also referred to herein as leads to a predominant nuclear localization. It should be noted that in some additional or alternative embodiments, such compound may increase the ratio of nuclear to cytosolic proteasome localization or lead to a ratio of nuclear to cytosolic proteasome localization that is greater than 1. Still further, in some additional or alternative embodiments, the compounds of the disclosed methods may be compounds that lead to reduction in Sestrin3 levels and/or activity. Still further, in some additional or alternative embodiments, the compound of the disclosed methods may be a compound that leads to activation of p38. In yet some further additional or alternative embodiments, a compound applicable in the disclosed methods may be a compound that leads to inhibition and/or reduction of Tyr705 of STAT3 phosphorylation. In in some additional or alternative embodiments, a compound applicable in the disclosed methods may be a compound that leads to a reduction in the levels and/or activity of p62 and/or NBR1. In some additional or alternative embodiments, a compound applicable in the disclosed methods may be a compound that modulates NUP93.

In some embodiments, the therapeutic regimen monitored by the personalized methods of the present disclosure may comprise any combination of the compounds indicated herein above.

In some embodiments, the therapeutic compound used in the treatment regimen monitored by the personalized methods of the present disclosure may comprise at least one of: (a), at least one tyrosine (Y) residue, any tyrosine mimetic, any salt or ester thereof, any multimeric and/or polymeric form of the tyrosine residue and/or of the tyrosine mimetic, and any combinations or mixtures thereof; (b), at least one tryptophan (W) residue, any tryptophan mimetic, any salt or ester thereof, any multimeric and/or polymeric form of the tryptophan residue and/or of the tryptophan mimetic, or any combination or mixture thereof; and (c), at least one phenylalanine (F) residue, any phenylalanine mimetic, any salt or ester thereof, any multimeric and/or polymeric form of the phenylalanine residue and/or of the phenylalanine mimetic, and any combinations or mixtures thereof.

In some embodiments of the disclosed monitoring and personalized therapeutic regimen determination methods, where the treatment regimen comprises at least one of Y, W, F or any combinations thereof, particularly, the YWF triade, the disclosed methods do not comprise the step of determining the proteasome cellular localization in at least one sample of the subject. In other words, the diagnostic step in the disclosed personalized therapeutic method that uses the YWF, or any peptide thereof or any composition or combinations thereof, as a therapeutic compound, does not comprise also determination of the proteasome cellular localization, unless it also concerns determining of one of the other parameters indicated herein. It should be understood that all other parameters as disclosed herein above in the present personalized methods are applicable also for a treatment regimen that comprises YWF. Specifically, in some embodiments, for a treatment regimen that comprises YWF, the personalized methods involve determining in at least one sample of the subject, at least one of: (i) mTOR activation and/or lysosomal association; (ii) activation of p38; and/or (iii) phosphorylation of Tyr705 of STAT3.

As shown by the present disclosure and in a recent publication of the inventor's previous patent application [17], a triad of aromatic amino acid residues act as modulating compounds that modulate proteasome dynamics in short term stress conditions and may therefore be used as a nutrient sensor. An aromatic amino acid (AAA) is an amino acid that includes a hydrophobic side chain, specifically, an aromatic ring. More specifically, a cyclic (ring-shaped), planar (flat) structures with a ring of resonance bonds that gives increased stability compared to other geometric or connective arrangements with the same set of atoms. An aromatic functional group or other substituent is called an aryl group. Aromatic amino acids absorb ultraviolet light at a wavelength above 250 nm and produce fluorescence. Among the 20 standard amino acids, the following are aromatic: phenylalanine, tryptophan and tyrosine.

“Aromatic amino acid” as used herein, includes natural as well as unnatural amino acids. Unnatural, aromatic amino acids comprise those that include an indole moiety in their amino acid side chain, wherein the indole ring structure can be substituted with one or more aryl group substituents. Additional examples of aromatic amino acids include but are not limited to 1-naphthylalanine, biphenylalanine, 2-napthylalananine, pentafluorophenylalanine, and 4-pyridylalaninc. More specifically, the term “aromatic” as used herein, refers to a mono-, bi-, or other multi-carbocyclic, aromatic ring system. The aromatic group may optionally be fused to one or more rings chosen from aromatics, cycloalkyls, and heterocyclyls. Aromatics can have from 5-14 ring members, such as, e.g., from 5-10 ring members. One or more hydrogen atoms may also be replaced by a substituent group selected from acyl, acylamino, acyloxy, alkenyl, alkoxy, alkyl, alkynyl, amino, aromatic, aryloxy, azido, carbamoyl, carboalkoxy, carboxy, carboxyamido, carboxyamino, cyano, cycloalkyl, disubstituted amino, formyl, guanidino, halo, heteroaryl, heterocyclyl, hydroxy, iminoamino, monosubstituted amino, nitro, oxo, phosphonamino, sulfinyl, sulfonamino, sulfonyl, thio, thioacylamino, thioureido, and ureido. Nonlimiting examples of aromatic groups include phenyl, naphthyl, indolyl, biphenyl, and anthracenyl.

As indicated above, in some particular embodiments, the aromatic amino acid provided by the present disclosure as effective modulating compound/s may be at least one of Tyrosine, Tryptophan and Phenylalanine, or any combinations thereof.

Thus, in some specific embodiments, the aromatic amino acid residue that may be provided as a selective inhibitor of proteasome translocation or as a proteasome dynamic modulating compound in the present disclosure is Tyrosine. Tyrosine (symbol Tyr or Y) or 4-hydroxyphenylalanine is a non-essential amino acid with a polar side group, having the formula C₉H₁₁NO₃. L-Tyrosine has the following chemical structure, as denoted by Formula I:

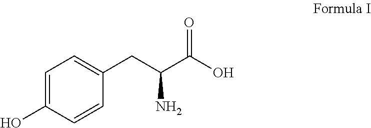 [SMILES: N[C@@H](Cc1ccc(O)cc1)C(=O)O]

While tyrosine is generally classified as a hydrophobic amino acid, it is more hydrophilic than phenylalanine. It is encoded by the codons UAC and UAU in messenger RNA (mRNA). Mammals synthesize tyrosine from the essential amino acid phenylalanine. The conversion of phe to tyr is catalyzed by the enzyme phenylalanine hydroxylase. In dopaminergic cells in the brain, tyrosine is converted to L-DOPA by the enzyme tyrosine hydroxylase (TH). TH is the rate-limiting enzyme involved in the synthesis of the neurotransmitter dopamine. Dopamine can then be converted into other catecholamines, such as norepinephrine (noradrenaline) and epinephrine (adrenaline).

The thyroid hormones triiodothyronine (T3) and thyroxine (T4) in the colloid of the thyroid are also derived from tyrosine.

In yet some further specific embodiments, the aromatic amino acid residue that may be provided as a proteasome dynamic modulating compound in the present disclosure is Tryptophan.

Tryptophan (symbol Trp or W) is an α-amino acid that is used in the biosynthesis of proteins, having the formula C₁₁H₁₂N₂O₂.

L-Tryptophan has the following chemical structure, as denoted by Formula II:

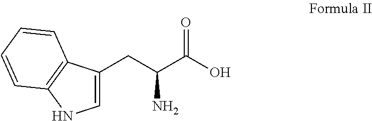 [SMILES: N[C@@H](Cc1c[nH]c2ccccc12)C(=O)O]

Tryptophan contains an α-amino group, an α-carboxylic acid group, and a side chain indole, making it a non-polar aromatic amino acid. It is encoded by the codon UGG. Like other amino acids, tryptophan is a zwitterion at physiological pH where the amino group is protonated (—NH₃⁺; pKₐ=9.39) and the carboxylic acid is deprotonated (—COO⁻; pKₐ=2.38).

Tryptophan functions as a biochemical precursor for the following compounds: Serotonin (a neurotransmitter), synthesized by tryptophan hydroxylase; Melatonin (a neurohormone) is in turn synthesized from serotonin, via N-acetyltransferase and 5-hydroxyindole-O-methyltransferase enzymes; Niacin, also known as vitamin B3, is synthesized from tryptophan via kynurenine and quinolinic acids; Auxins (a class of phytohormones) are synthesized from tryptophan. Tryptophan is also a precursor to the neurotransmitter serotonin, the hormone melatonin and vitamin B3.

Still further, in some specific embodiments, the aromatic amino acid that may be provided as a proteasome dynamics modulating compound in the methods of the present disclosure is Phenylalanine.

Phenylalanine (symbol Phe or F) is an essential α-amino acid with the formula C₉H11NO₂. It can be viewed as a benzyl group substituted for the methyl group of alanine, or a phenyl group in place of a terminal hydrogen of alanine.

L-Phenylalanine has the following chemical structure, as denoted by Formula III:

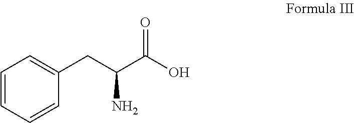 [SMILES: N[C@@H](Cc1ccccc1)C(=O)O]

This essential amino acid is classified as neutral, and nonpolar because of the inert and hydrophobic nature of the benzyl side chain. The L-isomer is used to biochemically form proteins, coded for by DNA. Phenylalanine is a precursor for tyrosine, the monoamine neurotransmitters dopamine, norepinephrine (noradrenaline), and epinephrine (adrenaline), and the skin pigment melanin. It is encoded by the codons UUU and UUC.

It should be noted that phenylalanine and tryptophan are essential amino acids. Essential amino acids, for example, phenylalanine and tryptophan, are amino acid residues that are not synthesized de novo in humans and other animals, and therefore must be provided by an external source. The proteasome dynamics modulating compound/S of the present disclosure comprise at least one of tyrosine, tryptophan and/or phenylalanine, that are interchangeably referred to herein as “tyrosine, tryptophan and/or phenylalanine”, “Tyr, Trp and/or Phe”, “Y, W and/or F”, or “YWF”. It should be noted that every amino acid (except glycine) can occur in two isomeric forms, because of the possibility of forming two different enantiomers (stereoisomers) around the central carbon atom. By convention, these are called L- and D-forms, analogous to left-handed and right-handed configurations. The amino acid residues used in the agonists of the invention can be in D-configuration or L-configuration (referred to herein as D- or L-enantiomers). In yet some further embodiments, the aromatic amino acids of the modulating compounds of present disclosure may comprise at least one amino acid residue in the D-form. As shown by the present disclosure, the L-form of the YWF triad, as well as the D-form of the YWF, effectively inhibited proteasome translocation to the cytosol, and at least one of: activated mTOR and/or increased the localization of mTOR to the lysosomal membrane; activated p38; reduced the phosphorylation of Tyr705 of STAT3; and/or reduced Sestrin3 levels, and/or activity and/or interaction of Sestrin3 with at least one regulatory complex. Moreover, the racemic mixture of both, D-isomers of YWF and L-isomers of YWF, efficiently inhibited proteasome recruitment to the cytosol.

More specifically, as shown by Formula I, II and III, the above-described aromatic amino acids i.e., Tyrosine, Tryptophan and Phenylalanine, possess all a general structure comprising a core structure of 2-aminopropionic acid (alanine) wherein the beta carbon of such structure is substituted with an optionally substituted aryl. In some embodiment, the of the invention must display at least one benzene ring and an Alanine equivalent structure.

In some embodiments, the optionally substituted aryl is a phenolic group wherein the beta carbon of the core structure is connected to such group in a para position relative to the hydroxyl of the phenolic group. Particular embodiments for such structure, may comprise tyrosine.

In some other embodiments, the aryl is a benzene ring. Particular embodiments for such structure, may comprise phenylalanine.

In yet some other embodiments, the aryl is indolyl which is connected to the beta carbon of the core structure via C3 of the indolic substituent. Particular embodiments for such structure, may comprise tryptophan.

Still further, the disclosure contemplates the use of any at least one Y mimetic, at least one W mimetic, or at least one F mimetic which is capable of modulating the proteasome dynamics either alone, or in combination, as measured by proteasome nuclear localization. “Amino acid mimetics”, as used herein, refers to chemical compounds having a structure that is different from the general chemical structure of an amino acid, but that functions in a manner similar to a naturally occurring amino acid.

As used herein “tyrosine mimetic” and “Y mimetic”, “tryptophan mimetic” and “W mimetic” and “phenylalanine mimetic” and “F mimetic”, are used interchangeably to refer to any agent that either emulates the biological effects of tyrosine, tryptophan and/or phenylalanine, on proteasome cellular localization, and/or mTOR activation in a cell, as measured by proteasome nuclear localization in response to the proteasome dynamics modulator/s of the present disclosure, or to any agent that increases, directly or indirectly, the level, and/or bio availability and/or stability of at least one of tyrosine, tryptophan and/or phenylalanine in a cell. The Y, W and/or F mimetic can be any kind of agent. Exemplary Y, W and/or F mimetics include, but are not limited to, small organic or inorganic molecules; L-tyrosine, L-tryptophan and/or L-phenylalanine, D-tyrosine, D-tryptophan and/or D-phenylalanine or any combinations thereof, a tyrosine, tryptophan and/or phenylalanine mimetic, saccharides, oligosaccharides, polysaccharides, a biological macromolecule that may be any one of peptides, non-standard peptides, polypeptides, non-standard polypeptides, proteins, non-standard proteins, peptide analogs and derivatives enriched for L-tyrosine, L-tryptophan and/or L-phenylalanine and/or tyrosine, tryptophan and/or phenylalanine mimetics, peptidomimetics, nucleic acids such as siRNAs, shRNAs, antisense RNAs, ribozymes, and aptamers that directly or indirectly alter the levels of at least one of Y, W, F; an extract made from biological materials selected from the group consisting of bacteria, plants, fungi, animal cells, and animal tissues; naturally occurring or synthetic compositions; and any combination thereof.

The disclosure further contemplates methods of identifying tyrosine, tryptophan and/or phenylalanine mimetics, for example by assessing the ability of a candidate agent to emulate the biological effects of tyrosine, tryptophan and/or phenylalanine on a selective inhibition of proteasome translocation or mTOR activation in a cell, that results in an increase in the nuclear localization of the proteasome. In some embodiments, methods of identifying tyrosine, tryptophan and/or phenylalanine mimetics include assessing the ability of a candidate agent to emulate the biological effects of tyrosine for example, when tyrosine is used in combination with tryptophan and phenylalanine to simulate a selective inhibition of proteasome translocation or mTOR activation, and thereby proteasome nuclear localization in a cell.

The term “protcasome dynamics modulating tyrosine, tryptophan and/or phenylalanine mimetic” as used herein means a mimetic of tyrosine, tryptophan and/or phenylalanine which, when administered to a subject alone(in the form of a single compound or as part of a non-standard peptide, non-standard polypeptide, or non-standard protein, enriched for such mimetic) or in combination with the other components utilized in the present disclosure causes an increase in proteasome nuclear localization, and at least one of increase in mTOR activity and/or lysosomal localization, and/or activation of p38 (specifically, p38 delta), and/or inhibition of STAT3, inhibition of SESN3, specifically, reduction in Sestrin3 levels, and/or activity and/or inhibition of SESN3 interaction with at least one member of the GATOR2 complex, and thereby to an increase in proteasome nuclear localization in one or more cells and/or tissues or cells of that subject, as compared with cytosolic localization of the proteasome and at least one of reduced mTOR activity and/or dissociation from the lysosome, inactivation or p38, activation of STAT3, Sestrin3 levels, and/or activity and/or association of SESN3 with at least one member of the GATOR2 complex, prior to administration of the mimetic. It should be noted that any methods and means may be used for determining the cellular localization of the proteasome. In some embodiments, any of the methods disclosed by the preset disclosure in connection with other aspects of the invention, are also applicable for the present aspect as well. In some embodiments, the subject is determined to be deficient in tyrosine, tryptophan and/or phenylalanine prior to administration. In some embodiments, a tyrosine, tryptophan and/or phenylalanine mimetic causes an increase in proteasome nuclear localization and/or all related parameters indicated above (e.g., mTOR activity and/or lysosomal localization, and/or activation of p38, and/or inhibition of STAT3, and/or inhibition of SESN3), that is between 50% and 500% of the increase caused by administering an equimolar amount of L-tyrosine, L-tryptophan and/or L-phenylalanine and/or D-tyrosine, D-tryptophan and/or D-phenylalanine, and any combinations thereof. In some embodiments, a tyrosine, tryptophan and/or phenylalanine mimetic causes an increase in proteasome nuclear localization and/or any of the related parameters discussed above, that is between 80% and 120% of the increase caused by administering an equimolar amount of L-tyrosine, tryptophan and/or phenylalanine. In some embodiments, a tyrosine, tryptophan and/or phenylalanine mimetic causes a selective inhibition of proteasome translocation and/or an increase in mTOR activity, and/or lysosomal localization and/or any of the related parameters discussed above, and thereby proteasome nuclear localization, that is equal to or greater than the increase caused by administering an equimolar amount of L-tyrosine, L-tryptophan and/or L-phenylalanine. In some embodiments, the Y, W and/or F mimetic is not the native amino acid tyrosine, tryptophan and/or phenylalanine. In some embodiments, the Y, W and/or F mimetic is not a naturally occurring source of tyrosine, tryptophan and/or phenylalanine. In some embodiments, the Y, W and/or F mimetic are not a dietary source of tyrosine, tryptophan and/or phenylalanine. In some embodiments, the Y, W and/or F mimetic comprise the native amino acid tyrosine, tryptophan and/or phenylalanine. As used herein, “native amino acid” refers to the L-form of the amino acid which naturally occurs in proteins; thus, the term “native amino acid tyrosine, tryptophan and/or phenylalanine” refers to L-tyrosine, L-tryptophan and/or L-phenylalanine. In some embodiments, the native amino acid tyrosine, tryptophan and/or phenylalanine is isolated and/or purified. In some embodiments, the amino acid residues can be in D-configuration or L-configuration (referred to herein as D- or L-enantiomers).

In some embodiments, the Y, W and/or F mimetic comprises the native amino acid tyrosine, tryptophan and/or phenylalanine (Y, W and/or F). In some embodiments, the native amino acid tyrosine, tryptophan and/or phenylalanine is isolated and/or purified.

In some embodiments, the Y, W and/or F mimetic comprises a polypeptide comprising the native amino acid tyrosine, tryptophan and/or phenylalanine or any mixture of native and non-native YWF. In some embodiments, the Y, W and/or F mimetic comprises a polypeptide comprising a derivative of the native amino acid tyrosine, tryptophan and/or phenylalanine. In some embodiments, the Y, W and/or F mimetic comprises a polypeptide comprising an analog of the native amino acid tyrosine, tryptophan and/or phenylalanine. In some embodiments, the Y, W and/or F mimetic comprises a polypeptide comprising a combination of the native amino acid tyrosine, tryptophan and/or phenylalanine, a derivative of the native amino acid tyrosine, tryptophan and/or phenylalanine and/or an analog of the native amino acid tyrosine, tryptophan and/or phenylalanine.

In some embodiments, the multimeric and/or polymeric form of the aromatic amino acid resides provided in the proteasome dynamics modulator of the present disclosure further encompass any peptide, non-standard peptide, polypeptide, non-standard polypeptide, protein or non-standard protein any of which is enriched for one, two, or all three aromatic amino acid residues or mimetics thereof, specifically, at least one of Y, W and/or F (tyrosine, tryptophan and/or phenylalanine), and/or any mimetic thereof.

As indicated herein, in some embodiments, the aromatic amino acid residues of the invention may be provided in, or as a polypeptide. A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, ornithine, citrulline, and homocysteine are naturally occurring non-standard amino acids that have important roles in mammalian metabolism. Exemplary nonstandard amino acids include, e.g., singly or multiply halogenated (e.g., fluorinated) amino acids, D-amino acids, homo-ammo acids, N-alkyl amino acids (other than proline), dehydroamino acids, aromatic amino acids (other than histidine, phenylalanine, tyrosine and tryptophan), and α,α disubstituted amino acids, An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides. One of ordinary skill in the art will understand that a protein may be composed of a single amino acid chain or multiple chains associated covalently or noncovalently.

More specifically, the polypeptide comprising the native amino acid tyrosine, tryptophan and/or phenylalanine (and/or analogs and/or derivatives of the native amino acid tyrosine, tryptophan and/or phenylalanine) can be of any length, specifically, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 4, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 250, 500, 1000, or more residues. In some embodiments, the polypeptide comprising tyrosine, tryptophan and/or phenylalanine consists entirely of tyrosine, tryptophan and/or phenylalanine residues. In some embodiments, the polypeptide comprising the native amino acid tyrosine, tryptophan and/or phenylalanine is polypeptide enriched for tyrosine, tryptophan and/or phenylalanine residues. In yet some further embodiments, the polypeptide may comprise any combination or ration of each of the aromatic amino acid resides, specifically, tyrosine, tryptophan and/or phenylalanine. Still further, the polypeptide may be composed one of the tyrosine, tryptophan and/or phenylalanine, and two or more such polypeptides may be combined together and/or administered together. In some embodiments, the polypeptide enriched for tyrosine, tryptophan and/or phenylalanine residues comprises at least 10% content of tyrosine, tryptophan and/or phenylalanine residues relative to other amino acid residues. In some embodiments, the polypeptide enriched for tyrosine, tryptophan and/or phenylalanine residues comprises at least 12%, at least 15%, at least 22%, at least 25%, at least 31%, at least 35%, at least 40%, at least 44%, at least 47%, at least 50%, at least 53%, at least 58%, at least 61%, at least 66%, at least 70%, at least 75%, or more content of tyrosine, tryptophan and/or phenylalanine residues. In some embodiments, the polypeptide enriched for tyrosine, tryptophan and/or phenylalanine residues comprises at least 80%, at least 85%, at least 90%, at least 91%, at least 92%, at least 93%, at least 94%, at least 95%, at least 96%, at least 97%, at least 98%, at least 99% content of tyrosine, tryptophan and/or phenylalanine residues. In yet some further embodiments, the selective modulator of proteasome shuttling, translocation, that also acts in some embodiments, as a modulator of proteasome dynamics in accordance with the present disclosure, may comprise two or more polypeptides each is enriched for at least one of Y, W, F, as discussed above.

In certain exemplary embodiments, disclosed herein is a synthetic oligopeptide, peptide, or polypeptide comprising YWF residues. Such synthetic YWF oligopeptides, peptides, and polypeptides can be of any length (e.g., 2-20 residues, 20-100 residues, 100-1,000 residues, 500-2,000 residues, 1,000-10,000 residues, or longer). The residues comprising such YWF oligopeptides, peptides, or polypeptides can ordered in any fashion, e.g., YWF, YFW, WFY, WYF, FYW, FWY. The residues comprising such YWF oligopeptides, peptides, or polypeptides can also be structured as repeats ordered in any fashion, such as YYY repeats, WWW repeats. FFF repeats, YWF repeats, in certain embodiments, the synthetic YWF oligopeptides, peptides, and polypeptides contains at least 20%, 30%, at least 35%, at least 40%, at least 45%, at least 50%, at least 60%, at least 70%, at least 80%, at least 90%, at least 95%, at least 97% or more, and even 100% YWF content. In some embodiments, the synthetic YWF oligopeptides, peptides, and polypeptides contain at least 10% YWF content. In some embodiments, the synthetic YWF oligopeptides, peptides, and polypeptides contain at least 15% YWF content. In some embodiments, the synthetic YWF oligopeptides, peptides, and polypeptides contain at least 20% YWF content. In some embodiments, the synthetic YWF oligopeptides, peptides, and polypeptides contain at least 25% YWF content. In some embodiments, the synthetic YWF oligopeptides, peptides, and polypeptides contain at least 30% YWF content. In some embodiments, the synthetic YWF oligopeptides, peptides, and polypeptides contain at least 35% YWF content. In some embodiments, the synthetic YWF oligopeptides, peptides, and polypeptides contain at least 40% YWF content. In some embodiments, the synthetic YWF oligopeptides, peptides, and polypeptides contain at least 45% YWF content. In some embodiments, the synthetic YWF oligopeptides, peptides, and polypeptides contain at least 50% YWF content. In some embodiments, the synthetic YWF oligopeptides, peptides, and polypeptides contain at least 55% YWF content. In some embodiments, the synthetic YWF oligopeptides, peptides, and polypeptides contain at least 60% YWF content. In some embodiments, the synthetic YWF oligopeptides, peptides, and polypeptides contain at least 65% YWF content. In some embodiments, the synthetic YWF oligopeptides, peptides, and polypeptides contain at least 70% YWF content. In some embodiments, the synthetic YWF oligopeptides, peptides, and polypeptides contain at least 75% YWF content. In some embodiments, the synthetic YWF oligopeptides, peptides, and polypeptides contain at least 80% YWF content. In some embodiments, the synthetic YWF oligopeptides, peptides, and polypeptides contain at least 85% YWF content. In some embodiments, the synthetic YWF oligopeptides, peptides, and polypeptides contain at least 90% YWF content. In some embodiments, the synthetic YWF oligopeptides, peptides, and polypeptides contain at least 95% YWF content. In some embodiments, the synthetic YWF oligopeptides, peptides, and polypeptides contain 100% YWF content.

In some embodiments, the polypeptide comprising tyrosine, tryptophan and/or phenylalanine is enriched for tyrosine, tryptophan and/or phenylalanine residues. In some embodiments, the polypeptide enriched for tyrosine, tryptophan and/or phenylalanine comprises a tyrosine, tryptophan and/or phenylalanine-rich repeat containing protein or a fragment thereof. Those skilled in the art will appreciate that a variety of methods exist for obtaining polypeptide comprising and/or enriched for tyrosine, tryptophan and/or phenylalanine, including, for example, isolating tyrosine, tryptophan and/or phenylalanine-rich repeats or fragments from polypeptide enriched for tyrosine, tryptophan and/or phenylalanine, synthetic routes, and recombinant methods (e.g., in vitro transcription and/or translation of nucleic acids comprising tyrosine, tryptophan and/or phenylalanine codons UAU, UAC (Tyr), UGG (Trp), UUU, UUC (Phe). Recombinant methods of producing a peptide through the introduction of a vector including nucleic acid encoding the peptide into a suitable host cell is well known in the art, such as is described in Sambrook et al, Molecular Cloning: A Laboratory Manual, 2d Ed, Vols 1 to 8, Cold Spring Harbor, NY (1989); M. W. Pennington and B. M. Dunn, Methods in Molecular Biology: Peptide Synthesis Protocols, Vol 35, Hurnana Press, Totawa, NJ. Peptides can also be chemically synthesized using methods well known in the art.

In some embodiments, a polypeptide comprising tyrosine, tryptophan and/or phenylalanine or enriched for tyrosine, tryptophan and/or phenylalanine is not a dietary source of tyrosine, tryptophan and/or phenylalanine. As used herein, “dietary source of tyrosine, tryptophan and/or phenylalanine” refers to a source of tyrosine, tryptophan and/or phenylalanine in which, prior to ingestion, chewing, or digestion, the tyrosine, tryptophan and/or phenylalanine is found in its natural state as part of an intact polypeptide within the source (e.g., meats (e.g., chicken, beef, etc.), legumes, grains, vegetables, dairy products (e.g., milk, cheese), eggs, nuts, seeds, seafood, etc.).

In some embodiments, a polypeptide comprising tyrosine, tryptophan and/or phenylalanine or enriched for tyrosine, tryptophan and/or phenylalanine does not include any non-essential amino acids other than tyrosine. In some embodiments, a polypeptide comprising tyrosine, tryptophan and/or phenylalanine or enriched for tyrosine, tryptophan and/or phenylalanine does not include any essential amino acids other than tryptophan and phenylalanine. In some embodiments, a polypeptide comprising tyrosine, tryptophan and/or phenylalanine or enriched for tyrosine, tryptophan and/or phenylalanine includes at least one non-native form of the amino acid tyrosine, tryptophan and/or phenylalanine.

In some embodiments, the Y, W and/or F mimetic comprises a derivative of the native amino acid tyrosine, tryptophan and/or phenylalanine. It is contemplated that any derivative of Y, W and/or F which lead to proteasome nuclear localization, can be used. Y, W, and/or F derivatives which leads to proteasome nuclear localization can be readily determined by the skilled artisan according to the teachings disclosed herein (e.g., assaying for Y, W, and/or F derivatives which increase proteasome nuclear localization either alone, or in combination with the amino acids tyrosine, tryptophan and phenylalanine or mimetics of tyrosine, tryptophan or phenylalanine). In some embodiments, the derivative of Y, W, and/or F comprises a C-terminus modification to Y, W, and/or F. As used herein, a “C-terminus modification” refers to the addition of a moiety or substituent group to the amino acid via a linkage between the carboxylic acid group of the amino acid and the moiety or substituent group to be added to the amino acid. The disclosure contemplates any C-terminus modification to Y, W, and/or F in which Y, W, and/or F retains the ability to lead to proteasome nuclear localization, when used alone, or in combination with any of the aromatic amino acids tyrosine, tryptophan and phenylalanine, as measured by proteasome nuclear localization. In some embodiments, the C-terminus modification to Y, W, and/or F comprises a carboxy alkyl of Y, W, and/or F. In some embodiments, the C-terminus modification to Y, W, and/or F comprises a carboxy alky ester of Y, W, and/or F. In some embodiments, the C-terminus modification to Y, W, and/or F comprises a carboxy alkyl ester. As used herein, the term “alkyl” refers to saturated non-aromatic hydrocarbon chain that may be a straight chain or branched chain, containing the indicated number of carbon atoms (these include without limitation methyl, ethyl, propyl, allyl, or propargyl), which may be optionally inserted with N, O, S, SS, S0₂, C(0), C(0)0, OC(O), C(0)N or NC(O). For example, Ci-Ce indicates that the group may have from 1 to 6 (inclusive) carbon atoms in it. In some embodiments, the C-terminus modification to L comprises a carboxy alkenyl ester. As used herein, the term “alkenyl” refers to an alkyl that comprises at least one double bond. Exemplary alkenyl groups include, but are not limited to, for example, ethenyl, propenyl, butenyl, 1-methyl-2-buten-1-yl and the like. In some embodiments, the C-terminus modification to Y, W, F comprises a carboxy alkynyl ester. As used herein, the term “alkynyl” refers to an alkyl that comprises at least one triple bond. In some embodiments, the carboxy ester comprises tyrosine, tryptophan and/or phenylalanine carboxy methyl ester. In some embodiments, the carboxy ester comprises tyrosine, tryptophan and/or phenylalanine carboxy ethyl ester.

In some embodiments, derivative of Y, W and/or F comprises an N-terminus modification to Y, W and/or F. As used herein, “N-terminus modification” refers to the addition of a moiety or substituent group to the amino acid via a linkage between the alpha amino group of the amino acid and the moiety or substituent group to be added to the amino acid. The disclosure contemplates any N-terminus modification to Y, W and/or F in which the N-terminus modified Y, W and/or F retains the ability to lead to proteasome nuclear localization either alone, or in combination with the amino acid tyrosine, tryptophan and phenylalanine, as measured by proteasome nuclear localization.

In some embodiments, the derivative of Y, W, and/or F comprises Y, W, and/or F modified by an amino bulky substituent group. As used herein “amino bulky substituent group” refers to a bulky substituent group which is linked to the amino acid via the alpha amino group. The disclosure contemplates the use of any Y, W, and/or F derivative comprising an amino bulky substituent group that retains its ability to stimulate, enhance and increase proteasome nuclear localization, for example by stimulation of mTOR activation, and/or lysosomal localization, and/or activation of p38, and/or inhibition of STAT3, inhibition of SESN3 and thereby, when used alone, or in combination with the amino acid residues tryptophan and phenylalanine, as measured by proteasome nuclear localization. An exemplary amino bulky substituent group is a carboxybenzyl (Cbz) protecting group. Accordingly, in some embodiments, the derivative of Y, W, and/or F comprises Y, W, and/or F modified by an amino carboxybenzyl (Cbz) protecting group. Other suitable amino bulky substituent groups are apparent to those skilled in the art.

In some embodiments, the derivative of Y, W and/or F comprises a side-chain modification to Y, W and/or F. As used herein “side-chain modification” refers to the addition of a moiety or substituent group to the sidechain of the amino acid via a linkage (e.g., covalent bond) between the side-chain and the moiety or chemical group to be added. The disclosure contemplates the use of any side-chain modification that permits the sidechain modified amino acid to retain its ability to modulate proteasome dynamics, when used alone, or in combination with any one of the amino acids tyrosine, tryptophan and phenylalanine or mimetics thereof, as measured by proteasome nuclear localization. An exemplary side-chain modification is a diazirine modification. Accordingly, in some embodiments, the Y, W and/or F derivative comprises a photo-crosslinkable Y, W, and/or F with a diazirine-modified side chain. In some embodiments, the derivative of Y, W, and/or F comprises an unnatural amino acid. In some embodiments, the derivative of Y, W, and/or F comprises a salt of Y, W, and/or F. In some embodiments, the derivative of Y, W, and/or F comprises a nitrate of Y, W, and/or F. In some embodiments, the derivative of Y, W, and/or F comprises a nitrite of Y, W, and/or F. In some embodiments, the Y, W, and/or F mimetic comprises an analog of the native amino acid tyrosine, tryptophan and/or phenylalanine. It is contemplated that any analog of Y, W, and/or F which modulate proteasome dynamics when used alone, or in combination with the amino acid tryptophan and phenylalanine, as measured by proteasome nuclear localization can be used. Y, W, and/or F analogs which modulate proteasome dynamics can be readily determined by the skilled artisan according to the teachings disclosed herein (e.g., assaying for Y, W, and/or F analogs which increase proteasome nuclear localization). It should be understood that the present disclosure further encompasses in some particular and non-limiting embodiments thereof, any Deuterated, Fluorinated, Acetylated or Methylated forms of any one of the L- or D-tyrosine, the L- or D-phenylalanine or L- or D-tryptophan. More specifically, deuterium-substituted amino acids (deuterated amino acids) applicable as analogs of the present invention may include but are not limited to L-Tyrosine-(phenyl-3,5-d₂), L-4-Hydroxyphenyl-2,3,5,6-d₄-alanin and L-Tryptophan-(indole-d₅). Methylated aromatic amino acids residues include but are not limited to any one of L-Tyrosine methyl ester, O-Methyl-L-tyrosine, α-Methyl-L-tyrosine, α-Methyl-DL-tyrosine methyl ester hydrochloride, α-Methyl-L-tyrosine, α-Methyl-DL-tyrosine, α-Methyl-DL-tryptophan, O-Methyl-L-tyrosine, N-Methyl-phenethylamine, β-Methylphenethylamine, N, N-Dimethylphenethylamine, 3-Methylphenethylamine, (R)-(+)-β-Methylphenethylamine, N-Methyl-N-(1-phenylethyl) amine, 2-methylphenethylamine, 4-Bromo-N-methylbenzylamine, 3-Bromo-N-methylbenzylamine, (S)-β-Methylphenethylamine, p-Chloro-β-methylphenethylamine hydrochl, α-Methyl-DL-tryptophan, L-Tryptophan methyl ester hydrochloride, D-Tryptophan methyl ester hydrochloride, L-Tryptophan ethyl ester hydrochloride, L-Tryptophan benzyl ester, L-Tyrosine methyl ester hydrochloride, L-Phenylalanine methyl ester hydrochlori, DL-tryptophan methyl ester, N-acetyl-l-tryptophan methyl ester. Still further, Fluorinated tyrosine, phenylalanine or tryptophan include but are not limited to any one of 5-Fluoro-L-tryptophan, 5-Fluoro-DL-tryptophan, 4-Fluoro-DL-tryptophan, 6-Fluoro-L-Tryptophan, 5-Methyl-DL-tryptophan, 5-Bromo-DL-tryptophan, 7-Azatryptophan, m-Fluoro-DL-tyrosine, p-Fluoro-L-phenylalanine, o-Fluoro-DL-phenylalanine, p-Fluoro-DL-phenylalanine, 4-Chloro-DL-phenylalanine, m-Fluoro-L-phenylalanine, 3-Nitro-L-tyrosine. In some further embodiments of the present disclosure Acetylated aromatic amino acids residues include but are not limited to any one of N-acetyl-L-tyrosine, N-Acetyl-L-phenylalanine, L-Phenylalanine methyl ester hydrochloride, N-Acetyl-D-phenylalanine, N-Acetyl-L-tryptophan.

Exemplary analogs of tyrosine and/or phenylalanine that may be applicable in accordance with the present disclosure include but are not limited to any one of (2R, 3S)/(2S, 3R)-Racemic Fmoc-β-hydroxyphenylalanine, Boc-2-cyano-L-phenylalanine, Boc-L-thyroxine, Boc-O-methyl L-tyrosine, Fmoc-β-methyl-DL-phenylalanine, Fmoc-2-cyano-L-phenylalanine, Fmoc 3,4-dichloro-L-phenylalanine, Fmoc-3,4-difluoro-L-phenylalanine, Fmoc-3,4-dihydroxy-L-phenylalanine, Fmoc-3,4-dihydroxy-phenylalanine, acetonide protected, Fmoc-3-amino-L-tyrosine, Fmoc-3-chloro-L-tyrosine, Fmoc-3-fluoro-DL-tyrosine, Fmoc-3-nitro-L-tyrosine, Fmoc-4-(Boc-amino)-L-phenylalanine, Fmoc-4-(Boc-aminomethyl)-L-phenylalanine, Fmoc-4-(phosphonomethyl)-phenylalanine, Fmoc-4-(phosphonomethyl)-phenylalanine, Fmoc-4-benzoyl-D-phenylalanine. Still further, in some embodiments, exemplary analogs of tryptophan that may be applicable in accordance with the present disclosure include but are not limited to any one of Boc-4-methyl-DL-tryptophan, Boc-4-methyl-DL-tryptophan, Boc-6-fluoro-DL-tryptophan, Boc-6-methyl-DL-tryptophan, Boc-DL-7-azatryptophan, Fmoc-(R)-7-Azatryptophan, Fmoc-5-benzyloxy-DL-tryptophan, Fmoc-5-bromo-DL-tryptophan, Fmoc-5-chloro-DL-tryptophan, Fmoc-5-fluoro-DL-tryptophan, Fmoc-5-fluoro-DL-tryptophan, Fmoc-5-hydroxy-L-tryptophan, Fmoc-5-hydroxy-L-tryptophan, Fmoc-5-methoxy-L-tryptophan, Fmoc-5-methoxy-L-tryptophan, Fmoc-6-chloro-L-tryptophan, Fmoc-6-methyl-DL-tryptophan, Fmoc-7-methyl-DL-tryptophan, Fmoc-DL-7-azatryptophan.

In some embodiments, the Y, W, and/or F mimetic comprises a metabolite of the native amino acid tyrosine. It is further contemplated that any metabolite of tyrosine that modulate proteasome dynamics thereby leading to nuclear localization of the proteasome and at least one of at least one of: activation of mTOR and/or increased localization of mTOR to the lysosomal membrane; activation of p38; reduced phosphorylation of Tyr705 of STAT3; and/or reduced interaction of Sestrin3 with at least one regulatory complex, either alone or in combination with the amino acid residues tryptophan and phenylalanine or mimetics thereof can be used. Y, W, and/or F derivatives which modulate proteasome dynamics thereby leading to nuclear localization of the proteasome can be readily determined by the skilled artisan according to the teachings disclosed herein (e.g., assaying for metabolites of Y, W, and/or F which increase proteasome nuclear localization when used alone, or in combination with tryptophan and phenylalanine or mimetics thereof.

It should be appreciated that the present disclosure provides the aromatic amino acid residues, specifically, tyrosine, tryptophan and/or phenylalanine and/or any serogates thereof, any salt, base, ester or amide thereof, any enantiomer, stereoisomer or disterioisomer thereof, or any combination or mixture thereof. Pharmaceutically acceptable salts include salts of acidic or basic groups present in compounds, specifically, the aromatic amino acid residues of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate(i.e., 1,l′-methylene-bis-(2-hydroxy-3-naphthoate)) salts. Certain aromatic amino acid residues of the present disclosure can form pharmaceutically acceptable salts. Suitable base salts include, but are not limited to, aluminum, calcium, lithium, magnesium, potassium, sodium, zinc, and diethanolamine salts. The present disclosure provides effective modulators of proteasome dynamics that may comprise either one aromatic amino acid residue, for example, any one of tyrosine, tryptophan and/or phenylalanine or any mimetics thereof, or any combination of at least two of tyrosine, tryptophan and/or phenylalanine and/or mimetics thereof. As such, the present disclosure further provides combinations, specifically combinations comprising at least two of tyrosine, tryptophan and/or phenylalanine, and/or any mimetics or derivatives thereof. In some embodiments, the effective amount of the at least one modulator of proteasome dynamics in the combination of the present disclosure is sufficient for modulating proteasome dynamics in at least one cell.

In some embodiments, the selective inhibitor of proteasome translocation, and/or modulator of proteasome dynamics, that further exhibits at least one of: activation of mTOR and/or increased localization of mTOR to the lysosomal membrane; activation of p38; reduced phosphorylation of Tyr705 of STAT3; and/or reduced interaction of Sestrin3 with at least one regulatory complex in accordance with the present disclosure may comprise at least one tyrosine residue, any mimetic, any salt or ester thereof, any multimeric and/or polymeric form thereof, and any combinations or mixtures thereof, and at least one tryptophane residue, any mimetic, any salt or ester thereof, any multimeric and/or polymeric form thereof, and any combinations or mixtures thereof. In some further embodiments, the modulator of proteasome dynamics in accordance with the invention may comprise at least one tyrosine residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. In yet some further embodiments, the proteasome dynamics modulator, that can be also an mTOR agonist in accordance with the invention may comprise at least one tryptophane residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof.

In some particular embodiments, the modulator of proteasome dynamics of the present disclosure may comprise the following three components: first component (a), comprises at least one tyrosine residue, any tyrosine mimetic, any salt or ester thereof, any multimeric and/or polymeric form of the tyrosine residue and/or of the tyrosine mimetic, and any combinations or mixtures thereof. The modulator of proteasome dynamics of the present disclosure further comprises component (b), at least one tryptophan residue, any tryptophan mimetic, any salt or ester thereof, any multimeric and/or polymeric form of the tryptophan residue and/or of said tryptophan mimetic, or any combination or mixture thereof. The modulator of proteasome dynamics disclosed herein further comprises component (c), phenylalanine residue, any phenylalanine mimetic, any salt or ester thereof, any multimeric and/or polymeric form of the phenylalanine residue and/or of the phenylalanine mimetic, and any combinations or mixtures thereof. It should be understood that the aromatic amino acid residues of the modulating compounds of the present disclose or any mimetics thereof, may be presented in a mixture of all three YWF, at any appropriate quantitative ratio. The quantitative ratio used may be for example, 1:1:1, 1:2:3, 1:10:100, 1:10:100:1000 etc, or any one of 1-106:1-106:1-106. In some embodiments the quantitative ratio may be any one of 1:1:1 1:1:2, 1:1:3, 1:1:4, 1:1:5, 1:1:6, 1:1:7, 1:1:8, 1:3, 1:4, 1:5, 1:6, 1:7, 1:8, 1:9, 1:10, 1:2:1, 1:3:1, 1:4:1, 1:5:1, 1:6:1, 1:7:1, 1:8:1, 1:9:1, 1:10:1, 2:1:1, 3:1:1, 4:1:1, 5:1:1, 6:1:1, 7:1:1, 8:1:1, 9:1:1, 10:1:1, or any other suitable ratio of the three aromatic amino acid residues.

To facilitate the therapeutic and non-therapeutic uses of the modulators of proteasome dynamics and combinations disclosed herein, the present disclosure further provides compositions comprising the modulators of proteasome dynamics and combinations of the disclosure.

In some embodiments, the disclosed compound that modulates proteasome dynamics and/or function (also referred to herein as the modulatory compound) useful in the disclosed methods may be, or may comprise at least one of: a nucleic acid-based molecule, an amino acid-based molecule, a small molecule or any combinations thereof. In yet some further additional or alternative embodiments, the modulatory compound may target at least one of the signaling molecule/s, at the nucleic acid sequence level or at the protein level. In some specific embodiments, the disclosed modulatory compound used in the methods of the present disclosure my target any one of the mediator/s of amino acid sensing (e.g., at least one member of the Sestrin family), the at least one member of the MAPKs, specifically, members of the p38 mitogen-activated protein kinases (p38 MAPK, p38), at least one nucleo-cytosolic shuttle protein/s, and/or at least one NPC, at the nucleic acid sequence level or at the protein level. In yet some more specific embodiments, the disclosed modulator useful in all methods and compositions of the present disclosure, may target any one of SESN3, p38 (particularly p38 delta), p62 and/or NBR1, NUP93, and/or STAT3 at the nucleic acid sequence level or at the protein level.

In more specific embodiments, in some embodiments, the modulatory compounds of the present disclosure specifically target the at least one signaling molecule (e.g., SESN3, p38, p62, NBR1, NUP93) at the nucleic acid level, thereby affecting the expression, distribution and/or splicing of such target signaling molecule. In yet some additional or alternative embodiments, the disclosed modulatory compound may specifically target the at least one signaling molecule (e.g., SESN3, p38, p62, NBR1, NUP93) at the protein level, thereby affecting the stability, activity, PTMs, and/or the interactions of such target signaling molecule with other signaling molecules. In some specific embodiment, useful modulatory compounds that may target the target signaling molecule at the nucleic acid level (a), thereby affecting the expression, distribution and/or splicing of the at least one target signaling molecules (thereby modulating proteasome dynamics in the cell). In some embodiments, such compound may be or may comprise gRNA, small interfering RNA (siRNA), short hairpin RNA (shRNA), microRNA (miRNA), MicroRNA-like RNAs (milRNA), and/or artificial miRNAs (amiRNA). Non-limiting embodiments for specific gRNAs, siRNAs, and/or shRNAs targeting each of the disclosed signaling molecules, are disclosed by the present disclosure. Non-limiting embodiments, include any of the compounds that comprise the nucleic acid sequences of any one of SEQ ID NO: 1 to 12, and 14 to 24, and SEQ ID NO: 49 to 60 or any derivatives and variants thereof.

In yet some further additional or alternative embodiments, the modulatory compounds that may target the target signaling molecule at the protein level (b). According to some further embodiments, the compound may reduce the stability of said target protein by targeted protein degradation (TPD), as discussed above. For example, TPD via proteasome (PROTAC, molecular glue, double-mechanism degrader, other PROTAC-based technologies: SARD, HIT, FT-PROTAC), or CHAMP (Chaperone-mediated Protein Degradation/Degrader); or TPD via lysosome.

In some embodiments, Sestrin3 levels and/or activity may be reduced by at least one of: (i) specifically targeting a nucleic acid sequence encoding the Sestrin3, or any parts thereof; (ii) specifically targeting a nucleic acid sequence involved directly or indirectly in regulation of the Sestrin3 gene expression; (iii) reducing the stability (increasing degradation) of the Sesn3 protein; and/or (iv) interfering with the interaction of Sestrin3 with at least one regulatory complex. In some optional embodiments, the Sestrin3 targeting compound used in the personalized methods may be, or may comprise any of the following compounds. In some embodiments, (I), the compound may target the nucleic acid sequence encoding Sestrin3, r alternatively, at least one nucleic acid sequence regulating and/or controlling the expression of Sestrin3. In some embodiments, such nucleic acid targeting compound may comprise at least one of: (a) at least one RNA guide (gRNA) that guides least one nucleic acid guided genome modifier protein to at least one target sequence within the Sestrin3 encoding nucleic acid sequence, or within a nucleic acid sequence involves directly or indirectly in regulation of the Sestrin3 gene expression; or at least one nucleic acid sequence encoding the nucleic acid guide. In some optional embodiments, the disclosed compound may further comprise (b), at least one nucleic acid guided genome modifier protein, or any chimeric protein, complex or conjugate thereof, or at least one nucleic acid sequence encoding the guided genome modifier protein or chimeric protein thereof. In yet some other alternative or additional embodiments (II), the compounds applicable in the present disclosure may be at least one compound that reduces the stability of Sesn3 by targeted protein degradation (TPD). In some further additional or alternative embodiments, the compound may be (III), at least one compound that interferes and/or blocks the inhibitory interaction of Sestrin3 with at least one member of the GATOR2 complex.

In some embodiments the compound used in the methods of the present disclosure may comprise a gene editing system that targets the nucleic acid sequence encoding Sestrin3, and/or any target nucleic acid sequence (e.g., cither coding or non-coding) that controls or regulate the expression and/or splicing of Sesrin3. Such target sequences may reside within promoters/enhancers, splice donor and/or acceptor sites, lncRNA, miRNA, and the like. Specifically, in some embodiments, the compound of the present disclosure is any gene editing system or any component/s thereof. Thus, the personalized treatment regimen monitored and determined by disclosed methods comprise the use of a gene editing system that targets the Sestrin3 coding and/or non-coding sequences (and/or any target nucleic acid sequence (e.g., cither coding or non-coding) that controls or regulate the expression and/or splicing of Sesrin3) thereby leading to reduction in the expression and/or activity thereof. In some specific embodiments, the compound used in the treatment regimen of the methods of the present disclosure comprise: (a) at least one RNA guide (gRNA) that guides least one nucleic acid guided genome modifier protein to at least one target sequence within the Sestrin3 encoding nucleic acid sequence, or within a nucleic acid sequence involves directly or indirectly in regulation of the Sestrin3 gene expression, or at least one nucleic acid sequence encoding said nucleic acid guide. The compound of the present disclosure may further comprise in some optional embodiments thereof (b), at least one nucleic acid guided genome modifier protein, or any chimeric protein, complex or conjugate thereof, or at least one nucleic acid sequence encoding the guided genome modifier protein or chimeric protein thereof. Non-limiting embodiments relate to the sgRNA compounds as disclosed by SEQ ID NO: 1, 2,3, and to the siRNA compounds directed against Sestrin 3 as denoted by SEQ ID NO: 49, 50, 51, 52, or any combinations thereof.

In yet some additional or alternative embodiments, the sestrin3 may be targeted functionally, by the compounds used in the treatment regimen of the methods of the present disclosure. More specifically, such compound may interfere with Sestrin3 function, in some embodiments, by blocking any downstream pathways and/or interactions thereof. Thus, in some embodiments, the compound of the disclosed in the treatment regimen monitored and determined by the personalized methods disclosed herein, may be any compound that interferes and/or blocks, and/or reduces the interaction of Sestrin3 with at least one regulatory complex. In some embodiments, the compounds used by the methods of the present disclosure may be any compound that interferes with, and/or blocks, and/or inhabits, and/or reduces, and/or decreases the inhibitory interaction of Sestrin3 with at least one member of the GATOR2 complex, specifically, with MIOS and/or WDR59. In yet some other alternative or additional embodiments, the compounds applicable in the present disclosure may be at least one compound that reduces the stability of Sesn3 by targeted protein degradation (TPD).

Still further, in some embodiments, the compound used as the therapeutic compound in the treatment regimen monitored and/or determined by the personalized methods of the present disclosure, may target the p38 protein. In some embodiments, p38 delta. In some embodiments, the compound applicable in the disclosed compounds may be p38 activator/s. More specifically, in some embodiments, the compound is a p38 activator that leads to phosphorylation of p38. In some embodiments, such activating phosphorylation is a phosphorylation in at least one of Thr180 (T180) and/or Tyr 182 (Y182) of p38. Still further, in some specific and non-limiting embodiments, a p38 activator useful in the disclosed personalized methods may be a compound elevating the levels and/or activity of MAP kinase kinase 3 (MKK3) and/or of MKK6. In yet some further additional or alternative embodiments, a p38 activator useful in the disclosed personalized methods may be at least one hyperosmotic agent. Non-limiting embodiments for such hyperosmotic agent, may be sorbitol. Thus, in some embodiments, any carbohydrates having a hyperosmotic effect may be used. To name but few, glycerin (glycerol), isosorbide, mannitol and urea may be used as the disclosed compounds. In some embodiments, the compound used by the personalized methods of the present disclosure may be sorbitol. Still further, in some additional or alternative embodiments, a p38 activator useful in the disclosed methods may be at least one DNA Synthesis Inhibitor. In some specific embodiments, such compound may be anisomycin.

In some embodiments, at least two compounds are used, specifically, at least one compound that reduces the level and/or activity of p62 and of NBR1. In some embodiments, such compounds may target the coding or non-coding nucleic acid sequences of each one of p62 and NBR1. Non-limiting embodiments for such compounds may be the siRNA molecules used in the present disclosure. In some embodiments, such siRNA compounds may comprise the nucleic acid sequence as denoted by any one of SEQ ID NO: 53, 54, 55, 56, 57, 58, 59, 60, and any combinations thereof.

In yet some further alternative or additional embodiments, the compound used as the therapeutic compound in the treatment regimen monitored and/or determined by the personalized methods of the present disclosure may target STAT3. More specifically, in some embodiments, the compound may be any STAT3 inhibitor, for example, any compound that inhibits and/or reduces phosphorylation of STAT3. In some particular embodiments, any compound that inhibits and/or reduces phosphorylation of Tyr705 of STAT3. In some specific and non-limiting embodiments, STAT3 inhibitors that may be useful in the methods disclosed herein may include small molecule compounds, specifically, Stattic (Stat three inhibitory compound), S31-201/NSC74859, BP-1-102, Niclosamide, peptide inhibitors (e.g., the peptide aptamer APT STAT3-9R, and the like). In some specific embodiments, Stattic may be used as the therapeutic compound in the treatment regimen monitored and/or determined by the personalized methods of the present disclosure. Additional inhibitors may be based on siRNA and/or shRNA molecules that specifically target the STAT3 encoding or non-encoding sequences, thereby leading to reduced or eliminated expression thereof. In yet some further embodiments, the prognostic method is applied on a subject suffering from a pathogenic disorder. In yet some further embodiments, the diagnosed subject is suffering from at least one of, at least one proliferative disorder, and/or at least one protein misfolding disorder or deposition disorder.

In some embodiments, the proliferative disorder relevant to the method of the invention may be at least one solid or non-solid cancer, or any metastasis thereof.

In some specific embodiments, a proliferative disorder may be at least one hematological malignancy, and any related condition. Still further, in some embodiments, a protein misfolding disorder or deposition disorder may be amyloidosis and any related conditions.

In some embodiments, the personalized methods of the present disclosure may be applicable for any disorder affected by proteasomal activity and/or cellular localization. In some specific embodiments, such disorder is at least one of: at least one neoplastic disorder and/or at least one protein misfolding disorder or deposition disorder. Still further, in some embodiments, the disclosed personalized methods may be applicable for any malignant and non-malignant neoplastic disorders. In some specific embodiments, the disclosed methods may be used for treating malignant neoplastic disorder.

A further aspect of the present disclosure relates to a screening method for identifying at least one modulator of proteasome dynamics and/or function. More specifically, the methods comprising the following steps. One step (a) involves determining in at least one cell contacted with a candidate compound, or in any fraction of the cell or any sample thereof, at least one of the following parameters (I). In some embodiments (i), mTOR activation and/or lysosomal association in the presence and/or absence of the candidate compound is examined. In yet some additional or alternative embodiments, (ii) activation of p38 in the presence and/or absence of the candidate compound is examined. Still further in some alternative or additional embodiments (iii), phosphorylation of Tyr705 of STAT3 in the presence and/or absence of the candidate compound is examined. In some further additional or alternative embodiments (iv), the cell viability, or in other words, the cytotoxicity, in the presence and/or absence of the candidate compound is examined. In some embodiments, cytotoxicity of the candidate compound may be evaluated by determining apoptosis in the cells. Still further, in some alternative or additional embodiments (v), the level of at least one cytosolic and/or nuclear substrate of the proteasome in the presence and/or absence of the candidate compound is examined. Still further, in some alternative or additional embodiments and/or (vi) Sestrin3 levels, and/or activity and/or the interaction of Sestrin3 with at least one regulatory complex; and optionally (II) the proteasome subcellular localization in the presence and/or absence of the candidate compound is examined.

In another step (b), the method involves determining that the candidate compound is:

Either (I), an inhibitor of proteasome translocation/recruitment and/or of proteasome assembly, if at least one of: (i) mTOR is activated and/or localized to the lysosomal membrane; (ii) p38 is activated (e.g., phosphorylated in at least one of T180 and Y182); (iii) phosphorylation of Tyr705 of STAT3 is inhibited or reduced; (iv) the cell display reduced viability; (v) the level of at least one cytosolic substrate of the proteasome is maintained; and/or (vi) Sestrin3 levels, and/or activity and/or the interaction of Sestrin3 with at least one regulatory complex are reduced; and optionally, the ratio of nuclear to cytosolic proteasome subcellular localization is greater than 1, e.g., in the examined cell (specifically, when compared with a cell that was not contacted with the candidate compound).

Alternatively (II), the candidate compound is determined as an enhancer of proteasome translocation/recruitment and/or of proteasome assembly, if at least one of: (i) mTOR is inactivated and/or dissociated from the lysosomal membrane; (ii) p38 is inactivated (e.g., de-phosphorylation of at least one of T180 and Y182); (iii) Tyr705 of STAT3 is phosphorylated; (iv) cell is viable; (v) the level of at least one cytosolic substrate of the proteasome is reduced; and/or (vi) Sestrin3 levels, and/or activity and/or the interaction of Sestrin3 with at least one regulatory complex are increased or unchanged; and optionally, the ratio of nuclear to cytosolic proteasome subcellular localization is smaller than or equal to 1, e.g., in the examined cell (specifically, when compared with a cell that was not contacted with the candidate compound).

In some embodiments, the screening methods of the invention is used for identifying p38 activators that modulate proteasome dynamics and/or function. Thus, in addition to p38 activation, the modulator should display at least one of dephosphorylation of Tyr705 of STAT3, mTOR activation and localization to the lysosomal membrane, and reduced cell viability.

In some embodiments, the screening methods of the present disclosure is used for identifying Sestrin3 inhibitors that modulate proteasome dynamics/function. Thus, in addition to knockdown of Sestrin3, the modulator should display at least one of p38 activation, dephosphorylation of Tyr705 of STAT3, mTOR activation and localization to the lysosomal membrane, and reduced cell viability.

As indicated herein, the present disclosure provides methods for screening for selective modulators of proteasome translocation. As used herein a “modulator”, “modulating compound”, “modulatory compound”, or “proteasome modulator”, that are interchangeably used herein, mean any compound leading, causing or facilitating a qualitative or quantitative change, alteration, or modification in a molecule, a process, pathway, or phenomenon of interest. Specifically, proteasome dynamics, e.g., translocation of the proteasome from nucleus to the cytosol. Without limitation, such change may be an increase, elevation, enhancement, augmentation of the translocation of the proteasome. In yet some alternative embodiments, the change may be decrease, reduction, inhibition, attenuation, of the proteasome translocation to the cytosol.

As indicated herein, the present disclosure further provides a screening method for at least one proteasome dynamics modulator/s. Such modulator may be used in some embodiments to direct the proteasome to the nucleus. Preferably, in various pathological and/or physiological conditions and processes. The method of the invention comprises the step of determining at least one of (i) mTOR lysosomal association; (ii) activation of p38; (iii) phosphorylation of Tyr705 of STAT3; (iv) the interaction of Sestrin3 with at least one regulatory complex; and (v) proteasome subcellular localization in at least one cell of the at least one sample, or in any fraction thereof; in at least one cell contacted with at least one candidate compound or with a plurality of candidate compounds. In some embodiments, the cell contacted with the candidate under basal conditions.

The candidate compound may be any inorganic or organic molecule, any small molecule, nucleic acid-based molecule, any aptamer, any peptide (L- as well as D-aa residues), any lipid, any carbohydrate or any combinations thereof. The candidate may be any natural or synthetic molecule. The candidate may be any chimeric or fusion protein, or any small molecule-peptide conjugate (e.g., PROTAC), or any of the compounds disclosed by the present disclosure. A compound to be tested may be referred to as a test compound or a candidate compound. Any compound may be used as a test or a candidate compound in various embodiments. In some embodiments a library of FDA approved compounds appropriate for human may be used.

Compound libraries are commercially available from a number of companies including but not limited to Maybridge Chemical Co. (Trevillet, Cornwall, UK), Comgenex (Princeton, NJ), Microsource (New Milford, CT), Aldrich (Milwaukee, WI), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, CA), ChemDiv, (San Diego, CA), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, NJ), Interbio screen (Moscow, Russia), Interchim (Montlucon, France), Life Chemicals, Inc. (Orange, CT), Microchemistry Ltd. (Moscow, Russia), Otava, (Toronto, ON), PharmEx Ltd. (Moscow, Russia), Princeton Biomolecular (Monmouth Junction, NJ), Scientific Exchange (Center Ossipee, NH), Specs (Delft, Netherlands), TimTec (Newark, DE), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. Compounds isolated from natural sources, such as animals, bacteria, fungi, plant sources, and marine samples may be tested for the presence of potentially useful pharmaceutical compounds, specifically, selective modulators of proteasome translocation. It will be understood that the agents to be screened could also be derived or synthesized from chemical compositions or man-made compounds. In some embodiments a library useful in the present invention may comprise at least 10,000 compounds, at least 50,000 compounds, at least 100,000 compounds, at least 250,000 compounds, or more.

In some specific embodiments, a candidate compound screened by the screening methods of the invention may be a small molecule. A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

The preset disclosure provides specific modulators of proteasome dynamics, and/or function, e.g., translocation and screening methods for identifying these specific modulators, for example, selective modulators, specifically, inhibitors of proteasome translocation into the cytoplasm. The present disclosure further demonstrated the therapeutic potential of such selective inhibitors (e.g., the YWF, triad), in selective killing of cancer cells. The invention therefore encompasses uses of any selective modulator/s of proteasome dynamics (e.g., compartmentalization), and specifically any selective inhibitors of proteasome translocation from the nucleus for selective induction of apoptosis and cell death of cancer cells. Thus, a further aspect of the present disclosure relates to a method for selective induction of apoptosis of cancer cells, by selective inhibition of proteasome translocation to the cytosol of these cells. In some embodiments, the method comprises contacting the cells with an effective amount of at least one selective inhibitor of proteasome translocation, or with any composition comprising said selective inhibitor.

A further aspect of the present disclosure relates to a method for modulating proteolysis in at least one cell. More specifically, the method comprising the step of contacting the cell with an effective amount of at least one compound that modulates proteasome dynamics and/or function or subjecting the cell to conditions that modulate the proteasome dynamics/function. In some embodiments, the compound and/or conditions are characterized by affecting at least one of: mTOR lysosomal association, proteasome cellular localization, the activity and/or level/s, and/or PTMs and/or localization of at least one signaling molecule participating directly or indirectly in at least one signaling pathway mediating said proteasome dynamics and/or function.

In some embodiments, at least one signaling molecule participating directly or indirectly in said signaling pathway mediating proteasome dynamics and/or function is at least one of: at least one stress-induced protein/s, at least one mediator of cellular response to environmental cues, at least one shuttle protein/s, and at least one NPC protein. Still further, in some embodiments, at least one signaling molecule participating directly or indirectly in the signaling pathway mediating proteasome dynamics and/or function may be at least one of: at least one mediator of metabolite sensing, at least one stress kinase, at least one nucleo-cytosolic shuttle protein, and at least one NPC protein.

In some embodiments of the proteolysis modulatory methods, the mediator of metabolite sensing is a mediator of amino acid sensing. In yet some alternative or additional embodiments, the stress kinase is at least one member of the MAPKs.

In some embodiments, at least one of: (i) the at least one mediator of amino acid sensing is at least one member of the Sestrin family; and/or (ii), the at least one member of the MAPKs is at least one member of the p38 MAPKs; and/or (iii), the at least one nucleo-cytosolic shuttle protein/s is p62 and NBR1, and/or (iv) the at least one NPC is NUP93. More specifically, in some embodiments, such signaling molecule affected by the compound used in the methods of the present disclosure, may be at least one stress-induced protein/s. In some embodiment, such proteins may be is at least one of Sestrin3, and at least one p38 mitogen-activated protein kinases (p38 MAPK, p38). Still further, in some embodiments, the signaling molecule affected by the compounds used in the disclosed methods may be a mediator of cellular response to environmental cues. More specifically, such mediator may be according to some embodiments, the Signal transducer and activator of transcription 3 (STAT3). In yet some further embodiments, the signaling molecule affected by the compound of the disclosed method may be at least one shuttle protein/s, such as for example, at least one of Sequestosome 1 (SQSTM1, p62) and NBR1, and the at least one NPC is NUP93.

In some embodiments of the disclosed methods, the desired modulation of proteolysis by the compound and/or conditions results in proteasome recruitment/translocation to the cytosol and increased cytosolic proteolysis. Accordingly, a compound or conditions useful in increasing cytosolic proteolysis may be any compound and/or conditions that lead to, or are characterized by, at least one of:

In some embodiments, a compound or conditions that lead to, or result in (i), specific substruction of at least one of the aromatic amino acid residue/s tyrosine (Y), tryptophan (W), and phenylalanine (F), or any combinations thereof.

In some alternative or additional embodiments, a compound or conditions that lead to or result in (ii) inhibition and/or silencing of mTOR (specifically, dissociation from the lysosomal membrane). In some additional or alternative embodiments, a compound or conditions that lead to or result in (iii), inhibition and/or silencing of p38 (specifically, dephosphorylation of at least one of T180/Y182). Still further, in some alternative or additional embodiments, a compound or conditions that lead to or result in (iv), activation of STAT3 (specifically, Y705 phosphorylation). In some alternative or additional embodiments, a compound or conditions that lead to or result in (v) inhibition and/or silencing of NUP93. In some alternative or additional embodiments, a compound or conditions that lead to or result in (vi), inhibition and/or silencing of protein/s participating and/or mediating nuclear import of the proteasome (AKIRIN2). Akirin-2 is a protein that in humans is encoded by the AKIRIN2 gene. It is involved in nuclear protein degradation by promoting import of proteasomes into the nucleus. Based on similarity (UniProt), Akirin-2 is assumed to directly bind to fully assembled 20S proteasomes at one end and to nuclear import receptor IPO9 at the other end, bridging them together and mediating the import of pre-assembled proteasome complexes through the nuclear pore. In some alternative or additional embodiments, a compound or conditions that lead to or result in (vii), increase in the association of Sestrin3 with at least one member of the GATOR2 complex.

In some embodiments, Torin 1 an mTOR inhibitor, may be used for increasing cytosolic proteolysis in a cell of the subject.

In some embodiments of the disclosed methods for increasing cytosolic proteolysis, the cell is of a subject suffering from a pathologic disorder associated with cytosolic accumulation of protein/s and/or polypeptides. Accordingly, the step of contacting the cell with a compound and/or subjecting the cell to conditions, is performed by administering to the subject a therapeutic effective amount of the at least one compound, and/or subjecting the subject to the conditions, as defined herein above.

Still further, in some embodiments, the disclosed methods may be applicable to any disorder associated with cytosolic accumulation of protein/s, for example, amyloidosis and aggregation diseases. In yet some further embodiments, the disclosed method may be applicable to any disorder where unfolded protein stress plays a role, for example, pulmonary viral infection and disease, asthma and pulmonary fibrosis, Liver steatosis, Diabetes and lipid disorders, and drug toxicity. In yet some alternative embodiments, the disclosed methods for modulation of proteolysis, are aimed at reducing cytosolic proteolysis and/or increasing nuclear proteolysis. Thus, in some embodiments, the modulatory methods disclosed herein may use compound/s and/or conditions that result in nuclear sequestration of the proteasome and increased nuclear proteolysis. In some embodiments, such compound/s and/or conditions lead to, or are characterized by, at least one of: In some alternative or additional embodiments, a compound or conditions that lead to or result in (i). specific elevation of the levels of at least one of the aromatic amino acid residue/s Y, W, and F, or any combinations thereof. In some alternative or additional embodiments, a compound or conditions that lead to or result in (ii), activation of mTOR (specifically, association to the lysosomal membrane). In some alternative or additional embodiments, a compound or conditions that lead to or result in (iii), inhibition and/or silencing of Sestrin3. In some alternative or additional embodiments, a compound or conditions that lead to or result in (iv), activation and/or upregulation of p38 (specifically, phosphorylation of at least one of T180/Y182). In some alternative or additional embodiments, a compound or conditions that lead to or result in (v), inhibition of STAT3 (specifically, Y705 dephosphorylation). In some alternative or additional embodiments, a compound or conditions that lead to or result in (vi), inhibition and/or silencing of at least one of p62 and NBR1. In some alternative or additional embodiments, a compound or conditions that lead to or result in (vii) proteasome inhibition (e.g., proteasome inhibitors).

In some alternative or additional embodiments, a compound or conditions that lead to or result in (viii), activation of MEK3 and/or MEK6. In some alternative or additional embodiments, a compound or conditions that lead to or result in (ix), reduction of the interaction of Sestrin3 with at least one regulatory complex.

In some embodiments of the disclosed methods for decreasing cytosolic proteolysis and/or increasing nuclear proteolysis, the cell is of a subject suffering from a pathologic disorder associated with nuclear accumulation of protein/s and/or polypeptides, and/or a disorder characterized with/deteriorated by cytosolic accumulation of the proteasome and/or increased cytosolic proteolysis. According to such embodiments, the step of contacting said cell with a compound and/or subjecting the cell to conditions, is performed by administering to the subject a therapeutic effective amount of the at least one compound, and/or subjecting the subject to said conditions, as defined herein above by the present disclosure.

In yet some further embodiments, the pathologic disorder is at least one of: disorders associated with nuclear accumulation of transcription factors and/or oncogene/s, disorders associated with accumulation of proteins in the nuclear lamina (e.g., Hutchinson-Gilford Progeria syndrome (HGPS), aging and premature-aging syndromes), disorder/s associated with/deteriorated by enhanced cytosolic proteolysis, specifically, neoplastic disorders. More specifically, Transcription factor (TF) (or sequence-specific DNA-binding factor) is a protein that controls the rate of transcription of genetic information from DNA to messenger RNA, by binding to a specific DNA sequence. The function of TFs is to regulate-turn on and off-genes in order to make sure that they are expressed in the desired cells at the right time and in the right amount throughout the life of the cell and the organism. Groups of TFs function in a coordinated fashion to direct cell division, cell growth, and cell death throughout life; cell migration and organization (body plan) during embryonic development; and intermittently in response to signals from outside the cell, such as a hormone. TFs work alone or with other proteins in a complex, by promoting (as an activator), or blocking (as a repressor) the recruitment of RNA polymerase (the enzyme that performs the transcription of genetic information from DNA to RNA) to specific genes. A defining feature of TFs is that they contain at least one DNA-binding domain, which attaches to a specific sequence of DNA adjacent to the genes that they regulate. TFs are grouped into classes based on their DNA-binding domains.

Still further, in some embodiments, the present methods may be applicable for disorders associated with accumulations of oncogenes. An oncogene is a gene that has the potential to cause cancer. In tumor cells, these genes are often mutated, or expressed at high levels. Most normal cells will undergo a programmed form of rapid cell death (apoptosis) when critical functions are altered and malfunctioning. Activated oncogenes can cause those cells designated for apoptosis to survive and proliferate instead. Most oncogenes began as proto-oncogenes: normal genes involved in cell growth and proliferation or inhibition of apoptosis. If, through mutation, normal genes promoting cellular growth are up-regulated (gain-of-function mutation), they will predispose the cell to cancer; thus, they are termed “oncogenes”.

Still further in some embodiments, the disclosed methods that enhance nuclear sequestration of the proteasome may be useful for subjects suffering from disorders associated with accumulation of proteins in the nuclear lamina. The nuclear lamina is a dense (˜30 to 100 nm thick) fibrillar network inside the nucleus of eukaryote cells. It is composed of intermediate filaments and membrane associated proteins. The nuclear lamina is similar in structure to the nuclear matrix, that extends throughout the nucleoplasm. Besides providing mechanical support to the nucleus, the nuclear lamina regulates important cellular events such as DNA replication, DNA repair, cell division, cell differentiation and apoptosis. Additionally, it participates in chromatin organization and it anchors the nuclear pore complexes embedded in the nuclear envelope.

In some embodiments, a disorder associated with accumulation of proteins in the nuclear lamina may be HGPS. More specifically, Hutchinson-Gilford syndrome (HGPS) is a rare autosomal dominant genetic disorder in which symptoms resembling aspects of aging are manifested at an early age. Its occurrence is usually the result of a sporadic germline mutation; although HGPS is genetically dominant, people rarely live long enough to have children, preventing them from passing the disorder on in a hereditary manner. HPGS is caused by mutations that weaken the structure of the cell nucleus, normal making cell division difficult.

The histone mark H4K20me3 is involved and caused by de novo mutations that occur in a gene that encodes lamin A. Lamin A is made but is not processed properly. This poor processing creates an abnormal nuclear morphology and disorganized heterochromatin. Patients also do not have appropriate DNA repair, and they also have increased genomic instability.

In some particular and non-limiting embodiments, a compound useful in a method for reducing cytosolic proteolysis and/or increasing nuclear proteolysis may be a compound being or comprising at least one of: (a), at least one tyrosine (Y) residue, any tyrosine mimetic, any salt or ester thereof, any multimeric and/or polymeric form of said tyrosine residue and/or of the tyrosine mimetic, and any combinations or mixtures thereof; (b), at least one tryptophan (W) residue, any tryptophan mimetic, any salt or ester thereof, any multimeric and/or polymeric form of the tryptophan residue and/or of said tryptophan mimetic, or any combination or mixture thereof; and (c) at least one phenylalanine (F) residue, any phenylalanine mimetic, any salt or ester thereof, any multimeric and/or polymeric form of said phenylalanine residue and/or of the phenylalanine mimetic, and any combinations or mixtures thereof. In some specific embodiments, a compound useful in methods for reducing cytosolic proteolysis and/or increasing nuclear proteolysis may comprise the YWF triade; and/or (d) a compound comprising (a), (b) and (c), or any formulation or peptide thereof.

In some embodiments of the disclosed proteasome modulatory methods, the modulatory compound may be (a), or may comprise at least one of: a nucleic acid-based molecule, an amino acid-based molecule, a small molecule or any combinations thereof. Alternatively, or additionally, the compound may target at least one of said signaling molecule/s at the nucleic acid sequence level or at the protein level.

In more specific embodiments, in some embodiments, the modulatory compounds of the present disclosure specifically target the at least one signaling molecule (e.g., SESN3, p38, p62, NBR1, NUP93) at the nucleic acid level, thereby affecting the expression, distribution and/or splicing of such target signaling molecule. In yet some additional or alternative embodiments, the disclosed modulatory compound may specifically target the at least one signaling molecule (e.g., SESN3, p38, p62, NBR1, NUP93) at the protein level, thereby affecting the stability, activity, PTMs, and/or the interactions of such target signaling molecule with other signaling molecules. In some embodiments, at least one of: (a) the compound targets at least one of the signaling molecule/s at the nucleic acid sequence level. In yet some further embodiments, the compound is, or comprises a nucleic acid-based molecule. In more specific embodiments, the nucleic acid molecule is at least one of: a nucleic acid guide, a double-stranded RNA (dsRNA), a single-stranded RNA (ssRNA), an antisense oligonucleotide, a Ribozyme, a deoxyribozymes (DNAzymes), and an aptamer. In some embodiments, such compound may be a small interfering RNA (siRNA), and short hairpin RNA (shRNA), microRNA (miRNA), MicroRNA-like RNAs (milRNA), artificial miRNAs (amiRNA). In yet some alternative embodiments, the compound targets at least one of the signaling molecule/s at the protein level and reduces the stability of the target protein by targeted protein degradation (TPD).

In some specific embodiments, a compound useful in methods for reducing cytosolic proteolysis and/or increasing nuclear proteolysis may be a compound that leads to reduction of Sestrin3 levels and/or activity by specifically targeting a nucleic acid sequence encoding said Sestrin3, or any parts thereof, and/or by interfering with the interaction of Sestrin3 with at least one regulatory complex.

In yet some further embodiments, a compound useful in methods for reducing cytosolic proteolysis and/or increasing nuclear proteolysis may be a compound that leads to reduction of Sestrin3 levels and/or activity by specifically targeting a nucleic acid sequence encoding Sestrin3, or any non-coding sequence controlling the expression and/or splicing of Sestrin3, or any parts thereof.

In yet some alternative or additional embodiments, such compound may be any compound that interferes and/or blocks, and/or reduces the interaction of Sestrin3 with at least one regulatory complex.

In some embodiments, the Sestrin3 levels and/or activity may be reduced by the disclosed modulators via at least one of: (i) specifically targeting a nucleic acid sequence encoding the Sestrin3, or any parts thereof. In some other embodiments, the disclosed modulatory compounds may (ii), specifically target a nucleic acid sequence involved directly or indirectly in regulation of the Sestrin3 gene expression. In yet some further embodiments (iii), the modulatory compound may reduce the stability (increasing degradation) of the Sesn3 protein. Still further, in some additional or alternative embodiments and/or (iv), the compound may act via interfering with the interaction of Sestrin3 with at least one regulatory complex.

In some embodiments, the compound used in the methods of the present disclosure may comprise a gene editing system that targets the nucleic acid sequence encoding Sestrin3 or any non-coding sequence controlling the expression and/or splicing of Sestrin3. Specifically, in some embodiments, the compound of the present disclosure is any gene editing system or any component/s thereof. Thus, the methods for reducing cytosolic proteolysis and/or increasing nuclear proteolysis may comprise the use of a gene editing system that targets the Sestrin3 thereby leading to reduction in the expression and/or activity thereof. In some specific embodiments, the compound used in the methods for reducing cytosolic proteolysis and/or increasing nuclear proteolysis of the present disclosure comprise: (a) at least one gRNA that guides least one nucleic acid guided genome modifier protein to at least one target sequence within said Sestrin3 encoding nucleic acid sequence, or within a nucleic acid sequence involves directly or indirectly in regulation of the Sestrin3 gene expression, or at least one nucleic acid sequence encoding said nucleic acid guide. The compound of the present disclosure may further comprise in some optional embodiments thereof (b), at least one nucleic acid guided genome modifier protein, or any chimeric protein, complex or conjugate thereof, or at least one nucleic acid sequence encoding the guided genome modifier protein or chimeric protein thereof.

In more specific embodiments, the at least one nucleic acid modifier component comprises at least one clustered regularly interspaced short palindromic repeat (CRISPR)-Cas protein, cas protein derived domain and/or any variant and mutant thereof.

In some embodiments, the compound used by the methods disclosed herein comprises at least one sgRNA that specifically recognizes and binds at least one target sequence within the sestrin3 gene, or any nucleic acid sequence encoding these at least one sgRNA. In yet some specific and non-limiting embodiments, the sgRNA comprises the nucleic acid sequence as denoted by any one of SEQ ID NO: 1, 2, and 3, and designated herein as sgSESN3_1, 2 and 3, respectively.

In some embodiments the modulators may comprise siRNA directed against Sestrin 3. Such siRNA molecules that may act as modulators of proteasome dynamics and/or function in accordance with some embodiments of the present disclosure may be the siRNA molecules that comprise the nucleic acid sequence as denoted by any one of SEQ ID NO: 49, 50, 51, 52. In some additional or alternative embodiments (II), the disclosed compound may be at least one compound that reduces the stability of the Sesn3 by targeted protein degradation (TPD). In yet some additional or alternative embodiments, the Sestrin3 may be targeted functionally, by a compound used in methods for reducing cytosolic proteolysis and/or increasing nuclear proteolysis of the present disclosure. More specifically, such compound may interfere with Sestrin3 function, in some embodiments, by blocking any downstream pathways and/or interactions thereof. Thus, in some embodiments (III), the compound of the disclosed methods for reducing cytosolic proteolysis and/or increasing nuclear proteolysis, may be any compound that interferes and/or blocks, and/or reduces the interaction of Sestrin3 with at least one regulatory complex. In some embodiments, the compound used by the methods of the present disclosure may be any compound that interferes with, and/or blocks, and/or inhabits, and/or reduces, and/or decreases the inhibitory interaction of Sestrin3 with at least one member of the GATOR2 complex, specifically, with MIOS and/or WDR59.

In yet some further embodiments, a compound useful in methods for reducing cytosolic proteolysis and/or increasing nuclear proteolysis may be a p38 activator that leads to phosphorylation of at least one of Thr180 (T180) and/or Tyr 182 (Y182) of p38.

In some embodiments, a useful p38 activator is at least one of: a compound elevating the levels and/or activity of MKK3 and/or MKK6, a hyperosmotic agent, and a DNA Synthesis Inhibitor. In yet some further additional or alternative embodiments, a p38 activator useful in the disclosed methods may be at least one hyperosmotic agent. Non-limiting embodiments for such hyperosmotic agent, may be sorbitol. Thus, in some embodiments, any carbohydrates having a hyperosmotic effect may be used. To name but few, glycerin (glycerol), isosorbide, mannitol and urea may be used as the disclosed compounds. In some embodiments, the compound used by the methods of the present disclosure may be sorbitol. Still further, in some additional or alternative embodiments, a p38 activator useful in the disclosed methods may be at least one DNA Synthesis Inhibitor. In some specific embodiments, such compound may be anisomycin.

In yet some further alternative or additional embodiments, the compound may inhibit and/or reduces the level and/or activity of p62 and of NBR1. In some embodiments, NUP93 may be also modulated (activated). Still further, in some embodiments, nucleic acid-based modulators may be used. More specifically, siRNA, shRNAs and the like, as disclosed by the following Examples. In yet some further embodiments, a compound useful in methods for reducing cytosolic proteolysis and/or increasing nuclear proteolysis may be a compound that inhibits and/or reduces phosphorylation of STAT3. More specifically, in some embodiments, the compound used in the disclosed methods may be any STAT3 inhibitor, for example, any compound that inhibits and/or reduces phosphorylation of STAT3. In some particular embodiments, any compound that inhibits and/or reduces phosphorylation of Tyr705 of STAT3. In some specific and non-limiting embodiments, STAT3 inhibitors that may be useful in the methods disclosed herein may include small molecule compounds, specifically, Stattic (Stat three inhibitory compound), S3I-201/NSC74859, BP-1-102, Niclosamide, peptide inhibitors (e.g., the peptide aptamer APT STAT3-9R, and the like). In some specific embodiments, Stattic may be used as the compound of the methods of the present disclosure.

In yet some further aspects thereof, the present disclosre provides in vivo and in vitro modulatory methods having further therapeutic and non-therapeutic applications. The non-therapeutic applications of such modulatorry methods may encompass cosmetic and agricultural uses of the proteasome dynamics modulator/s of the invention.

More specifically, in some embodiments the at least one aromatic amino acid residue, SESN3 inhibitors, STAT3 inhibitors, p38 activators as disclosed by the present disclosure or any combinations thereof disclosed herein, may be used in the methods disclosed herein to promote muscle anabolism, improve muscle function, increase muscle mass, reverse muscle atrophy or to prevent muscle atrophy. In some embodiments, the proteasome dynamics modulator/s of the invention may be applicable in therapeutic methods for disorder/s characterized by muscle atrophy that may be any one of aging, bony fractures, weakness, cachexia, denervation, diabetes, dystrophy, exercise-induced skeletal muscle fatigue, fatigue, frailty, immobilization, inflammatory myositis, malnutrition, metabolic syndrome, neuromuscular disease, obesity, post-surgical muscle weakness, post-traumatic muscle weakness, sarcopenia, and toxin exposure. In some embodiments, the methods of the invention may be used to reverse muscle atrophy or to prevent muscle atrophy due to inactivity, immobilization, or age of the subject or a disease or condition suffered by the subject. In some embodiments, the methods of the present disclosure may be used to reverse muscle atrophy or to prevent muscle atrophy due to a broken bone, a severe burn, a spinal injury, an amputation, a degenerative disease, a condition wherein recovery requires bed rest for the subject, a stay in an intensive care unit, or long-term hospitalization. The term “bed rest” as used herein means that the subject is confined or required by a doctor to remain in bed, sitting and/or lying down for at least 80% of the day for at least 3 days. The term “long-term hospitalization” as used herein means a stay in a hospital or other health care facility for at least five days.

Still further, the methods of the invention may be applicable for preventing or reversing cardiac muscle atrophy (e.g., where a subject is suffering from or has suffered from heart attack, congestive heart failure, heart transplant, heart valve repair, atherosclerosis, other major blood vessel or ischemic disease, and heart bypass surgery.

In yet some further embodiments of the methods disclosed herein, the subject is suffering from a disease or condition known to be associated with cachexia for example, from cancer, viral infections, specifically, AIDS (HIV infection), SARS (SARS COV infection), and COVID 19 (SARS COV2 infection), chronic heart failure, COPD, rheumatoid arthritis, liver disease, kidney disease and trauma. In some embodiments, the subject is suffering from a disease or condition known to be associated with malabsorption. In some embodiments, such malabsorption the disease or condition may be any one of Crohn's disease, irritable bowel syndrome, celiac disease, and cystic fibrosis. In some embodiments, the methods of the present disclosure are applicable for subjects suffering from malnutrition, sarcopenia, muscle denervation, muscular dystrophy, an inflammatory myopathy, Spinal Muscle Atrophy, ALS, or myasthenia gravis.

More specifically, Muscular atrophy is the loss of skeletal muscle mass that can be caused by immobility, aging, malnutrition, medications, or a wide range of injuries or diseases that impact the musculoskeletal or nervous system. Muscle atrophy leads to muscle weakness and causes disability. Disuse causes rapid muscle atrophy and often occurs during injury or illness that requires immobilization of a limb or bed rest. Depending on the duration of disuse and the health of the individual, this may be fully reversed with activity. Malnutrition first causes fat loss but may progress to muscle atrophy in prolonged starvation and can be reversed with nutritional therapy. In contrast, cachexia is a wasting syndrome caused by an underlying disease such as cancer that causes dramatic muscle atrophy and cannot be completely reversed with nutritional therapy. Sarcopenia is the muscle atrophy associated with aging and can be slowed by exercise. Finally, diseases of the muscles such as muscular dystrophy or myopathies can cause atrophy, as well as damage to the nervous system such as in spinal cord injury or stroke.

Muscle atrophy results from an imbalance between protein synthesis and protein degradation, although the mechanisms are variable depending on the cause. Muscle loss can be quantified with advanced imaging studies. Treatment depends on the underlying cause but will often include exercise and adequate nutrition. Anabolic agents may have some efficacy but are not often used due to side effects. Still further, in some embodiments, a subject suffering from a disorder, condition, or symptom associated with muscle atrophy is a subject whose skeletal muscle mass has decreased by at least a 5% as a result of the disorder, condition, or symptom. In some embodiments, such subject may display a decrease in the skeletal muscle mass of at least about 5%, 8%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more as a result of the disorder, condition, or symptom. In some embodiments, a subject suffering from a disorder, condition, or symptom associated with muscle atrophy is a subject whose muscle weight relative to body weight ratio decreased by at least a 2%, at least a 3%, at least a 4%, at least a 5%, at least 6%, at least 7%, at least 8%, at least 9%, at least 10%, at least a 15%, at least a 16%, at least a 20%, at least a 25%, at least 30%, at least 35%, or at least 40% or more as a result of the disorder, condition, or symptom.

In some embodiments, any of the methods of increasing at least one of mTOR activation/activity and/or lysosomal localization, and/or activation of p38, and/or inhibition of STAT3, inhibition of SESN3, and thereby, increasing proteasome nuclear localization set forth herein, can be used for increasing skeletal muscle mass. Still further, as used herein, “increasing skeletal muscle mass” refers to a statistically significant increase in the skeletal muscle mass. In some embodiments of various aspects, increasing skeletal muscle mass refers to a reversal of skeletal muscle loss. In some embodiments of various aspects, increasing skeletal muscle mass refers to an increase in skeletal muscle mass of at least 5%, at least 7%, at least 12%, at least 15%, at least 18%, at least 20%, at least 21%, at least 25%, at least 27%, at least 30%, at least 33% or more, relative to the skeletal muscle mass prior to contacting the skeletal muscle with the proteasome dynamics modulator/s of the invention, specifically, at least one of tyrosine, tryptophan, and/or phenylalanine, any mimetics or composition thereof, any of the SESN3 inhibitors, STAT3 inhibitors, p38 activators as disclosed by the present disclosure, and/or to administration to the subject. In some embodiments, increasing skeletal muscle mass refers to an increase in skeletal muscle mass of a subject to within 35%, within 33%, within 30%, within 28%, within 24%, within 22%, within 18%, within 15%, within 12%, within 10%, within 9%, within 8%, within 7%, within 6%, within at least 5% or more of the skeletal muscle before onset of the disorder, condition, or symptom associated with muscle atrophy, or onset of the muscle atrophy itself.

The disclosure thus provides therapeutic and non-therapeutic methods of increasing skeletal muscle mass, comprising contacting skeletal muscle or skeletal muscle cells with the at least one proteasome dynamics modulator/s of the invention, specifically, at least one of the SESN3 inhibitors, STAT3 inhibitors, p38 activators as disclosed by the present disclosure, as well as the tyrosine, tryptophan and/or phenylalanine, or any mimetics, combinations and compositions thereof.

In some embodiments, the proteasome dynamics modulator/s of the invention stimulate mTOR activation and the associated proteasome nuclear localization in the skeletal muscle or skeletal muscle cells, thereby promoting skeletal muscle anabolism and increasing skeletal muscle mass.

In yet some further embodiments, the disclosure provides a method of increasing skeletal muscle mass in a subject, comprising administering to the subject an effective amount of any one of the proteasome dynamics modulator/s of the invention, specifically, at least one of tyrosine, tryptophan and/or phenylalanine or any mimetics thereof, and/or any of the SESN3 inhibitors, STAT3 inhibitors, p38 activators as disclosed by the present disclosure, or an effective amount of a composition comprising at least one of tyrosine, tryptophan and/or phenylalanine or any mimetics thereof, optionally, in at least one dosage form. In some embodiments, the at least one of tyrosine, tryptophan and/or phenylalanine or any mimetics thereof stimulate mTOR activation and the associated proteasome nuclear localization in the subject, thereby promoting skeletal muscle anabolism and increasing the subject's skeletal muscle mass.

As indicated herein, the method of increasing skeletal muscle mass may lead to an increase in muscle-to-fat ratio. The methods disclosed herein may therefore have additional and non-therapeutic applications, for example, cosmetic and/or agricultural uses.

More specifically, in some embodiments, the method of increasing skeletal muscle mass is used for agricultural purpose, specifically, to increase skeletal muscle mass (or increase the muscle-to fat ratio) in a non-human animal, such as livestock, fish, poultry or insects. In these embodiments, each of the proteasome dynamics modulator/s of the invention, specifically, at least one aromatic amino acid residues, more specifically, at least one of Y, W and/or F, and any mimetics thereof, may be administered as an additive to the feed of the non-human animal, used as pets and in food industry.

A further aspect of the present disclosure relates to a method for treating, preventing, inhibiting, reducing, eliminating, protecting or delaying the onset of at least one condition or at least one pathologic disorder in a subject in need thereof. In some embodiments, the method comprises the steps of:

In step (a), determining in at least one sample of the subject, at least one of: (i) mTOR activation and/or lysosomal association; (ii) activation of p38, specifically, p38 delta; (iii) phosphorylation of Tyr705 of STAT3; and/or (iv) Sestrin3 levels, and/or activity and/or the interaction of Sestrin3 with at least one regulatory complex; and optionally, (v) the proteasome subcellular localization in at least one cell of the at least one sample, or in any fraction thereof.

The next step (b), involves classifying the subject as:

(I) a responder subject to the treatment regimen, if at least one of: (i) mTOR is activated and/or localized to the lysosomal membrane; (ii) p38 is activated; (iii) phosphorylation of Tyr705 of STAT3 is inhibited or reduced; and/or (iv) the Sestrin3 levels, and/or activity and/or the interaction of Sestrin3 with at least one regulatory complex are reduced; and optionally, (v) the ratio of nuclear to cytosolic proteasome subcellular localization is greater than 1; or
(II) a non-responder subject or a poor responder to said treatment regimen if at least one of: (i) mTOR is inactivated and/or dissociated from the lysosomal membrane; (ii) p38 is inactivated; (iii) Tyr705 of STAT3 is phosphorylated; and/or (iv) Sestrin3 levels, and/or activation and/or the interaction of Sestrin3 with at least one regulatory complex are maintained or increased; and optionally, (v) the ratio of nuclear to cytosolic proteasome subcellular localization is smaller than, or equal to 1. The next step (c), involves administering to a subject classified as a responder a treatment regimen comprising a therapeutic effective amount of at least one compound that modulates proteasome dynamics and/or function in a mammalian cell, increasing the dose of the compound in subject exhibiting a mild or poor response, or ceasing the treatment regimen for a subject classified as a non-responder or poor responder; thereby treating the subject.

A further aspect relates to a therapeutic compound that modulates proteasome dynamics and/or function in a mammalian cell, or any composition thereof. More specifically, the compound is characterized by affecting at least one of: mammalian target of rapamycin (mTOR) lysosomal association, the activity and/or level/s and/or the post translational modification/s (PTM/s), and/or subcellular localization of at least one signaling molecule participating directly or indirectly in at least one pathway mediating said proteasome dynamics/function. Optionally, the modulating compound may further modulate proteasome cellular localization.

In some specific embodiments, the compound may lead to reduction of Sestrin3 levels and/or activity by at least one of: (i) specifically targeting a nucleic acid sequence encoding said

Sestrin3, or any parts thereof; (ii) specifically targeting a nucleic acid sequence involves directly or indirectly in regulation of the Sestrin3 gene expression; (iii) reducing the stability (increasing degradation) of the Sesn3 protein; and/or (iv) interfering with the interaction of Sestrin3 with at least one regulatory complex.

In some specific embodiments, the disclosed compound comprises:

(a) at least one gRNA that guides least one nucleic acid guided genome modifier protein to at least one target sequence within said Sestrin3 encoding nucleic acid sequence, or within a nucleic acid sequence involves directly or indirectly in regulation of the Sestrin3 gene expression; or at least one nucleic acid sequence encoding said nucleic acid guide; and optionally

(b) at least one nucleic acid guided genome modifier protein, or any chimeric protein, complex or conjugate thereof, or at least one nucleic acid sequence encoding said guided genome modifier protein or chimeric protein thereof. In yet some further specific embodiments, the at least one gRNA useful in the present disclosure may comprise the nucleic acid sequence of any one of SEQ ID NO: 1, 2, 3, or any combinations thereof.

In yet some further embodiments, the compounds of the present disclosure my comprise at least one siRNA molecules directed against Sestrin 3, or any composition thereof. In some embodiments, the siRNAs may comprise at least one of the nucleic acid sequence as denoted by SEQ ID NO: 49, 50, 51, 52, or any combinations or compositions thereof.

It should be understood that the delivery of siRNA molecules and/or the CRISPR systems discussed above, may involve the use of any nanoparticle or any nucleic acid vector (e.g., viral vectors such as AAV and the like). Still further, lipid nanoparticles (LNPs) and conjugates (e.g., GalNAc-siRNA, cell penetrating peptides (CPP)-siRNA conjugates, and GalNAc, or N-acetyl-galactosamine), may be also used by the present disclosure. The term “non-human animal” as used herein includes any organism, specifically all vertebrates, any non-mammal organism (e.g., fish, chickens, amphibians, reptiles and insects) and mammals, such as non-human primates, domesticated and/or agriculturally useful animals, e.g., sheep, dog, cat, cow, pig, etc. The term “livestock”, as used herein refers to any farmed animal. Preferably, livestock is one or more of ruminants such as cattle (e.g., cows or bulls (including calves)), mono-gastric animals such as poultry (including broilers, chickens and turkeys), pigs (including piglets), birds, or sheep (including lambs).

It should be appreciated that the present disclosure further encompasses methods, compositions and kits for modulating proteasome dynamics in a cell, and/or in a subject in need thereof. Thus, the present disclosure further encompasses modulatory methods that may be performed in vivo, in vitro or ex vivo. In some embodiments, the method of the present disclosure may comprise the step of contacting the cell, or at least one cell in a subject, with a modulatory effective amount of the proteasome dynamics modulator/s of the invention or any composition, combinations or kits thereof. As used herein “modulating” means causing or facilitating a qualitative or quantitative change, alteration, or modification in a molecule, a process, pathway, or phenomenon of interest. For example, cellular localization of the proteasome, localization of mTOR to the lysosomal membrane, activation of p38, inhibition of Sestrin3 and/or inhibition of STAT3 phosphorylation of Y705. Without limitation, such change may be an increase, decrease, a change in nuclear or cytosolic proteasome localization characteristics, or change in relative strength or activity of different components or branches of the process, pathway, or phenomenon.

In yet some further embodiments, the proteasome dynamics modulator/s of the invention as well as any combinations, compositions, kits and methods thereof, increase proteasome nuclear localization in a cell. As used herein “increasing”, “increased”, “increase”, “stimulate”, “enhance” or “activate” are all used herein to generally mean an increase by a statistically significant amount; for the avoidance of any doubt, the terms “increased”, “increase”, “stimulate”, “enhance” or “activate” means an increase of at least 10% as compared to a reference level of the proteasome nuclear localization. For example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level, of the proteasome nuclear localization.

As indicated above, the methods of the invention involve the step of contacting the cell/s with the proteasome dynamics modulator/s of the present disclosure. As used herein “contacting the cell” and the like, refers to any means of introducing at least one agent described herein, specifically, the proteasome dynamics modulator/s of the invention, more specifically, any of the SESN3 inhibitors, STAT3 inhibitors, p38 activators as disclosed by the present disclosure, and/or the at least one aromatic amino acid, any mimetics thereof, or any compound or agent that directly or indirectly increase the level, stability and/or bioavailability of the at least one aromatic amino acid residue/s, or a composition comprising at least one proteasome dynamics modulator/s described herein into a target cell in vitro, ex vivo or in vivo, including by chemical and physical means, whether directly or indirectly or whether the at least one proteasome dynamics modulator/s or the composition comprising the at least one proteasome dynamics modulator/s physically contacts the cell directly or is introduced into an environment (e.g., culture medium, body cavity, organ and/or tissue) in which the cell is present or to which the cell is added. It is to be understood that the cells contacted with the at least one agent or composition comprising the at least one agent described herein (e.g., the p38 activator/s, Sestrin3 inhibitors, STAT3 inhibitors and/or the Y, W and/or F) can also be simultaneously or subsequently contacted with another compound, such as a growth factor or other differentiation agent to stabilize and/or to differentiate the cells further. Contacting also is intended to encompass methods of exposing a cell, delivering to a cell, or ‘loading’ a cell with a proteasome dynamics modulator/s by viral or non-viral vectors, and wherein such proteasome dynamics modulator/s is bioactive upon delivery.

The method of delivery will be chosen for the particular agent and use (e.g., disorder characterized by or associated with processed involving short-term stress conditions as disclosed herein).

Parameters that affect delivery, as is known in the art, can include, inter alia, the cell type affected (e.g., epithelial cells, bone marrow lymphocytes, myocytes, neuronal cells and the like), and cellular location. In some embodiments, “contacting” includes administering the at least one proteasome dynamics modulator/s (e.g., the p38 activator/s, Sestrin3 inhibitors, STAT3 inhibitors and/or the Y, W and F and/or mimetics thereof) or a composition comprising the at least one proteasome dynamics modulator/s to an individual. In some embodiments, “contacting” refers to exposing a cell or an environment in which the cell is located to one or more of the p38 activator/s, Sestrin3 inhibitors, STAT3 inhibitors and/or Y, W, and F or any mimetic thereof described in the present disclosure. It should be understood that in some embodiments, the term “contacting” is not intended to include the in vivo exposure of cells to the agents or compositions disclosed herein that may occur naturally (i.e., as a result of digestion of an ordinary meal).

It should be appreciated that the cell can be contacted with any one of the at least one proteasome dynamics modulator/s of the present disclosure, specifically, any of the SESN3 inhibitors, STAT3 inhibitors, p38 activators as disclosed by the present disclosure, and/or the at least one aromatic amino acid residue, more specifically, at least one of tyrosine, tryptophan, phenylalanine, and/or any mimetics thereof, together or separately. In one exemplary embodiment, a cell can be contacted with an oligopeptide, a peptide, or polypeptide comprising the at least one of tyrosine, tryptophan, phenylalanine, and/or any mimetics thereof, for example, a synthetic oligopeptide, peptide, or polypeptide containing only Y, W, and/or F residues.

In practicing the subject methods, any cell that expresses at least one of mTOR, GATOR2 complex, SESN3, p38, STAT3, p62 and/or NBR1, and any other molecule that participates directly or indirectly in proteasome dynamics as discussed herein, can be targeted for modulation of proteasome dynamics, Non-limiting examples of specific cell types in which any of the targets disclosed above can be modulated thereby modulating proteasome dynamics, include fibroblast, cells of skeletal tissue (bone (e.g., proliferative and hypertrophic chondrocytes) and cartilage), cells of epithelial tissues (e.g. liver, lung, breast, skin, bladder and kidney), cardiac and smooth muscle cells (e.g., cardiomyocytes), neural cells (glia and neurons), cells of the hypothalamus, hippocampal cells, endocrine cells (adrenal, pituitary, pancreatic islet alpha and beta cells), exocrine pancreatic cells (e.g., acinar cells), melanocytes, many different types of hematopoietic cells (e.g., macrophages, cells of B-cell or T-cell lineage, neutrophils, red blood cells, and their corresponding stem and progenitor cells, lymphoblasts), cells of both white adipose tissue and brown adipose tissue (e.g., adipocytes), and intestinal cells (e.g., Paneth cells, enterocytes, goblet cells). In some embodiments, the cell is a mammalian cell, derived from a mammalian subject. In some embodiments, the cell is a human cell.

Still further, the disclosure provides a method of modulating various target proteins that are involved with proteasome dynamics, for example, increasing mTOR activity and/or lysosomal localization, thereby increasing proteasome nuclear localization in a subject. Such method comprising administering to a subject in need thereof at least one proteasome dynamics modulator/s comprising the p38 activator/s, Sestrin3 inhibitors, STAT3 inhibitors and/or at least one aromatic amino acid residue, any mimetic thereof, any salt or ester thereof, any multimeric and/or polymeric form of the at least one aromatic amino acid residue and/or of the aromatic amino acid residue mimetic, any compound that modulates directly or indirectly at least one of the levels, stability and bioavailability of the at least one aromatic amino acid residue, any combinations or mixtures thereof, any vehicle, matrix, nano- or micro-particle thereof, or any composition or kit comprising the same.

In yet some further embodiments, the compositions disclosed herein, and any compositions used by any of the methods of the present disclosure, may comprise in addition to, or instead of, the at least one aromatic amino acid residue or any mimetics thereof, any compound that modulates directly or indirectly at least one of the levels, stability and bioavailability of the at least one aromatic amino acid residue, optionally in at least one dosage unit form. Non-limiting examples for such compound include Nitisinone, that may increase the levels of tyrosine and/or phenylalanine.

Still further, it should be understood that any of the proteasome dynamics modulator/sof the present disclosure, used in any of the methods and compositions disclosed herein, for example, any of the aromatic amino acid residues disclosed herein (either the D-isomers of YWF, the L-isomers of YWF or any mixtures thereof) or any mimetics thereof, or any peptide or protein comprising the at least one aromatic amino acid residues of the invention or any mimetics thereof, e.g., any of the SESN3 inhibitors, STAT3 inhibitors, p38 activators as disclosed by the present disclosure, may be in certain embodiments, associated with, combined with or conjugated with at least one “enhancing” moiety. Such moiety may be any moiety that increases the modulatory effect thereof, and specifically, in some embodiments, promotes and/or enhances proteasome nuclear localization, and/or activity, either by facilitating cell penetration, targeting to specific cell target and/or by increasing stability and reducing clearance thereof. The term “associated with” as used herein in reference to a half-life increasing moiety, a cell penetration moiety, a specific tissue or organ-directing moiety or a specific cell type directing moiety means that such moiety may be linked non-covalently, or covalently bound to, conjugated to, cross-linked to, incorporated within (e.g., such as an amino acid sequence within a peptide, polypeptide or protein that comprise at least one of the aromatic amino acid residues of the invention or any mimetics thereof), or present in the same composition as the at least one aromatic amino acid residue (specifically, Y, W and/or F), any mimetics thereof, a peptide comprising the at least one amino acid residues, non-standard peptide, polypeptide, non-standard polypeptide, protein or non-standard protein comprising the aromatic amino acid residues of the invention, and/or any proteasome dynamics modulator/s, e.g., any of the SESN3 inhibitors, STAT3 inhibitors, p38 activators as disclosed by the present disclosure, in such a way as to allow such moiety to carry out its function. The term “cell penetration moiety” as used herein means a moiety that enhances the ability of the peptide, non-standard peptide, polypeptide, non-standard polypeptide, protein or nonstandard protein thereof with which it is associated to penetrate the cell membrane. In some embodiments, the “cell penetration moiety” may be an amino acid sequence within or connected to a peptide comprising at least one of the aromatic amino acid residues of the invention, non-standard peptide, polypeptide, non-standard polypeptide, protein or non-standard protein, or connected either directly or indirectly to any of the other modulators, e.g., any of the SESN3 inhibitors, STAT3 inhibitors, p38 activators as disclosed by the present disclosure. Examples of cell penetration sequences include, but are not limited to, Arg-Gly-Asp (RGD), Tat peptide, oligoarginine, MPG peptides, Pep-land the like.

The term “specific organ directing moiety” as used herein means a moiety that enhances the ability of the aromatic amino acid residue/s of the invention or any mimetics thereof, peptide, non-standard peptide, polypeptide, non-standard polypeptide, protein or non-standard protein thereof, with which it is associated to be targeted to a specific organ. In some embodiments, the “specific organ directing moiety” is an amino acid sequence, small molecule or antibody that binds to a cell type present in the specific organ. In some embodiments, the “specific organ directing moiety” is an amino acid sequence, small molecule or antibody that binds to a receptor or other protein characteristically present in the specific organ.

The term “specific cell-type directing moiety” as used herein means a moiety that enhances the ability of the aromatic amino acid reside, or any peptide, non-standard peptide, polypeptide, non-standard polypeptide, protein or non-standard protein thereof, with which it is associated to be targeted to a specific cell type. In some embodiments, the “specific cell-type directing moiety” is an amino acid sequence, small molecule or antibody that binds to a specific receptor or other protein characteristically present in or on the surface of the specific target cell type.

The proteasome dynamics modulator/s of the present disclosure, specifically, the p38 activator/s, Sestrin3 inhibitors, STAT3 inhibitors and the at least one of tyrosine, tryptophan and/or phenylalanine (Y, W and/or F), and mimetics thereof, any dosage form or any dosage unit form thereof, may be formulated into a pharmaceutically acceptable composition or a nutraceutical composition. Such composition may, for example, be designed for any suitable administration mode, that may be adapted to any desired tissue, organ or cell. Non-limiting examples for administration modes include but are not limited to, parenteral, enteral, intra-muscular, direct to brain, or oral administration. Further relevant administration modes are discussed herein after. In a more specific aspect, at least one of the proteasome dynamics modulator/s or any dosage form or dosage unit form thereof, is formulated into a controlled release formulation. In this connection, the use of implant that acts to retain the active dose at the site of implantation, is also encompassed by the invention. The active agent may be formulated for immediate activity, or alternatively, or it may be formulated for sustained release as mentioned herein.

In another more specific aspect, at least one of the proteasome dynamics modulator/s is formulated into a composition to promote absorption from a specific portion of the target organ. In even more specific embodiments, any of the compositions of the present disclosure may be formulated as a pharmaceutical composition for delivery to a specific organ or cell type (e.g., brain, muscle, fibroblasts, bone, cartilage, liver, lung, breast, skin, bladder, kidney, heart, smooth muscle, adrenal, pituitary, pancreas, melanocytes, blood, adipose, and intestine). It will be understood that formulation for delivery to the brain requires the ability of the active components to cross the blood-brain barrier or to be directly administered to the brain or CNS.

In some embodiments, the at least one proteasome dynamics modulator/s of the composition disclosed herein may be formulated as an oral dosage form. In yet some further embodiments, the composition disclosed herein may be formulated in an oral dosage unit form. In yet some alternative embodiments, the at least one proteasome dynamics modulator/s may be formulated as an injectable dosage form. In yet some further embodiments, the composition disclosed herein may be formulated in an injectable dosage unit form.

In some embodiments, the oral dosage form may be administered orally, for example, as a solution (e.g., syrup), or as a powder, tablet, capsule, and the like. In some further embodiments, the oral dosage form may be provided in a formulation adapted for add-on to a solid, semi-solid or liquid food, beverage, food additive, food supplement, medical food, drug and/or a pharmaceutical composition.

In certain embodiments the composition of the invention may be formulated in a formulation adapted for add-on to a solid, semi-solid or liquid food, beverage, food additive, food supplement, medical food, botanical drug, drug and/or any type of pharmaceutical compound.

In some embodiments, the add-on composition according to the invention may be formulated as a food additive, food supplement or medical food. In other embodiment, such add-on composition of the invention may be further added or combined with drugs or any type of pharmaceutical products. The term ‘add-on’ as used herein is meant a composition or dosage unit form of the at least one proteasome dynamics modulator/s of the present disclosure that may be added to existing compound, composition or material (e.g., food or beverage), enhancing desired properties thereof or alternatively, adding specific desired property to an existing compound, composition, food or beverage.

More specifically, in certain embodiments, the at least one proteasome dynamics modulator/s of the present disclosure, or any dosage form or composition thereof may be an add-on to a food supplement, or alternatively, may be used as a food supplement. A food supplement, the term coined by the European Commission for Food and Feed Safety, or a dietary supplement, an analogous term adopted by the FDA, relates to any kind of substances, natural or synthetic, with a nutritional or physiological effect whose purpose is to supplement normal or restricted diet. In this sense, this term also encompasses food additives and dietary ingredients. Further, under the Dietary Supplement Health and Education Act of 1994 (DSHEA), a statute of US Federal legislation, the term dietary supplement is defined as a product intended to supplement the diet that bears or contains one or more of the following dietary ingredients: a vitamin, a mineral, an herb or other botanical, a dietary substance for use by a subject to supplement the diet by increasing the total dietary intake, or a concentrate, metabolite, constituent, extract, or combination of any of the aforementioned ingredients

Food or dietary supplements are marketed a form of pills, capsules, powders, drinks, and energy bars and other dose forms. Unlike drugs, however, they are mainly unregulated, i.e., marketed without proof of effectiveness or safety. Therefore, the European and the US laws regulate dietary supplements under a different set of regulations than those covering “conventional” foods and drug products. According thereto, a dietary supplement must be labeled as such and be intended for ingestion and must not be represented for use as conventional food or as a sole item of a meal or a diet. However, the add-on dosage form or composition that comprise the at least one modulating compound provided herein, may be added to a meal or beverage consumed by the subject.

In yet some further embodiments, the proteasome dynamics modulator/s or any composition thereof, in accordance with the present disclosure may be an add-on to medical foods or may be consumed as a medical food. Further in this connection should be mentioned medical foods, which are foods that are specially formulated and intended for the dietary management of a disease that has distinctive nutritional needs that cannot be met by normal diet alone.

A medical food, as defined in section 5 (b) (3) of the Orphan Drug Act (21 U.S.C. 360cc (b) (3)), is “a food which is formulated to be consumed or administered enterally under the supervision of a physician and which is intended for the specific dietary management of a disease or condition for which distinctive nutritional requirements, based on recognized scientific principles, are established by medical evaluation.” FDA considers the statutory definition of medical foods to narrowly constrain the types of products that fit within this category of food (21 CFR 101.9 (j) (8)). Medical foods are distinguished from the broader category of foods for special dietary use by the requirement that medical foods be intended to meet distinctive nutritional requirements of a disease or condition, used under medical supervision, and intended for the specific dictary management of a disease or condition. Medical foods are not those simply recommended by a physician as part of an overall dict to manage the symptoms or reduce the risk of a disease or condition. Not all foods fed to patients with a disease, including diseases that require dictary management, are medical foods. Instead, medical foods are foods that are specially formulated and processed (as opposed to a naturally occurring foodstuff used in a natural state) for a patient who requires use of the product as a major component of a disease or condition's specific dietary management.

It is a specially formulated and processed product (as opposed to a naturally occurring foodstuff used in its natural state) for the partial or exclusive feeding of a patient by means of oral intake or other feeding means (e.g., a tube or catheter).

Also pertinent to the present context are any type of drugs or therapeutic compounds, that may be available as (but not limited to) a solution (e.g., tea), powder, tablet, capsule, elixir, topical, or injection. Thus, in further embodiments, the at least one proteasome dynamics modulator/s, any dosage form, dosage unit form, or composition thereof, may be an add-on to any type of drugs or therapeutic compounds administered orally, intravenously, intradermaly, by inhalation or intrarectaly.

In some embodiments, the at least one proteasome dynamics modulator/s, any dosage form, dosage unit form, or composition thereof may be adapted for add-on a food and/or beverage.

In this context, a beverage is any beverage including for example fruit or fruit-flavored drinks, flavored water or sodas, energy drinks, coffees, teas, milk, chocolate milk and nonalcoholic wines and beers. Food, as used herein is any dry, semi-dry, or liquid edible substance providing nutrients and or calories to the consuming subject. Food may be composed of natural or synthetic ingredients and any combinations thereof, and may provide carbohydrates, fat, fibers, vitamins and other nutrients. Exemplary food products can be, but are not limited to bakery products, such as bread, biscuits, cookies, cakes, pastries and the like; confectionery products such as chocolate or vegetarian or vegan chocolate, candy, gummy; dates products; dairy or dairy like (vegetarian) products such as yoghurt, cheeses, ice creams; formula such as infant formula; garnishes such as mayonnaise, ketchup and the like; frozen foods; protein and energy bars; savory snacks; and the like.

As indicated above, in connection with the proteasome dynamics modulator/s of the present disclosure, specifically, the p38 activator/s, Sestrin3 inhibitors, STAT3 inhibitors and/or each of the aromatic amino acid residues may be provided in a dosage form or in a dosage unit form. Dosage forms, as used herein, are pharmaceutical drug products in the form in which they are marketed for use, with a specific mixture of active ingredients (e.g., the p38 activator/s, Sestrin3 inhibitors, STAT3 inhibitors and/or the YWF, and/or any mimetics thereof) and optionally, inactive components (excipients), in a particular configuration (such as a capsule shell, for example), and apportioned into a particular dose. In some embodiments, the term dosage form can also refer in some embodiments only to the pharmaceutical formulation of a drug product's constituent drug substance(s) and any blends involved.

As used interchangeably herein, “dosage units”, “dosage forms”, “oral or injectable dosage units”, “dosage unit forms”, “oral or injectable dosage unit forms” and the like refer to both, solid dosage forms as known in the art, or to a liquid dosage form. The dosage forms are intended for peroral use, i.e., to be swallowed (ingested), or even injected or applicated in any other means, either by a subject in need thereof, or for administration by a medical practitioner. The terms “active substance” or “active ingredient”, used herein interchangeably, refer to a therapeutically or physiologically active substance, specifically, the modulating compounds disclosed herein, that provides a therapeutic/physiological effect to a patient, and can also refer to a mixture of at least two thereof.

In some embodiments, any of the proteasome dynamics modulator/s of the present disclosure, as well as any formulations, dosage forms, dosage unit forms, compositions, kits methods and uses thereof may be adapted for, or may involve at least one systemic and/or at least one non-systemic administration. The term “non-systemically” as herein defined refers to a localized route of administration, namely a route of administration which is not via the digestive tract and not parenterally. In embodiments of the disclosure, the non-systemic administration may be any administration mode, for example, intrathecal, intra-nasal, intra-ocular, intraneural, intra-cerebral, intra-ventricular, intra-cerebroventricular, intra-cranial, and subdural administration. In yet some further embodiments, the of the disclosure, the systemic administration may be any administration mode, for example, oral, intravenous, intramuscular, subcutaneous, topical, enteral (e.g., gastrointestinal tract, specifically, oral, rectal, sublingual, sublabial or buccal, by any one of injection, enema, catheter, applicator, or any oral or topical formulation), or parenteral. In yet some further embodiments, the modulating compounds of the present disclosure, as well as any formulations, dosage forms, dosage unit forms, compositions, kits methods and uses thereof may be formulated as injectable formulations, that may be used either for systemic or for non-systemic, or local administration. In further embodiments of the disclosure the said injectable formulation, specifically, aqueous or liquid formulation, is designed for administration to said subject by bolus administration. In other embodiments of the disclosure the said aqueous injectable formulation is designed for administration to the subject by infusion of no less than one minute and no more than 24 hours.

Thus, the present disclosure further provides an injectable aqueous formulation for non-systemic administration to a subject in need thereof, said formulation comprising as active ingredient the at least one modulating compounds of the present disclosure or any combinations or formulations thereof, that may comprise in some embodiments, the concentration of from about 0.01 mM of each of the p38 activator/s, Sestrin3 inhibitors, STAT3 inhibitors and the Y, W, F of the present disclosure or any mimetics thereof, to about 30 mM or each of said aromatic amino acids Y, W, F, or any mimetics thereof. In yet some further embodiments, the concentration is no more than 100 mM for each of the aromatic amino acid residues.

In the disclosed methods of treatment, the injectable formulation as herein defined is administered once, twice or more a day, every other day, a week, every two weeks, every three weeks, once, twice or more every four weeks, once every 5, 6, 7 or 8 weeks, once a month, once every two months, once every three months, once every four months, once every five months or once every six months, or even once twice or more a year.

As indicated herein, the composition or any dosage form or dosage unit form disclosed herein may be provided in an injectable formulation. The term “injection” or “injectable” as used herein refers to a bolus injection (administration of a discrete amount of the at least one modulating compound disclosed herein, for raising its concentration in a bodily fluid), slow bolus injection over several minutes, or prolonged infusion, or several consecutive injections/infusions that are given at spaced apart intervals. Such spaced apart injections per a single administration are also referred to herein as “per administration injection”, or in other words, a single administration can include several injections or prolonged infusion. The injectable aqueous formulation for non-systemic administration to a subject in need thereof as herein defined may be administered using a drug-device combination, for example a mechanical or electro-mechanical device, more preferably an electro-mechanical infusion pump. The electro-mechanical pump, for example, consists of a reservoir for housing a medication, a catheter having a proximal portion coupled to the pump and having a distal portion adapted for administering a medication to the desired site.

Still further, the composition of the present disclosure, as well as any product or use of the proteasome dynamics modulator/s of the present disclosure, specifically, the p38 activator/s, Sestrin3 inhibitors, STAT3 inhibitors and the YWF disclosed herein may be provided and/or used in an effective amount. More specifically, the compositions of the invention may comprise an effective amount of at least one proteasome dynamics modulator/s of the invention as disclosed herein and/or any vehicle, matrix, nano- or micro-particle thereof. The term “effective amount” relates to the amount of an active agent present in a composition, specifically, the proteasome dynamics modulator/s of the invention as described herein that is needed to provide a desired level of active agent in the bloodstream or at the site of action in an individual (e.g., the specific site of the tumor) to be treated to give an anticipated physiological response when such composition is administered. The precise amount will depend upon numerous factors, e.g., the active agent, the activity of the composition, the delivery device employed, the physical characteristics of the composition, intended patient use(i.e., the number of doses administered per day), patient considerations, and the like, and can readily be determined by one skilled in the art, based upon the information provided herein. An “effective amount” of the proteasome dynamics modulator/s of the invention can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

An effective amount in accordance with the modulating compounds of the present disclosure, specifically proteasome dynamics modulator/s comprising the p38 activator/s, Sestrin3 inhibitors, STAT3 inhibitors and the at least two aromatic amino acid residues, and more specifically, all three amino acid residues Tyrosine, Tryptophane and Phenylalanine, as used in the present disclosure (e.g., in the modulating compounds, compositions, kits and methods disclosed herein), may be presented in any amount effective for selective and specific modulation of the proteasome dynamics, specifically, in modulating proteasome dynamics in a cell, as discussed herein. In yet some further embodiments, the amount of the aromatic amino acid residues is any amount effective for specific and selective inhibition of proteasome recruitment or translocation from the nucleus to the cytosol. Still further, in some embodiments, an effective amount is an amount effective for specifically and selectively maintaining nuclear localization of the proteasome in cells of a subject in need. In yet some further embodiments, an effective amount is an amount effective for specifically and selectively requiring the proteasome into the nucleus and modulating proteasome dynamics such that the proteasome localization is predominantly nuclear in cells of the treated subject.

Thus, in some embodiments, the combinations, combined compositions and compositions of the invention comprise the p38 activator/s, Sestrin3 inhibitors, STAT3 inhibitors and/or the least one tyrosine (Y) residue, at least one tryptophan (W) residue, and at least one phenylalanine (F) residue, or any mimetic, salt or ester thereof, any multimeric and/or polymeric form thereof, and any combinations or mixtures thereof, and any dosage forms or dosage unit form thereof, in an amount effective for selective modulation of proteasome localization, specifically, selective and specific inhibition of proteasome translocation, specifically, inhibition of proteasome translocation to the cytosol, and optionally, selective and specific enhancement of recruitment of the proteasome to the nucleus, in at least one cell of at least one subject treated by the modulating compounds, dosage forms, dosage unit forms, compositions, kits and methods disclosed herein.

As shown in the present disclosure, the three aromatic amino acid residues of the invention, specifically, the p38 activator/s, Sestrin3 inhibitors, STAT3 inhibitors and/or the tyrosine, tryptophan, and phenylalanine (YWF), effectively and selectively, inhibit proteasome translocation to the cytosol in cells, and moreover, in some embodiments maintains and recruit proteasome to the nucleus. This has been demonstrated by the present disclosure in vitro and in vivo, when the aromatic amino acids of the invention were administered locally to the tumor, or systemically. Most importantly, when provided systemically, either by injectable or oral compositions, the p38 activator/s, Sestrin3 inhibitors, STAT3 inhibitors and/or the triad, p62 and NBR1 inhibitors, YWF, inhibited tumor cell growth, as well as tumor mass and tumor volume.

The pharmaceutical compositions disclosed by the present disclosure, as well as any combinations and combined compositions of the invention can be administered and dosed by the methods of the invention, in accordance with good medical practice, systemically, for example by parenteral, e.g., intrathymic, into the bone marrow, peritoneal or intraperitoneal, specifically administered to any peritoneal cavity, and any direct administration to any cavity or organ, specifically, the pleural cavity (mesothelioma, invading lung) the urinary bladder and to the brain. It should be noted however that the invention may further encompass any additional administration modes. In other examples, the pharmaceutical composition can be introduced to a site by any suitable route including subcutaneous, transcutaneous, topical, intramuscular, intraarticular, subconjunctival, or mucosal, intravenous, e.g., oral, intranasal, intraocular administration, or intra-tumor as well. Still further, local administration to the area in need of treatment may be achieved by, for example, by local infusion during surgery, or using any permanent or temporary infusion device, topical application, direct injection into the specific organ, etc. More specifically, the compositions disclosed herein, that are also used in any of the methods of the invention, described in connection with other aspects of the present disclosure, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). In some optional embodiment, the proteasome dynamics modulator/s of the present invention as well as any formulations thereof may be administered directly to the central nervous system (CNS). Examples of direct administration into the CNS include intrathecal administration, and direct administration into the brain, such as intra-cerebral, intra-ventricular, intra-cerebroventricular, intra-cranial or subdural routes of administration. Such routes of administration may be particularly beneficial for diseases involving or requiring cytosolic proteasome accumulation and/or increased activity of the proteasome in the cytosol, that may in some embodiments affect the central nervous system (e.g., benign or malignant tumors of any neuronal or brain tissue).

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the proteasome dynamics modulator/s of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations, or formulations comprising any other nan- or micro-particles or any matrix comprising the at least one proteasome dynamics modulator/s disclosed herein.

It should be understood that in addition to the ingredients particularly mentioned above, the formulations may also include other agents conventional in the art having regard to the type of formulation in question.

As indicated above, pharmaceutical preparations are compositions that include one or more proteasome dynamics modulator/spresent in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in any organism, specifically any vertebrate organism, for example, any mammal such as human. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of the proteasome dynamics modulator/s of the invention can be achieved in any of the various ways disclosed by the invention.

As noted above, the present invention involves the use of different active ingredients, specifically, the modulating compounds of the present disclosure, for example, the tyrosine, tryptophan and phenylalanine, and optionally, at least one UPS-modulating agent, for example, at least one proteasome inhibitor, and/or any additional therapeutic compound that may enhance stress condition or process, that may be administered through different routes, dosages and combinations. More specifically, the treatment of disorders associated with at least one short term stress condition, as well as any conditions associated therewith, with a combination of active ingredients may involve separate administration of each active ingredient. Therefore, a kit providing a convenient modular format for the combined therapy using the modulating compounds of the invention, specifically, the at least one aromatic amino acid residues, tyrosine, tryptophan and phenylalanine, required for treatment, would allow the desired or preferred flexibility in the above parameters.

As discussed above, the proteasome dynamics modulator/s detailed above in the context of the previously mentioned methods, compositions and kits of the invention are relevant for use in a method for treating, preventing, inhibiting, reducing, eliminating, protecting or delaying the onset of at least one pathologic disorder affected by proteasome activity and/or cellular localization. It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease. The term “treatment or prevention” as used herein, refers to the complete range of therapeutically positive effects of administrating to a subject including inhibition, reduction of, alleviation of, and relief from, pathologic disorder affected by proteasome activity and/or cellular localization and any associated condition, illness, symptoms, undesired side effects or related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the at least one short term cellular stress condition/process and their symptoms, slowing their progress, further exacerbation or development, so as to appear later than in the absence of the treatment according to the invention.

As indicated above, the methods and compositions provided by the present invention may be used for the treatment of a “pathological disorder”, i.e., pathologic disorder or condition involved with at least one short term cellular stress condition/process, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods, kits and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

A further aspect of the present disclosure relates to a combination or a combined composition comprising at least two of the proteasome dynamics and/or function modulators disclosed by the present disclosure. More specifically, in some embodiments, the disclosed modulators may be any compound that leads to at least one of: mTOR activation and localization to the lysosomal membrane and/or preventing dissociation of mTOR from the lysosomal membrane, increase in the ratio of nuclear to cytosolic proteasome localization, also referred to herein as a predominant nuclear localization, reduction in Sestrin3 levels and/or activity, activation of p38, inhibition and/or reduction of Tyr705 of STAT3 phosphorylation, reduction in the levels and/or activity of p62 and/or NBR1, and modulation of NUP93.

In some embodiments, the compound used as a modulator for the at least two modulators of the disclosed combination may be a compound comprising: (a), at least one Y residue, any tyrosine mimetic, any salt or ester thereof, any multimeric and/or polymeric form of the Y residue and/or of said Y mimetic, and any combinations or mixtures thereof; (b), at least one W residue, any tryptophan mimetic, any salt or ester thereof, any multimeric and/or polymeric form of the W residue and/or of said W mimetic, or any combination or mixture thereof; and (c), at least one F residue, any phenylalanine mimetic, any salt or ester thereof, any multimeric and/or polymeric form of the F residue and/or of said F mimetic, and any combinations or mixtures thereof. In some embodiments, the composition further comprises at least one autophagy-targeting agent.

Thus, in some specific embodiments, the disclosed combination or combined composition comprise as an active ingredient at least one proteasome dynamics modulator/s comprising at least one aromatic amino acid residue, any compound that modulates directly or indirectly at least one of the levels, stability and bioavailability of the at least one aromatic amino acid residue, any combinations or mixtures thereof, any vehicle, matrix, nano- or micro-particle thereof, optionally in a least one dosage form or at least one dosage unit form. In some specific embodiments, the combined composition of the invention may comprise any of the proteasome dynamics modulator/s of the invention, specifically, any of the proteasome dynamics modulator/s disclosed herein, or any vehicle, matrix, nano- or micro-particle thereof. In some embodiments, the composition may optionally further comprise at least one pharmaceutically acceptable carrier/s, excipient/s, auxiliaries, and/or diluent/s.

In yet some more specific embodiments, the proteasome dynamics modulator/s comprised within the combination or combined composition provided by the present disclosure may comprise at least one aromatic amino acid residue or a combination of at least two aromatic amino acid residues or any mimetics thereof, any compound that modulates directly or indirectly at least one of the levels, stability and bioavailability of the at least one aromatic amino acid residue, any combinations or mixtures thereof, or any vehicle, matrix, nano- or micro-particle thereof. In some specific embodiments, the proteasome dynamics modulator/s of the compositions disclosed herein may comprise at least two of the following components, optionally, in at least one dosage form or at least one dosage unit form. First component (a), comprises at least one tyrosine (Y) residue, any tyrosine mimetic, any salt or ester thereof, any multimeric and/or polymeric form of the tyrosine residue and/or of the tyrosine mimetic, and any combinations or mixtures thereof. The proteasome dynamics modulator/s may comprise in some embodiments as the second component (b), at least one tryptophan (W) residue, any tryptophan mimetic, any salt or ester thereof, any multimeric and/or polymeric form of the tryptophan residue and/or of the tryptophan mimetic, or any combination or mixture thereof. In yet some further embodiments, the proteasome dynamics modulator/s of the invention may comprise (c), at least one phenylalanine (F) residue, any mTOR agonistic phenylalanine mimetic, any salt or ester thereof, any multimeric and/or polymeric form of the phenylalanine residue and/or of the phenylalanine mimetic, and any combinations or mixtures thereof.

In some embodiments, the proteasome dynamics modulator/s in accordance with the composition of the invention may comprise at least one tyrosine residue, any mimetic, any salt or ester thereof, any multimeric and/or polymeric form thereof, and any combinations or mixtures thereof, and at least one tryptophane residue, any mimetic, any salt or ester thereof, any multimeric and/or polymeric form thereof, and any combinations or mixtures thereof. In some further embodiments, the proteasome dynamics modulator/s in accordance with the composition of the invention may comprise at least one tyrosine residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. In yet some further embodiments, the proteasome dynamics modulator/sin accordance with the composition of the invention may comprise at least one tryptophane residue, any mimetic, any salt or ester thereof, any multimeric and/or polymeric form thereof, and any combinations or mixtures thereof, and at least one phenylalanine residue, any mimetic, any salt or ester thereof, any multimeric and/or polymeric form thereof, and any combinations or mixtures thereof. Still further, in some specific embodiments, the proteasome dynamics modulator/s of the combined composition of the present disclosure may comprise a combination of the following three components, optionally, in at least one dosage form or at least one dosage unit form, or alternatively, in two or three dosage unit forms. More specifically, the composition may comprise: a first component (a), comprising at least one tyrosine residue, any tyrosine mimetic, any salt or ester thereof, any multimeric and/or polymeric form of the tyrosine residue and/or of the tyrosine mimetic, and any combinations or mixtures thereof, optionally, in a dosage unit form. The proteasome dynamics modulator/s of the invention further comprises component (b), comprising at least one tryptophan residue, any tryptophan mimetic, any salt or ester thereof, any multimeric and/or polymeric form of the tryptophan residue and/or of the tryptophan mimetic, or any combination or mixture thereof, optionally, in a dosage unit form. The proteasome dynamics modulator/s of the composition of the present disclosure further comprises component (c), comprising at least one phenylalanine residue, any phenylalanine mimetic, any salt or ester thereof, any multimeric and/or polymeric form of the phenylalanine residue and/or of the phenylalanine mimetic, and any combinations or mixtures thereof, optionally, in at least one dosage form or at least one a dosage unit form. Still further, in some embodiments, the present disclosure provides any combination of the disclosed at least one aromatic amino acid residue with at least one of the disclosed modulators, for example, any of the SESN3 inhibitors, STAT3 inhibitors, p38 activators as disclosed by the present disclosure.

In some embodiments, the compound used as the proteasome dynamics and/or function modulator of the disclosed combined composition acts as an inhibitor of proteasome translocation/requitement. In more specific embodiments, the modulator in the disclosed combination or combined composition of at least two modulators may be a compound that leads to reduction of Sestrin3 levels and/or activity by specifically targeting a nucleic acid sequence encoding Sestrin3, or any parts thereof, and/or by interfering with the interaction of Sestrin3 with at least one regulatory complex.

Thus, in some embodiments, the compound used as a modulator, specifically, an inhibitor of proteasome translocation/requitement in the disclosed combination or combined composition may comprises a gene editing system that targets the nucleic acid sequence encoding Sestrin3. Specifically, in some embodiments, the compound of the present disclosure is any gene editing system or any component/s thereof. Thus, the combinations and/or the combined compositions of the present disclosure may comprise a gene editing system that targets the Sestrin3 thereby leading to reduction in the expression and/or activity thereof. In some specific embodiments, the compound used in the combinations and/or the combined compositions of the present disclosure comprise: (a) at least one nucleic acid guide that targets at least one target sequence within the Sestrin3 encoding nucleic acid sequence, or at least one nucleic acid sequence encoding said nucleic acid guide. The compound of the present disclosure may further comprise in some optional embodiments thereof (b), at least one nucleic acid guided genome modifier protein, or any chimeric protein, complex or conjugate thereof, or at least one nucleic acid sequence encoding the guided genome modifier protein or chimeric protein thereof.

In more specific embodiments, the at least one nucleic acid modifier component comprises at least one clustered regularly interspaced short palindromic repeats (CRISPR)-Cas protein, cas protein derived domain and/or any variant and mutant thereof. In some embodiments, the compound used by the combinations and/or the combined compositions disclosed herein comprises at least one sgRNA that specifically recognizes and binds at least one target sequence within the sestrin3 gene, or any nucleic acid sequence encoding these at least one sgRNA. In yet some specific and non-limiting embodiments, the sgRNA comprises the nucleic acid sequence as denoted by any one of SEQ ID NO: 1, 2, and 3, and designated herein as sgSESN3_1, 2 and 3, respectively.

In yet some further embodiments the present disclosure further provides siRNA specific for Sestrin 3 and uses thereof. In some embodiments, the siRNAs may comprise the nucleic acid sequence as denoted by SEQ ID NO: 49-52, and any combinations thereof.

In yet some additional or alternative embodiments, the Sestrin3 may be targeted functionally, by a compound used in the combinations and/or the combined compositions of the present disclosure. More specifically, such compound may interfere with Sestrin3 function, in some embodiments, by blocking any downstream pathways and/or interactions thereof. Thus, in some embodiments, the compound of the disclosed combinations and/or the combined compositions, may be any compound that interferes and/or blocks, and/or reduces the interaction of Sestrin3 with at least one regulatory complex. In some embodiments, the compound used by the combinations and/or the combined compositions of the present disclosure may be any compound that interferes with, and/or blocks, and/or inhabits, and/or reduces, and/or decreases the inhibitory interaction of Sestrin3 with at least one member of the GATOR2 complex, specifically, with MIOS and/or WDR59.

In some embodiments, the compound used as a modulator, specifically as an inhibitor of proteasome translocation/requitement in the disclosed combination or combined composition may be a compound is a p38 activator that leads to phosphorylation of at least one of Thr180 (T180) and/or Tyr 182 (Y182) of p38.

Still further, in some embodiments, p38 activator useful in the disclosed combinations and/or combined compositions may a compound elevating the levels and/or activity of MAP kinase kinase 3 (MKK3) and/or MKK6. In yet some further additional or alternative embodiments, a p38 activator useful in the disclosed combinations and/or combined compositions may be at least one hyperosmotic agent. Non-limiting embodiments for such hyperosmotic agent, may be sorbitol. Thus, in some embodiments, any carbohydrates having a hyperosmotic effect may be used, for example, glycerin (glycerol), isosorbide, mannitol and urea may be used as the disclosed compounds. In some embodiments, the compound used by the combinations and/or the combined compositions of the present disclosure may be sorbitol. Still further, in some additional or alternative embodiments, a p38 activator useful in the disclosed combination or combined composition may be at least one DNA Synthesis Inhibitor. In some specific embodiments, such compound may be anisomycin.

In some embodiments, the compound used as a modulator, specifically as an inhibitor of proteasome translocation/requitement in the disclosed combination or combined composition may be any STAT3 inhibitor, for example, any compound that inhibits and/or reduces phosphorylation of STAT3. In some particular embodiments, any compound that inhibits and/or reduces phosphorylation of Tyr705 of STAT3.

In some specific and non-limiting embodiments, STAT3 inhibitors useful in the disclosed combination or combined composition may include small molecule compounds, specifically, Stattic (Stat three inhibitory compound), S31-201/NSC74859, BP-1-102, Niclosamide, peptide inhibitors (e.g., the peptide aptamer APT STAT3-9R, and the like). In some specific embodiments, Stattic may be used in the combination or combined composition of the present disclosure.

In some embodiments, the modulator, specifically, the at least one inhibitor of proteasome translocation/requitement used for the disclosed combinations or combined compositions is a compound that leads to at least one of: mTOR activation and localization to the lysosomal membrane, proteasome nuclear localization and/or predominant nuclear localization, reduction in Sestrin3 levels and/or activity, activation of p38, inhibition and/or reduction of Tyr705 of STAT3 phosphorylation, reduction in the levels and/or activity of p62 and/or NBR1, and modulation of NUP93.

Still further, an additional aspect of the present disclosure relates to a kit comprising at least two of the proteasome dynamics and/or function modulators disclosed by the present disclosure, as disclosed herein above. In some embodiments, each of the disclosed modulators may be provided in at least one first and a second dosage unit form. Another aspect of the present disclosure provides a method for treating, preventing, inhibiting, reducing, eliminating, protecting or delaying the onset of at least one condition or at least one pathologic disorder affected by protcasomal activity and/or cellular localization, in a subject in need thereof. The method comprising the step of administering to the subject a therapeutic effective amount of a combination of at least two of the proteasome dynamics and/or function modulators disclosed herein. In some embodiments, any combination or combined composition or kits useful in the disclosed methods are any of the combinations and combined composition as defined by the present disclosure.

It should be understood that for any of the diagnostic steps and/or screening methods of candidate compounds, a sample as used herein any biological sample that comprises any body fluids (blood, plasma, tissue extracts, urine saliva), or any tissues, organs and/or cells, biopsies and the like. The term “co-administered”, as used herein means that all components utilized in the methods of this invention may be administered together as part of a single dosage form (such as a single composition of this invention comprising such components) or in two or three(if the third component is utilized) separate dosage forms. Alternatively, each component may be administered prior to, consecutively with, or following the administration of another component utilized in the methods of this invention as long as all components are administered within sufficient time of one another to achieve the desired effect (e.g., increased activation and/or lysosomal association of mTOR, and/or activation of p38, and/or inhibition of STAT3, inhibition of SESN3, and the resulting increased nuclear localization of the proteasome). In such combination therapy treatment, or non-therapeutic applications each component is administered by conventional, but not necessarily the same, methods. The administration of a composition comprising two or more components utilized in the methods of this invention does not preclude the separate administration of one or more of the same components to said subject at another time during a course of treatment. In some embodiment, all components that are co-administered are all administered within less than 12 hours of each other. In some embodiment, all components that are co-administered are all administered within less than 8, 6, 4, 3, 2, 1, 0.5, or 0.25 hours of each other. In some embodiments, all components are administered simultaneously (e.g., at the same time) or consecutively (e.g., onc right after the other). In some embodiments, the therapeutic methods of the invention comprise the step of administering an effective amount of the proteasome dynamics modulator/s of the present disclosure to a subject in need. An effective amount in accordance with the invention comprise any amount of the p38 activator/s, Sestrin3 inhibitors, STAT3 inhibitors and/or each of the aromatic amino acid residues tyrosine, tryptophan, and phenylalanine (YWF), effective to inhibit proteasome translocation in cells of a subject in need, for example, a subject suffering from cancer. This effective amount in some embodiments may lead to reduction in tumor mass and volume. In yet some further embodiments, an effective amount provided to a subject may range between about 0.01 gr to about 10 gr per day/per kg of body weight.

It should be appreciated that the present disclosure further encompasses any of the disclosed modulators, or any composition comprising at least one of the disclosed modulators. Still further, in some embodiments such modulators may be any p38 activator, with the proviso that the modulator is not at least one of sorbitol and/or Anisomycin. In yet some further alternative and/or additional embodiments, the disclosed modulators may be any STAT3 inhibitor, with the proviso that the STAT3 is not Stattic. As indicated herein, the methods of the invention involve the step of determining at least one of (i) mTOR lysosomal association; (ii) activation of p38; (iii) phosphorylation of Tyr705 of STAT3; and (i) proteasome subcellular localization in at least one cell of said at least one sample, or in any fraction thereof; in at least one cell in a sample. Biological sample is any sample obtained from the subject that comprise at least one cell or any fraction thereof. In some specific embodiments, sample applicable in the methods of the invention may include bone marrow, lymph fluid, blood cells, blood, scrum, plasma, semen, spinal fluid or CSF, the external secretions of the skin, respiratory, intestinal, and genitourinary tracts, any sample obtained from any organ or tissue, any sample obtained by lavage, optionally of the breast ductal system, or of the uterus, plural effusion, samples of in vitro or ex vivo cell culture and cell culture constituents. In some specific embodiments, the biological sample may result from a biopsy. A biopsy is a medical test commonly performed by a surgeon. The process involves extraction of sample cells or tissues from the patient. The tissue obtained is generally examined under a microscope by a pathologist for initial assessment and may also be analyzed for protcasome localization as discussed by the present disclosure. When an entire lump or suspicious area is removed, the procedure is called an excisional biopsy. An incisional biopsy or core biopsy samples a portion of the abnormal tissue without attempting to remove the entire lesion or tumor. When a sample of tissue or fluid is removed with a needle in such a way that cells are removed without preserving the histological architecture of the tissue cells, the procedure is called a needle aspiration biopsy. Still further, the sample/s may be obtained from the described tissues ectomized from a patient (e.g., in case of therapeutic ectomy).

It should be appreciated that the methods, kits and compositions of the present disclosure may be suitable for any subject that may be any multicellular organism, specifically, any vertebrate subject, and more specifically, a mammalian subject, avian subject, fish or insect. In some specific embodiments, the prognostic as well as the therapeutic, cosmetic and agricultural methods presented by the enclosed disclosure may be applicable to mammalian subjects, specifically, human subjects. By “patient” or “subject” it is meant any mammal that may be affected by the above-mentioned conditions, and to whom the treatment and prognosis methods herein described is desired, including human, bovine, equine, canine, murine and feline subjects. Specifically, the subject is a human.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to +10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

TABLE 1
reagents

| Reagents | Supplier | Concentration | |
|---|---|---|---|
| Leptomycin B | Sigma | 2.5 | ng/ml |
| Ivermectin | Sigma | 20 | μM |
| Cycloheximide | Sigma | 100 | μg/ml |
| Phenformin | Sigma | 0.5 | μM |
| Ionomycin | Sigma | 1 | μM |
| 2-DG | Sigma | 2 | mM |
| Torin1 | Tocris | 250 | nM |
| MG-132 | Millipore | 10 | μM |
| Anisomycin | Santa Cruz | 20 | μM |
| Sorbitol | Sigma | 300 | mM |
| LysoTracker | Thermo | 50 | nM |
| Stattic | Sigma | 10 | μM |
| N-Butyl-N-(4- | Santa Cruz | 0.05% | (W/V) |
| hydroxybutyl)nitrosamine | | | |
| 3-Methylcholanthrene | Santa Cruz | 200 | μg/mouse |

TABLE 2
antibodies.

| | | |
|---|---|---|
| Santa Cruz | Cell Signaling: | Abcam: |
| Biotechnology: | Rpn13 | SESN2 |
| β4 | LC3B | CASTOR1 |
| Rpn8 | p70-S6K | NPRL3 |
| Rpn1 | P-p70-S6K | PROX1 |
| Rpn2 (WB) | mTOR | SLC38A9 |
| Lamin A/C | PIK3CA | NBR1 |
| p65 | p38 | GCN2 |
| APC | P-p38 | YY1 |
| NUP93 | p38δ | Tocris: |
| SESN3 | LAMP1 | Rpn2 (IF) |
| Atlas Antibodies: | STAT3 | Sigma: |
| AKIRIN2 | P-STAT3 | α6 |
| Proteintech: | TSC1 | α2 (IF) |
| WDR24 | TSC2 | Millipore: |
| Bio-Techne: | SESN1 | Tubulin |
| AKT | FLCN | |
| | RagA | |
| | RagB | |
| | p62 | |
| | Cleaved | |
| | Caspase3 | |
| | α5 | |
| | α2 (IHC) | |

TABLE 3
sgRNAs used to silence
the respective genes via CRISPR

| sgRNA | Oligo | SEQ ID NO: |
|---|---|---|
| SESN3_1 | CCTGGCACATTATCATGCTT | 1 |
| SESN3_2 | TGATATTAGCCTGAATCCAT | 2 |
| SESN3_3 | GTGTCTCAACCCTTGACAAG | 3 |
| SESN1 | GGCCGTGTACGCCTCGTTCG | 4 |
| SESN2 | ATGGCCGAGTTTCTGCAGAC | 5 |
| CASTOR1 | CCGTTCGGTCATCGCGCCAC | 6 |
| NPRL3 | CAGCCCCATCAGCGTGATTC | 7 |
| FLCN | TGCTCCGACCGAGGATACCT | 8 |
| RagA | GGATGGCCTCCAGACACGAC | 9 |
| RagB | TTGTCCTTAGGTGCTGTTGA | 10 |
| SLC38A9 | CACTCACATGGTTACTAAAC | 11 |
| WDR24 | CACGAACTGTTCCTCCTCGA | 12 |
| sgCont | GCACTACCAGAGCTAACTCA | 13 |

TABLE 4
shRNAs used to silence the respective genes

| shRNA | Oligo | SEQ ID NO: |
|---|---|---|
| mTOR | GGCCGCATTGTCTCTATCAAG | 14 |
| GCN2_1 | GCCTAACTGGTGAAGAAGTAT | 15 |
| GCN2_2 | CCCTAAAGAACTGTCGTTAAC | 16 |
| GCN2_3 | CCAAAGGTCTATCAAATGAAA | 17 |
| PIK3CA_1 | GCATTAGAATTTACAGCAAGA | 18 |
| PIK3CA_2 | GCACAATCCATGAACAGCATT | 19 |
| PIK3CA_3 | GAATTGGAGATCGTCACAATA | 20 |
| AKT_1 | GGACAAGGACGGGCACATTAA | 21 |
| AKT_2 | GGACTACCTGCACTCGGAGAA | 22 |
| NUP93_1 | GCAAGTGAAACAGCGAATTCT | 23 |
| NUP93_2 | GGACTCCACGTTCTATCTTCT | 24 |
| shCont | ATCTCGCTTGGGCGAGAGTAAG | 25 |

TABLE 5
siRNAs used to silence the respective genes

| siRNA | Oligo | SEQ ID NO: |
|---|---|---|
| Sestrin 3 | UGUCAAAGUUUAGCCGUUU | 49 |
| | UCUGAUGUCUCUCGAUAUA | 50 |
| | GCCGGAAAGUGCUGCGGAA | 51 |
| | GGUCAUGAGUUUACACACU | 52 |
| p62 | GAAAUGGGUCCACCAGGAA | 53 |
| | GAUCUGCGAUGGCUGCAAU | 54 |
| | GCAUUGAAGUUGAUAUCGA | 55 |
| | GAAGUGGACCCGUCUACAG | 56 |
| NBR1 | GAGAACAAGUGGUUAACGA | 57 |
| | CCACAUGACAGUCCUUUAA | 58 |
| | GAACGUAUACUUCCCAUUG | 59 |
| | AGAAGCCACUUGCACAUUA | 60 |

It should be noted that each is a mix of 4. were commercially available via Dharmacon (Horizon).

In addition, the siRNAs for TSC1, TSC2, p388, AKIRIN2, and TFEB, each is a mix of 4, were commercially available via Dharmacon (Horizon).

### Experimental Procedures

### Cells and Fly Models

HeLa, RT4, U-2 OS, MEF, and HEK293 cells (ATCC) were grown at 37° C. in DMEM medium, supplemented with 10% fetal bovine serum, 1% penicillin/streptomycin, and 1% sodium pyruvate. MDA-MB-231 cells (ATCC) were grown at 37° C. in RPMI-1640 medium, supplemented with 10% fetal bovine serum, 1% penicillin/streptomycin, and 1% sodium pyruvate. MCF10A cells (ATCC) were grown at 37° C. in DMEM: F12 (1:1) medium, supplemented with 5% horse serum, 1% penicillin/streptomycin, 0.5 ug/ml hydrocortisone, 0.1 ug/ml cholera toxin, 10 μg/ml insulin, and 10 ng/ml endothelial growth factor. HAP1 cells (Horizon) were grown at 37° C. in IMDM medium, supplemented with 10% fetal bovine serum, 1% penicillin/streptomycin, and 1% sodium pyruvate.

D. melanogaster flies from the iso-1 strain (Bloomington Stock Centre: y1; Gr22biso-1 Gr22diso-1 cn1 CG33964iso-1 bw1 sp1; LysCiso-1 MstProxiso-1 GstD5iso-1 Rh61) were kept at 25° C. at 75% relative humidity and were fed with yeast-cornmeal-molasses-malt extract medium.

### Cell Transfection and Protein Overexpression

CalFectin™ (SignaGen) transfection reagent was used to transfect cDNAs. Lipofectamine™ RNAiMAX (Invitrogen) was used to transfect siRNA oligonucleotides. Transfections were carried out according to the manufacturers' instructions.

### Cell Fractionation

Cells were incubated for 20 min in fractionation buffer [20 mM HEPES pH 7.3, 10 mM KCl, 5 mM ATP, 5 mM MgCl₂, and protease inhibitor cocktail (Roche)], followed by the addition of Digitonin (0.01%). They were then mixed thoroughly and centrifuged at 1,000×g for 5 min. The supernatant was collected as cytosolic fraction, and the pellet was washed with fractionation buffer supplemented with NP-40 (0.05%) and centrifuged, and the pellet (nuclei) was resuspended in fractionation buffer supplemented with sodium deoxycholate (0.5%), followed by its sonication.

### Cell Lysates and Western Blotting

Cells were washed twice with ice cold PBS and scraped into lysis buffer (50 mM Tris-HCl, pH 7.4, 130 mM NaCl, 0.5% NP-40) supplemented with freshly added protease inhibitor cocktail, 5 mM ATP, 10 mM iodoacetamide, and 5 mM N-ethyl maleimide. Protein concentration was measured by the BCA assay according to the manufacturer's instructions (Pierce, Rockford, IL). 30 μg of cellular protein were resolved via SDS-PAGE, transferred to a nitrocellulose membrane and immunoblotted with the appropriate antibody.

### Drosophila Gut Imaging

WT flies were maintained on either a yeast-cornmeal-molasses-malt extract medium (Cont.) or 5% sucrose solution (St.) for 6 h. Dissection, fixation and staining of intestines were carried out as described previously [R. L. Shaw et al., Development 137, 4147-4158 (2010)].

### Immunofluorescence Microscopy

Cells were seeded on glass cover slips for 36 h. Following the indicated treatments, they were fixed with 4% PFA for 15 min, washed with phosphate-buffered saline (PBS) and incubated in PBS containing 10% goat serum for 1 h at room temperature, followed by 2 h incubation with the indicated primary antibody. Following extensive wash with PBS, the fixed cells were incubated with the relevant secondary antibody for 1 h, washed and mounted. Images were acquired using Zeiss LSM 700 confocal microscope (Zeiss, Oberkochen, Germany).

### Measurement of Degradation Rates

Cells were labeled with [³⁵S]-methionine and cysteine (20 μCi/ml) for 16 h. This was followed by extensive washing and further incubation in a medium containing 2 mM of the two unlabeled amino acids for 8 h. Degradation rates were assessed by determining the release of labeled amino acids to the incubation medium relative to the radioactivity remained in the cellular proteins (using Trichloroacetic acid precipitation to separate between the two) [R. Gropper et al., Biomed Biochim Acta 50, 321-332 (1991)]. The figure presents mean+/−SD and Student's T-Test p-values of three replicates.

### Monitoring Proteasome Shuttling Using Photoconvertible Fluorescent Protein

Cells stably expressing the photoconvertible protein Dendra2 fused to the proteasomal subunit β4 were seeded on a glass-bottomed plate. Two days later, the plate was placed in an environmental controlled chamber within a Zeiss LSM 700 confocal microscope. To achieve conversion of the green fluorescence of Dendra2 to red, excitation was performed using a 405 nm laser. Following acquisition of the newly generated red fluorescent proteasome, cells were starved for 8 hours while kept in the microscope's chamber to maintain the same field of view. Proteasome localization was then imaged again, followed by replenishment of amino acids, further incubation and acquisition after additional 8 and 12 hours.

### Fluorescence-Based Proteasome Activity Assays

Live cell proteasome activity was followed as previously described [C. R. Berkers et al., Mol Pharm 4, 739-748 (2007)]. In brief, Me₄BodipyFL-Ahx₃Leu₃VS was added to the medium to a final concentration of 1 μM. Following incubation for 15 min, the cells were visualized by a Zeiss LSM 700 confocal microscope. In vitro proteasome activity assay was carried out as previously described [O. Braten et al., Proc. Natl. Acad. Sci. 113, E4639-47 (2016)]. In brief, cellular fractions were incubated at 37° C. for 30 min with 5 μM Suc-LLVY-AMC (Succinyl-Leu-Leu-Val-Tyr-amido-4-methylcoumarin) in a reaction buffer (40 mM Tris-HCl pH 7.2, 2 mM DTT, 5 mM MgCl₂, 10 mM creatine phosphate, 0.1 mg/ml creatine phosphate kinase, 5 mM ATP). Reactions were stopped by adding 1% SDS, and fluorescence was measured at 360/460 nm (ex/em).

### Monitoring Autophagic Flux

Cells stably expressing the fusion of RFP-GFP-LC3 were treated as described and were visualized live, using high-throughput fluorescence microscopy (IXM-C, Molecular Devices) under controlled environment (21% O2, 5% CO₂, 37° C.). Analysis of autophagic flux was made based on the differential signal in the green and red channels, as previously described [Nicklin, et al. (2009). Cell 136, 521-534] Due to the instability of GFP in acidic pH, only RFP retains its fluorescence in the acidic lysosome, while both are visible in the autophagosome. The RFP: GFP puncta ratio is indicating the extent of autophagy activation, or flux. The figure presents mean+/−SD and Student's T-Test p-values of three replicates.

### Sample Preparation for Protein Mass Spectrometry

2-3 mg of cell extract protein in 8 M Urea and 100 mM ammonium bicarbonate, were incubated with DTT (2.8 mM; 30 min at 60° C.), modified with iodoacetamide (8.8 mM; 30 min at room temperature in the dark), and digested (overnight at 37° C.) with modified trypsin (Promega; 1:50 enzyme-to-substrate ratio) in 2 M Urea and 25 mM ammonium bicarbonate. Additional second trypsinization was carried out for 4 h. The tryptic peptides were desalted using Sep-Pak C18 (Waters) and dried. 10 μg of protein were used for proteome analysis as described under Mass spectrometry.

Enrichment of ubiquitinated peptides: 2% were set aside for proteomics analysis. The rest of the sample was dried and enriched for ubiquitinated peptides using the PTMScan® Ubiquitin Remnant Motif (K-8-GG) Kit (Cell Signaling) according to the manufacture instructions.

### Proteins Mass Spectrometry

Tryptic peptides were analyzed by LC-MS/MS using a Q Exactive plus mass spectrometer (Thermo Fisher Scientific) fitted with a capillary HPLC (easy nLC 1000, Thermo). The peptides were loaded onto a C18 trap column (0.3×5 mm, LC-Packings) connected on-line to a home-made capillary column (20 cm, internal diameter 75 microns) packed with Reprosil C18-Aqua (Dr. Maisch GmbH, Germany) in solvent A (0.1% formic acid in water). The peptides mixture was resolved with a 5-28% linear gradient of solvent B (95% acetonitrile with 0.1% formic acid in water) for 180 min followed by a 5 min gradient of 28-95% and 25 min at 95% acetonitrile with 0.1% formic acid at a flow rate of 0.15 μl/min. Mass spectrometry was performed in a positive mode (m/z 350-1800, resolution 70,000) using repetitively full MS scan followed by collision-induced dissociation (HCD at 35 normalized collision energy) of the 10 most dominant ions (>1 charges) selected from the first MS scan. A dynamic exclusion list was enabled with exclusion duration of 20 sec.

The ubiquitinated peptides were analyzed with a similar gradient (a linear 180 minutes gradient of 5 to 28% acetonitrile followed by a 15 minutes gradient of 28 to 95% and 25 minutes at 95% acetonitrile with 0.1% formic acid in water at flow rates of 0.15 μl/min), by the Q Executive HFX mass spectrometer (Thermo) in a positive mode (m/z 350-1200, resolution 120,000 for MS1 and 15,000 for MS2) using repetitively full MS scan followed by collision induced dissociation (HCD, at 27 normalized collision energy) of the 30 most dominant ions (>1 charges) selected from the first MS scan. The AGC settings were 3×106 for the full MS and 1×105 for the MS/MS scans. A dynamic exclusion list was enabled with exclusion duration of 20 s.

### Proteomics Data Analysis

The mass spectrometry raw data were analyzed by the MaxQuant software (version 1.4.1.2, http://www.maxquant.org) for peak picking and quantification. This was followed by identification of the proteins using the Andromeda engine, searching against the human UniProt database with mass tolerance of 20 ppm for the precursor masses and for the fragment ions. Met oxidation, N-terminal acetylation, N-ethylmaleimide and carbamidomethyl on Cys, GlyGly on Lys, and phosphorylation on Ser, Thr and Tyr residues, were set as variable post-translational modifications. Minimal peptide length was set to six amino acids and a maximum of two mis-cleavages was allowed. Peptide and protein levels false discovery rates (FDRs) were filtered to 1% using the target-decoy strategy. Protein tables were filtered to eliminate identifications from the reverse database and from common contaminants. The MaxQuant software was used for label-free semi-quantitative analysis [based on extracted ion currents (XICs) of peptides], enabling quantification from each LC/MS run for each peptide identified in any of the experiments. In samples that were SILAC-labeled, quantification was also carried out using the MaxQuant software. Data merging and statistical tests were done by the Perseus 1.4 software.

The data of the ubiquitinated peptides was quantified by label free analysis using the same software. Statistical analysis of the identification and quantization results was done using Perseus 1.6.2.2 software. The figure presents means, and the indicated number of replicates for each condition.

### Mass Spectrometric Analysis of Proteasome Sub-Complexes' Composition

Analysis of proteasome assembly and sub-complex composition was performed using the MCP20 antibody against the α6 proteasome subunit, as previously described using the MCP21 antibody against α2 [B. Fabre et al., Mol. Cell. Proteomics 12, 687-699 (2013)]. Briefly, following the indicated treatments, cell lysates were immunoprecipitated using the α6 antibody, and the precipitates were subjected to proteomic mass spectrometry. Intensities of the different proteasomal subunits were normalized according to the median of intensities measured for all 20S subunits. Ratios were calculated for each condition relative to untreated cells (control). The color scale represents Log 2 fold-change.

### Amino Acids Level Measurement

Metabolic analysis was carried out as previously described [G. M. MacKay et al., 1st Ed. (Elsevier Inc., 2015)]. Briefly, cells were rapidly washed 3 times with ice-cold PBS and extracted with an aqueous solution of 50% Methanol, and 30% Acetonitrile. Samples were centrifuged at 16,000×g for 10 min at 4° C., and the supernatants were analyzed using HPLC-MS (Q-Exactive Orbitrap Mass Spectrometer (Thermo Scientific) coupled to Thermo Scientific UltiMate 3000 HPLC system). The HPLC setup consisted of a ZIC-PHILIC column (SeQuant, 150×2.1 mm, 5 μm, Merck) with a ZIC-PHILIC guard column (SeQuant, 20×2.1 mm). The aqueous mobile phase solvent was 20 mM ammonium carbonate adjusted to pH 9.4 with 0.1% ammonium hydroxide. The organic mobile phase was acetonitrile. Amino acids and other metabolites were separated over a 15 min linear gradient from 80% organic to 80% aqueous. The column temperature was 45° C., the flow rate 200 μl/min, and the run time 27 min. All metabolites were detected across a mass range of 75-1,000 m/z using the Q-Exactive mass spectrometer at a resolution of 35,000 (at 200 m/z) with electrospray ionization and polarity switching mode. Mass accuracy obtained for all metabolites was below 5 ppm. Data were acquired with Thermo Xcalibur software. The peak areas of different amino acids were determined using Thermo TraceFinder software through which metabolites were identified by the exact mass of the singly charged ion and the known retention time on the HPLC column. Commercially available standard compounds had been analyzed before to determine ion masses and retention times on the ZIC-PHILIC column. Protein quantitation based on the Lowry method was performed for normalization. The figure presents mean+/−SD of three replicates.

### Cell Survival, Nucleo-Cytoplasmatic Translocation, and Ubiquitination Assays

Cells were seeded in a 96-well plate at a density of 15,000 cells/well. ˜36 h later, cells were treated as described and were visualized live, using high-throughput fluorescence microscopy (IXM-C, Molecular Devices) under a controlled environment (21% O₂, 5% CO₂, 37° C.). Hoechst 33342 was used to stain nuclei of all cells, and SYTOX™ (Thermo) was used to stain dead cells. Data analysis was performed using the Live/Dead module of the MetaXpress software (Molecular Devices). For nuclear export analysis, the percent of cells exhibiting nuclear vs cytosolic predominance was measured using the Translocation-Enhanced module of the MetaXpress software (Molecular Devices), with DAPI used for the demarcation of nuclei. For ubiquitination, the inventors used in-cell Western blotting using an anti-Ub-conj [O. Braten et al., Proc. Natl. Acad. Sci. 113, E4639-47 (2016), and a secondary fluorescent antibody. Intensities were quantified using Li-Cor imager and software (Odyssey).

### Xenografts

MDA-MB-231 (ATCC® HTB-26™) or RT4 (ATCC® HTB-2TM) cells were dissociated with trypsin, washed with PBS, and brought to a concentration of 70×10⁶ cells/ml. Cell suspension (7×10⁶/0.1 ml) was inoculated subcutaneously at both flanks of 12 weeks old NOD.Cg-Prkdc^{scid}II2rg^{tm1Wjl}/SzJ (NSG) mice, JAX stock #005557. Following formation of a palpable mass, 500 μl of either saline, saline supplemented with 25 mM/each of YWF, or saline supplemented with 25 mM/each of QLR, was injected subcutaneously 3 times a week at both flanks (adjacent to the growing tumor). For oral treatment, YWF were dissolved in the drinking water at a concentration of 6 mM each. After the largest tumor in each experiment has reached the maximal size allowed by the guidelines for animal care, all mice were sacrificed and xenografts were resected, weighed, and fixed in formalin. Paraffin-embedded sections were stained using standard immunohistochemistry protocol as described previously [Y. Kravtsova-Ivantsiv et al., Cell 161, 333-347 (2015)]. Apoptotic cells were detected using terminal deoxynucleotidyl transferase dUTP nick end labeling (TUNEL) according to the manufacturer's protocol, and via immunofluorescence against the apoptotic marker cleaved-Caspase3. Volumetric monitoring of tumors was carried out using a caliper twice a week. All animal experiments were carried out under the supervision of the accredited Animal Care Committee of the Technion.

### Spontaneous Tumor Models

For the colorectal tumor model, APC^{fl/fl} mice were kindly provided by E. Fearon (University of Michigan, Ann Arbor, MI). CDX2-CreER^{T2} mice were purchased from the Jackson laboratory. All transgenic mice were on C57BL/6J background. The mice were crossed to generate APC^{fl/fl} CDX2-CreER^{T2} mice. To get discrete colonic tumors The inventors calibrated a model based on a low dose tamoxifen. Tamoxifen (Sigma) was diluted in corn oil (Sigma) and injected intraperitoneally in a single dose of 20 mg/kg. For YWF treatment, YWF at a concentration of 6 mM was administered in the drinking water starting 10 Days after tamoxifen injection, and mice were sacrificed 7 weeks later. Colonic tumors exceeding 0.5 mm were measured using a digital caliper. Tumors in the cecum are hard to measure due to the irregularity of the surrounding cecal tissue. Thus, cecum mass was measured and compared to APC^{fl/fl} non-induced mice, to estimate the change in tumoral mass in the cecum. Tissue was fixed in 4% formaldehyde and FFPE blocks were prepared for histologic analysis.

Bladder carcinoma was induced adding the carcinogen N-Butyl-N-(4-hydroxybutyl) nitrosamine (BBN) to the drinking water as previously described [M. Degoricija et al., J. Transl. Med. 2019 171 17, 1-13 (2019)]. Sarcomas were induced by a single subcutaneous injection of 3-Methylcholanthrene (3-MCA), as previously described [Y. Krelin et al., Cancer Res. 67, 1062-1071 (2007)].

### Metastatic Triple Negative Breast Cancer Model

Nine weeks old BALB/c female mice (Envigo, Jerusalem, Israel) were injected subcutaneously with 4×105 4Tl mCherry-expressing cells in 50 μl PBS to the lower left mammary fat pad. Mouse weights were monitored, and tumor dimensions were measured by a caliper 3 times a week. Tumor volume was defined as (length)×(width)²/2.

Metastases detection: following mice sacrificing, the livers were harvested and imaged using IVIS Spectrum CT Pre-Clinical In Vivo Imaging System (PerkinElmer, MA, USA) at ex/em of 570/620 nm, binning of 2, f-stop of 2, and a 10 s exposure time to detect mCherry metastases. Quantitative data from the images were obtained using ROI tool in Living Image software. Non-inoculated control mice were used for the analysis, and the average radiance of their unaffected livers was used as a baseline.

### Example 1

Amino Acid Starvation Induces Active Translocation of the 26S Proteasome from the Nucleus to the Cytosol

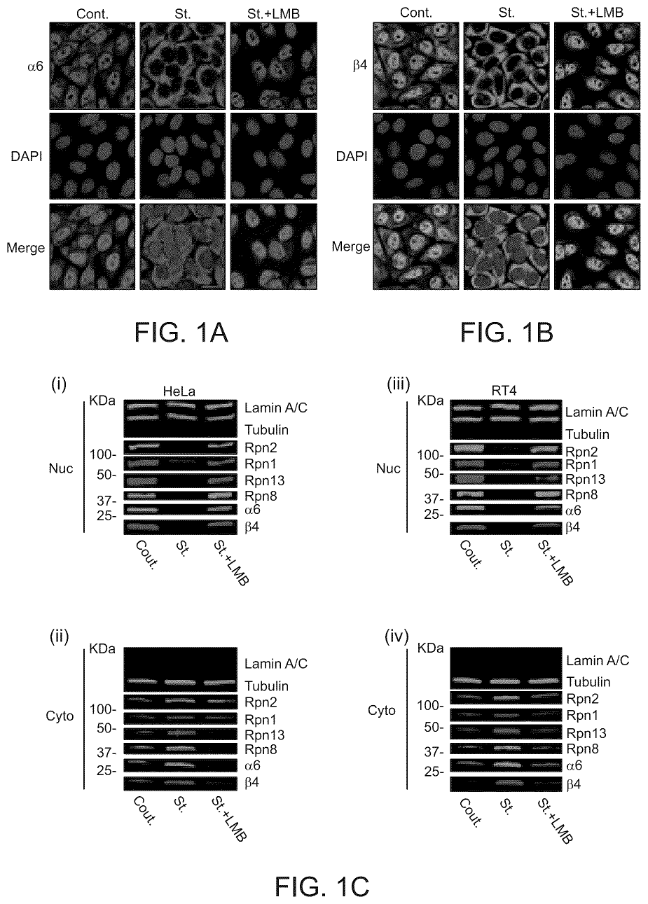

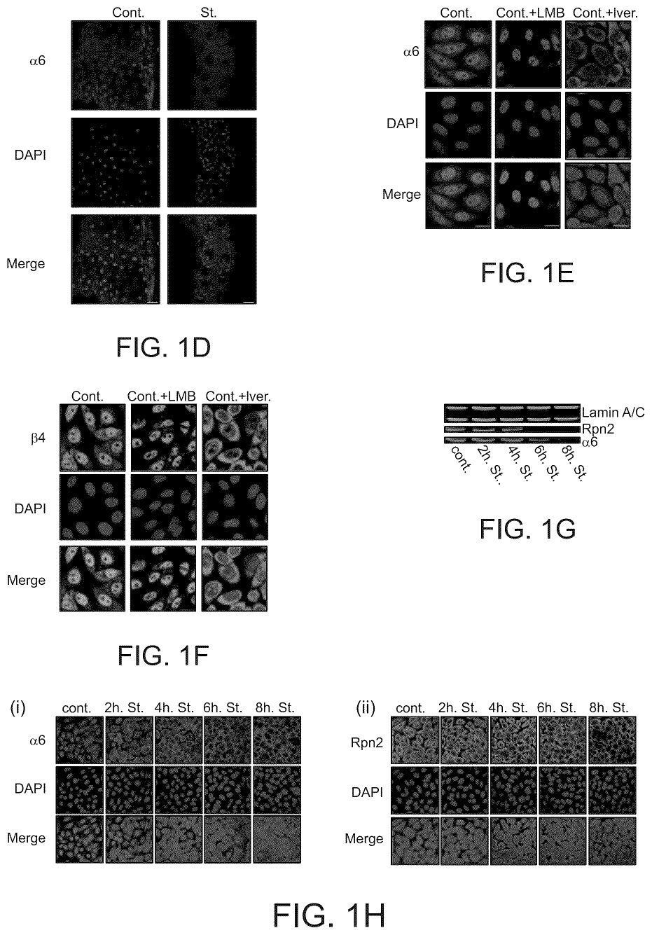

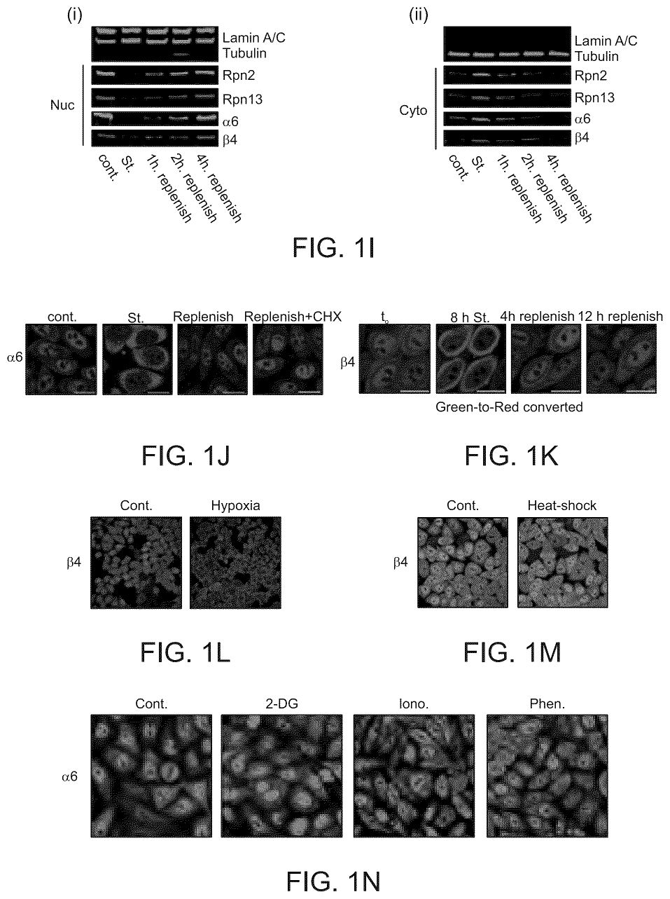

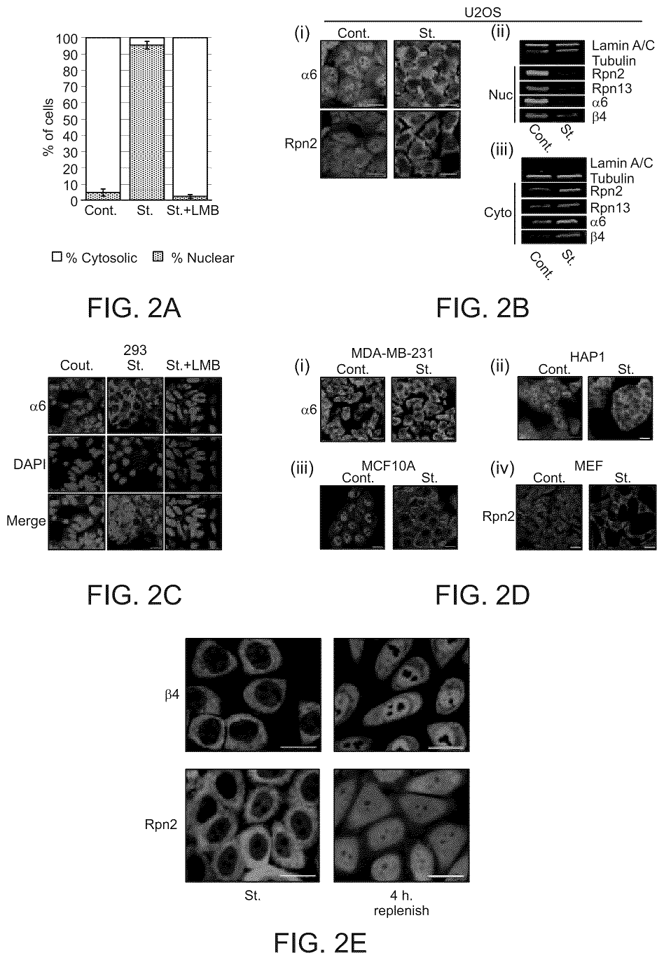

The inventors have shown previously that the proteasome undergoes autophagic degradation following amino acid starvation for longer than 24 h [V. Cohen-Kaplan et al., Proc. Natl. Acad. Sci. 113, E7490-99 (2016)]. To shed light on the fate of the proteasome following a shorter period of stress, the subcellular localization of both the 20S and 19S complexes (FIG. 1) was monitored. Following amino acid starvation for 8 h, the nuclear proteasome-which constitutes a large fraction of the cellular enzyme—is translocated to the cytosol (FIGS. 1A, 1B and 1C). Treating starved cells with Leptomycin B (LMB), an exportin1 inhibitor [N. Kudo et al., Exp. Cell Res. 242, 540-547 (1998)], resulted in inhibition of the translocation, suggesting that the recruitment is active (FIGS. 1A, 1B and 1C). This inversion of localization of the proteasome is common to nearly all the cells in the dish (FIG. 2A). The stress-induced translocation is not unique to a single cell type and was observed in other malignant and non-malignant cell lines (FIG. 2B-2D).

Proteasome dynamics following starvation in vivo, was next monitored by visualizing the proteasome in the gut of starved fruit flies. Localization of the proteasome in control flies was clearly nuclear, whereas in flies deprived of amino acids, it was translocated to the cytosol (FIG. 1D).

It should be emphasized that the proteasome shuttles between the nucleus and the cytosol all the time, probably fine-tuning proteolysis in the ever-changing metabolic and environmental conditions: LMB treatment resulted in nuclear accumulation of the proteasome in non-stressed cells, while treatment with Ivermectin, an inhibitor of nuclear import [K. M. Wagstaff et al., J. Biomol. Screen. 16, 192-200 (2011)], resulted in its cytosolic accumulation (FIGS. 1E and 1F), strongly suggesting that the localization of the proteasome is the result of a dynamic steady state. Interestingly, the 20S and 19S proteasome complexes display a different basal distribution between the cellular compartments, with the 20S being more concentrated in the nucleus relative to the 19S, though also the 19S responds to both export stimulation under starvation, as well as to nuclear accumulation in the presence of LMB (FIGS. 1A, 1B and 1C). This was further corroborated by monitoring the kinetics of proteasome translocation: Western blot analysis of nuclear fractions and confocal microscopy similarly showed that while both the 20S and 19S proteasome are recruited from the nucleus, the 19S (presented by RPNs) which is a priori more cytosolic reaches a minimal nuclear level faster than its 20S (presented by α6 and β4) counterpart (FIGS. 1G and 1H). This finding is in agreement with previous reports that only part of the two sub-complexes of the proteasome are assembled at a given time [B. Fabre et al., Mol. Cell. Proteomics 12, 687-699 (2013); B. Fabre et al., J. Proteome Res. 13, 3027-3037 (2014)]. It makes a physiological sense to enforce a tighter compartmentalization of the catalytic sub-complex.

### Example 2

### Proteasome Recruitment is Reversible and Specific, Yet is not Limited to Nutritional Stress

The reversibility of proteasomal redistribution was next tested, especially in light of the inventor's previous finding that long-term starvation results in autophagic degradation of the complex [Cohen-Kaplan et al. (2016)]. Replenishment of amino acids to 8 hours-starving cells restored the nuclear proteasomal pool within ˜4 hours (FIGS. 1I and 2E). Replenishment of amino acids in the presence of the protein synthesis inhibitor cycloheximide (CHX) did not prevent restoration of the nuclear proteasomal pool (FIG. 1J), demonstrating that it does not require de novo protein synthesis; it is rather the same pool of proteasome that was translocated to the cytosol which returns now to the nucleus. To further corroborate this finding, the proteasome was tagged with a photo-convertible fluorophore, allowing conversion of pre-existing proteasomes from green to red, therefore monitoring only complexes synthesized prior to the amino acid deprivation. Live imaging of the same field demonstrated that the stress induces translocation of the proteasome to the cytosol, while their replenishment results in re-localization of the previously migrated complexes back to the nucleus (FIG. 1K).

It was then important to test whether proteasome recruitment in response to starvation is stimulus specific. It was found that while hypoxia also induced proteasomal translocation (FIG. 1L), neither heat shock (FIG. 1M) nor inducers of autophagy via AMPK (FIG. 1N) resulted in proteasome export. These observations further distinguish the newly identified amino acids starvation-induced translocation in mammalian cells, from the formation of proteasome storage granules in yeast following glucose starvation—which is mediated via AMPK [Burcoglu et al., Cells 4, 387-405 (2015)]. Taken together, nucleo-cytoplasmic proteasome shuttling seems specific, and most probably serves a pathophysiological role (see below).

### Example 3

An as Yet Unidentified mTOR Signaling Pathway Regulates Stress-Induced Proteasome Dynamics

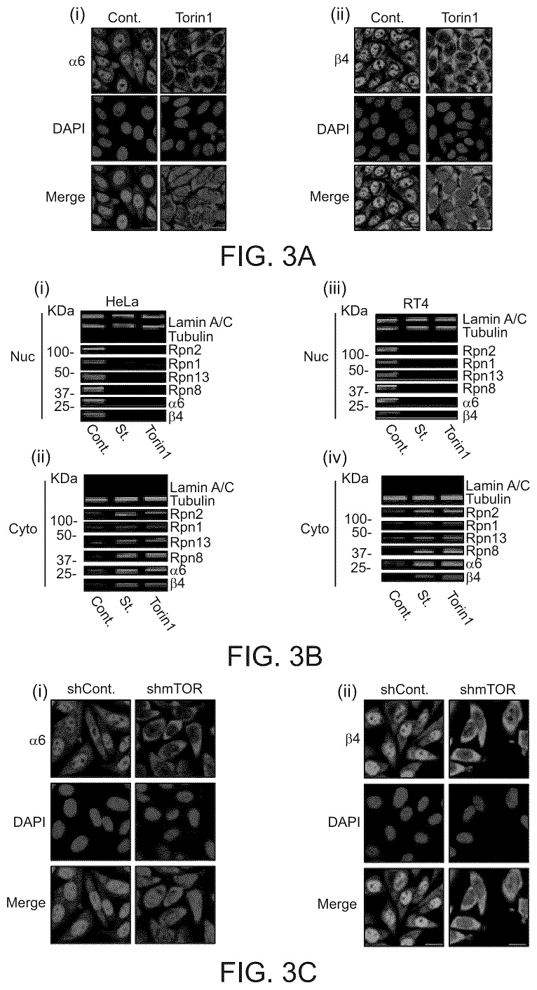

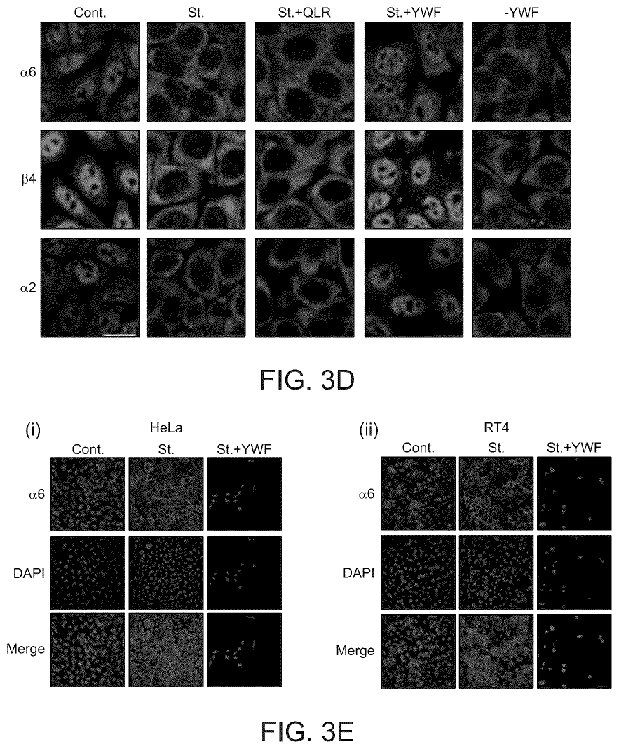

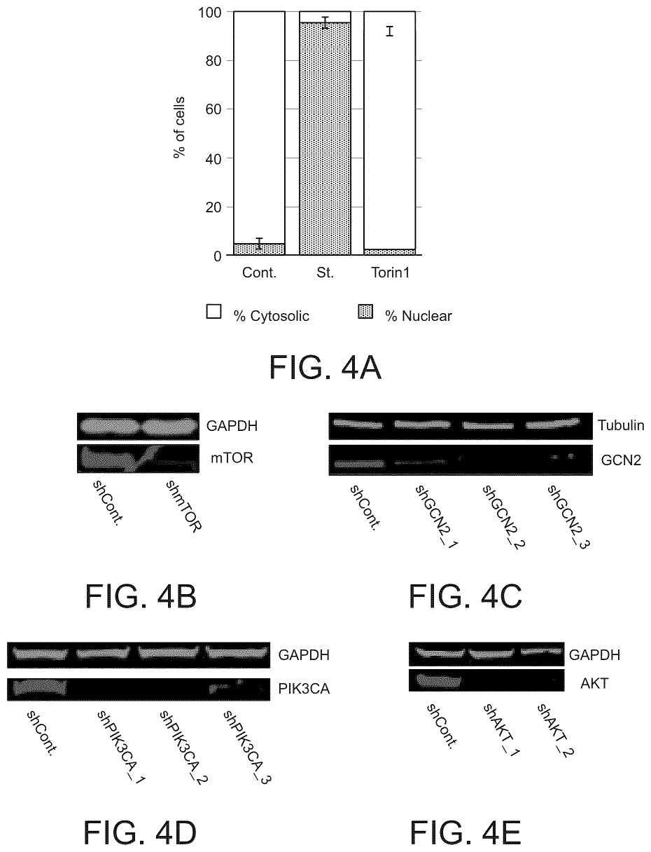

Since amino acids sensing is largely mediated by the mTOR signaling network [J. Heitman et al., Science 253, 905-9 (1991); D. M. Sabatini et al., Cell 78, 35-43 (1994)], Torin1—an mTOR-specific inhibitor [D. A. Guertin et al., Sci. Signal. 2, 1-7 (2009)]—was used to test whether this pathway is responsible also for starvation-induced proteasome translocation. Similar to amino acid starvation, Torin1 induced nuclear export of both 20S and 19S sub-complexes in the presence of complete growth medium (FIGS. 3A, 3B and 4A). Similarly, knockdown of mTOR expression (FIG. 4B) also results in proteasome translocation to the cytosol (FIG. 3C).

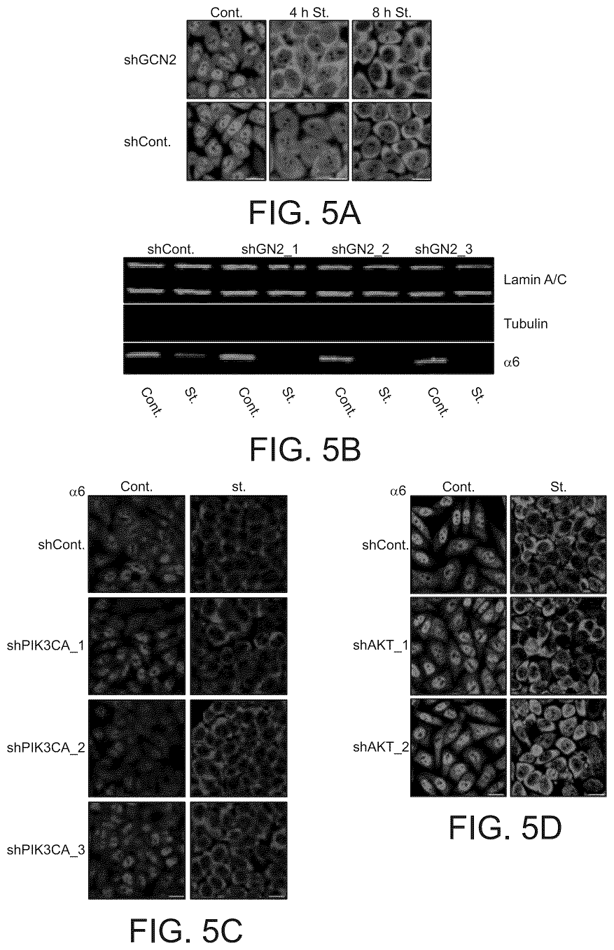

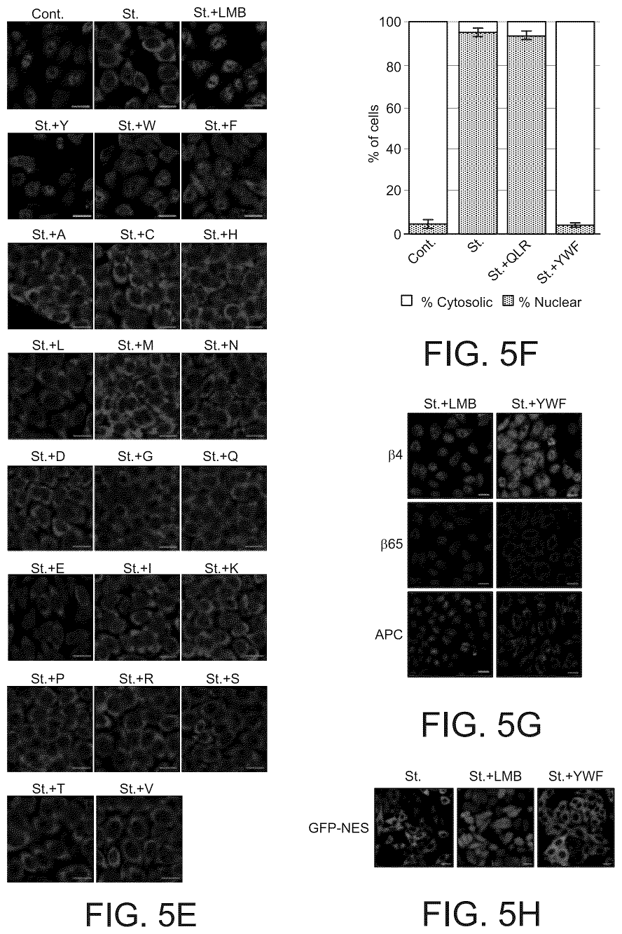

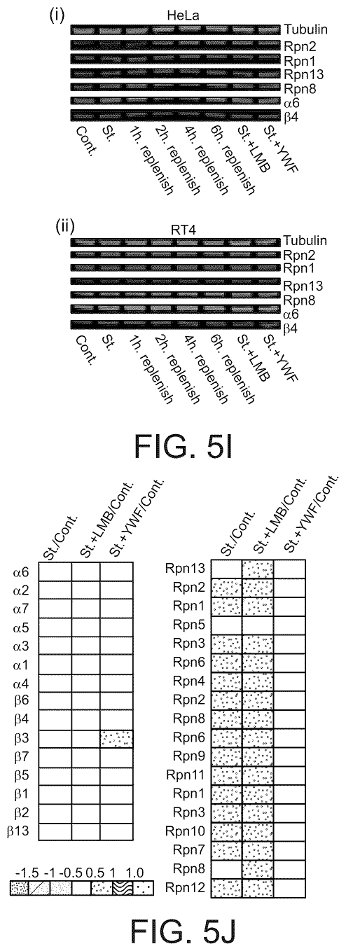

Since mTOR is not the only sensor of cellular amino acid pool, the inventors tested whether other reported pathways-GCN2, a sensor for uncharged tRNAs [S. A. Wek et al., Mol. Cell. Biol. 15, 4497-4506 (1995)], and PIK3CA/AKT [I. Tato et al., J. Biol. Chem. 286, 6128-6142 (2011); M. Wolfner et al., J. Mol. Biol. 96, 273-290 (1975); T. E. Dever et al., Cell 68, 585-596 (1992)]—are required for proteasome export following amino acid starvation. Silencing GCN2, PIK3CA, or AKT (FIGS. 4C, 4D, 4E, respectively), had no effect on proteasome translocation (FIG. 5A, 5B, 5C, 5D). Taken together, the findings strongly suggest a role for mTOR signaling in proteasome dynamics.

To date, the amino acids which were shown to modulate mTOR activity are Gln, Leu, and Arg (QLR) [R. L. Wolfson et al., Cell Metab. 26, 301-309 (2017); A. González et al., EMBO J. 36, 397-408 (2017)]. These ‘canonical’ amino acids were shown to regulate several mTOR downstream pathways, such as TFEB and ULKI-mediated autophagy [C. Settembre et al., Science 332, 1429-1433 (2011); C. H. Jung et al., Mol. Biol. Cell 20, 1992-2003 (2009); H. W. S. Tan et al., Nat Commun 8, 338 (2017)], and p70-S6K and 4EBP-mediated regulation of translation [D. J. Price et al., Science 257, 973-7 (1992); S. R. von Manteuffel et al., Proc. Natl. Acad. Sci. U.S.A. 93, 4076-80 (1996)]. The inventors found that unlike their ability to activate mTOR in the context of autophagy and translation [J. L. Jewell et al., Science 347, 194-198 (2015)], the addition of QLR to the starvation medium did not prevent mTOR-mediated proteasome export (FIG. 3D). The finding that none of the currently known mTOR agonistic amino acids are involved in stress-induced translocation of the proteasome, prompted a systematic search for the one(s) involved. Screening the entire repertoire of amino acids identified Tyr, Trp, and Phe (YWF)—the three aromatic amino acids: the addition of each of them to the starvation medium had an inhibitory effect on proteasome translocation, while the entire triad had a significantly stronger effect (FIGS. 3D and 5E). A quantitative analysis of proteasome translocation demonstrated both the extent of proteasome recruitment-seen in virtually all the cells, as well as the ‘universality’ of its inhibition by YWF, as shown by the complete reversal back to the nuclear predominance observed in well fed cells (FIG. 5F). In a complementary experiment, the inventors showed that subtracting only YWF from the complete medium (leaving the remaining 17 amino acids, including QLR) is sufficient to stimulate proteasome recruitment to the cytosol (FIG. 3D). Intriguingly, the inventors noticed that unlike the starved cells—which did not seem to differ in number from the control group, most of the starved cells to which YWF were added died, which appears to be in correlation with their nuclear proteasomal sequestration (FIG. 2E; see herein after for detailed analyses). Importantly, YWF inhibit specifically proteasome export, and do not affect the export machinery at large: while LMB inhibited nuclear export of two known substrates of exportin1—the p65 subunit of NF-κB and the tumor suppressor adenomatous polyposis coli (APC), YWF had no effect on the localization of the two (FIG. 5G). Similarly, while addition of LMB leads to nuclear accumulation of GFP fused to a nuclear export signal (NES), YWF has no effect on its cytosolic dominance (FIG. 5H).

Of note is that under the tested conditions, the levels of both sub-complexes of the 26S proteasome remained unchanged (FIG. 5I), underscoring previous report by the inventors, that the complex is stable during a short-term stress [Cohen-Kaplan et al., (2016)], and all changes reported herein are due to its redistribution within the cell.

Testing the effect of YWF on ‘canonical’ mTOR functions, it was found that supplementing cells with the triad partially inhibited the upregulation of autophagy induced by starvation (assessed via monitoring LC3 lipidation), similar to previous observations made regarding Gln, Leu, or Arg [González et al., (2017)]. Interestingly, subtraction of only YWF-which was sufficient to induce proteasome recruitment (FIG. 3D), did not stimulate autophagy, probably due to the presence of Gln, Leu, and Arg in the medium. This is probably due to supplementation of amino acids originating from accelerated proteasome-mediated degradation of cytosolic proteins (see below). Taken together, these findings place QLR and YWF as distinct stimuli affecting different pathways downstream of mTOR. Of note is that it was described already that in response to different inhibitors, mTOR does not act in a binary ON/OFF manner, but rather relays different downstream signals-depending on the nature of the inhibitor [C. C. Thorcen et al., J. Biol. Chem. 284, 8023-8032 (2009)]. It was also suggested that different amino acids may exert different inputs on mTORC1, resulting in differential downstream outputs [D. C. I. Goberdhan et al., Cell Metab. 23, 580-589 (2016)]. The present invention now shows that this may be the case with regard to the ‘canonical’ amino acids (QLR) and the newly described YWF, as well as their downstream effects. Notably, the addition of either protcasome or autophagy inhibitors to starvation media did not rescue p70 phosphorylation, further suggesting that YWF exert their signaling effect at the mTOR level and not downstream through, for example, a direct effect on the proteasome or the autophagic machinery. This signal (i.e. starvation) and its inhibition (by YWF) also affected proteasome assembly. Starvation resulted in upregulation of the assembly between the 20S and 19S sub-complexes (FIG. 5J). Importantly, while inhibiting the translocation “mechanically” using LMB did not reverse the effect of stress on proteasome assembly, the addition of YWF did (FIG. 5J), suggesting that the triad has ablated altogether the stimulation of proteasome-mediated protein breakdown.

### Example 4

Sestrin3 is Required for mTOR-Dependent Proteasome Recruitment

Since the inventors showed that the formerly known mTOR agonistic amino acids-QLR-do not affect proteasome dynamics under stress, it was reasonable to assume that also the sensing of the newly identified tried is carried out by an as yet unidentified mediator. To identify the putative mediator, an array of proteins implicated with the mTOR sensing machinery was next knocked out (KO) (FIG. 6A(i)-6A(iii)). The silencing of Sestrin3 (SESN3; FIG. 6A(ii)), which was also shown to interact with the GATOR2 complex upstream to mTOR, while having negligible role in Leu sensing [13, 14], ablated the effect of starvation on proteasome recruitment to the cytosol (FIGS. 7A and 7B). None of the other KO proteins, including SESN2, showed an effect on proteasome localization (FIG. 7A). In addition to the lack proteasome recruitment following starvation, SESN3 KO also increased the nuclear concentration of the proteasome (FIG. 7B). Similar results were also shown when siRNA against SESN3 was used, leading to increased basal nuclear concentration of the proteasome, and lack of proteasome translocation to the cytosol under starvation (FIG. 6P). Importantly, overexpression of the silenced gene rescued both the basal distribution of the proteasome and its recruitment to the cytosol under starvation (FIG. 7C).

To further link the stimulus generated by YWF to the signaling of SESN3 upstream of mTOR, the effect of YWF on the interaction of SESN3 with the GATOR2 complex was tested. YWF strongly inhibited SESN3 interaction with WDR59 and MIOS-two members of the GATOR2 complex (FIG. 7E), similar to the effect of Leu on SESN2 [13, 14]. Taken together, these findings identify an as yet undescribed signaling cascade that mediates the sensing of YWF via the SESN3 and the GATORs' pathway upstream of mTOR.

In addition to amino acid deprivation, genotoxic stress was also shown to inhibit mTOR via a p53-mediated induction of SESN1 and SESN2, but not of SESN3. SESN1/2 were shown to activate

AMPK and TSC2 under these conditions, thereby inhibiting mTOR [11]. Furthermore, mice lacking only SESN2 had an abnormal AMPK response to stress, failing to inhibit mTOR [A. V. Budanov et al., Cell 134, 451-460 (2008)], constituting another instance in which different members of the Sestrins family do not compensate for the lack of one another. After showing that AMPK signaling is not involved in mTOR-mediated proteasome dynamics (FIG. 1N), both TSC1 and TSC2 were next silenced (FIG. 6B(i)) to test whether their inhibitory effect on mTOR is required for proteasome recruitment. It was found that the absence of both TSCs did not affect proteasome translocation under starvation (FIG. 6B(ii)), further supporting the inventor's previous findings showing that the SESN1/2-AMPK-TSCs pathway does not regulate proteasome dynamics (FIGS. 1N, 7A, and 6A and 6B). In a complementary experiment, it was found that the lack of SESN3 did not affect the downregulation in p70-S6K phosphorylation in response to amino acid starvation or Torin1 (FIG. 6C).

The finding that SESN3 KO abrogated proteasome recruitment pointed out that its function in this context is nonredundant with that of SESN1 and SESN2, at least not in their endogenous levels. Similarly, SESN1 KO (accompanied by no change in SESN2 and SESN3 levels) was shown to result in enhanced muscle wasting [J. Segalés et al., Nat. Commun. 2020 111 11, 1-13 (2020)]. While the other two Sestrins did not fill SESN1's role in its absence, overexpression of SESN2 in muscles did provide some protection from the atrophy caused by SESN1 KO [J. Segalés et al., (2020)]. The inventors therefore tested whether supra-physiologic expression of either SESN1 or SESN2 may overcome the effect of SESN3 KO. It was found that SESN1 overexpression was able to compensate for the absence of SESN3 to some extent, while SESN2 did not (FIG. 6D (i)-(ii)). In summary, findings of others as well as of the present disclosure suggest that while the similarities between the three Sestrins could have suggested an overlap in function, some of their unique qualities and their naturally occurring levels (which may be tissue- and/or stress-dependent), render the different Sestrins as largely non-redundant mediators of stress signaling.

### Example 5

mTOR Stimulation by YWF Involves p38 Activation

One pathway by which amino acids stimulate mTORC1 activation was shown to involve the MAPK p38, which is phosphorylated in response to amino acids supplementation [16]. It was found that the addition of YWF, even to well-fed cells, stimulated phosphorylation of p38 (FIG. 7F). This led us to hypothesize that p38 activation is part of mTOR activation by YWF. Interestingly, QLR did not stimulate p38 activation (FIG. 6E), suggesting that this previously described mechanism of amino acids sensing is in fact selective only for certain amino acids including YWF, but not to QLR—the ‘canonical’ mTOR-agonistic ones. Since YWF appear to act as p38 activators, the effect of known p38 activators-anisomycin and hyperosmotic stress [G. Remy et al., Cell. Signal. 22, 660-667 (2010)], was next tested on proteasome dynamics. Under both treatments, the proteasome was locked in the nucleus and many of the cells died (FIG. 7G). Overexpression of a constitutively active MEK3 which activates p38 (FIG. 6F), also resulted in proteasome nuclear accumulation and inhibition of its recruitment under stress (FIG. 7H). These results underscore the role of p38 activation in proteasome dynamics, and as part of the signaling cascade responsible for its regulation by YWF. Importantly, silencing p38 (FIG. 6G) largely prevented nuclear sequestration of the proteasome—otherwise induced by YWF (FIG. 7I). As mentioned above, sequestration of the proteasome in the nucleus of starved cells led to their death (FIG. 3E). In contrast, preserving the cytosolic proteasomal pool by p38 silencing not only inhibited proteasome accumulation in the nucleus, but at the same time prevented cell death-despite the presence of YWF (FIG. 7I; see also below). Interestingly, SESN3 KO which prevented proteasome recruitment under stress, also seems to induce p38 activation (FIG. 6H), similar to the effect of YWF, thereby establishing a link between these newly identified components of the YWF sensing mechanism.

### Example 6

mTOR Localization to the Lysosome is Independent of its Kinase Activity, and is Sufficient to Prevent Proteasome Recruitment

Next, the inventors aimed to test the effect of YWF supplementation on mTOR inhibition by Torin1, which was found to induce proteasome recruitment (FIGS. 3A and 3B). Since downstream substrates phosphorylated by mTOR respond differently to different inhibitors [C. C. Thorcen et al., (2009)] as well as to different sets of agonistic amino acids, another known effect of mTOR inhibition, namely its localization to the lysosome [R. Zoncu et al., Science 334, 678-683 (2011)], was monitored. Of note is that mTORC1 localization to the lysosome surface was shown to be regulated by p38 [16].

While Torin1 treatment resulted—similar to starvation—in dissociation of mTOR from the lysosome, the addition of YWF reversed this effect (FIGS. 7J and 6I). At the same time, YWF also inhibited Torin1-induced proteasome recruitment (FIG. 7K). It was also found that like YWF supplementation, also SESN3 KO prevented Torin1-induced dissociation of mTOR from the lysosome (FIGS. 7D and 6J) despite amino acid deprivation as well as proteasome translocation from the nucleus (FIG. 7L). Intriguingly, a global screen of genes regulating the response to Torin 1 treatment in yeast found that those displaying the highest resistance to Torin1 are regulators of transport to the vacuole, the yeast lysosome [S. Lie et al., Open Biol. 8 (2018)]. It was suggested that localization of TORC1 to the vacuole stimulates its local activation also in the presence of Torin1. Of note is that the above Torin1-resistant responses were identified under a 5 μM concentration of the inhibitor [S. Lic et al., (2018)]]—much higher than those used in the experimental setup (0.25 μM). It was shown that also in human cancer cells, mediators upstream of mTORC1 can confer resistance to Torin1 [Q. Xia et al., iScience 24, 103528 (2021)]. Other mechanisms for resistance to mTOR kinase-inhibitors include the alteration of feedback loops and upregulation of alternative/parallel signaling cascades [L. Formisano et al., Crit. Rev. Oncol. Hematol. 147, 102886 (2020)].

Amino acid starvation also results in detachment of mTOR from the lysosome. Adding only YWF to the starvation medium reverses this effect, resulting in mTOR localization to the lysosomal membrane (FIG. 7J). This finding underscores the newly identified role of YWF as mTOR activators, expanding the repertoire of known mTOR agonistic amino acids. Notably, it was previously suggested that different amino acids exert different input stimuli on mTOR, which result also in differential downstream outputs [Goberdhan, D. C. I., et al. 2016. Cell Metab 23, 580-589]. The inventor's findings show that this is the case with regard to QLR and YWF.

The mechanism(s) underlying the findings that YWF and SESN3 KO overcome the effect of Torin1 on the localization of both mTOR and the proteasome (FIGS. 7J, 7K and 7D, 6I and 6J) is still elusive. It was shown that in some instances, the inhibition of mTOR downstream effects by Torin 1 is incomplete and is rather dependent on upstream regulators of mTOR for a maximal effect [Q. Xia et al., (2021); P. P. Hsu et al., Science 332, 1317-1322 (2011)]. It is also possible that one or more of the aromatic amino acids competes directly with Torin1; that some of the effect of YWF and/or SESN3 occurs downstream to mTOR-perhaps as part of a feedback loop; that some of their effect is relayed through parallel signaling pathway(s); or that the regulation of proteasome dynamics by YWF and SESN3 is mediated via a kinase-independent mTOR function. Notably, other roles of mTOR, as well as some functions of other kinases which regulate proteolysis, were shown to occur independently of their kinase activity [K. W. Kalim et al., PLoS One 12, 1-15 (2017); E. Erbay et al., J. Biol. Chem. 276, 36079-36082 (2001); F. Tang et al., Nat. Commun. 2019 101 10, 1-17 (2019); V. Risson et al., J. Cell Biol. 187, 859-874 (2009); Y. Ge et al., J. Biol. Chem. 287, 43928-43935 (2012)]. In any case, the findings that both YWF and SESN3 KO retain mTOR position on the lysosomal membrane point out that at least some of their effect is exerted at the level of mTOR and/or upstream to it.

Nonetheless, the above findings strongly link proteasome dynamics—as well as its regulation by YWF—to SESN3 and mTORC1.

### Example 7

### Proteasome Recruitment is Mediated by STAT3

Signal transducer and activator of transcription 3 (STAT3) is a mediator of cellular response to environmental cues, including growth factors [J. E. Darnell, Science 277, 1630-1635 (1997)]. STAT3 was recently shown to localize to the lysosomal surface and to interact with its V-ATPase—a component of the mTOR pathway. V-ATPase is responsible for mTORC1 localization to the lysosomal membrane and for its activation in response to amino acids [R. Zoncu et al., (2011)]. Two key phosphorylation sites of STAT3 are Tyr705 (Y705) and Ser727 (S727), the latter being phosphorylated by mTOR as well as p38 [Huynh J, et al., (2019). Nat Rev Cancer. 19 (2): 82-96.]. Interestingly, STAT3 and p38 were suggested to mutually repress one another [J. Huynh et al., Nat. Rev. Cancer 2018 192 19, 82-96 (2018)].

While the implications of each of these two phosphorylations for STAT3 activity are largely context- and stimulus-dependent, it was shown that mTOR inhibitors or nutrient deprivation, as well as mutating residue S727, upregulate Y705 phosphorylation [S. Yoon et al., Autophagy 6, 1125-1138 (2010); S. Bhattacharya et al., Res. Sq., pre-print (2021)]. In turn, this modification was shown to promote stress-induced protein breakdown by the UPS [J. F. Ma et al., EMBO Mol. Med. 9, 622-637 (2017)], as well as proteasome-mediated muscle loss in cancer-induced cachexia [A. Bonetto et al., PLOS One 6, c22538 (2011)]. Such stimulation of proteasome activity is blocked by STAT3-specific inhibitors, Stattic for example [J. R. Vangala et al., J. Biol. Chem. 289, 12612-12622 (2014)], which prevent muscle mass loss under cachexia [K. A. S. Silva et al., J. Biol. Chem. 290, 11177-11187 (2015)]. Intriguingly, it was shown that phosphorylated S727 antagonizes STAT3 activation mediated by Y705 phosphorylation [J. Yang et al., Int. Immunol. 32, 73-88 (2020)], while mutating Y705 resulted in localization of STAT3 almost exclusively to the lysosome [B. Liu et al., Cell Res. 2018 2810 28, 996-1012 (2018)].

that the inventors next examined if STAT3 may be involved in proteasome recruitment which is induced by mTOR inhibition—as does its Y705 phosphorylation. Indeed, treating starved cells with Stattic prevented proteasome translocation from the nucleus to the cytosol (FIG. 7M). Importantly, the inventors show that Y705 phosphorylation is upregulated following amino acid deprivation or by subtracting YWF only, while it is inhibited almost entirely following the addition of YWF (FIGS. 7N and 6K). Interestingly, STAT3 Y705 phosphorylation was also shown to correlate with poor prognosis in tumors such as glioblastoma, breast cancer, and lung adenocarcinoma [S. Susman et al., Diagn. Pathol. 14, 1-8 (2019); A. P. Morelli et al., Neoplasia 23, 1048-1058 (2021); S. J. Kim et al., Cancer Lett. 500, 147-160 (2021)]. In fact, it was shown that in non-small cell lung carcinoma, STAT3 KO can sensitize cancer cells to mTOR inhibitors, suggesting that out of the many downstream effects of mTOR inhibition—that mediated by STAT3 Y705 phosphorylation is a cell-protective one [A. P. Morelli et al., (2021)]. In line with these studies, the inventors found that STAT3 response to Torin1 was the same as that observed following starvation, namely upregulation of Y705 phosphorylation (FIG. 7N). Importantly, also this effect of Torin1 was reversed by the addition of YWF (FIG. 7N), further supporting the notion that mTOR regulates protcasome dynamics independently of its kinase activity. The fact that the outcome of mTOR (kinase) inhibition in this case is the hyper-phosphorylation of a downstream effector rather than dephosphorylation of an mTOR substrate, may explain how such output signals—STAT3 Y705 phosphorylation and proteasome recruitment-seem to be independent of mTOR kinase activity.

Taken together, these results demonstrate that STAT3, a known downstream effector of mTOR signaling, an interactor of the lysosomal V-ATPase, and a regulator of proteasome activity, is involved in mTORC1-mediated proteasome recruitment under stress.

### Example 8

### Stress-Induced Proteasome Translocation Across the Nuclear Membrane is Carried Out by the Shuttle Proteins p62 and NBR1

A still open question was the identity of the protein(s) responsible for proteasome shuttling. mTOR directly phosphorylates numerous proteins, thereby regulating many cellular functions, including protein breakdown and synthesis [Liu, G. Y., and Sabatini, D. M. 2020. Nat Rev Mol Cell Biol. 10.1038/s41580-019-0199-y]. The inventors therefore tested the effect of YWF on p62 (SQSTM1), a shuttling protein involved both in autophagy- and UPS-mediated protein degradation [Moscat, J., et al. 2009. Cell 137, 1001-1004, Fu, A., et al. 2021. Proceedings of the National Academy of Sciences 118, Cohen-Kaplan, V. et al. 2016. Oncotarget 7], and the Transcription Factor EB (TFEB), a master regulator of autophagy [Settembre, C., et al. 2011. Science 332, 1429-1433].

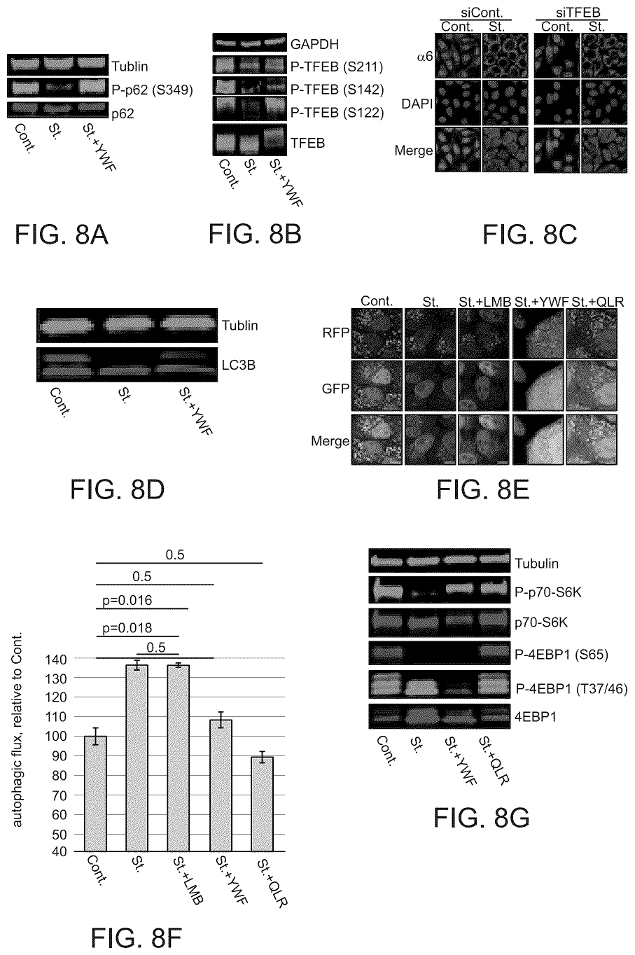

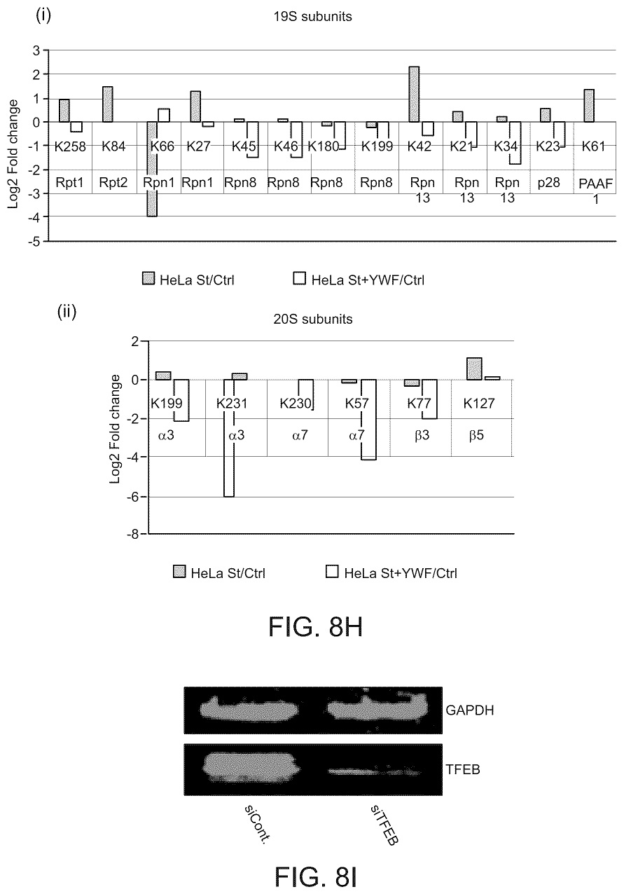

p62 is phosphorylated by mTOR on Ser349 as part of autophagy regulation [Ichimura, et al. 2013. Mol Cell 51, 618-631]. While this phosphorylation was downregulated under starvation, YWF supplementation restored p62 phosphorylation (FIG. 8A). Since p62 is known to bind the proteasome, as well as to facilitate protein nuclear export via its NES motif that interacts with Exportin1, p62's potential role in proteasome export was tested. The inventors silenced p62 (FIG. 6L) and found that in its absence, starvation-induced translocation of the proteasome was inhibited only partially (FIG. 7O). Searching for a potential complementary partner for p62, the inventors identified NBR1-its close homolog, known to interact with p62 [V. Kirkin et al., Mol. Cell 33, 505-516 (2009)]: silencing of both shuttle proteins (FIG. 6L) inhibited the translocation almost completely (FIG. 7O). Similar to the effect of SESN3 KO under normal metabolic conditions (FIG. 7B), the silencing of both p62 and NBR1 results in near-complete concentration of the proteasome in the nucleus (FIG. 7O).

As for TFEB, mTOR phosphorylate it on three Ser residues: S122, S142, and S211 [Martina, J. A., et al. 2012. Autophagy 8, 903-914, Settembre, C., et al. 2012. EMBO J 37, 1095-1108, Vega-Rubin-de-Celis, S., et al. 2017. Autophagy 13, 464-472]. Monitoring these 3 residues phosphorylated by mTOR, it was found that TFEB phosphorylation is inhibited under starvation (FIG. 8B). Importantly, the inventors found that the modification of Ser122 is rescued by YWF supplementation (FIG. 8B). The less prominent effect on Ser211, and the almost lack of effect on Ser142 suggests a link between TFEB's role in different downstream functions and its phosphorylation on different residues.

Of note it that although TFEB is largely referred to as “master regulator of autophagy”, previous studies showed that its absence had little to no effect on autophagic activity, which may be attributed, in part, to some overlap with other transcription factors [Mansueto, G., et al. 2017. Cell Metab 25, 182-196, Xia, M., et al. 2022. Proc Natl Acad Sci USA 119]. The inventors next tested whether TFEB is essential for proteasome translocation by silencing its expression (FIG. 8I), and found that its knockdown had little effect on proteasome translocation (FIG. 8C). It is possible that TFEB phosphorylation on Ser122, stimulated by YWF, plays a role in protcasome translocation, yet is not essential-similar to the case of autophagy.

As mentioned, both TFEB and p62 are downstream substrates of mTOR responsible for the regulation of autophagy. The inventors therefore tested the effect of YWF on this proteolytic machinery. While autophagy is upregulated under starvation, the inventors found that it is inhibited to a large extent by the addition of YWF (FIG. 8D-F). To quantitatively compare the effect of YWF on autophagy relative to that of the ‘canonical’ mTOR agonistic amino acids, QLR, the tandem-fluorescent-LC3 protein (GFP-RFP-LC3) was used to monitor autophagic flux [Nicklin, P., et al. 2009. Cell 136, 521-534]. As can be seen both qualitatively (FIG. 8E) and quantitatively (FIG. 8F), autophagy is upregulated by starvation, and is inhibited by the addition of YWF. Interestingly, the most substantial inhibition of autophagy is observed following addition of QLR (FIGS. 8E and 8F), suggesting that while this triad does not regulate proteasome translocation (FIG. 3D), its selective effect towards autophagy is also stronger compared to that of YWF. In addition, autophagy is not inhibited by LMB which inhibits proteasome translocation (FIG. 1), indicating that proteasome export is not required for autophagy.

The inventors next aimed to check whether the two triads also differ in their effect on other ‘canonical’ mTOR substrates. It was found that YWF stimulate mTOR activity towards p70-S6K, but has no effect on the phosphorylation of 4EBP1, while QLR restored this phosphorylation (FIG. 8G).

Taken together, YWF regulation of mTOR activity towards at least three of its bona fide substrates, alongside the inhibition of autophagy, clearly establish YWF as mTOR agonistic amino acids. The above findings also present mTOR signaling branches that are both common (phosphorylation of TFEB, p62, and p70-S6K; regulation of autophagy, and mTOR lysosomal localization) and distinctive (phosphorylation of 4EBP1 (QLR); proteasome translocation (YWF)) to the two triads of amino acids-QLR and YWF.

It was previously shown that proteasomal subunits are ubiquitinated in response to stress [Cohen-Kaplan, V., et al. (2016), Proceedings of the National Academy of Sciences 113, E7490-99; Cohen-kaplan, V., Ciechanover, A., and Livneh, I. (2017), Autophagy 13, 1-2]. The inventors now looked for proteasomal ubiquitination also in response to YWF. It was found that for several ubiquitination sites, the effect of YWF is opposite to that of starvation (FIG. 8H (i)-(ii)). The exact mechanism(s) that underlie these alterations, and in particular their possible role in stress-induced proteasome translocation, are subjects for future studies.

It was recently reported that AKIRIN2 plays an important role in nuclear import of proteasomes following mitosis [M. de Almeida et al., Nature 599, 491-496 (2021)]. It was therefore important to test whether it also plays a role in amino acid-mediated proteasome dynamics. To that end, AKIRIN2 was silenced (FIG. 6M) and proteasome localization was monitored. Interestingly, cells lacking AKIRIN2 demonstrate a cytosolic preference in proteasome distribution, showing that it plays a role in protcasome basal distribution (FIG. 7P). In contrast, it was found that proteasome import in response to YWF stimulation is independent of AKIRIN2, pointing to distinct import mechanisms between mitosis and YWF- and mTOR-mediated stimulation (FIG. 7P).

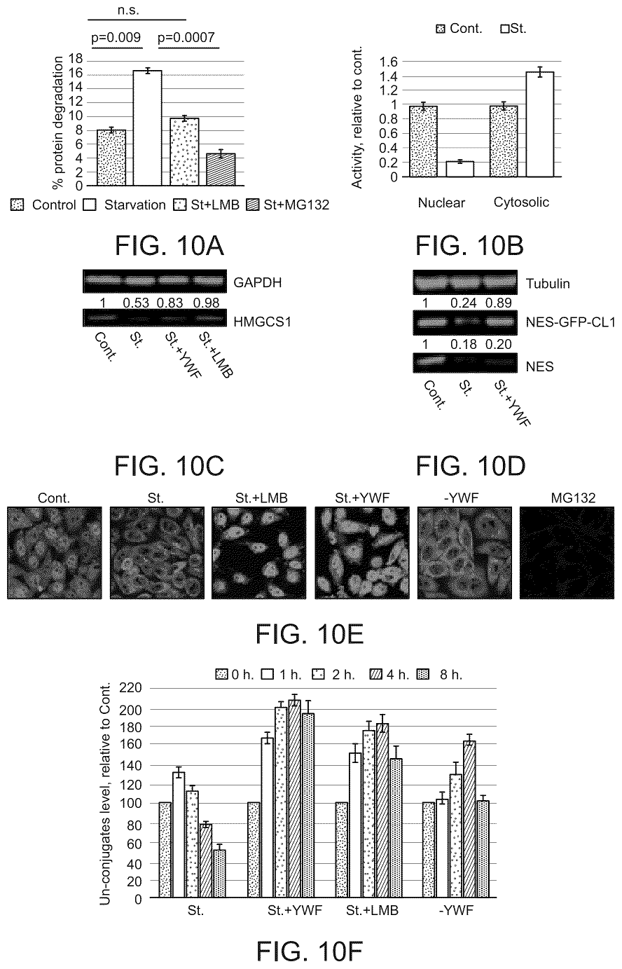

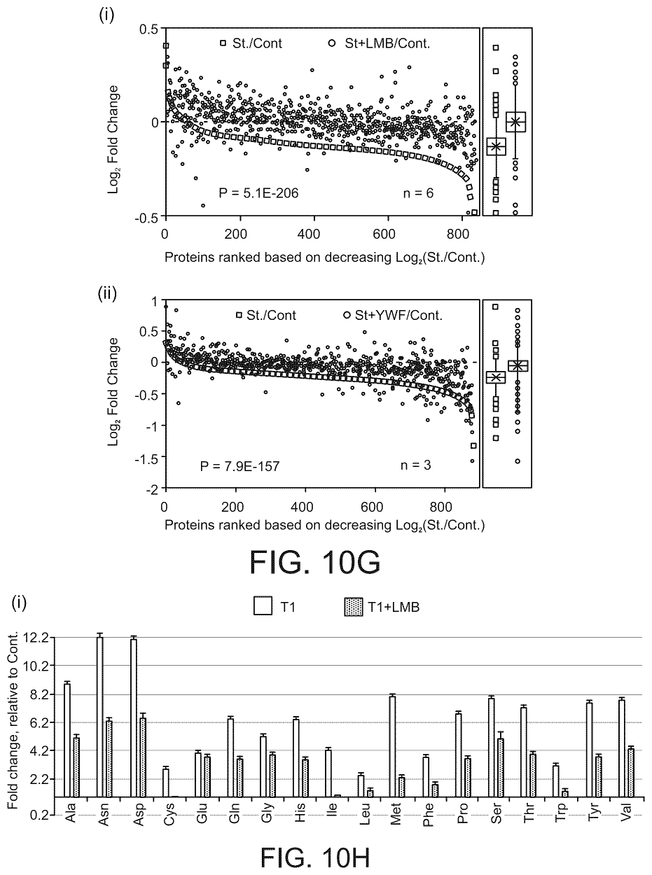

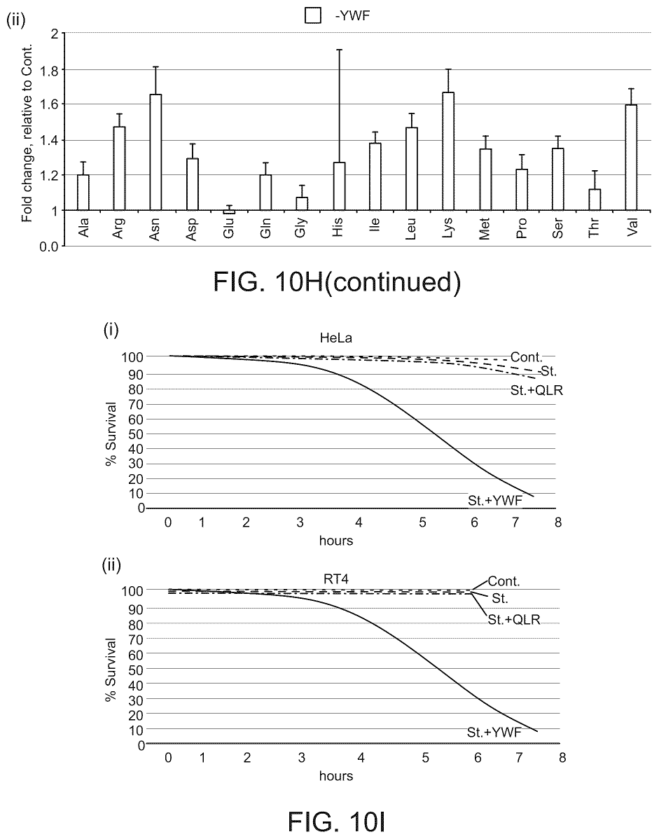

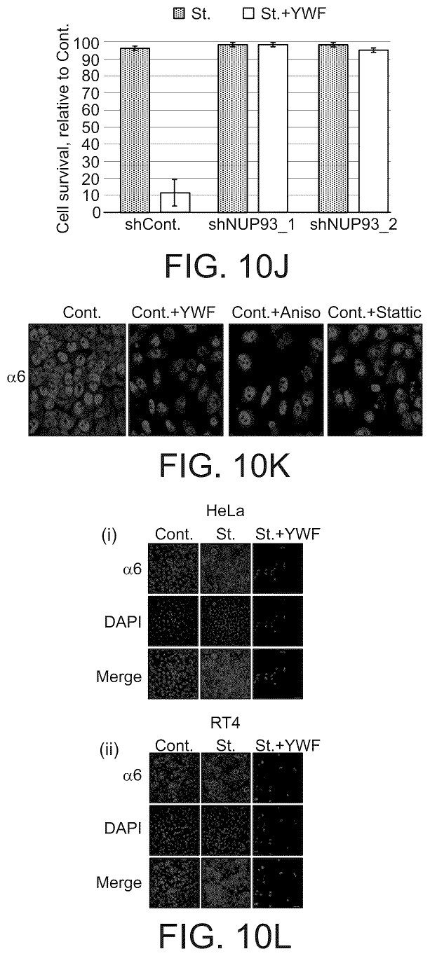

Next, it was important to look for the role of the Nuclear Pore Complex (NPC) in proteasome shuttling. NUP93, a component of the NPC which was reported to selectively facilitate the nuclear import of Smads, but not that of NLS-harboring proteins [X. Chen et al., Mol. Cell. Biol. 30, 4022-34 (2010)], was therefore silenced (FIG. 6N). The inventors found that its silencing resulted in a predominant cytosolic distribution of the proteasome, and that under starvation the proteasome concentration in the cytosol was further increased (FIG. 7Q). Importantly, administration of YWF to NUP93-lacking cells, affected only slightly the largely cytosolic distribution of the proteasome (FIG. 7Q). Moreover, as a result of the preservation of the cytosolic proteasomal pool, the cells were rescued from the deadly YWF effect (FIGS. 7Q and 10J; compare with FIGS. 10I and 10L). This underscores that the cytotoxic effect of YWF can be attributed to their ability to empty the cytosol from the proteasome and its catalytic activity, which are essential for survival under stress. The inventors validated that NUP93 silencing did not impair NLS-mediated nuclear import at large via monitoring the localization of NLS-fused GFP (FIG. 60), demonstrating that the effect of its knockdown on proteasome translocation is not common to all proteins entering the nucleus.

Considering the findings described above, and without being bound by any theory, the inventors cautiously suggest a potential mechanism for mTORC1 regulation of proteasome dynamics, while acknowledging that additional factors and effectors are still missing. According to the proposed model, the sensing of amino acid scarcity is: (1) mediated by SESN3; (2) leads to mTOR dissociation from the lysosomal membrane; (3) stimulates STAT3 Y705 phosphorylation; and (4) subsequently induces proteasome shuttling from the nucleus to the cytoplasm in an exportin 1- and p62/NBR1-dependent manner. Reciprocally, the sensing of YWF excess is: (1) mediated by the p38 MAPK; (2) leads to mTOR localization to the lysosomal membrane; (3) inhibits STAT3 Y705 phosphorylation; and (4) induces proteasome sequestration into the nucleus, aided in part by NUP93.

### Example 9

Stimulation of mTOR by YWF Involves Blocking/Reduction of the Inhibitory Interaction of SESN3 with GATOR2

In the presence of sufficient amounts of amino acids, recombination activating gene (Rag, a heterodimeric GTPase complex, RagA/B and RagC/D) recruits/anchors Raptor containing mTORC1 kinase complex to the surface of the lysosome, where mTORC1 is activated. Rag GTPase is regulated by GATOR, that is a multiprotein complex composed of two subcomplexes GATOR1 and GATOR2. The GATOR I complex is composed of three proteins, DEPDC5, NPRL2 and NPRL3, and inhibits Rag GTPase, while GATOR2 is composed of five protein components, MIOS, WDR24, WDR59, SEHIL and SEC13, and suppresses the inhibitory function of GATOR1 (GTPase activating protein) toward Rag GTPase. Sestrin2 is a physiological regulator of GATOR complexes. By physically interacting with GATOR2, Sestrin2 releases GATOR1 from GATOR2-mediated inhibition. GATOR1 then inhibits RagB GTPase and subsequently prevents mTORC1 activation by amino acids. As shown by FIG. 9A, in the presence of amino acids, specifically, Leucine, the inhibitory interaction of SESN2 with GATOR2 is blocked. Released from SESN2 inhibitory effect, the GATOR2 complex suppresses the inhibitory function of GATOR1 on mTOR, thereby acting as mTOR agonist. The inventors now show that interaction of YWF with SESN3 blocks its inhibitory interaction with GATOR2, thereby leading to suppression of the inhibitory function of GATOR1 on mTOR. More specifically, to further characterize the effect of YWF on SESN3 and mTORC1, Hela and RT4 cells were transfected with a Flag-SESN3 encoding plasmid. The Flag-SESN3-expressing cells were treated as indicated in FIGS. 9B and 7E (starved in the presence or absence of YWF). Cells were then harvested and lysed, and lysates were incubated with beads conjugated to anti-Flag antibodies thereby immunoprecipitating Flag-SESN3 and its interacting proteins. Next, the washed immunoprecipitates were subjected to either (FIG. 9B) mass spectrometric analysis or (FIG. 7E) Western blotting. As shown by FIGS. 9B(i)-(iv) and 7E, following amino acid starvation, the inhibitory interaction of SESN3 with members of the GATOR2 complex (specifically, Mios, WDR59, SEHIL and SEC13) is elevated-leading to mTOR inhibition. This interaction is inhibited in the presence of YWF. The Western blot analysis of FIG. 7E, indicates that the interaction of SESN3 with Mios and WDR59, is significantly inhibited in the presence of YWF. These results demonstrate that the inhibitory effect of the YWF on SESN3 is mediated by the GATOR2 complex, and thus, YWF alone can lead to mTOR activation.

### Example 10

### Proteasome Translocation is Required to Enhance Proteolysis of Cytosolic Proteins

To unravel the role of proteasome translocation under amino acid starvation, protein breakdown was monitored in cells and found that it is stimulated ˜2-fold (FIG. 10A). To link the enhanced proteolysis to the enrichment of the cytosol with nuclear proteasome, LMB-which has no effect on autophagy [R. Huang et al., Mol. Cell 57, 456-467 (2015)], was used to inhibit proteasome recruitment. This resulted in near annulment of protein breakdown stimulated by short-term starvation, while treatment with a proteasome inhibitor inhibited both the stress-induced and the basal level of proteolysis (FIG. 10A). These findings are in agreement with a previous report that during short amino acid deprivation, the proteasome is the key proteolytic machinery responsible for protein degradation and amino acid turnover [R. M. Vabulas et al., Science 310, 1960-1963 (2005)].

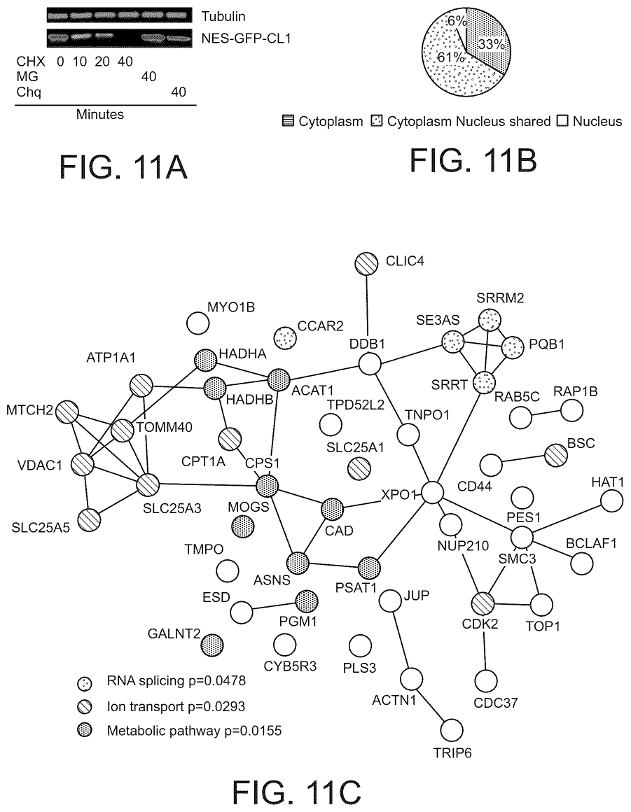

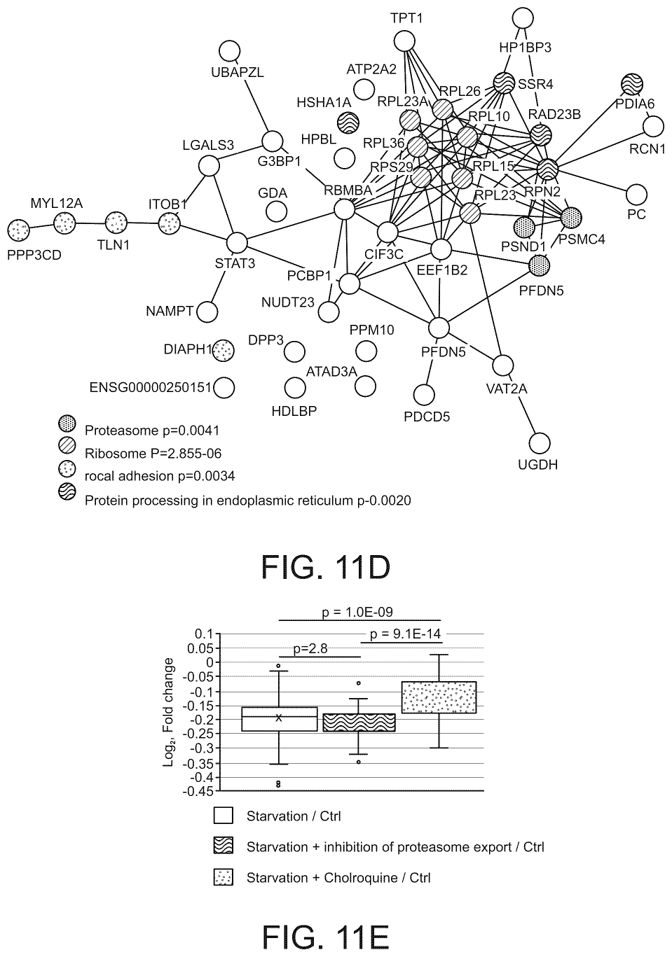

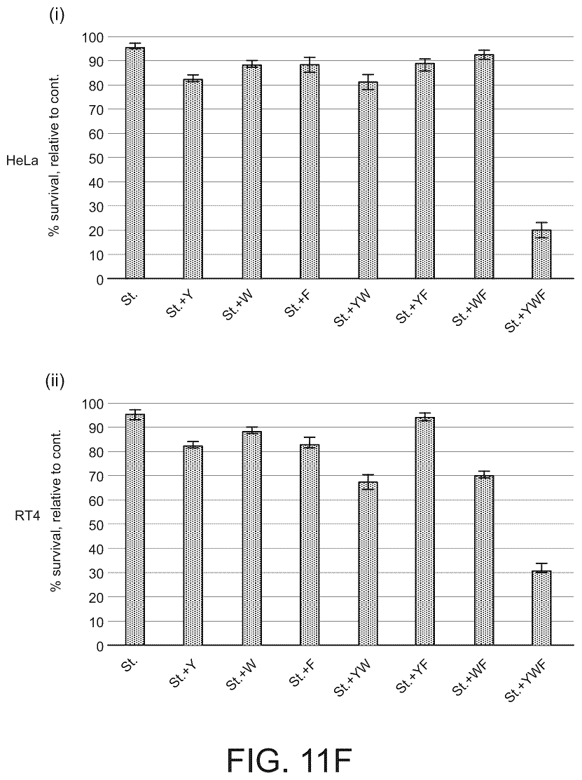

The proteasomal activity was then monitored in both the nuclear and cytosolic fractions, showing that its nuclear activity diminishes following starvation, with a concomitant increase in cytosolic activity (FIG. 10B). In parallel, the inventors demonstrated that the stress-induced removal of HMGCS1—a bona fide cytosolic substrate of the proteasome that was shown to be degraded following mTOR inhibition [J. Zhao et al., Proc. Natl. Acad. Sci. 112, 15790-15797 (2015)], is largely dependent on proteasome export (FIG. 10C). A similar finding was observed monitoring a GFP tagged both with an NES that rendered it cytosolic, and a CLI motif-which promoted its rapid ubiquitination and proteasomal degradation [T. Gilon et al., EMBO J. 17, 2759-2766 (1998)], yet not its autophagic removal (FIG. 11A). The inventors found that while this cytosolic GFP species is stimulated under starvation, it is rather stabilized when proteasome recruitment is blocked. In contrast, RFP—which is removed mostly by autophagy [D. Berko et al., Mol. Cell 48, 601-611 (2012); P. K. Kim et al., Proc. Natl. Acad. Sci. 105, 20567-20574 (2008)], is nevertheless degraded under conditions where the proteasome is confined to the nucleus (FIG. 10D).

Using live imaging of a fluorescent proteasome activity probe [C. R. Berkers et al., Mol. Pharm. 4, 739-748 (2007)], the inventors were able to directly localize the activity of the proteasome, while also comparing its extent in the nucleus vs the cytosol. Once more, it was found that starvation, as well as subtraction of YWF, resulted in translocation of proteasomal activity from the nucleus to the cytosol, leading to an inversion in the relative dominance between the two compartments (FIG. 10E). Addition of either LMB or YWF to the starvation medium inhibited the “migration” of proteasomal activity, “accumulating” it in the nucleus and preventing the shift in its dominance from the nucleus to the cytosol (FIG. 10E).

It was shown that during mTOR-mediated stress, ubiquitination is initially upregulated-probably due to increased activity of ligases-which is followed by a decrease in the level of the conjugates mediated by their proteasomal removal [J. Zhao et al., (2015)]. It was found that preventing export of the protcasome from the nucleus by either YWF or LMB, inhibited degradation and depletion of ubiquitin adducts (FIG. 10F), underscoring the central share of nuclear-originated proteasome in cytosolic proteolysis under stress.

To assess the effect of proteasome translocation on the stability of the population of cellular proteins, a proteomic assay was conducted, monitoring changes in their level following stimulation and inhibition of proteasome recruitment. It was found that upon proteasome translocation, stimulated by amino acid starvation, the level of ˜900 proteins decreased, while their accelerated degradation was prevented by inhibition of proteasome export by LMB or YWF (FIG. 10G (i), (ii)). The proteins identified under the different conditions and their dynamics overlapped to a large extent. Analysis of those that were most affected by inhibition of export (i.e., their degradation was prevented) showed that 94% are cytosolic-either exclusively or residing in both the cytoplasm and the nucleus (FIG. 11B). Further analysis of the cellular pathways that are enriched in the group of these proteins, revealed key mediators of metabolic pathways (FIG. 11C). That, in contrast to proteins that are unaffected by proteasome dynamics-among which are ribosomal proteins-which are degraded mostly via autophagy (FIGS. 11D and 11E).

### Example 11

Proteasome Recruitment to the Cytosol Provides Cells with Amino Acids which are Essential for Cell Survival Under Stress

To directly assess the contribution of proteasome recruitment to the amino acid pool in stressed cells, LC-MS was employed to resolve and measure the relative abundance of the different amino acids under the different experimental conditions. In order to measure the change in amino acids following proteasome export, the inventors measured the gain in their level following treatment with the mTOR inhibitor Torin1, either in the absence or presence of LMB. While Torin1 stimulated both autophagy and proteasome recruitment, LMB inhibits only the latter [R. Huang et al., (2015)]. The measurements showed that inhibition of proteasome export by LMB significantly inhibited the gain in amino acids produced by Torin1 (FIG. 10H(i)). This demonstrates an important role for the translocated proteasome in replenishing the cell with amino acids during short-term deprivation. Similarly, incubation of cells in a medium containing all amino acids except for YWF, which stimulated proteasome translocation with no effect on autophagy (FIG. 3D), resulted in an increased level of all detectable amino acids except for Glu (FIG. 10H(ii)). Interestingly, Glu was also unaffected by the addition of LMB to Torin1-treated cells, further supporting the validity of our findings. This increase in the cellular amino acid pool can explain the increment in p70-S6K phosphorylation observed following subtraction of YWF from the medium.

The inventors next monitored the effect of proteasome translocation, increased cellular proteolysis and supply of amino acids on cell survival. Monitoring cell viability via a live time-lapse of two different cell lines, shows that while starvation to the entire repertoire of amino acids is well tolerated, inhibiting proteasome recruitment by the addition of YWF results in cell death (FIG. 10I (i), for Hela, and (ii) for RT4 cells). Assessing the effect of different combinations of these 3 amino acids on apoptosis-individually as well as in pairs—it was found that the cytotoxic effect of the trio is significantly stronger than any other combination (FIG. 11F (i), for Hela, and (ii) for RT4 cells). These quantitative analyses of apoptosis explain the observations described above regarding cell death due to proteasome sequestration in the nucleus (FIGS. 3D and 3E).

Importantly, the inventors observed that silencing key components involved in YWF sensing (p38, FIG. 7I) or proteasome import (NUP93, FIG. 7Q) rescued the cytosolic proteasomal pool-despite the presence of YWF that would otherwise drive the proteolytic complex into the nucleus with subsequent cell death. Indeed, monitoring apoptosis in NUP93-lacking cells demonstrated that alongside rescuing the cytoplasmic proteasomal pool, the cells were rescued as well (FIG. 10J). These findings demonstrate that the cytotoxic effect of YWF can be attributed to their ability to empty the cytosol from the proteasome.

Taken together, these findings further underscore the observation that stress-induced cell death caused by YWF is due to their inhibitory effect on proteasome translocation from the nucleus to the cytosol, and that its migration to the cytosol—where it stimulates proteolysis and replenish the depleted amino acids pool—is essential for cell survival.

### Example 12

Stimulation of the YWF-Sensing Mechanism Modulates Proteasome Dynamics also in Non-Starved Cells

As described above, the addition of YWF counteracted the effect of stress on proteasome recruitment (FIGS. 3D and 3E). The inventors found that the addition of excess YWF to well-fed cells, which stimulated their sensing mechanism also in the presence of all other amino acids (FIGS. 7F and 7N), also resulted in further accumulation of the proteasome in the nucleus, and consequently in cell death (FIG. 10K). The same was true for the stimulation of p38 by anisomycin and inhibition of STAT3 using Stattic (FIG. 10K). These observations underscore the importance of proteasome presence in the cytosol for cell survival also in non-starved cells, while showing that intracellular distribution of the proteasome is dictated not only by the presence or absence of certain amino acids, but also by their concentration relative to the others. Such observation also presents an opportunity for modulating proteasome dynamics in animal models, where numerous factors are at play: while complete deprivation of cells from specific nutrients is impractical in the setting of an intact organism—it may not be necessary either.

### Example 13

### Proteasome Recruitment is Essential for Tumor Growth, and its Inhibition Results in Cell Death and Reduction in Tumor Size

The possible effect of YWF on proteasome dynamics was next examined in animal tumor models. The inventors postulated that the metabolic requirements of rapidly dividing tumor cells will render them more vulnerable to forced nuclear sequestration of the proteasome by YWF, which was found to be cytotoxic also to non-starved cells (FIG. 10K). Notably, solid tumors are also characterized by poor perfusion and oxygenation, which result in areas in the tumor core that are shorter in nutrients and are more stressed compared to its periphery [A. I. Minchinton et al., Nat. Rev. Cancer 6, 583-592 (2006)]. The inventors hypothesized that the stress in such regions will serve as a stimulus for proteasome recruitment, which will further sensitize them to nuclear proteasomal accumulation in response to YWF administration.

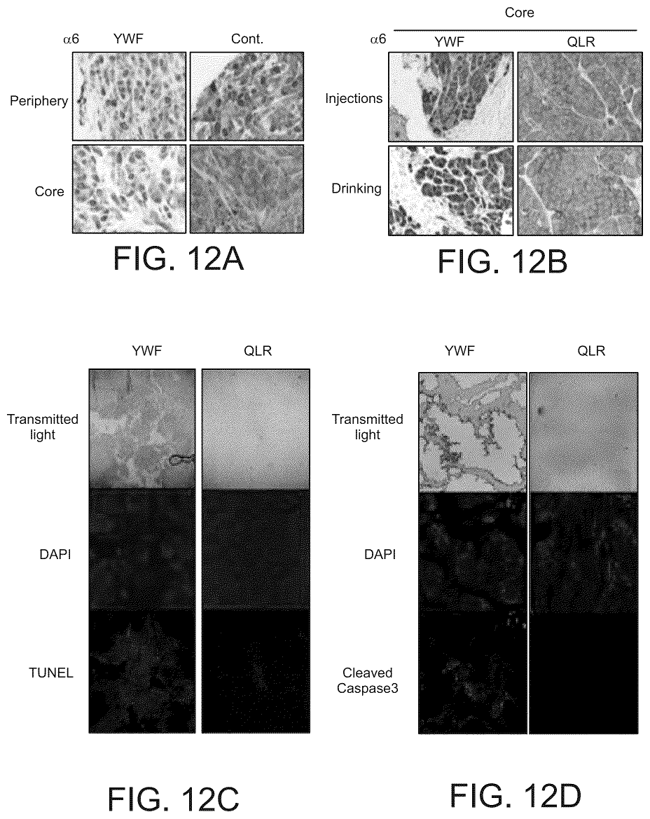

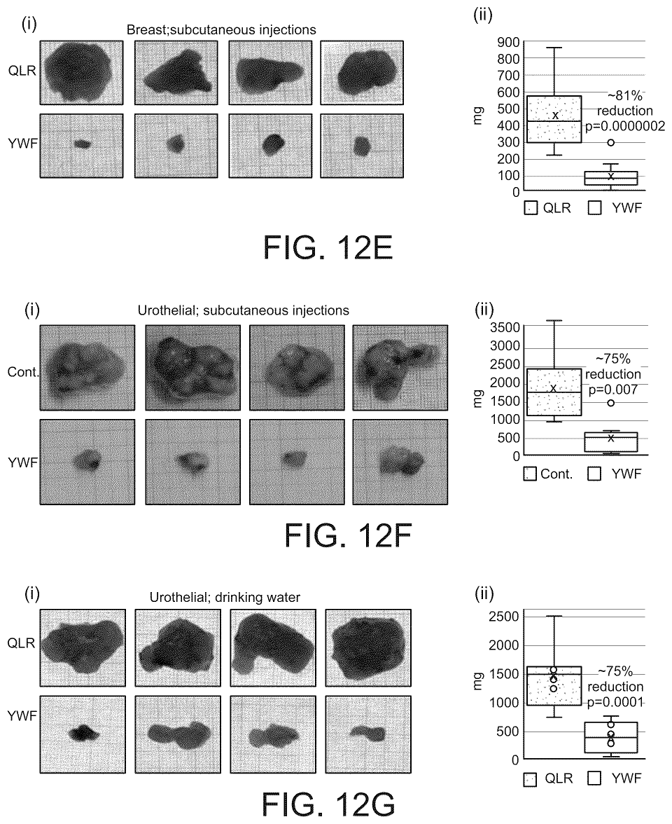

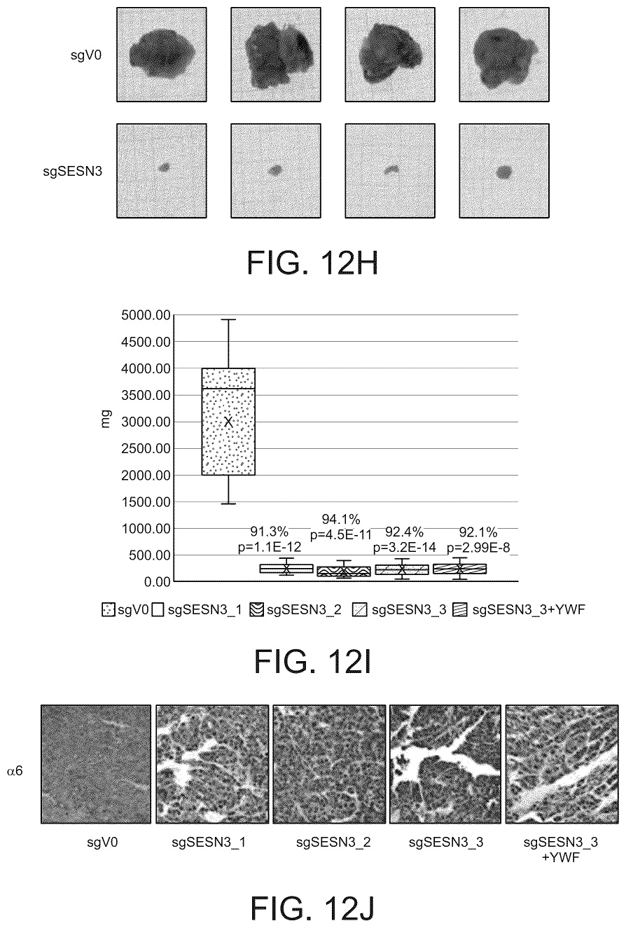

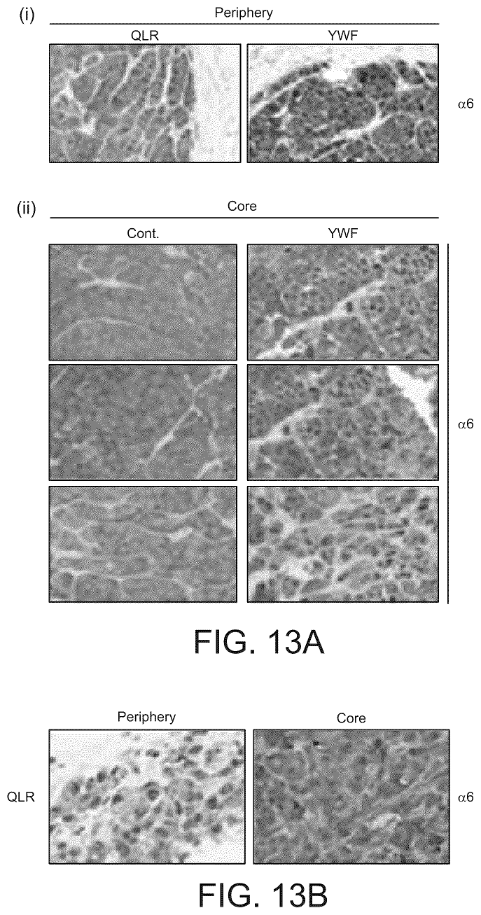

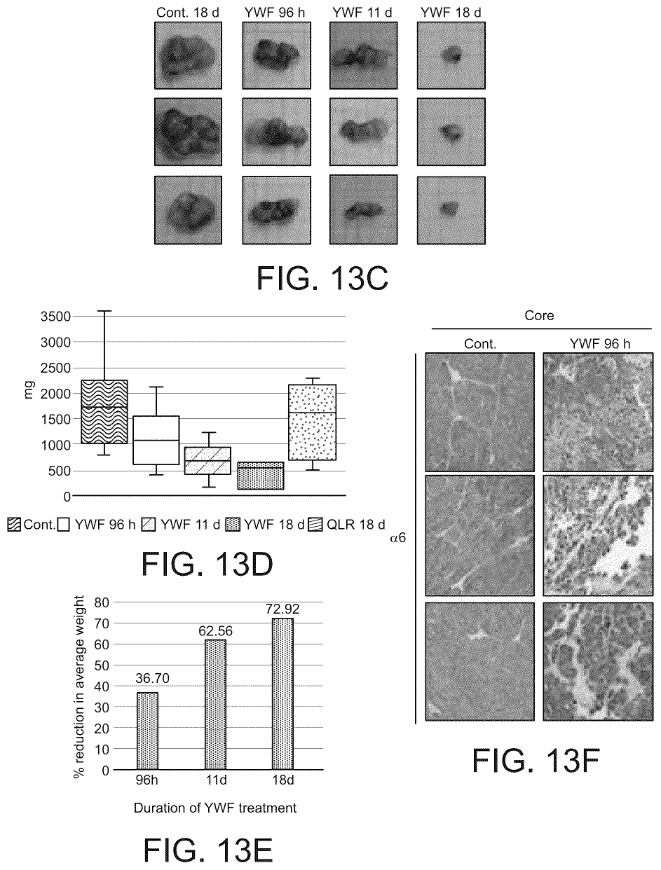

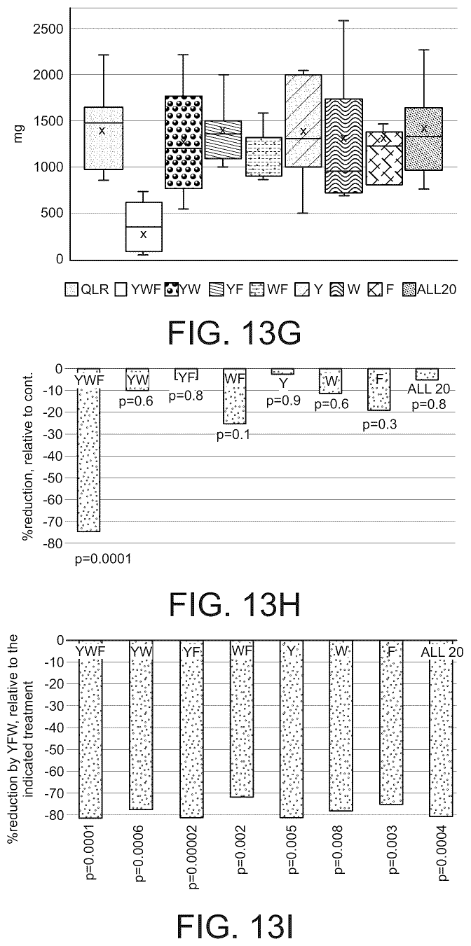

Using human breast and urothelial tumor models in mice, it was found that in the non-stressed periphery of the tumor, the proteasome is largely nuclear, in contrast to its core where the proteasome is more enriched in the cytosol (FIG. 12A). Following injection of YWF (subcutaneously to the tumor bed), a clear nuclear localization of the proteasome was observed also in the tumor's core, as well as a stronger nuclear dominance in its periphery (FIGS. 12A and 12B, FIG. 13A(i) vs. (ii)). In contrast, injection of QLR did not affect proteasome distribution (FIGS. 12B and 13B). Oral administration of YWF—via drinking water—had the same effect on proteasome localization as subcutaneous injections (FIG. 12B). Importantly, the inventors observed large areas of tumor tissue destruction in xenografts treated with YWF (FIG. 12B; see also below). To shed light on the destructive process, tumors were stained for the apoptotic markers TUNEL and cleaved-Caspase3. It was found that concomitantly with their induction of proteasome nuclear accumulation, YWF exerted also a wide cytotoxic effect on tumor cells, and that areas stained positive for the apoptotic markers also demonstrated an architecture typical to damaged tissue (FIGS. 12C and 12D).

Observing the tumors macroscopically and comparing their weight, the inventors found that the effect of YWF at the cellular level (i.e., proteasome nuclear retainment and apoptosis) is accompanied also by a significant reduction of up to ˜80% in tumor size, compared to control tumors (FIGS. 12E, 12F, 12G). YWF inhibited efficiently tumor growth regardless of their route of administration (subcutaneously injected or dissolved in drinking water). YWF were effective even when administrated late in the course of tumor development, in which case tumors were allowed to reach a significantly large size prior to the initiation of treatment (FIG. 13C, 13D, 13E). Importantly, also when initiated in large tumors and administrated for a short period, YWF treatment resulted in nuclear accumulation of proteasome, cell death, and tissue disintegration (FIG. 13F).

Next, mice were treated through their drinking water with all combinations of Tyr, Trp, and Phe-individual amino acids as well as all possible pairs. As in cultured cells (FIG. 11F), it was found that only the three of them together induced a significant reduction in tumor size (FIGS. 13G and 13H), and that the trio was far superior to any other combination (FIG. 13I). Importantly, administration of all twenty amino acids had no effect on tumor growth (FIGS. 13G and 13H), underscoring that an excess of the trio relative to the other amino acids is key for the anti-tumoral effect.

### Example 14

### Sestrin3 KO Results in Proteasome Sequestration and Inhibition of Tumor Growth

The role SESN3 plays in animal tumor models was next tested. Since in cultured cells lacking SESN3 the proteasome was not recruited to the cytosol under stress, one may postulate that tumors in which SESN3 is knocked out will fail to withstand the metabolic stress experienced by cancer cells. Therefore, three independent clones of SESN3-KO RT4 cells, which were implanted as xenografts, were used. The inventors found that tumors lacking SESN3 are dramatically smaller, and as expected—the proteasome is localized mostly to their cell nuclei without any treatment (FIG. 12H, 12I, 12J). Since the ability of cells to cope with stress by recruiting the proteasome to the cytosol is abrogated in the absence of SESN3, treating such tumors with YWF had no additive effect, compared to that seen by SESN3 KO (FIG. 12I).

### Example 15

Sequestering the Proteasome in the Nucleus is an Effective Anti-Tumoral Treatment also in Endogenous and Metastatic Neoplasms in Mice

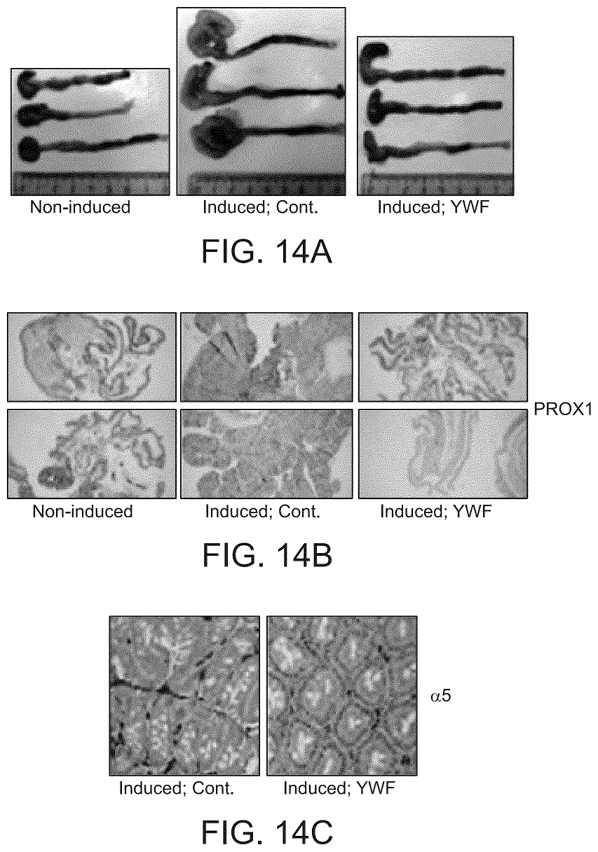

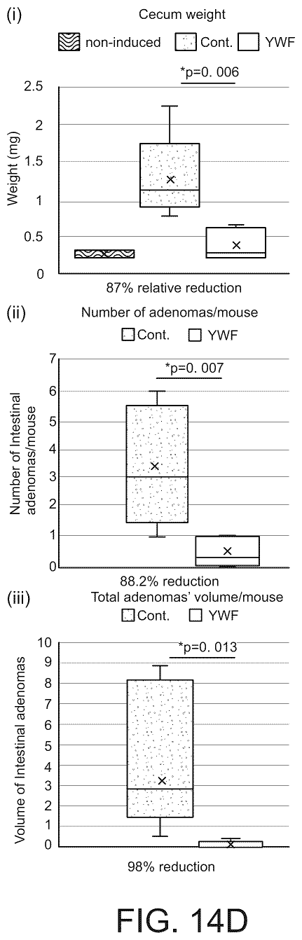

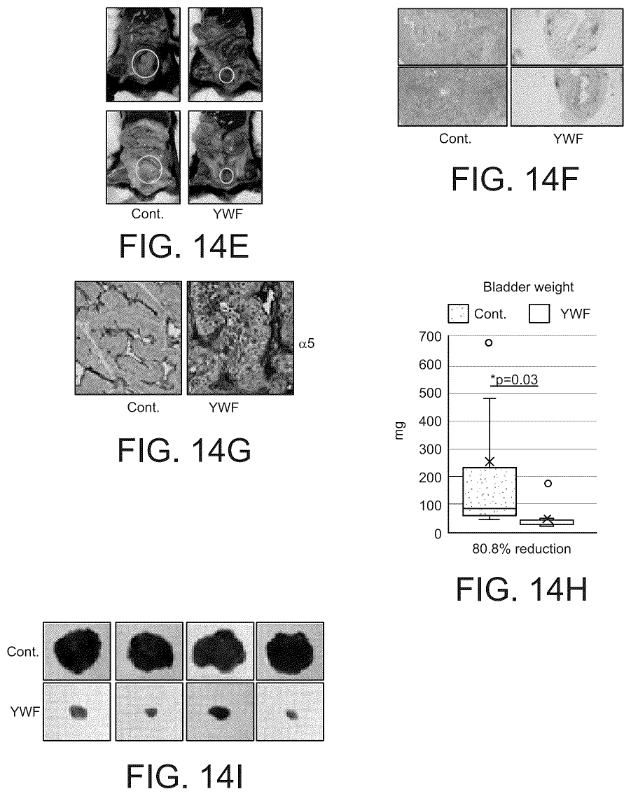

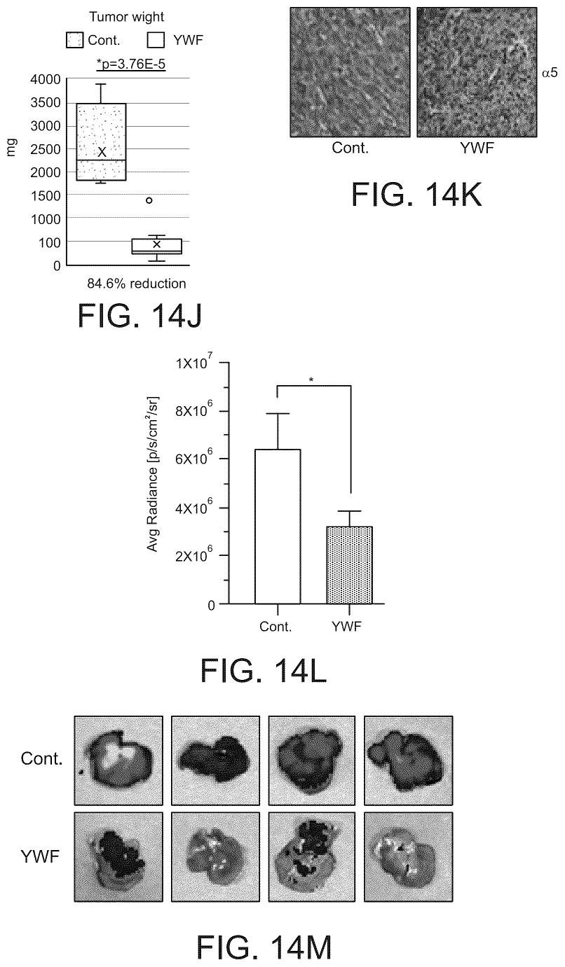

Although xenografts serve as a well-established cancer model in animals, they have some inherent limitations. While providing information about tumors from human origin, their vascular and stromal structures do not mimic that of endogenous tumors. The inventors therefore tested these findings also in endogenous tumor models in mice. First, a model for colorectal cancer (CRC) induced by loss of the Adenomatous Polyposis Coli (APC) tumor suppressor was used, as can be found in most cases of CRC in humans [Q. Liu et al., J. Med. Chem. 53, 7146-7155 (2010)]. While inducing APC loss resulted in tumors both in the cecum and along the colon of mice, YWF administration reduced the size of the main cecal mass, as well as the number and size of the smaller neoplasms along the colon (FIGS. 14A and 14D(i)-(iii)). Staining for PROX1, a marker for high grade dysplasia, demonstrated that YWF reduces the extent of neoplastic tissue in the intestine (FIG. 14B). Staining for the proteasome validated that as expected, YWF treatment resulted in proteasome seclusion in the nucleus (FIG. 14C).

In another model, the carcinogen N-Butyl-N-(4-hydroxybutyl) nitrosamine (BBN), which induces bladder carcinoma following prolonged administration [M. Degoricija et al., J. Transl. Med. 2019 171 17, 1-13 (2019)], was used. After 14 weeks of continuous administration of BBN into the drinking water, a time by which carcinoma in situ and early invasion are present [M. Degoricija et al., (2019)], the inventors have initiated treatment with YWF. After a total of 25 weeks the mice were sacrificed, and their bladders were examined. While significant tumors developed as a result of the BBN treatment, monitoring the bladders macroscopically and under a small magnification, showed that the YWF treatment resulted in bladders of nearly normal size (FIGS. 14E and 14F). Staining for the proteasome showed its nuclear sequestration following treatment (FIG. 14G), and weighing the different bladders demonstrated a significant reduction in tumor mass (FIG. 14H). In a third model, sarcoma, a soft tissue neoplasm, which was induced by a single subcutaneous injection of the carcinogen 3-Methylcholanthrene (3-MCA) was used. YWF treatment, initiated at the point where the sarcomas were palpable, resulted in smaller tumors and nuclear proteasome sequestration (FIG. 14I, 14J, 14K).

Last, the effect of inhibiting proteasome recruitment on metastases, was tested using the 4T1 triple-negative breast cancer model [M. Kaduri et al., Sci. Adv. 7 (2021)]. These aggressive and rapidly metastasizing breast carcinoma cells (expressing mCherry) were injected into the mammary gland of WT female mice, which were then monitored—at this stage without any treatment. Notably, although the primary tumor in this model is the result of exogenously injected cells, the metastatic phase shares common features with human metastatic breast cancer, including the tropism of cells to the lungs, and to a lesser (and later) extent also to the liver [G. V. Echeverria et al., Nat. Commun. 2018 91 9, 1-17 (2018)]. In accordance with this characteristic, three mice were sacrificed on the 15^{th} day after cell inoculation and monitored their lungs and liver ex vivo using IVIS (in vivo imaging system) to detect mCherry fluorescence. Metastases were clearly detected in the lungs of all three animals. Therefore, treatment was initiated in the different experimental groups, with the aim of monitoring the progression of metastases to the liver. Besides for the control group, a second group was treated with YWF in the drinking water. The animals were treated and monitored for 20 days.

The inventors found that in addition to the inhibition of the growth rate and size of the primary tumor, also the extent of liver metastases was significantly lower following YWF treatment (FIGS. 14L and 14M).

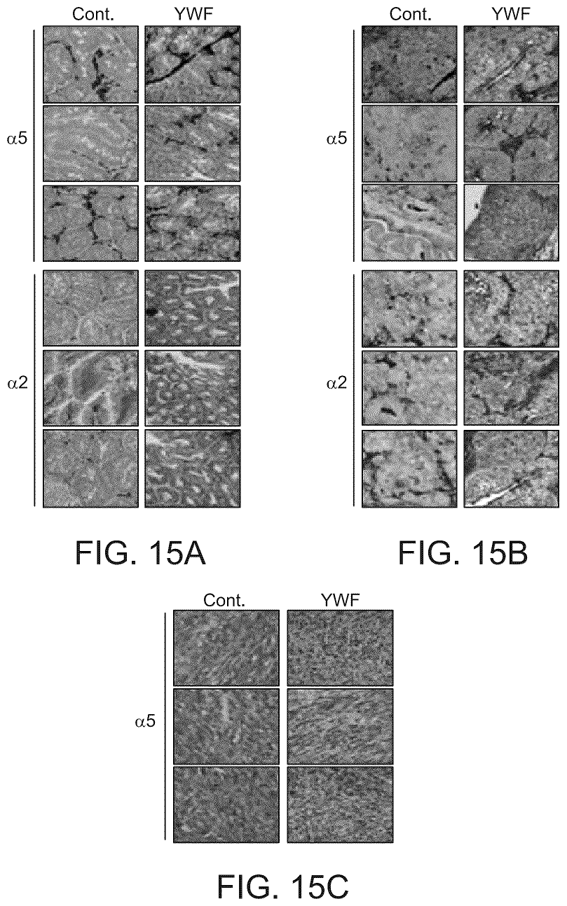

In summary, the findings of the present disclosure unravel a key role for proteasome dynamics as an essential stress-coping mechanism in solid tumors, and a potential target for the development of therapeutic modalities. Similar results are also shown in FIG. 15A, 15B, 15C.

1-50. (canceled)

51. A method for treating, preventing, inhibiting, reducing, eliminating, protecting or delaying the onset of at least one condition or at least one pathologic disorder in a subject in need thereof, the method comprising the step of administering to said subject a therapeutic effective amount of at least one compound that modulates proteasome dynamics and/or function in a mammalian cell, wherein said compound is characterized by affecting at least one of: mammalian target of rapamycin (mTOR) activation and/or lysosomal association, the activity and/or level/s and/or the post translational modification/s (PTM/s), and/or subcellular localization of at least one signaling molecule participating directly or indirectly in at least one pathway mediating said proteasome dynamics/function, and optionally, the proteasome cellular localization.

52. The method according to claim 51, wherein at least one of:
(I) said at least one signaling molecule participating directly or indirectly in said at least one pathway mediating said proteasome dynamics and/or function is at least one of: at least one mediator of metabolite sensing, at least one stress kinase, at least one nucleo-cytosolic shuttle protein, optionally, ubiquitin and/or proteasome interacting shuttle proteins, and/or at least one Nuclear Pore Complex (NPC) protein;
(II) wherein: (i) said mediator of metabolite sensing is a mediator of amino acid sensing; and/or (ii) said stress kinase is at least one member of the Mitogen-activated protein kinases (MAPKs);
(III) wherein at least one of: (i) said at least one mediator of amino acid sensing is at least one member of the Sestrin family; (ii) said at least one member of the MAPKs is at least one member of the p38 mitogen-activated protein kinases (p38 MAPKs—p38α, p38β, p38γ, p38δ); (iii) said at least one nucleo-cytosolic shuttle protein/s is at least one of Sequestosome 1 (SQSTM1, p62) and Neighbor of BRCA1 gene 1 protein (NBR1); and/or (iv) said at least one NPC is Nucleoporin 93 (NUP93); and
(IV) wherein at least one member of the Sestrin family is Sestrin3 (SESN3); and/or wherein said at least one member of the p38 MAPK family, is p388 (p38 delta, MAPK13).

53. The method according to claim 51, wherein said at least one compound leads to:
(I) at least one of:
(i) mTOR activation and/or localization to the lysosomal membrane;
(ii) reduction in Sestrin3 levels and/or activity, and/or interaction with at least one regulatory complex;
(iii) activation of p38 delta;
(iv) reduction in the levels and/or activity of p62 and NBR1; and/or
(v) modulation of NUP93; and optionally,
(II) proteasome nuclear localization.

54. The method according to claim 51, wherein at least one of:
(I) at least one of:
(a) said compound is, or comprises at least one of: a nucleic acid-based molecule, an amino acid-based molecule, a small molecule or any combinations thereof; and
(b) said compound targets at least one of said signaling molecule/s at the nucleic acid sequence level or at the protein level; and
(II) at least one of:
(a) said compound targets at least one of said signaling molecule/s at the nucleic acid sequence level; said compound is, or comprises a nucleic acid-based molecule, said nucleic acid molecule is at least one of: a nucleic acid guide, a double-stranded RNA (dsRNA), a single-stranded RNA (ssRNA), an antisense oligonucleotide, a Ribozyme, a deoxyribozymes (DNAzymes), and an aptamer; and/or
(b) said compound targets at least one of said signaling molecule/s at the protein level, and wherein said compound reduces the stability of said signaling molecule/s by targeted protein degradation (TPD), and/or reduces the activity of said signaling molecule/s.

55. The method according to claim 51, comprising administering to said subject at least one compound that reduces the level and/or activity of Sestrin3, wherein said compound leads to reduction of Sestrin3 levels and/or activity and/or interaction with at least one regulatory complex by at least one of: (i) specifically targeting a nucleic acid sequence encoding said Sestrin3, or any parts thereof; (ii) specifically targeting a nucleic acid sequence involved directly or indirectly in regulation of the Sestrin3 gene expression; (iii) reducing the stability of the Sesn3 protein; and/or (iv) interfering with the interaction of Sestrin3 with at least one regulatory complex.

56. The method according to claim 55, wherein:
(I) said compound comprises:
(a) at least one RNA guide (gRNA) that guides least one nucleic acid guided genome modifier protein to at least one target sequence within said Sestrin3 encoding nucleic acid sequence, or within a nucleic acid sequence involved directly or indirectly in regulation of the Sestrin3 gene expression; or at least one nucleic acid sequence encoding said nucleic acid guide; and optionally
(b) at least one nucleic acid guided genome modifier protein, or any chimeric protein, complex or conjugate thereof, or at least one nucleic acid sequence encoding said guided genome modifier protein or chimeric protein thereof; or
(II) wherein at least one of:
(a) said compound reduces the stability of said Sesn3 by targeted protein degradation (TPD); and/or
(b) wherein said regulatory complex is the GAP activity towards Rags 2 (GATOR2) complex, and wherein said compound interferes and/or blocks the inhibitory interaction of Sestrin3 with at least one member of the GATOR2 complex.

57. The method according to claim 51, wherein at least one of:
(I) the method comprising administering to said subject at least one compound that increases the level and/or activity of p38, wherein said compound is a p38 activator that leads to phosphorylation of at least one of Thr180 (T180) and/or Tyr 182 (Y182) of p38, optionally, wherein said p38 activator is at least one of: a compound elevating the levels and/or activity of MAP kinase kinase 3 (MKK3) and/or MKK6; a hyperosmotic agent; and/or a DNA Synthesis Inhibitor; and
(II) the method comprising administering to said subject at least one compound that reduces the level and/or activity of p62 and at least one compound that reduces the level and/or activity of NBR1.

58. The method according to claim 51, wherein said pathologic disorder is a disorder affected by proteasomal activity and/or cellular localization, said disorder is at least one of: at least one neoplastic disorder and/or at least one protein misfolding disorder or deposition disorder, optionally, wherein said neoplastic disorder is cancer.

59. A method for treating, inhibiting, reducing, eliminating, protecting or delaying the onset of at least one condition or at least one pathologic disorder in a subject in need thereof, and for determining a personalized treatment regimen for said subject, by assessing responsiveness of said subject to a treatment regimen comprising at least one therapeutic compound, determining dosage of said compound, and/or monitoring disease progression of said subject, the method comprising the steps of:
(a) determining in at least one sample of said subject, at least one of:
(i) mTOR activation and/or lysosomal association;
(ii) activation of p388;
(iii) phosphorylation of Tyr705 of STAT3; and/or
(iv) Sestrin3 levels, and/or activity and/or the interaction of Sestrin3 with at least one regulatory complex; and optionally,
(v) the proteasome subcellular localization in at least one cell of said at least one sample, or in any fraction thereof;
(b) classifying said subject as:
(I) a responder subject to said treatment regimen, if at least one of: (i) mTOR is activated and/or localized to the lysosomal membrane; (ii) p38 is activated; (iii) phosphorylation of Tyr705 of STAT3 is inhibited or reduced; and/or (iv) Sestrin3 levels, and/or activity and/or the interaction of Sestrin3 with at least one regulatory complex is reduced; and optionally, (v) the ratio of nuclear to cytosolic proteasome subcellular localization is greater than 1; or
(II) a non-responder subject or a poor responder to said treatment regimen if at least one of: (i) mTOR is inactivated and/or dissociated from the lysosomal membrane; (ii) p38 delta is inactivated; (iii) Tyr705 of STAT3 is phosphorylated; and/or (iv) Sestrin3 levels, and/or activity and/or the interaction of Sestrin3 with at least one regulatory complex are maintained or increased; and optionally, (v) the ratio of nuclear to cytosolic proteasome subcellular localization is smaller than, or equal to 1;
(c) Initiating or maintaining said treatment regimen for a subject classified as a responder, increasing the dose of said compound in subject exhibiting a mild or poor response, or ceasing said treatment regimen for a subject classified as a non-responder or poor responder; thereby determining a treatment regimen to said subject.

60. The method according to claim 59, wherein at least one of:
(I) said monitoring disease progression comprises predicting and determining disease relapse and/or assessing a remission interval, and wherein said method further comprises the steps of:
(d) repeating step (a) to determine at least one of (i) to (iv), and optionally, (v), for at least one more temporally separated sample of said subject; and
(e) predicting and/or determining disease relapse in said subject, if said at least one temporally separated sample displays at least one of: (i) inactivation and/or dissociation of mTOR from the lysosomal membrane; (ii) loss of p38 T180/Y182 phosphorylation; (iii) increased and/or maintained phosphorylation of Tyr705 of STAT3; and/or (iv) increase in Sestrin3 levels, and/or activity and/or the interaction of Sestrin3 with at least one regulatory complex; and optionally, (v) loss of proteasome nuclear localization or maintained cytosolic localization, and/or reduction in the ratio of nuclear to cytosolic proteasome localization in at least one cell of said sample; and
(II) said at least one more temporally separated sample is obtained after the initiation of said at least one treatment regimen comprising said at least one therapeutic compound.

61. The method according to claim 59, wherein at least one of:
(I) said compound is a compound that modulates at least one pathway mediating proteasome dynamics and/or function, said compound is characterized by affecting at least one of: mTOR activation and/or lysosomal association, the activity and/or level/s and/or PTMs, and/or subcellular localization of at least one signaling molecule participating directly or indirectly in said at least one pathway mediating said proteasome dynamics and/or function; and optionally, the proteasome cellular localization;
(II) said at least one signaling molecule participating directly or indirectly in said at least one pathway mediating said proteasome dynamics and/or function is at least one of: at least one mediator of metabolite sensing, at least one stress kinase, at least one nucleo-cytosolic shuttle protein, specifically, ubiquitin and/or proteasome interacting shuttle proteins, and/or at least one NPC protein;
(III) wherein at least one of: (i) said mediator of metabolite sensing is a mediator of amino acid sensing; and/or (ii) wherein said stress kinase is at least one member of the MAPKs; and
(IV) wherein at least one of: said at least one mediator of amino acid sensing is at least one member of the Sestrin family, said at least one member of the MAPKs is at least one member of the p38 MAPKs (p38 MAPKs-p38α, p38B, p38y, p388), said at least one nucleo-cytosolic shuttle protein/s is p62 and NBR1, and/or wherein said at least one NPC is NUP93.

62. The method according to claim 59, wherein at least one of:
(A) said compound leads to:
(I) at least one of:
(i) mTOR activation and/or localization to the lysosomal membrane; (ii) reduction in Sestrin3 levels and/or activity; (iii) activation of p388; (iv) reduction in the levels and/or activity of p62 and NBR1; and/or (v) activation of NUP93; and optionally,
(II) proteasome nuclear localization;
(B) said compound comprises at least one of:
(a) at least one tyrosine (Y) residue, any tyrosine mimetic, any salt or ester thereof, any multimeric and/or polymeric form of said tyrosine residue and/or of said tyrosine mimetic, and any combinations or mixtures thereof;
(b) at least one tryptophan (W) residue, any tryptophan mimetic, any salt or ester thereof, any multimeric and/or polymeric form of said tryptophan residue and/or of said tryptophan mimetic, or any combination or mixture thereof; and/or
(c) at least one phenylalanine (F) residue, any phenylalanine mimetic, any salt or ester thereof, any multimeric and/or polymeric form of said phenylalanine residue and/or of said phenylalanine mimetic, and any combinations or mixtures thereof; or
(d) any combination of (a), (b), (c), or any peptide thereof, or any composition thereof; and
(C) at least one of:
(a) said compound is, or comprises at least one of: a nucleic acid-based molecule, an amino acid-based molecule, a small molecule or any combinations thereof; and
(b) said compound targets at least one of said signaling molecule/s at the nucleic acid sequence level and/or at the protein level.

63. The method according to claim 59, wherein at least one of:
(A) said compound reduces Sestrin3 levels and/or activity by at least one of: (i) specifically targeting a nucleic acid sequence encoding said Sestrin3, or any parts thereof; (ii) specifically targeting a nucleic acid sequence involves directly or indirectly in regulation of the Sestrin3 gene expression; (iii) reducing the stability of the Sestrin3 protein; and/or (iv) interfering with the interaction of Sestrin3 with at least one regulatory complex, optionally, said compound comprises:
(I) at least one compound that targets the nucleic acid sequence encoding Sestrin3, comprising at least one of:
(a) at least one RNA guide (gRNA) that guides least one nucleic acid guided genome modifier protein to at least one target sequence within said Sestrin3 encoding nucleic acid sequence, or within a nucleic acid sequence involves directly or indirectly in regulation of the Sestrin3 gene expression; or at least one nucleic acid sequence encoding said nucleic acid guide; and optionally
(b) at least one nucleic acid guided genome modifier protein, or any chimeric protein, complex or conjugate thereof, or at least one nucleic acid sequence encoding said guided genome modifier protein or chimeric protein thereof; and/or
(II) at least one compound that reduces the stability of said Sestrin3 by targeted protein degradation (TPD); and/or
(III) at least one compound that interferes and/or blocks the inhibitory interaction of Sestrin3 with at least one member of the GATOR2 complex;
(B) wherein said compound is a p38 activator that leads to phosphorylation of at least one of Thr180 (T180) and/or Tyr 182 (Y182) of p38; optionally, said p38 activator is at least one of: a compound elevating the levels and/or activity of MAP kinase kinase 3 (MKK3) and/or MKK6, a hyperosmotic agent, and/or a DNA Synthesis Inhibitor; and
(C) said pathologic disorder is a disorder affected by proteasomal activity and/or cellular localization, said disorder is at least one of: at least one neoplastic disorder and/or at least one protein misfolding disorder or deposition disorder, optionally, wherein said neoplastic disorder is a malignant or non-malignant neoplastic disorder and wherein said malignant neoplastic disorder is cancer.

64. A method for modulating proteolysis in at least one cell, the method comprising the step of contacting said cell with an effective amount of at least one compound that modulates proteasome dynamics and/or function, or subjecting said cell to conditions that modulate said proteasome dynamics/function, wherein said compound and/or conditions are characterized by affecting at least one of: mTOR activation and/or lysosomal association, the activity and/or level/s, and/or PTMs and/or localization of at least one signaling molecule participating directly or indirectly in at least one signaling pathway mediating said proteasome dynamics and/or function; and optionally, proteasome cellular localization.

65. The method according to claim 64, wherein at least one of:
(I) said at least one signaling molecule participating directly or indirectly in said signaling pathway mediating proteasome dynamics and/or function is at least one of: at least one mediator of metabolite sensing, at least one stress kinase, at least one nucleo-cytosolic shuttle protein, and at least one NPC protein;
(II) wherein at least one of: (i) said at least one mediator of amino acid sensing is at least one member of the Sestrin family; (ii) said at least one member of the MAPKs is at least one member of the p38 MAPKs; (iii) said at least one nucleo-cytosolic shuttle protein/s is p62 and NBR1, and/or (iv) wherein said at least one NPC is NUP93;
(III) wherein modulation of proteolysis by said compound and/or conditions results in proteasome recruitment/translocation to the cytosol and increased cytosolic proteolysis, and wherein said compound and/or conditions lead to, or are characterized by, at least one of:
(i) specific subtraction of at least one of the aromatic amino acid residue/s tyrosine (Y), tryptophan (W), and phenylalanine (F), or any combinations thereof;
(ii) inhibition and/or silencing of mTOR;
(iii) inhibition and/or silencing of p38;
(iv) activation of STAT3;
(v) inhibition and/or silencing of NUP93;
(vi) inhibition and/or silencing of protein/s participating and/or mediating nuclear import of the proteasome (AKIRIN2); and
(vii) increase in Sestrin3 levels, and/or activity and/or the association of Sestrin3 with at least one member of the GATOR2 complex; and
(IV) wherein said cell is of a subject suffering from a pathologic disorder associated with cytosolic accumulation of protein/s and/or polypeptides, and wherein said step of contacting said cell with a compound and/or subjecting the cell to conditions, is performed by administering to said subject a therapeutic effective amount of said at least one compound that modulates the proteasome dynamics and/or function, and/or subjecting said subject to said conditions, as defined in (III).

66. The method according to claim 64, wherein at least one of:
(I) modulation of proteolysis by said compound and/or conditions results in nuclear sequestration of the proteasome and increased nuclear proteolysis, and wherein said compound/s and/or conditions lead to, or are characterized by, at least one of:
(i) specific elevation of the levels of at least one of the aromatic amino acid residue/s Y, W, and F, or any combinations thereof;
(ii) activation of mTOR and/or association to the lysosomal membrane;
(iii) inhibition and/or silencing of Sestrin3;
(iv) activation and/or upregulation of p38;
(v) inhibition of STAT3;
(vi) inhibition and/or silencing of p62 and NBR1;
(vii) proteasome inhibition;
(viii) activation of MEK3 and/or MEK6; and/or
(ix) reduction of Sestrin3 activity and/or the interaction of Sestrin3 with at least one regulatory complex; and
(II) said cell is of a subject suffering from a pathologic disorder associated with nuclear accumulation of protein/s and/or polypeptides, and/or a disorder characterized with and/or deteriorated by cytosolic accumulation of the proteasome and/or increased cytosolic proteolysis; and wherein said step of contacting said cell with a compound and/or subjecting the cell to conditions, is performed by administering to said subject a therapeutic effective amount of said at least one compound that modulates the proteasome dynamics and/or function, and/or subjecting said subject to said conditions, as defined in (I), optionally, wherein said pathologic disorder is at least one of: disorders associated with nuclear accumulation of transcription factors and/or oncogene/s, disorders associated with accumulation of proteins in the nuclear lamina (e.g., Hutchinson-Gilford Progeria syndrome (HGPS), aging and premature-aging syndromes), disorder/s associated with and/or deteriorated by enhanced cytosolic proteolysis, specifically, neoplastic disorders.

67. The method according to claim 66, wherein said compound is at least one of:
(a) at least one tyrosine (Y) residue, any tyrosine mimetic, any salt or ester thereof, any multimeric and/or polymeric form of said tyrosine residue and/or of said tyrosine mimetic, and any combinations or mixtures thereof;
(b) at least one tryptophan (W) residue, any tryptophan mimetic, any salt or ester thereof, any multimeric and/or polymeric form of said tryptophan residue and/or of said tryptophan mimetic, or any combination or mixture thereof; and
(c) at least one phenylalanine (F) residue, any phenylalanine mimetic, any salt or ester thereof, any multimeric and/or polymeric form of said phenylalanine residue and/or of said phenylalanine mimetic, and any combinations or mixtures thereof; or
(d) a compound comprising (a), (b) and (c), or any formulation or peptide thereof.

68. The method according to claim 66, wherein at least one of:
(a) said compound is, or comprises at least one of: a nucleic acid-based molecule, an amino acid-based molecule, a small molecule or any combinations thereof; and
(b) said compound targets at least one of said signaling molecule/s at the nucleic acid sequence level or at the protein level.

69. The method according to claim 66, wherein Sestrin3 levels and/or activity are reduced by at least one of: (i) specifically targeting a nucleic acid sequence encoding said Sestrin3, or any parts thereof; (ii) specifically targeting a nucleic acid sequence involves directly or indirectly in regulation of the Sestrin3 gene expression; (iii) reducing the stability of the Sesn3 protein; and/or (iv) interfering with the interaction of Sestrin3 with at least one regulatory complex, optionally, wherein said compound comprises:
(I) at least one compound that targets the nucleic acid sequence encoding and/or regulating the expression of Sestrin3, comprising at least one of:
(a) at least one gRNA that guides least one nucleic acid guided genome modifier protein to at least one target sequence within said Sestrin3 encoding nucleic acid sequence, or within a nucleic acid sequence involves directly or indirectly in regulation of the Sestrin3 gene expression; or at least one nucleic acid sequence encoding said nucleic acid guide; and optionally
(b) at least one nucleic acid guided genome modifier protein, or any chimeric protein, complex or conjugate thereof, or at least one nucleic acid sequence encoding said guided genome modifier protein or chimeric protein thereof; and/or
(II) at least one compound that reduces the stability of said Sestrin3 by targeted protein degradation (TPD); and/or
(III) at least one compound that interferes and/or blocks the inhibitory interaction of Sestrin3 with at least one member of the GATOR2 complex.

70. The method according to claim 66, wherein at least one of:
(I) said compound is a p38 activator that leads to phosphorylation of at least one of Thr180 (T180) and/or Tyr 182 (Y182) of p38, optionally, wherein said p38 activator is at least one of: a compound elevating the levels and/or activity of MKK3 and/or MKK6, a hyperosmotic agent, and/or a DNA Synthesis Inhibitor; and
(II) wherein said compound inhibits and/or reduces the level and/or activity of p62 and of NBR1.


SEQUENCE LISTING (60 sequences)

SEQ ID NO: 1 - DNA, 20 bp, synthetic construct
  source 1..20: mol_type = other DNA; note = sgSESN3_1
cctggcacattatcatgctt

SEQ ID NO: 2 - DNA, 20 bp, synthetic construct
  source 1..20: mol_type = other DNA; note = sgSESN3_ 2
tgatattagcctgaatccat

SEQ ID NO: 3 - DNA, 20 bp, synthetic construct
  source 1..20: mol_type = other DNA; note = sgSESN3_ 3
gtgtctcaacccttgacaag

SEQ ID NO: 4 - DNA, 20 bp, synthetic construct
  source 1..20: mol_type = other DNA; note = SESN1; note = sgRNA
ggccgtgtacgcctcgttcg

SEQ ID NO: 5 - DNA, 20 bp, synthetic construct
  source 1..20: mol_type = other DNA; note = sgRNA; note = SESN2
atggccgagtttctgcagac

SEQ ID NO: 6 - DNA, 20 bp, synthetic construct
  source 1..20: mol_type = other DNA; note = CASTOR1; note = sgRNA
ccgttcggtcatcgcgccac

SEQ ID NO: 7 - DNA, 20 bp, synthetic construct
  source 1..20: mol_type = other DNA; note = sgRNA; note = NPRL3
cagccccatcagcgtgattc

SEQ ID NO: 8 - DNA, 20 bp, synthetic construct
  source 1..20: mol_type = other DNA; note = sgRNA; note = FLCN
tgctccgaccgaggatacct

SEQ ID NO: 9 - DNA, 20 bp, synthetic construct
  source 1..20: mol_type = other DNA; note = sgRNA; note = RagA
ggatggcctccagacacgac

SEQ ID NO: 10 - DNA, 20 bp, synthetic construct
  source 1..20: mol_type = other DNA; note = RagB; note = sgRNA
ttgtccttaggtgctgttga

SEQ ID NO: 11 - DNA, 20 bp, synthetic construct
  source 1..20: mol_type = other DNA; note = SLC38A9; note = sgRNA
cactcacatggttactaaac

SEQ ID NO: 12 - DNA, 20 bp, synthetic construct
  source 1..20: mol_type = other DNA; note = WDR24; note = sgRNA
cacgaactgttcctcctcga

SEQ ID NO: 13 - DNA, 20 bp, synthetic construct
  source 1..20: mol_type = other DNA; note = sgRNA; note = sgCont
gcactaccagagctaactca

SEQ ID NO: 14 - DNA, 21 bp, synthetic construct
  source 1..21: mol_type = other DNA; note = shRNA; note = mTOR
ggccgcattgtctctatcaag

SEQ ID NO: 15 - DNA, 21 bp, synthetic construct
  source 1..21: mol_type = other DNA; note = shRNA; note = GCN2_1
gcctaactggtgaagaagtat

SEQ ID NO: 16 - DNA, 21 bp, synthetic construct
  source 1..21: mol_type = other DNA; note = GCN2_2; note = shRNA
ccctaaagaactgtcgttaac

SEQ ID NO: 17 - DNA, 21 bp, synthetic construct
  source 1..21: mol_type = other DNA; note = GCN2_3; note = shRNA
ccaaaggtctatcaaatgaaa

SEQ ID NO: 18 - DNA, 21 bp, synthetic construct
  source 1..21: mol_type = other DNA; note = PIK3CA_1; note = shRNA
gcattagaatttacagcaaga

SEQ ID NO: 19 - DNA, 21 bp, synthetic construct
  source 1..21: mol_type = other DNA; note = shRNA; note = PIK3CA_2
gcacaatccatgaacagcatt

SEQ ID NO: 20 - DNA, 21 bp, synthetic construct
  source 1..21: mol_type = other DNA; note = shRNA; note = PIK3CA_3
gaattggagatcgtcacaata

SEQ ID NO: 21 - DNA, 21 bp, synthetic construct
  source 1..21: mol_type = other DNA; note = shRNA; note = AKT_1
ggacaaggacgggcacattaa

SEQ ID NO: 22 - DNA, 21 bp, synthetic construct
  source 1..21: mol_type = other DNA; note = AKT_2; note = shRNA
ggactacctgcactcggagaa

SEQ ID NO: 23 - DNA, 21 bp, synthetic construct
  source 1..21: mol_type = other DNA; note = shRNA; note = NUP93_1
gcaagtgaaacagcgaattct

SEQ ID NO: 24 - DNA, 21 bp, synthetic construct
  source 1..21: mol_type = other DNA; note = NUP93_2; note = shRNA
ggactccacgttctatcttct

SEQ ID NO: 25 - DNA, 22 bp, synthetic construct
  source 1..22: mol_type = other DNA; note = shCont; note = shRNA
atctcgcttgggcgagagtaag

SEQ ID NO: 26 - DNA, 7650 bp, Homo sapiens
  source 1..7650: mol_type = genomic DNA; note = https://www.ncbi.nlm.nih.gov/CCDS/CcdsBrowse.cgi?RE; QUEST = CCDS&GO=MainBrowse&DATA=CCDS127.1; note = mTOR; CCDS 127.1
atgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaa

SEQ ID NO: 27 - protein, 2549 aa, Homo sapiens
  source 1..2549: note = mTOR; Uniprot number: P42345
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 28 - DNA, 1323 bp, Homo sapiens
  source 1..1323: mol_type = genomic DNA; note = p62; CCDS 34317.1; note = https://www.ncbi.nlm.nih.gov/CCDS/CcdsBrowse.cgi?RE; QUEST = CCDS&GO=MainBrowse&DATA=CCDS34317.1
atggcgtcgctcaccgtgaaggcctaccttctgggcaaggaggacgcggcgcgcgagattcgccgcttcagcttctgctgcagccccgagcctgaggcggaagccgaggctgcggcgggtccgggaccctgcgagcggctgctgagccgggtggccgccctgttccccgcgctgcggcctggcggcttccaggcgcactaccgcgatgaggacggggacttggttgccttttccagtgacgaggaattgacaatggccatgtcctacgtgaaggatgacatcttccgaatctacattaaagagaaaaaagagtgccggcgggaccaccgcccaccgtgtgctcaggaggcgccccgcaacatggtgcaccccaatgtgatctgcgatggctgcaatgggcctgtggtaggaacccgctacaagtgcagcgtctgcccagactacgacttgtgtagcgtctgcgagggaaagggcttgcaccgggggcacaccaagctcgcattccccagccccttcgggcacctgtctgagggcttctcgcacagccgctggctccggaaggtgaaacacggacacttcgggtggccaggatgggaaatgggtccaccaggaaactggagcccacgtcctcctcgtgcaggggaggcccgccctggccccacggcagaatcagcttctggtccatcggaggatccgagtgtgaatttcctgaagaacgttggggagagtgtggcagctgcccttagccctctgggcattgaagttgatatcgatgtggagcacggagggaaaagaagccgcctgacccccgtctctccagagagttccagcacagaggagaagagcagctcacagccaagcagctgctgctctgaccccagcaagccgggtgggaatgttgagggcgccacgcagtctctggcggagcagatgaggaagatcgccttggagtccgaggggcgccctgaggaacagatggagtcggataactgttcaggaggagatgatgactggacccatctgtcttcaaaagaagtggacccgtctacaggtgaactccagtccctacagatgccagaatccgaagggccaagctctctggacccctcccaggagggacccacagggctgaaggaagctgccttgtacccacatctcccgccagaggctgacccgcggctgattgagtccctctcccagatgctgtccatgggcttctctgatgaaggcggctggctcaccaggctcctgcagaccaagaactatgacatcggagcggctctggacaccatccagtattcaaagcatcccccgccgttgtga

SEQ ID NO: 29 - protein, 440 aa, Homo sapiens
  source 1..440: note = Sequestosome-1 (p62); Uniprot Number: Q13501
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 30 - DNA, 1071 bp, Homo sapiens
  source 1..1071: mol_type = genomic DNA; note = p62 2nd isoform; CCDS 47355.1; note = https://www.ncbi.nlm.nih.gov/CCDS/CcdsBrowse.cgi?RE; QUEST = CCDS&GO=MainBrowse&DATA=CCDS47355.1
atggccatgtcctacgtgaaggatgacatcttccgaatctacattaaagagaaaaaagagtgccggcgggaccaccgcccaccgtgtgctcaggaggcgccccgcaacatggtgcaccccaatgtgatctgcgatggctgcaatgggcctgtggtaggaacccgctacaagtgcagcgtctgcccagactacgacttgtgtagcgtctgcgagggaaagggcttgcaccgggggcacaccaagctcgcattccccagccccttcgggcacctgtctgagggcttctcgcacagccgctggctccggaaggtgaaacacggacacttcgggtggccaggatgggaaatgggtccaccaggaaactggagcccacgtcctcctcgtgcaggggaggcccgccctggccccacggcagaatcagcttctggtccatcggaggatccgagtgtgaatttcctgaagaacgttggggagagtgtggcagctgcccttagccctctgggcattgaagttgatatcgatgtggagcacggagggaaaagaagccgcctgacccccgtctctccagagagttccagcacagaggagaagagcagctcacagccaagcagctgctgctctgaccccagcaagccgggtgggaatgttgagggcgccacgcagtctctggcggagcagatgaggaagatcgccttggagtccgaggggcgccctgaggaacagatggagtcggataactgttcaggaggagatgatgactggacccatctgtcttcaaaagaagtggacccgtctacaggtgaactccagtccctacagatgccagaatccgaagggccaagctctctggacccctcccaggagggacccacagggctgaaggaagctgccttgtacccacatctcccgccagaggctgacccgcggctgattgagtccctctcccagatgctgtccatgggcttctctgatgaaggcggctggctcaccaggctcctgcagaccaagaactatgacatcggagcggctctggacaccatccagtattcaaagcatcccccgccgttgtga

SEQ ID NO: 31 - protein, 356 aa, Homo sapiens
  source 1..356: note = p62 2nd isoform; CCDS 47355.1- Translation:
MAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

SEQ ID NO: 32 - DNA, 1098 bp, Homo sapiens
  source 1..1098: mol_type = genomic DNA; note = p38DELTA; CCDS 4818.1; note = https://www.ncbi.nlm.nih.gov/CCDS/CcdsBrowse.cgi?RE; QUEST = CCDS&GO=MainBrowse&DATA=CCDS4818.1
atgagcctcatccggaaaaagggcttctacaagcaggacgtcaacaagacagcctgggagctgcccaagacctacgtgtccccgacgcacgtcggcagcggggcctatggctccgtgtgctcggccatcgacaagcggtcaggggagaaggtggccatcaagaagctgagccgaccctttcagtccgagatcttcgccaagcgcgcctaccgggagctgctgctgctgaagcacatgcagcatgagaacgtcattgggctcctggatgtcttcaccccagcctcctccctgcgcaacttctatgacttctacctggtgatgcccttcatgcagacggatctgcagaagatcatggggatggagttcagtgaggagaagatccagtacctggtgtatcagatgctcaaaggccttaagtacatccactctgctggggtcgtgcacagggacctgaagccaggcaacctggctgtgaatgaggactgtgaactgaagattctggattttgggctggcgcgacatgcagacgccgagatgactggctacgtggtgacccgctggtaccgagcccccgaggtgatcctcagctggatgcactacaaccagacagtggacatctggtctgtgggctgtatcatggcagagatgctgacagggaaaactctgttcaaggggaaagattacctggaccagctgacccagatcctgaaagtgaccggggtgcctggcacggagtttgtgcagaagctgaacgacaaagcggccaaatcctacatccagtccctgccacagacccccaggaaggatttcactcagctgttcccacgggccagcccccaggctgcggacctgctggagaagatgctggagctagacgtggacaagcgcctgacggccgcgcaggccctcacccatcccttctttgaacccttccgggaccctgaggaagagacggaggcccagcagccgtttgatgattccttagaacacgagaaactcacagtggatgaatggaagcagcacatctacaaggagattgtgaacttcagccccattgcccggaaggactcacggcgccggagtggcatgaagctgtag

SEQ ID NO: 33 - protein, 365 aa, Homo sapiens
  source 1..365: note = p38DELTA, Uniprot number: O15264
MSLIRKKGFYKQDVNKTAWELPKTYVSPTHVGSGAYGSVCSAIDKRSGEKVAIKKLSRPFQSEIFAKRAYRELLLLKHMQHENVIGLLDVFTPASSLRNFYDFYLVMPFMQTDLQKIMGMEFSEEKIQYLVYQMLKGLKYIHSAGVVHRDLKPGNLAVNEDCELKILDFGLARHADAEMTGYVVTRWYRAPEVILSWMHYNQTVDIWSVGCIMAEMLTGKTLFKGKDYLDQLTQILKVTGVPGTEFVQKLNDKAAKSYIQSLPQTPRKDFTQLFPRASPQAADLLEKMLELDVDKRLTAAQALTHPFFEPFRDPEEETEAQQPFDDSLEHEKLTVDEWKQHIYKEIVNFSPIARKDSRRRSGMKL

SEQ ID NO: 34 - DNA, 1479 bp, Homo sapiens
  source 1..1479: mol_type = genomic DNA; note = SESN3; CCDS 8303.1; note = https://www.ncbi.nlm.nih.gov/CCDS/CcdsBrowse.cgi?RE; QUEST = CCDS&GO=MainBrowse&DATA=CCDS8303.1
atgaaccggggcggcggcagcccgtcggccgccgccaactacctgctctgtaccaactgccggaaagtgctgcggaaggataaaagaatcagagtgtctcaacccttgacaagaggaccaagtgcctttattccagagaaggaagttgtccaagcaaacacagtggatgaacgtactaactttcttgtggaagaatactctacatccggtcgtctggacaacatcacacaggtcatgagtttacacactcagtacctggagtctttcttgcggagccagttttacatgttgcgcatggatggtccccttcctctaccatacaggcactatattgcaataatggctgcagctagacatcagtgttcttacttaataaacatgcatgtggatgaatttttaaagactggaggtattgctgagtggttgaatggtttggaatatgtgccacaaagactgaaaaatcttaatgaaattaataagctgctagcacatcgaccttggctgatcacaaaagagcacattcagaaacttgtcaaaactggagaaaataattggtctctgcctgaactggtacatgctgtggtcctcctggcacattatcatgctttggcaagctttgtttttggtagtggtatcaatccagagagagatccagaaatctccaatggattcaggctaatatcagtcaacaatttctgcgtttgtgatcttgctaatgacaacaacatagagaatgcatctctttcaggcagcaactttgggattgtggattctctaagtgagctagaggccttaatggaaaggatgaaaagacttcaagaagaaagggaagatgaagaggcgtctcaagaagaaatgagcactcgttttgaaaaggagaagaaagaaagtctttttgtggtctctggagatacttttcattcatttcctcattcagattttgaggatgacatgattataacatctgatgtctctcgatatattgaagaccctggttttgggtatgaagactttgccagacgaggagaagagcatttgccaacattccgagctcaggactatacctgggaaaatcatgggttctccctggtgaacagactttattctgacattggacatcttcttgatgaaaagtttcggatggtctacaatctcacatataacactatggccacccatgaggatgttgacacaaccatgctgcgcagagctttatttaactatgttcactgtatgtttggaatcaggtatgatgactatgattatggagaagttaatcaattacttgaaagaagcctgaaggtttacattaagacagtgacctgctatcctgagagaactacaaaacgcatgtatgatagttactggcggcagttcaaacactcagaaaaagttcatgtcaatctacttttaatggaagcacgaatgcaagctgaacttctttatgctcttcgtgccataactcggcatttgacctga

SEQ ID NO: 35 - protein, 492 aa, Homo sapiens
  source 1..492: note = SESN3; Uniport number: P58005-1
MNRGGGSPSAAANYLLCTNCRKVLRKDKRIRVSQPLTRGPSAFIPEKEVVQANTVDERTNFLVEEYSTSGRLDNITQVMSLHTQYLESFLRSQFYMLRMDGPLPLPYRHYIAIMAAARHQCSYLINMHVDEFLKTGGIAEWLNGLEYVPQRLKNLNEINKLLAHRPWLITKEHIQKLVKTGENNWSLPELVHAVVLLAHYHALASFVFGSGINPERDPEISNGFRLISVNNFCVCDLANDNNIENASLSGSNFGIVDSLSELEALMERMKRLQEEREDEEASQEEMSTRFEKEKKESLFVVSGDTFHSFPHSDFEDDMIITSDVSRYIEDPGFGYEDFARRGEEHLPTFRAQDYTWENHGFSLVNRLYSDIGHLLDEKFRMVYNLTYNTMATHEDVDTTMLRRALFNYVHCMFGIRYDDYDYGEVNQLLERSLKVYIKTVTCYPERTTKRMYDSYWRQFKHSEKVHVNLLLMEARMQAELLYALRAITRHLT

SEQ ID NO: 36 - DNA, 1062 bp, Homo sapiens
  source 1..1062: mol_type = genomic DNA; note = SESN3 2nd isoform; CCDS 60938.1; note = https://www.ncbi.nlm.nih.gov/CCDS/CcdsBrowse.cgi?RE; QUEST = CCDS&GO=MainBrowse&DATA=CCDS60938.1
atgagtttacacactcagtacctggagtctttcttgcggagccagttttacatgttgcgcatggatggtccccttcctctaccatacaggcactatattgcaataatgaaacttgtcaaaactggagaaaataattggtctctgcctgaactggtacatgctgtggtcctcctggcacattatcatgctttggcaagctttgtttttggtagtggtatcaatccagagagagatccagaaatctccaatggattcaggctaatatcagtcaacaatttctgcgtttgtgatcttgctaatgacaacaacatagagaatgcatctctttcaggcagcaactttgggattgtggattctctaagtgagctagaggccttaatggaaaggatgaaaagacttcaagaagaaagggaagatgaagaggcgtctcaagaagaaatgagcactcgttttgaaaaggagaagaaagaaagtctttttgtggtctctggagatacttttcattcatttcctcattcagattttgaggatgacatgattataacatctgatgtctctcgatatattgaagaccctggttttgggtatgaagactttgccagacgaggagaagagcatttgccaacattccgagctcaggactatacctgggaaaatcatgggttctccctggtgaacagactttattctgacattggacatcttcttgatgaaaagtttcggatggtctacaatctcacatataacactatggccacccatgaggatgttgacacaaccatgctgcgcagagctttatttaactatgttcactgtatgtttggaatcaggtatgatgactatgattatggagaagttaatcaattacttgaaagaagcctgaaggtttacattaagacagtgacctgctatcctgagagaactacaaaacgcatgtatgatagttactggcggcagttcaaacactcagaaaaagttcatgtcaatctacttttaatggaagcacgaatgcaagctgaacttctttatgctcttcgtgccataactcggcatttgacctga

SEQ ID NO: 37 - protein, 353 aa, Homo sapiens
  source 1..353: note = SESN3 2nd isoform; CCDS 60938.1 - Translation
MSLHTQYLESFLRSQFYMLRMDGPLPLPYRHYIAIMKLVKTGENNWSLPELVHAVVLLAHYHALASFVFGSGINPERDPEISNGFRLISVNNFCVCDLANDNNIENASLSGSNFGIVDSLSELEALMERMKRLQEEREDEEASQEEMSTRFEKEKKESLFVVSGDTFHSFPHSDFEDDMIITSDVSRYIEDPGFGYEDFARRGEEHLPTFRAQDYTWENHGFSLVNRLYSDIGHLLDEKFRMVYNLTYNTMATHEDVDTTMLRRALFNYVHCMFGIRYDDYDYGEVNQLLERSLKVYIKTVTCYPERTTKRMYDSYWRQFKHSEKVHVNLLLMEARMQAELLYALRAITRHLT

SEQ ID NO: 38 - DNA, 2901 bp, Homo sapiens
  source 1..2901: mol_type = genomic DNA; note = https://www.ncbi.nlm.nih.gov/CCDS/CcdsBrowse.cgi?RE; QUEST = CCDS&GO=MainBrowse&DATA=CCDS45694.1; note = NBR1; CCDS 45694.1
atggaaccacaggttactctaaatgtgacttttaaaaatgaaattcaaagctttctggtttctgatccagaaaatacaacttgggctgatatcgaagctatggtaaaagtttcatttgatctgaatactattcaaataaaatacctggatgaggaaaatgaagaggtatccatcaacagtcaaggagaatatgaagaagcgcttaagatggcagttaaacagggaaaccaactgcagatgcaagtccacgaagggcaccatgtcgttgatgaagccccacccccagttgtaggagcaaaacgactagctgccagggcagggaagaagccacttgcacattactcttcactggtgagagtcttgggatcagacatgaagaccccagaggatcctgcagtgcagtcgtttccacttgttccatgtgacacagaccagcctcaagacaagcccccagactggttcacaagctacctggagacgttcagagaacaagtggttaacgaaacggttgagaagcttgaacagaaattacatgaaaagcttgtcctccagaacccatccttgggttcttgtccctcagaagtctcaatgcctacttcagaagaaacattgtttttgccagaaaaccagttcagctggcatattgcttgcaacaactgccaaagaaggattgttggtgtccgctaccagtgtagcctatgcccatcctacaatatctgtgaagattgtgaagcagggccatatggccatgacactaaccacgtcctgctgaagttgcggagacctgttgtgggctcctctgaaccgttctgtcactcaaagtactctactcctcgtcttcctgctgctctggaacaagtcaggctccagaaacaggttgataagaactttcttaaagcagaaaagcaaaggttgcgagctgagaagaaacaacgtaaagcagaggtcaaggaacttaaaaagcagcttaaactccataggaaaattcacctgtggaattcaatccatggactccagagccccaagtctcctttaggccgacctgagagcttgctccagtctaataccctgatgctccctttgcagccctgtacctccgttatgccaatgctcagtgcagcatttgtggatgagaatttgcctgatgggactcaccttcagccaggaaccaagtttatcaaacactggaggatgaaaaatacaggaaatgtaaagtggagtgcagacacaaagctcaagttcatgtggggaaacctgactttggcttccacagaaaagaaggatgttttggttccctgcctcaaggccggccatgtgggagttgtatctgtggagttcattgccccagccttggagggaacgtatacttcccattggcgtctttctcacaaaggccagcaatttgggcctcgggtctggtgcagtatcatagtagatcctttcccctccgaagagagccctgataacattgaaaagggcatgatcagctcaagcaaaactgatgatctcacctgccagcaagaggaaacttttcttctggctaaagaagaaagacagcttggtgaagtgactgagcagacagaagggacagcagcctgcatcccacagaaggcaaaaaatgttgccagtgagagggagctctacatcccatctgtggatcttctgactgcccaggacctgctgtcctttgagctgttggatataaacattgttcaagagttggagagagtgccccacaacacccctgtggatgtgactccctgcatgtctcctctgccacatgacagtcctttaatagagaagccaggcttggggcagatagaggaagagaatgaaggggcaggatttaaagcacttcctgattctatggtgtcagtaaagaggaaggctgagaacattgcttctgtggaggaagcagaagaagacctgagtgggacccagtttgtgtgtgagacagtaatccgatcccttaccttggatgctgccccagaccacaaccctccttgcagacagaagtccttgcagatgacatttgccttgcctgaaggaccacttggaaatgagaaggaggagattatccatatcgctgaggaagaagctgtcatggaggaggaggaggatgaggaggatgaggaggaggaggatgagctcaaagatgaagttcaaagtcagtcctctgcttcctcagaggattacatcatcatcctgcctgagtgctttgataccagccgccccctgggggattctatgtacagctctgcgctctcacagccaggcctggagcgaggtgctgaaggcaagcctggggttgaggctgggcaggaaccagctgaggctggggaaagactccctggaggggagaaccagccacaggagcacagcataagtgacatcctcacgacctcacagactctggaaacagtgcccctaatcccagaggtagtggagcttccaccgtcactgcccaggagctctccttgtgtacatcatcatggttccccaggagtggatttaccagttaccataccagaagtttcttcagtccctgatcagatcagaggagagcccagaggctcatcaggacttgtaaacagcagacagaagagctatgaccactcaaggcaccatcatgggagcagcattgctggaggactggtgaagggggctttgtctgttgctgcctctgcatacaaggccctgtttgctgggccaccagtcactgcacagccaataatttctgaagatcagacagcagccctgatggcccatctctttgaaatgggattctgtgacaggcagctgaacctacggctgctgaagaaacacaattacaatatcctgcaggttgtgacagaacttcttcagttaaacaacaacgactggtacagccaacgctattga

SEQ ID NO: 39 - protein, 966 aa, Homo sapiens
  source 1..966: note = NBR1; Uniprot number: Q14596
MEPQVTLNVTFKNEIQSFLVSDPENTTWADIEAMVKVSFDLNTIQIKYLDEENEEVSINSQGEYEEALKMAVKQGNQLQMQVHEGHHVVDEAPPPVVGAKRLAARAGKKPLAHYSSLVRVLGSDMKTPEDPAVQSFPLVPCDTDQPQDKPPDWFTSYLETFREQVVNETVEKLEQKLHEKLVLQNPSLGSCPSEVSMPTSEETLFLPENQFSWHIACNNCQRRIVGVRYQCSLCPSYNICEDCEAGPYGHDTNHVLLKLRRPVVGSSEPFCHSKYSTPRLPAALEQVRLQKQVDKNFLKAEKQRLRAEKKQRKAEVKELKKQLKLHRKIHLWNSIHGLQSPKSPLGRPESLLQSNTLMLPLQPCTSVMPMLSAAFVDENLPDGTHLQPGTKFIKHWRMKNTGNVKWSADTKLKFMWGNLTLASTEKKDVLVPCLKAGHVGVVSVEFIAPALEGTYTSHWRLSHKGQQFGPRVWCSIIVDPFPSEESPDNIEKGMISSSKTDDLTCQQEETFLLAKEERQLGEVTEQTEGTAACIPQKAKNVASERELYIPSVDLLTAQDLLSFELLDINIVQELERVPHNTPVDVTPCMSPLPHDSPLIEKPGLGQIEEENEGAGFKALPDSMVSVKRKAENIASVEEAEEDLSGTQFVCETVIRSLTLDAAPDHNPPCRQKSLQMTFALPEGPLGNEKEEIIHIAEEEAVMEEEEDEEDEEEEDELKDEVQSQSSASSEDYIIILPECFDTSRPLGDSMYSSALSQPGLERGAEGKPGVEAGQEPAEAGERLPGGENQPQEHSISDILTTSQTLETVPLIPEVVELPPSLPRSSPCVHHHGSPGVDLPVTIPEVSSVPDQIRGEPRGSSGLVNSRQKSYDHSRHHHGSSIAGGLVKGALSVAASAYKALFAGPPVTAQPIISEDQTAALMAHLFEMGFCDRQLNLRLLKKHNYNILQVVTELLQLNNNDWYSQRY

SEQ ID NO: 40 - DNA, 2814 bp, Homo sapiens
  source 1..2814: mol_type = genomic DNA; note = NBR1 2nd isoform; CCDS77037.1; note = https://www.ncbi.nlm.nih.gov/CCDS/CcdsBrowse.cgi?RE; QUEST = CCDS&GO=MainBrowse&DATA=CCDS77037.1
atggaaccacaggttactctaaatgtgacttttaaaaatgaaattcaaagctttctggtttctgatccagaaaatacaacttgggctgatatcgaagctatggtaaaagtttcatttgatctgaatactattcaaataaaatacctggatgaggaaaatgaagaggtatccatcaacagtcaaggagaatatgaagaagcgcttaagatggcagttaaacagggaaaccaactgcagatgcaagtccacgaagggcaccatgtcgttgatgaagccccacccccagttgtaggagcaaaacgactagctgccagggcagggaagaagccacttgcacattactcttcactggtgagagtcttgggatcagacatgaagaccccagaggatcctgcagtgcagtcgtttccacttgttccatgtgacacagaccagcctcaagacaagcccccagactggttcacaagctacctggagacgttcagagaacaagtggttaacgaaacggttgagaagcttgaacagaaattacatgaaaagcttgtcctccagaacccatccttgggttcttgtccctcagaagtctcaatgcctacttcagaagaaacattgtttttgccagaaaaccagttcagctggcatattgcttgcaacaactgccaaagaaggattgttggtgtccgctaccagtgtagcctatgcccatcctacaatatctgtgaagattgtgaagcagggccatatggccatgacactaaccacgtcctgctgaagttgcggagacctgttgtgggctcctctgaaccgttctgtcactcaaagtactctactcctcgtcttcctgctgctctggaacaagtcaggctccagaaacaggttgataagaactttcttaaagcagaaaagcaaaggttgcgagctgagaagaaacaacgtaaagcagaggtcaaggaacttaaaaagcagcttaaactccataggaaaattcacctgtggaattcaatccatggactccagagccccaagtctcctttaggccgacctgagagcttgctccagtctaataccctgatgctccctttgcagccctgtacctccgttatgccaatgctcagtgcagcatttgtggatgagaatttgcctgatgggactcaccttcagccaggaaccaagtttatcaaacactggaggatgaaaaatacaggaaatgtaaagtggagtgcagacacaaagctcaagttcatgtggggaaacctgactttggcttccacagaaaagaaggatgttttggttccctgcctcaaggccggccatgtgggagttgtatctgtggagttcattgccccagccttggagggaacgtatacttcccattggcgtctttctcacaaaggccagcaatttgggcctcgggtctggtgcagtatcatagtagatcctttcccctccgaagagagccctgataacattgaaaagggcatgatcagctcaagcaaaactgatgatctcacctgccagcaagaggaaacttttcttctggctaaagaagaaagacagcttggtgaagtgactgagcagacagaagggacagcagcctgcatcccacagaaggcaaaaaatgttgccagtgagagggagctctacatcccatctgtggatcttctgactgcccaggacctgctgtcctttgagctgttggatataaacattgttcaagagttggagagagtgccccacaacacccctgtggatgtgactccctgcatgtctcctctgccacatgacagtcctttaatagagaagccaggcttggggcagatagaggaagagaatgaaggggcaggatttaaagcacttcctgattctatggtgtcagtaaagaggaaggctgagaacattgcttctgtggaggaagcagaagaagacctgagtgggacccagtttgtgtgtgagacagtaatccgatcccttaccttggatgctgccccagaccacaaccctccttgcagacagaagtccttgcagatgacatttgccttgcctgaaggaccacttggaaatgagaaggaggagattatccatatcgctgaggaagaagctgtcatggaggaggaggaggatgaggaggatgaggaggaggaggatgagctcaaagatgaagttcaaagtcagtcctctgcttcctcagaggattacatcatcatcctgcctgagtgctttgataccagccgccccctgggggattctatgtacagctctgcgctctcacagccaggcctggagcgaggtgctgaaggcaagcctggggttgaggctgggcaggaaccagctgaggctggggaaagactccctggaggggagaaccagccacaggagcacagcataagtgacatcctcacgacctcacagactctggaaacagtgcccctaatcccagaggtagtggagcttccaccgtcactgcccaggagctctccttgtgtacatcatcatggttccccaggagtggatttaccagttaccataccagaagtttcttcagtccctgatcagatcagaggagagcccagaggctcatcaggacttgtaaacagcagacagaagagctatgaccactcaaggcaccatcatgggagcagcattgctggaggactggtgaagggggctttgtctgttgctgcctctgcatacaaggccctgtttgctgggccaccagtcactgcacagggtctctggggcttgctgtcattccttcatctggccaaaaaatgcttcttcctgaaagccccgagtgaagcattctcctggttctaa

SEQ ID NO: 41 - protein, 937 aa, Homo sapiens
  source 1..937: note = NBR1 2nd isoform; CCDS77037.1 - Translation
MEPQVTLNVTFKNEIQSFLVSDPENTTWADIEAMVKVSFDLNTIQIKYLDEENEEVSINSQGEYEEALKMAVKQGNQLQMQVHEGHHVVDEAPPPVVGAKRLAARAGKKPLAHYSSLVRVLGSDMKTPEDPAVQSFPLVPCDTDQPQDKPPDWFTSYLETFREQVVNETVEKLEQKLHEKLVLQNPSLGSCPSEVSMPTSEETLFLPENQFSWHIACNNCQRRIVGVRYQCSLCPSYNICEDCEAGPYGHDTNHVLLKLRRPVVGSSEPFCHSKYSTPRLPAALEQVRLQKQVDKNFLKAEKQRLRAEKKQRKAEVKELKKQLKLHRKIHLWNSIHGLQSPKSPLGRPESLLQSNTLMLPLQPCTSVMPMLSAAFVDENLPDGTHLQPGTKFIKHWRMKNTGNVKWSADTKLKFMWGNLTLASTEKKDVLVPCLKAGHVGVVSVEFIAPALEGTYTSHWRLSHKGQQFGPRVWCSIIVDPFPSEESPDNIEKGMISSSKTDDLTCQQEETFLLAKEERQLGEVTEQTEGTAACIPQKAKNVASERELYIPSVDLLTAQDLLSFELLDINIVQELERVPHNTPVDVTPCMSPLPHDSPLIEKPGLGQIEEENEGAGFKALPDSMVSVKRKAENIASVEEAEEDLSGTQFVCETVIRSLTLDAAPDHNPPCRQKSLQMTFALPEGPLGNEKEEIIHIAEEEAVMEEEEDEEDEEEEDELKDEVQSQSSASSEDYIIILPECFDTSRPLGDSMYSSALSQPGLERGAEGKPGVEAGQEPAEAGERLPGGENQPQEHSISDILTTSQTLETVPLIPEVVELPPSLPRSSPCVHHHGSPGVDLPVTIPEVSSVPDQIRGEPRGSSGLVNSRQKSYDHSRHHHGSSIAGGLVKGALSVAASAYKALFAGPPVTAQGLWGLLSFLHLAKKCFFLKAPSEAFSWF

SEQ ID NO: 42 - DNA, 2460 bp, Homo sapiens
  source 1..2460: mol_type = genomic DNA; note = NUP93; CCDS 10769.1; note = https://www.ncbi.nlm.nih.gov/CCDS/CcdsBrowse.cgi?RE; QUEST = CCDS&GO=MainBrowse&DATA=CCDS10769.1
atggatactgaggggtttggtgagctccttcagcaagctgaacagcttgctgctgagactgagggcatctcagagcttccccatgtggaacggaacttacaggagatccagcaggcgggagagcgcctgcgttcccgtaccctaacacgcacgtcccaggagacggcagatgtcaaggcgtcagttctcctcgggtctcggggacttgacatatcccacatctcccagcgattggagagtctgagtgcagccaccacctttgagcctcttgagcctgtgaaggacactgacattcagggcttcctgaagaatgagaaggacaatgccctgctgtctgccatcgaagagtcccggaagaggaccttcggcatggctgaggagtaccatcgggagtcaatgttggttgagtgggagcaagtgaaacagcgaattctgcacacactgctggcatcaggagaagacgcccttgactttactcaagaaagcgagccaagctacatcagtgatgtgggaccccctggtcgaagctctctggataacatcgagatggcctatgcgcggcaaatttatatctataatgagaaaattgtaaatggacacctgcagcctaacctggtggacctttgtgcttccgtcgcagagctcgatgataagagcatttccgacatgtggaccatggtaaaacaaatgacagacgtgttgttgacaccggcaacggatgccctgaagaaccgcagcagcgtggaagtgcgcatggagtttgtcaggcaggccttggcgtaccttgagcagagttataagaattacacccttgtgactgtctttggaaatttgcatcaggcccagctgggcggggtgcctgggacttaccaattggttcgaagtttcctgaacattaaactgccagctcccttgcctggactacaggatggagaggtggaaggccatcctgtgtgggcgctaatttactactgcatgcgctgtggagacctgcttgccgcttcacaggtagttaatcgagcccagcaccagctgggagagtttaaaacctggttccaggagtacatgaacagcaaggacagaagattgtccccagctacggaaaacaagctccggctgcattaccgtagggccctcaggaacaatacagatccctacaagcgggccgtgtactgtatcattggcagatgtgacgtcaccgacaaccagagtgaagtggcggacaaaactgaggattacctgtggctgaagttgaaccaagtgtgttttgacgacgatggcaccagctccccacaagacaggctcactctctcacagttccagaagcagttgttggaagactatggcgagtcccactttacggtgaaccagcaacccttcctctacttccaagtcctgttcctgacagcgcagtttgaagcagcagttgcctttcttttccgcatggagcggctgcgctgccatgctgtccatgtagcactggtgctgtttgagctgaagctgcttttaaagtcctctggacagagtgctcagctcctcagccacgagcctggtgaccctccttgcttgcggcggctgaacttcgtgcggctcctcatgctgtacacccggaagtttgagtccacggacccaagggaggccctccagtacttctatttcctcagggatgagaaagatagtcaaggagaaaacatgtttctgcgctgtgtgagtgagcttgtgattgaaagccgagagttcgatatgattcttgggaaactagagaatgacggaagtagaaagcctggagtcatagataagtttactagtgacacaaagcctattatcaacaaagttgcttctgtggcagaaaataaaggactgtttgaagaggcagcaaagctgtatgaccttgccaagaatgctgacaaggtactggagctgatgaacaaactgctgagccctgtcgtcccccagatcagtgccccgcaatccaacaaggagaggctgaagaacatggcactctccattgccgaacggtatagggctcaaggaataagcgcaaataaatttgtggactccacgttctatcttcttttggacttgatcaccttttttgacgagtatcatagtggtcatattgatagagcttttgatatcattgagcgcttgaagctggtgcccctgaatcaggaaagtgtggaagagagagtggctgccttcagaaatttcagtgatgaaatcaggcacaacctctcagaagtgcttcttgccaccatgaacatcttgttcacacagtttaagaggctcaaggggacaagtccatcctcgtcatccaggccccagcgagtcatcgaggaccgcgactctcaactccgaagtcaagcccgcactctgattacctttgctggaatgataccataccgaacgtctggggacaccaatgcgaggctggtgcagatggaggtcctcatgaattaa

SEQ ID NO: 43 - protein, 819 aa, Homo sapiens
  source 1..819: note = NUP93; Uniprot number: Q8N1F7-1
MDTEGFGELLQQAEQLAAETEGISELPHVERNLQEIQQAGERLRSRTLTRTSQETADVKASVLLGSRGLDISHISQRLESLSAATTFEPLEPVKDTDIQGFLKNEKDNALLSAIEESRKRTFGMAEEYHRESMLVEWEQVKQRILHTLLASGEDALDFTQESEPSYISDVGPPGRSSLDNIEMAYARQIYIYNEKIVNGHLQPNLVDLCASVAELDDKSISDMWTMVKQMTDVLLTPATDALKNRSSVEVRMEFVRQALAYLEQSYKNYTLVTVFGNLHQAQLGGVPGTYQLVRSFLNIKLPAPLPGLQDGEVEGHPVWALIYYCMRCGDLLAASQVVNRAQHQLGEFKTWFQEYMNSKDRRLSPATENKLRLHYRRALRNNTDPYKRAVYCIIGRCDVTDNQSEVADKTEDYLWLKLNQVCFDDDGTSSPQDRLTLSQFQKQLLEDYGESHFTVNQQPFLYFQVLFLTAQFEAAVAFLFRMERLRCHAVHVALVLFELKLLLKSSGQSAQLLSHEPGDPPCLRRLNFVRLLMLYTRKFESTDPREALQYFYFLRDEKDSQGENMFLRCVSELVIESREFDMILGKLENDGSRKPGVIDKFTSDTKPIINKVASVAENKGLFEEAAKLYDLAKNADKVLELMNKLLSPVVPQISAPQSNKERLKNMALSIAERYRAQGISANKFVDSTFYLLLDLITFFDEYHSGHIDRAFDIIERLKLVPLNQESVEERVAAFRNFSDEIRHNLSEVLLATMNILFTQFKRLKGTSPSSSSRPQRVIEDRDSQLRSQARTLITFAGMIPYRTSGDTNARLVQMEVLMN

SEQ ID NO: 44 - DNA, 2091 bp, Homo sapiens
  source 1..2091: mol_type = genomic DNA; note = NUP93 2nd isoform; CCDS 55996.1; note = https://www.ncbi.nlm.nih.gov/CCDS/CcdsBrowse.cgi?RE; QUEST = CCDS&GO=MainBrowse&DATA=CCDS55996.1
atggctgaggagtaccatcgggagtcaatgttggttgagtgggagcaagtgaaacagcgaattctgcacacactgctggcatcaggagaagacgcccttgactttactcaagaaagcgagccaagctacatcagtgatgtgggaccccctggtcgaagctctctggataacatcgagatggcctatgcgcggcaaatttatatctataatgagaaaattgtaaatggacacctgcagcctaacctggtggacctttgtgcttccgtcgcagagctcgatgataagagcatttccgacatgtggaccatggtaaaacaaatgacagacgtgttgttgacaccggcaacggatgccctgaagaaccgcagcagcgtggaagtgcgcatggagtttgtcaggcaggccttggcgtaccttgagcagagttataagaattacacccttgtgactgtctttggaaatttgcatcaggcccagctgggcggggtgcctgggacttaccaattggttcgaagtttcctgaacattaaactgccagctcccttgcctggactacaggatggagaggtggaaggccatcctgtgtgggcgctaatttactactgcatgcgctgtggagacctgcttgccgcttcacaggtagttaatcgagcccagcaccagctgggagagtttaaaacctggttccaggagtacatgaacagcaaggacagaagattgtccccagctacggaaaacaagctccggctgcattaccgtagggccctcaggaacaatacagatccctacaagcgggccgtgtactgtatcattggcagatgtgacgtcaccgacaaccagagtgaagtggcggacaaaactgaggattacctgtggctgaagttgaaccaagtgtgttttgacgacgatggcaccagctccccacaagacaggctcactctctcacagttccagaagcagttgttggaagactatggcgagtcccactttacggtgaaccagcaacccttcctctacttccaagtcctgttcctgacagcgcagtttgaagcagcagttgcctttcttttccgcatggagcggctgcgctgccatgctgtccatgtagcactggtgctgtttgagctgaagctgcttttaaagtcctctggacagagtgctcagctcctcagccacgagcctggtgaccctccttgcttgcggcggctgaacttcgtgcggctcctcatgctgtacacccggaagtttgagtccacggacccaagggaggccctccagtacttctatttcctcagggatgagaaagatagtcaaggagaaaacatgtttctgcgctgtgtgagtgagcttgtgattgaaagccgagagttcgatatgattcttgggaaactagagaatgacggaagtagaaagcctggagtcatagataagtttactagtgacacaaagcctattatcaacaaagttgcttctgtggcagaaaataaaggactgtttgaagaggcagcaaagctgtatgaccttgccaagaatgctgacaaggtactggagctgatgaacaaactgctgagccctgtcgtcccccagatcagtgccccgcaatccaacaaggagaggctgaagaacatggcactctccattgccgaacggtatagggctcaaggaataagcgcaaataaatttgtggactccacgttctatcttcttttggacttgatcaccttttttgacgagtatcatagtggtcatattgatagagcttttgatatcattgagcgcttgaagctggtgcccctgaatcaggaaagtgtggaagagagagtggctgccttcagaaatttcagtgatgaaatcaggcacaacctctcagaagtgcttcttgccaccatgaacatcttgttcacacagtttaagaggctcaaggggacaagtccatcctcgtcatccaggccccagcgagtcatcgaggaccgcgactctcaactccgaagtcaagcccgcactctgattacctttgctggaatgataccataccgaacgtctggggacaccaatgcgaggctggtgcagatggaggtcctcatgaattaa

SEQ ID NO: 45 - protein, 696 aa, Homo sapiens
  source 1..696: note = NUP93 2nd isoform; CCDS 55996.1 - Translation
MAEEYHRESMLVEWEQVKQRILHTLLASGEDALDFTQESEPSYISDVGPPGRSSLDNIEMAYARQIYIYNEKIVNGHLQPNLVDLCASVAELDDKSISDMWTMVKQMTDVLLTPATDALKNRSSVEVRMEFVRQALAYLEQSYKNYTLVTVFGNLHQAQLGGVPGTYQLVRSFLNIKLPAPLPGLQDGEVEGHPVWALIYYCMRCGDLLAASQVVNRAQHQLGEFKTWFQEYMNSKDRRLSPATENKLRLHYRRALRNNTDPYKRAVYCIIGRCDVTDNQSEVADKTEDYLWLKLNQVCFDDDGTSSPQDRLTLSQFQKQLLEDYGESHFTVNQQPFLYFQVLFLTAQFEAAVAFLFRMERLRCHAVHVALVLFELKLLLKSSGQSAQLLSHEPGDPPCLRRLNFVRLLMLYTRKFESTDPREALQYFYFLRDEKDSQGENMFLRCVSELVIESREFDMILGKLENDGSRKPGVIDKFTSDTKPIINKVASVAENKGLFEEAAKLYDLAKNADKVLELMNKLLSPVVPQISAPQSNKERLKNMALSIAERYRAQGISANKFVDSTFYLLLDLITFFDEYHSGHIDRAFDIIERLKLVPLNQESVEERVAAFRNFSDEIRHNLSEVLLATMNILFTQFKRLKGTSPSSSSRPQRVIEDRDSQLRSQARTLITFAGMIPYRTSGDTNARLVQMEVLMN

SEQ ID NO: 46 - protein, 770 aa, Homo sapiens
  source 1..770: note = STAT3; Uniprot number: P40763-1
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 47 - protein, 382 aa, Homo sapiens
  source 1..382: note = MKK3; Uniprot number: Q16644
MDGETAEEQGGPVPPPVAPGGPGLGGAPGGRREPKKYAVTDDYQLSKQVLGLGVNGKVLECFHRRTGQKCALKLLYDSPKARQEVDHHWQASGGPHIVCILDVYENMHHGKRCLLIIMECMEGGELFSRIQERGDQAFTEREAAEIMRDIGTAIQFLHSHNIAHRDVKPENLLYTSKEKDAVLKLTDFGFAKETTQNALQTPCYTPYYVAPEVLGPEKYDKSCDMWSLGVIMYILLCGFPPFYSNTGQAISPGMKRRIRLGQYGFPNPEWSEVSEDAKQLIRLLLKTDPTERLTITQFMNHPWINQSMVVPQTPLHTARVLQEDKDHWDEVKEEMTSALATMRVDYDQVKIKDLKTSNNRLLNKRRKKQAGSSSASQGCNNQ

SEQ ID NO: 48 - protein, 356 aa, Homo sapiens
  source 1..356: note = MKK6; Uniprot number: Q9FJV0
MVKIKSNLKQLKLSVPAQESPISSFLTASGTFHDGDFLLNQKGLRLTSDEKQSRQSDSKELDFEITAEDLETVKVIGKGSGGVVQLVRHKWVGKFFAMKVIQMNIQEEIRKQIVQELKINQASSQCPHVVVCYHSFYHNGAFSLVLEYMDRGSLADVIRQVKTILEPYLAVVCKQVLLGLVYLHNERHVIHRDIKPSNLLVNHKGEVKISDFGVSASLASSMGQRDTFVGTYNYMSPERISGSTYDYSSDIWSLGMSVLECAIGRFPYLESEDQQNPPSFYELLAAIVENPPPTAPSDQFSPEFCSFVSACIQKDPPARASSLDLLSHPFIKKFEDKDIDLGILVGTLEPPVNYLR

SEQ ID NO: 49 - RNA, 19 nt, synthetic construct
  source 1..19: mol_type = other RNA; note = siRNA (I) sequence for SESN3; note = uracil
tgtcaaagtttagccgttt

SEQ ID NO: 50 - RNA, 19 nt, synthetic construct
  source 1..19: mol_type = other RNA; note = siRNA (II) sequence for SESN3; note = uracil
tctgatgtctctcgatata

SEQ ID NO: 51 - RNA, 19 nt, synthetic construct
  source 1..19: mol_type = other RNA; note = siRNA (III) sequence for SESN3; note = uracil
gccggaaagtgctgcggaa

SEQ ID NO: 52 - RNA, 19 nt, synthetic construct
  source 1..19: mol_type = other RNA; note = siRNA (IV) sequence for SESN3; note = uracil
ggtcatgagtttacacact

SEQ ID NO: 53 - RNA, 19 nt, synthetic construct
  source 1..19: mol_type = other RNA; note = siRNA (I) sequence for p62; note = uracil
gaaatgggtccaccaggaa

SEQ ID NO: 54 - RNA, 19 nt, synthetic construct
  source 1..19: mol_type = other RNA; note = siRNA (II) sequence for p62; note = uracil
gatctgcgatggctgcaat

SEQ ID NO: 55 - RNA, 19 nt, synthetic construct
  source 1..19: mol_type = other RNA; note = siRNA (III) sequence for p62; note = uracil
gcattgaagttgatatcga

SEQ ID NO: 56 - RNA, 19 nt, synthetic construct
  source 1..19: mol_type = other RNA; note = siRNA (IV) sequence for p62; note = uracil
gaagtggacccgtctacag

SEQ ID NO: 57 - RNA, 19 nt, synthetic construct
  source 1..19: mol_type = other RNA; note = siRNA (I) sequence for NBR1; note = uracil
gagaacaagtggttaacga

SEQ ID NO: 58 - RNA, 19 nt, synthetic construct
  source 1..19: mol_type = other RNA; note = siRNA (II) sequence for NBR1; note = uracil
ccacatgacagtcctttaa

SEQ ID NO: 59 - RNA, 19 nt, synthetic construct
  source 1..19: mol_type = other RNA; note = siRNA (I) sequence for NBR1; note = uracil
gaacgtatacttcccattg

SEQ ID NO: 60 - RNA, 19 nt, synthetic construct
  source 1..19: mol_type = other RNA; note = siRNA (I) sequence for NBR1; note = uracil
agaagccacttgcacatta